(19) United States
(12) Patent Application Publication
(10) Pub. No.: US 2025/0207138 A1
(43) Pub. Date: Jun. 26, 2025

(54) RNAI CONSTRUCTS FOR INHIBITING PNPLA3 EXPRESSION AND METHODS OF USE THEREOF

(71) Applicant: AMGEN INC., Thousand Oaks, CA (US)

(72) Inventors: Bryan MEADE, Westlake Village, CA (US); Justin K. MURRAY, Moorpark, CA (US); Ingrid RULIFSON, Palo Alto, CA (US); Oliver HOMANN, Berkeley, CA (US)

(73) Assignee: AMGEN INC., Thousand Oaks, CA (US)

(21) Appl. No.: 18/848,796

(22) PCT Filed: Mar. 21, 2023
(86) PCT No.: PCT/US2023/064765

§ 371 Date: Sep. 19, 2024

Related U.S. Application Data
(60) Provisional application No. 63/322,845, filed on Mar. 23, 2022.

(51) Int. Cl. C12N 15/113

(52) U.S. Cl. CPC C12N 15/1137; C12Y 301/01004; C12N 2310/14; C12N 2310/315; C12N 2310/321; C12N 2310/322

(57) ABSTRACT

The disclosure relates to RNAi constructs, such as siRNA, for reducing expression of the PNPLA3 gene. Methods of using such RNAi constructs to treat or prevent liver disease, such as nonalcoholic fatty liver disease (NAFLD), are also described.

### CROSS-REFERENCE TO RELATED APPLICATIONS

This application claims the benefit of U.S. Provisional Application No. 63/322,845, filed on Mar. 23, 2022.

### FIELD OF THE INVENTION

The present disclosure relates to compositions and methods for modulating liver expression of patatin-like phospholipase domain-containing 3 (PNPLA3). In particular, the present disclosure relates to nucleic acid-based therapeutics for reducing PNPLA3 expression via RNA interference and methods of using such nucleic acid-based therapeutics to treat or prevent liver disease, such as nonalcoholic fatty liver disease (NAFLD).

### INCORPORATION-BY-REFERENCE OF MATERIAL SUBMITTED ELECTRONICALLY

Incorporated by reference in its entirety herein is a nucleotide/amino acid sequence listing submitted concurrently herewith and identified as follows: One 1,930 kilobyte XML document named “A-2910-WO01-SEC.xml,” created on Mar. 20, 2023.

### BACKGROUND

Comprising a spectrum of hepatic pathologies, nonalcoholic fatty liver disease (NAFLD) is the most common chronic liver disease in the world, the prevalence of which doubled in the last 20 years and now is estimated to affect approximately 20% of the world's population (Sattar et al. (2014) BMJ 349: g4596; Loomba and Sanyal (2013) Nature Reviews Gastroenterology & hepatology 10(11):686-690; Kim and Kim (2017) Clin Gastroenterol Hepatol 15(4):474-485; Petta et al. (2016) Dig Liver Dis 48(3):333-342). NAFLD begins with the accumulation of triglyceride in the liver and is defined by the presence of cytoplasmic lipid droplets in more than 5% of hepatocytes in an individual 1) without a history of significant alcohol consumption and 2) in which the diagnosis of other types of liver disease have been excluded (Zhu et al (2016) World J Gastroenterol 22(36):8226-33; Rinella (2015) JAMA 313(22):2263-73; Yki-Jarvinen (2016) Diabetologia 59(6):1104-11). In some individuals the accumulation of ectopic fat in the liver, called steatosis, triggers inflammation and hepatocellular injury leading to a more advanced stage of disease called nonalcoholic steatohepatitis (NASH) (Rinella, supra). As of 2015, 75-100 million Americans are predicted to have NAFLD, with NASH accounting for approximately 10-30% of NAFLD diagnoses (Rinella, supra; Younossi et al (2016) Hepatology 64(5):1577-1586).

Patatin-like phospholipase domain-containing 3 (PNPLA3), formerly known as adiponutrin (ADPN) and calcium-independent phospholipase A2-epsilon (iPLA(2)ε), is a type II transmembrane protein (Wilson et al (2006) J Lipid Res 47(9):1940-9; Jenkins et al (2004) J Biol Chem 279(47):48968-75). Initially identified in adipose cells as a membrane-associated, adipose-enriched protein induced during adipogenesis in mice, it is now well characterized to be expressed in other tissues, including the liver (Wilson et al, supra; Baulande et al. (2001) J Biol Chem 276(36):33336-44; Moldes et al. (2006) Eur J Endocrinol 155(3):461-8; Faraj et al. (2006) J Endocrinol 191(2):427-35; Liu et al. (2004) J Clin Endocrinol Metab 89(6):2684-9; Lake et al. (2005) J Lipid Res 46(11):2477-87). In cell-free biochemical systems, recombinant PNPLA3 protein can exhibit either triacylglycerol lipase or transacylation activity (Jenkins et al., supra; Kumari et al. (2012) Cell Metab 15(5):691-702; He et al. (2010) J Biol Chem 285(9):6706-15). In hepatocytes, PNPLA3 is expressed on the endoplasmic reticulum and lipid membranes and predominantly exhibits triacylglycerol hydrolase activity (He et al., supra; Huang et al. (2010) Proc Natl Acad Sci USA 107(17):7892-7; Ruhanen et al. (2014) J Lipid Res 55(4):739-46; Pingitore et al. (2014) Biochim Biophys Acta 1841(4):574-80). Although PNPLA3 lacks a secretory signal, data indicates that it is secreted and can be found in human plasma as disulfide-bond dependent multimers (Winberg et al. (2014) Biochem Biophys Res Commun 446(4):1114-9).

Currently, NAFLD symptoms are managed via weight loss and treatment of any secondary conditions, as no pharmacologic treatments have been approved. Thus, there is a need for compositions and methods that treat NAFLD in affected individuals.

### SUMMARY OF THE INVENTION

The present disclosure provides an RNAi construct comprising a sense strand and an antisense strand, wherein the antisense strand comprises a region having a sequence that is complementary to a PNPLA3 mRNA sequence, such as a PNPLA3 mRNA sequence set forth in Table 1, and wherein the RNAi construct inhibits the expression of PNPLA3. In certain embodiments, the RNAi construct comprises a region having at least 15 contiguous nucleotides differing by no more than 3 nucleotides from an antisense sequence selected from SEQ ID NOs: 565-1068 and SEQ ID NOs: 2329-3588. In some embodiments, the antisense strand hybridizes to a PNPLA3 mRNA sequence listed in Table 1.

In some embodiments, the sense strand of the RNAi constructs described herein comprises a sequence that is sufficiently complementary to the sequence of the antisense strand to form a duplex region of about 15 to about 30 base pairs in length. In these and other embodiments, the sense and antisense strands each are about 15 to about 30 nucleotides in length. In some embodiments, the RNAi constructs comprise at least one blunt end. In other embodiments, the RNAi constructs comprise at least one nucleotide overhang. Such nucleotide overhangs may comprise at least 1 to 6 unpaired nucleotides and can be located at the 3′ end of the sense strand, the 3′ end of the antisense strand, or the 3′ end of both the sense and antisense strand. In certain embodiments, the RNAi constructs comprise an overhang of two unpaired nucleotides at the 3′ end of the sense strand and the 3′ end of the antisense strand. In other embodiments, the RNAi constructs comprise an overhang of two unpaired nucleotides at the 3′ end of the antisense strand and a blunt end of the 3′ end of the sense strand/5′ end of the antisense strand.

The RNAi constructs of the disclosure may comprise one or more modified nucleotides, including nucleotides having modifications to the ribose ring, nucleobase, or phosphodiester backbone. In some embodiments, the RNAi constructs comprise one or more 2′-modified nucleotides. Such 2′-modified nucleotides can include 2′-fluoro modified nucleotides, 2′-O-methyl modified nucleotides, 2′-O-methoxyethyl modified nucleotides, 2′-O-allyl modified nucleotides, bicyclic nucleic acids (BNA), glycol nucleic acids (GNAs), inverted bases (e.g. inverted adenosine) or combinations thereof. In one particular embodiment, the RNAi constructs comprise one or more 2′-fluoro modified nucleotides, 2′-O-methyl modified nucleotides, or combinations thereof. In some embodiments, all of the nucleotides in the sense and antisense strand of the RNAi construct are modified nucleotides.

In some embodiments, the RNAi constructs comprise at least one backbone modification, such as a modified internucleotide or internucleoside linkage. In certain embodiments, the RNAi constructs described herein comprise at least one phosphorothioate internucleotide linkage. In particular embodiments, the phosphorothioate internucleotide linkages may be positioned at the 3′ or 5′ ends of the sense and/or antisense strands.

In some embodiments, the antisense strand and/or the sense strand of the RNAi constructs of the disclosure may comprise or consist of a sequence from the antisense and sense sequences listed in FIG. 1 or 2. In certain embodiments, the RNAi construct may comprise a duplex compound comprising an antisense strand and a sense strand, wherein the antisense strand comprises a sequence selected from SEQ ID NOs: 565-1068 and SEQ ID NOs: 2329-3588 and the sense strand comprises a sequence selected from SEQ ID NOs: 61-564 and SEQ ID NOs: 1069-2328.

The disclosure also provides a composition comprising the aforementioned RNAi construct and a pharmaceutically acceptable carrier, excipient, or diluent, as well as methods of reducing the expression of PNPLA3 in a patient in need thereof comprising administering to the patient the aforementioned RNAi construct or composition.

### BRIEF DESCRIPTION OF THE DRAWINGS

FIG. 1 is a table listing unmodified and modified siRNA sequences directed to PNPLA3 tested in the in vitro experiments described in Example 2. All sequences are shown in 5′-3′ orientation.

FIG. 2 is a table listing unmodified and modified siRNA sequences directed to PNPLA3 tested in the in vivo studies described in Example 3. All sequences are shown in 5′-3′ orientation.

FIG. 3 is a table showing results of siRNA inhibition of PNPLA3 in an AAV-based mouse model containing the full human PNPLA3 coding sequence (CDS).

FIG. 4 is a table showing results of siRNA inhibition of PNPLA3 in an AAV-based mouse model containing portions of the 3′ untranslated region (UTR) of human PNPLA3.

FIG. 5 is a table showing results of siRNA inhibition of PNPLA3 in a homozygous hPNPLA3^{I148M} knock-in (hPNPLA3^{I148M} KI) mouse model.

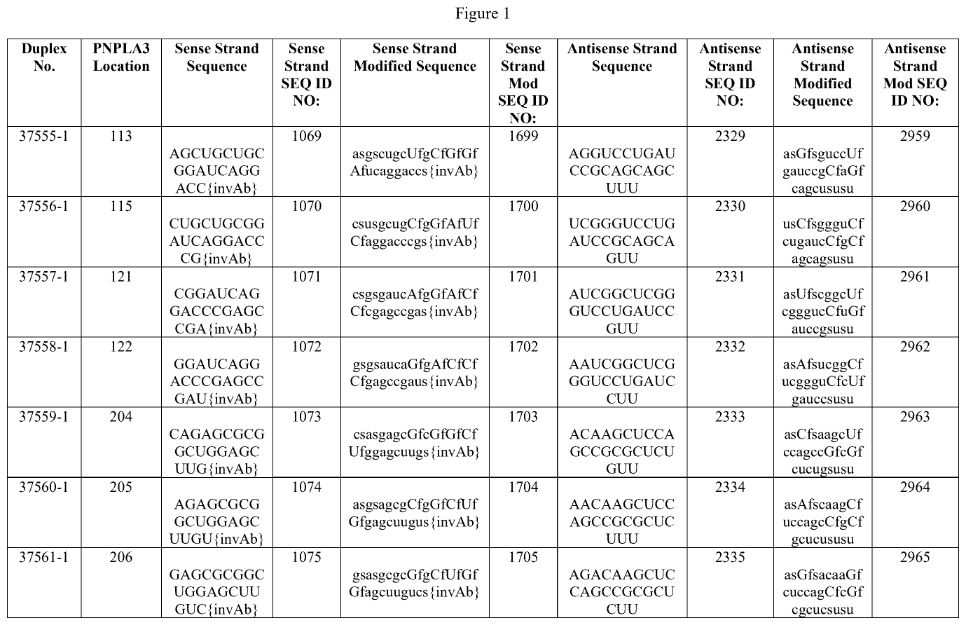

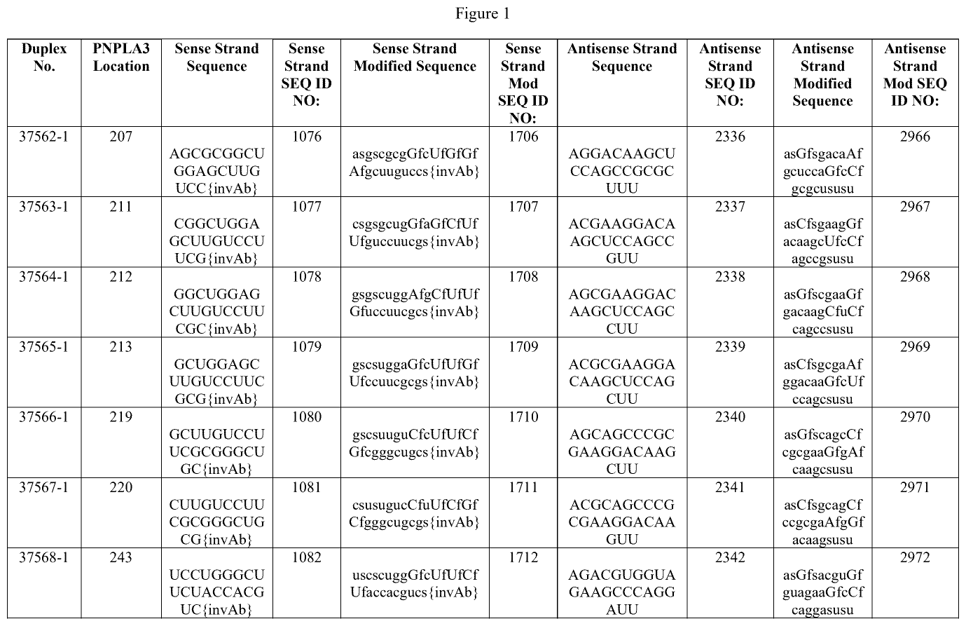

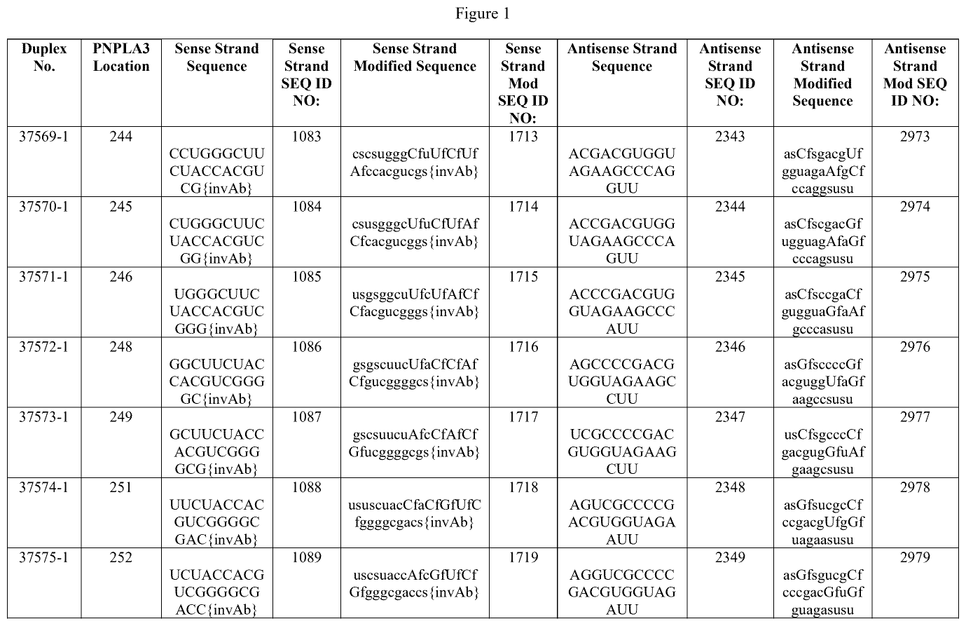

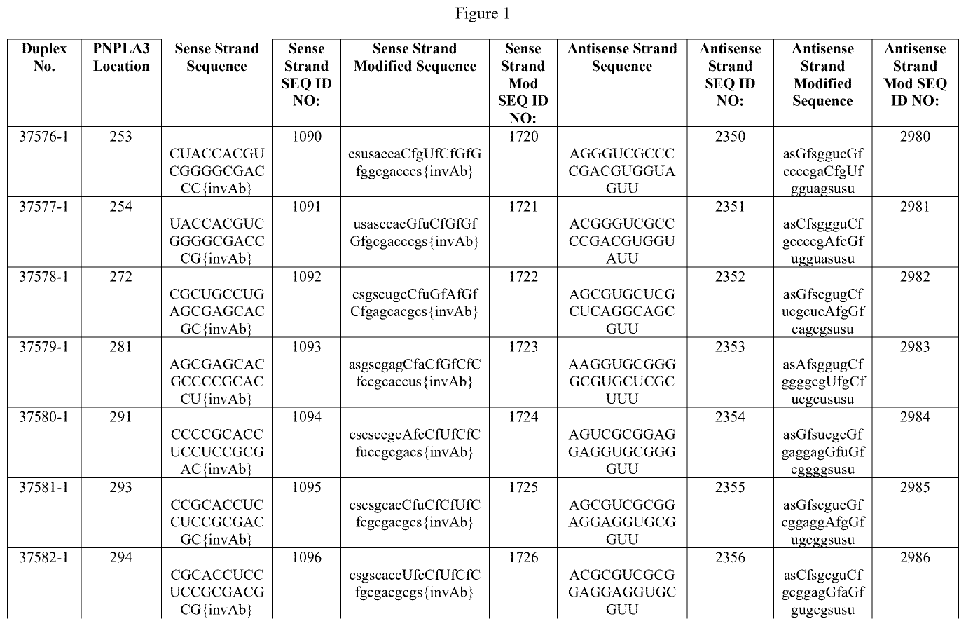

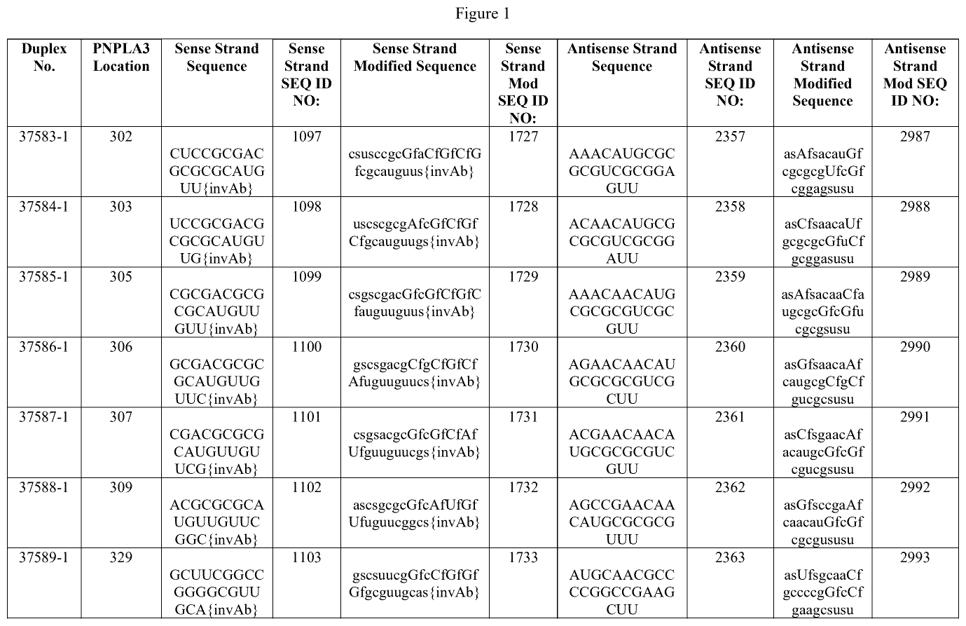

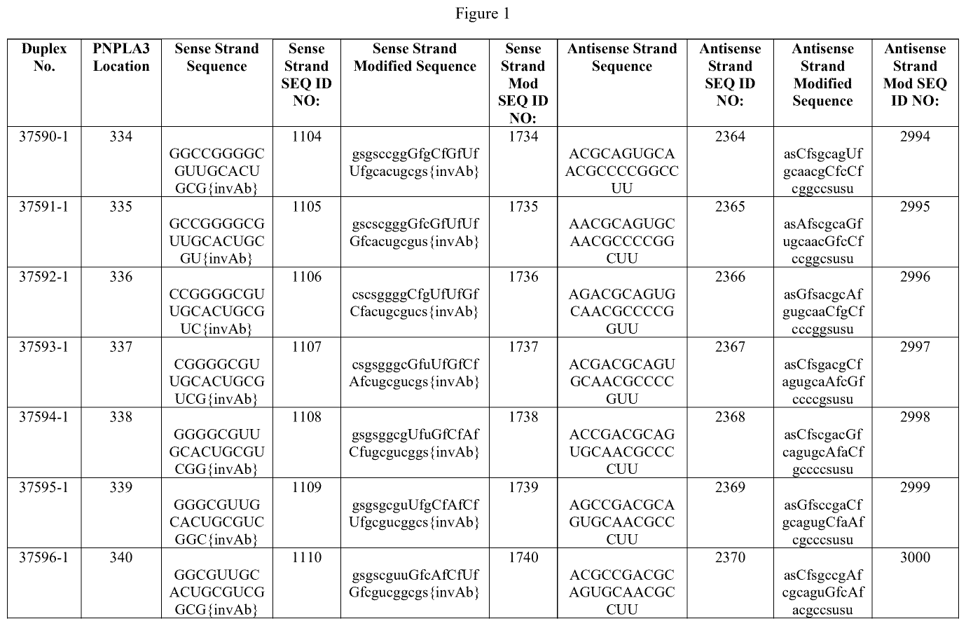

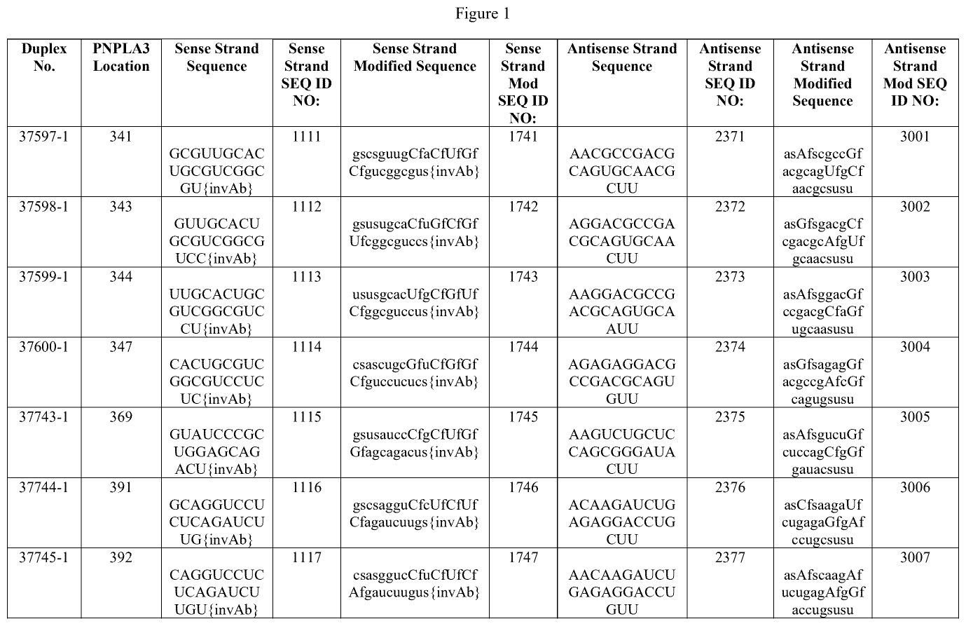

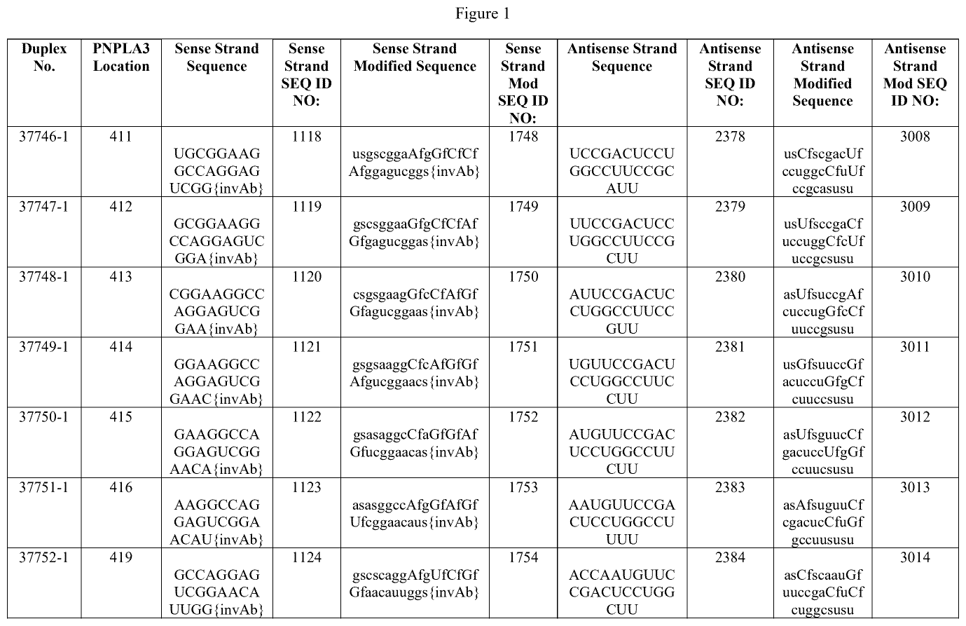

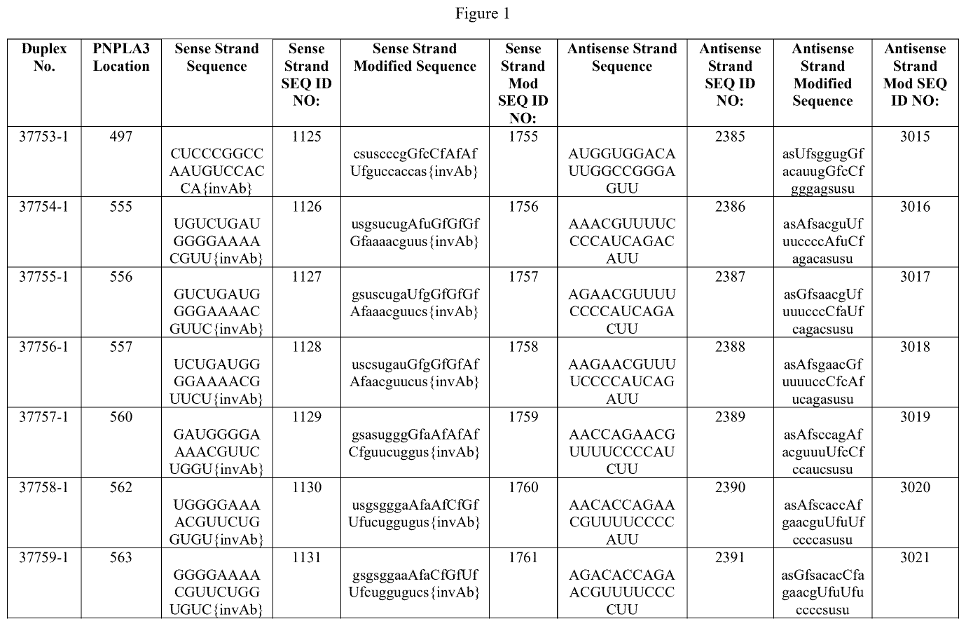

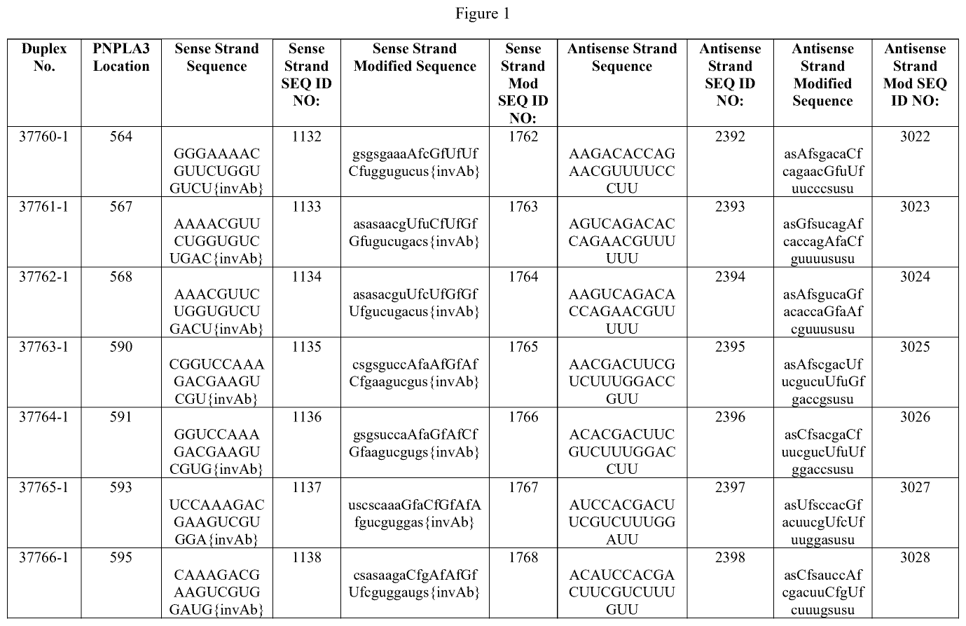

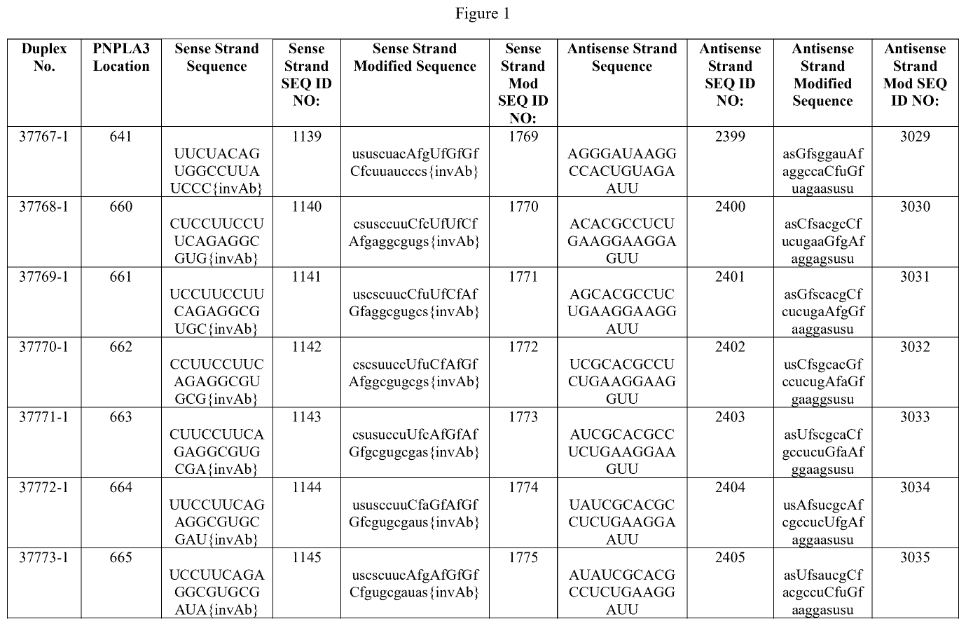

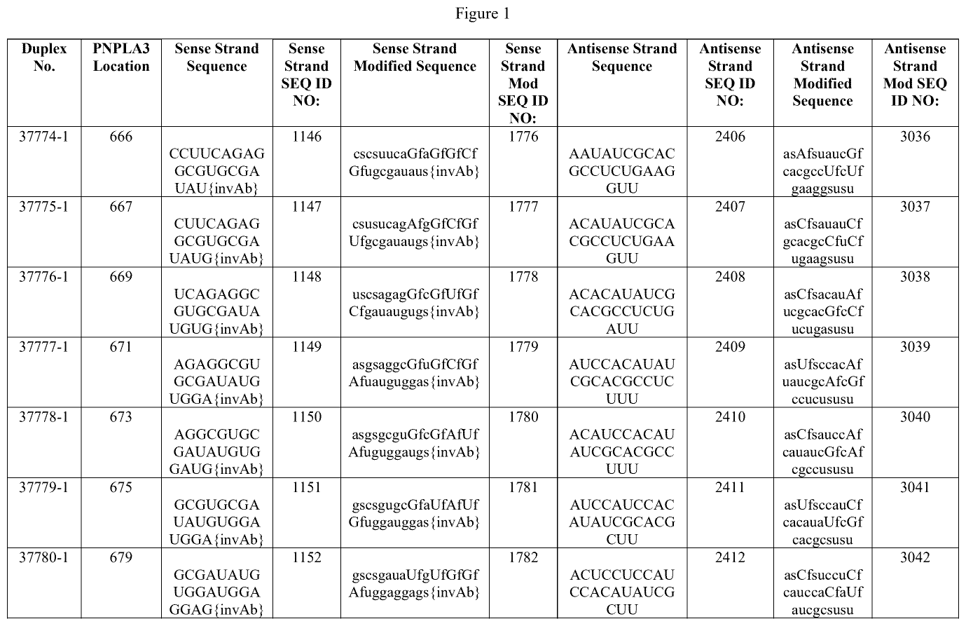

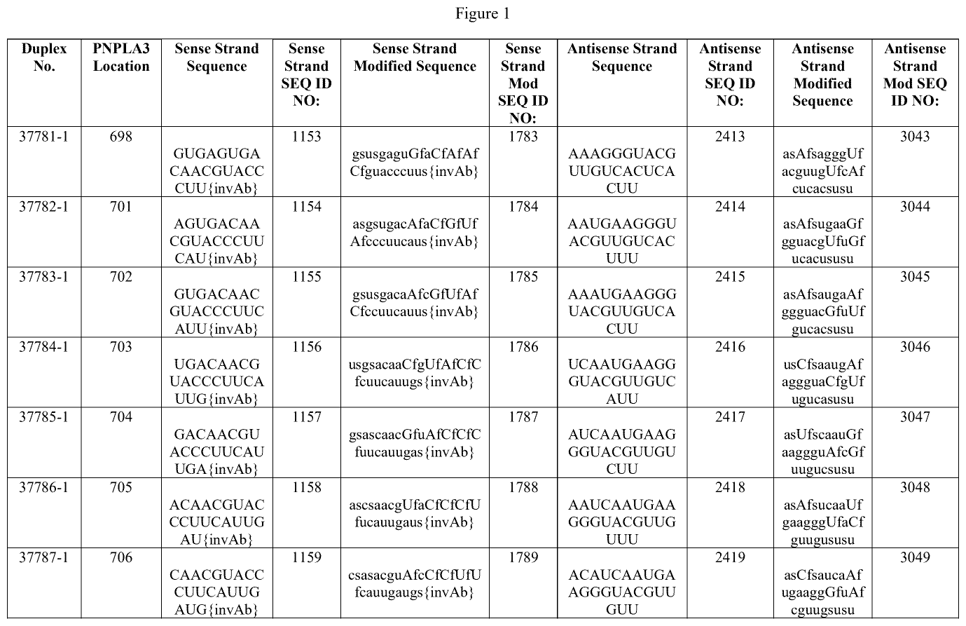

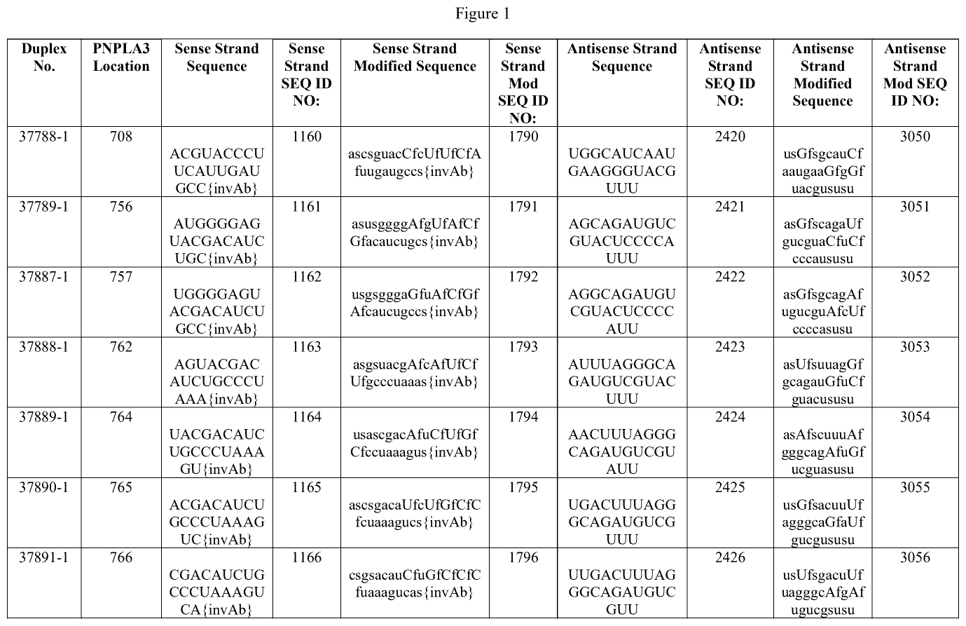

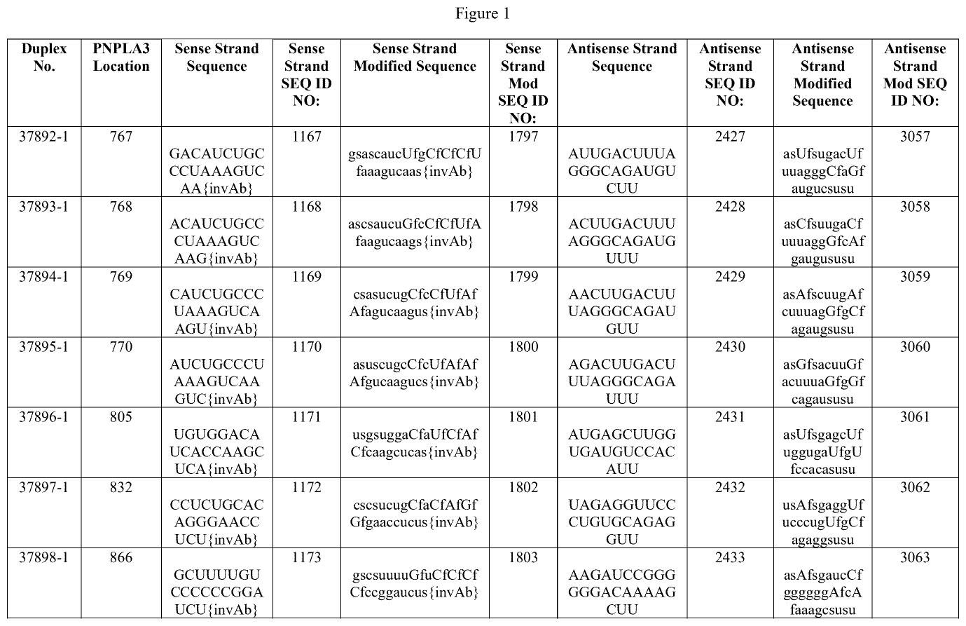

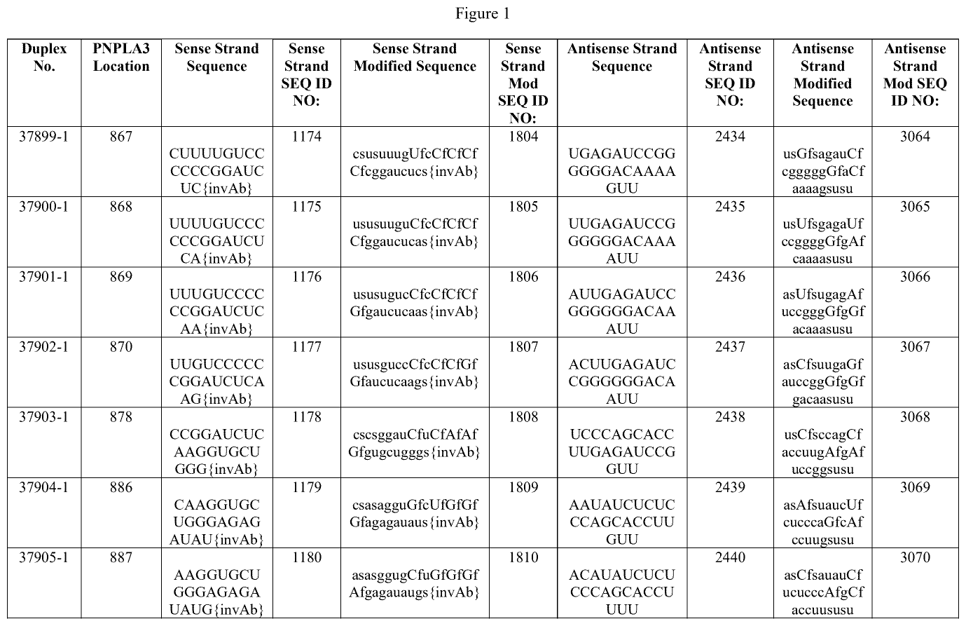

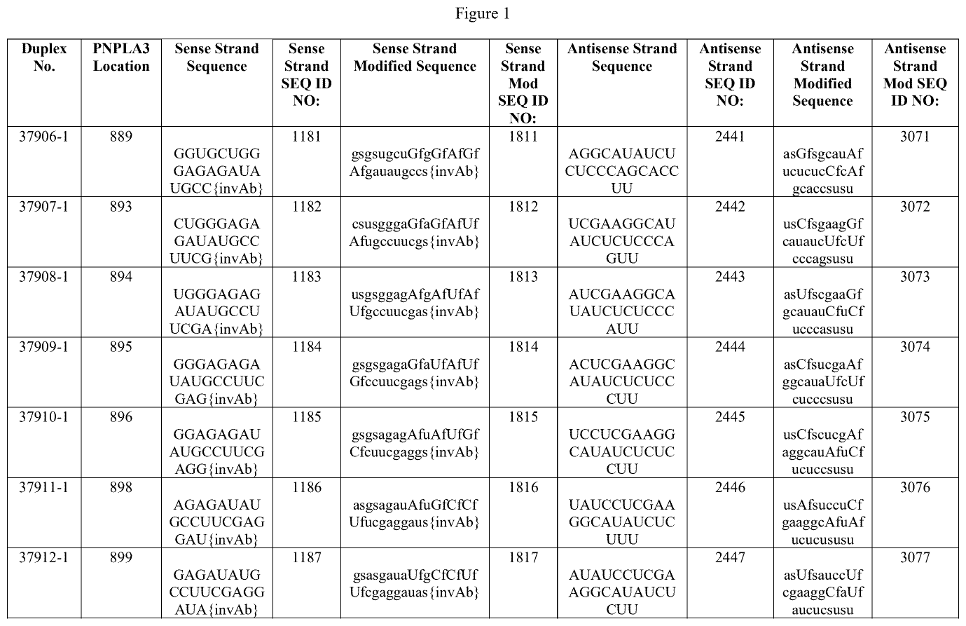

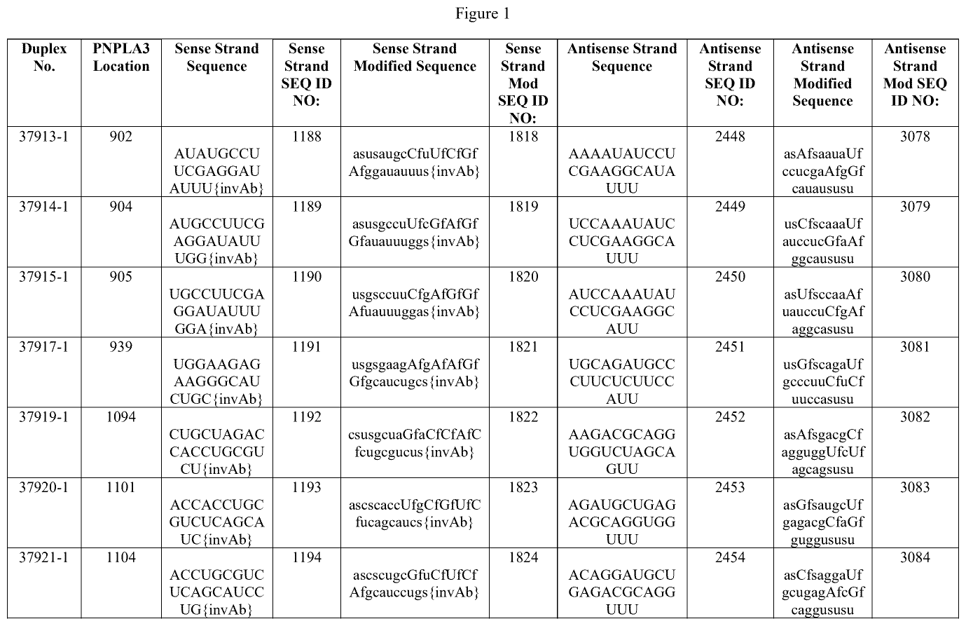

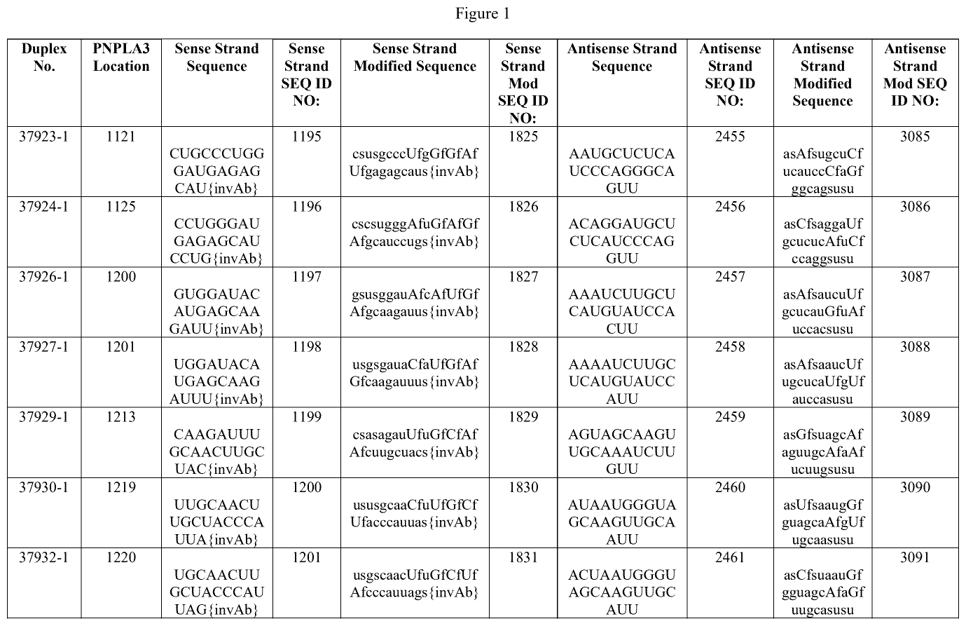

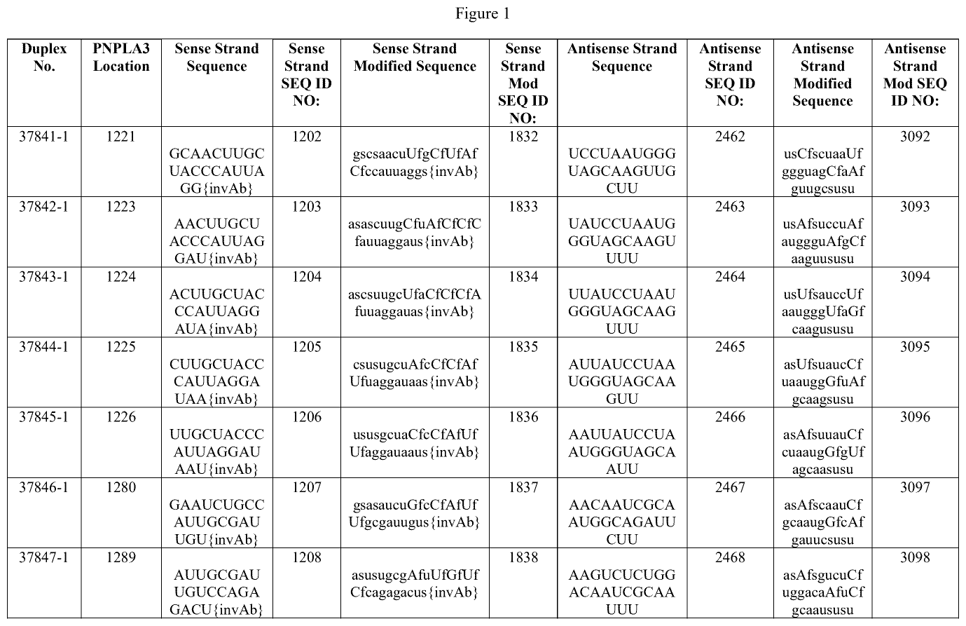

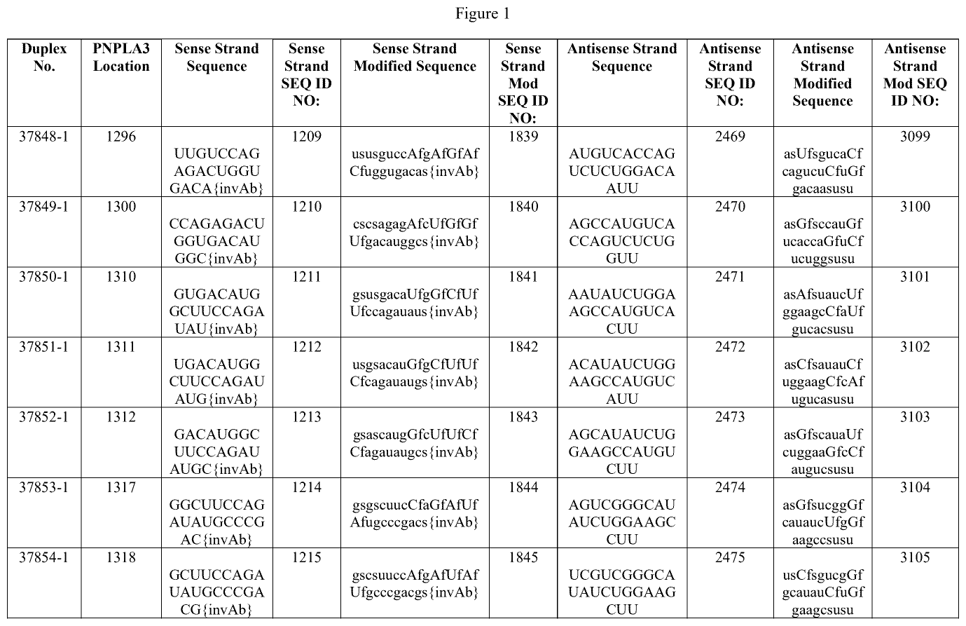

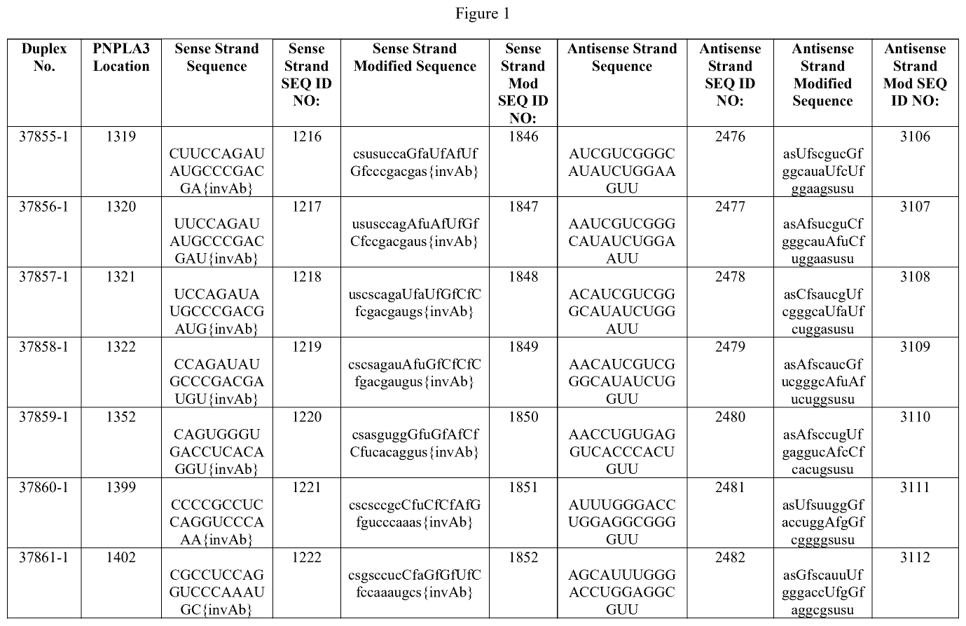

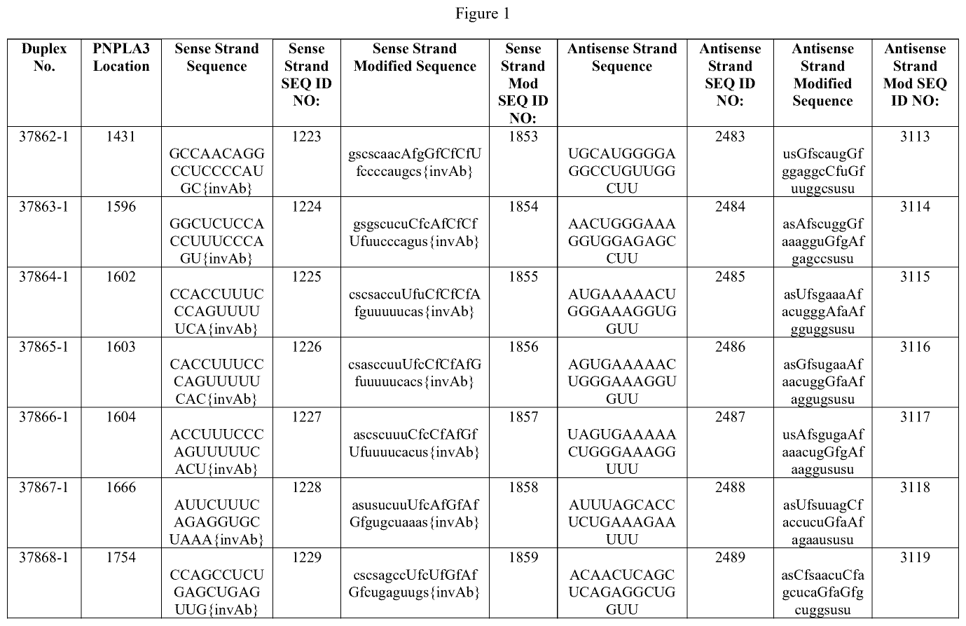

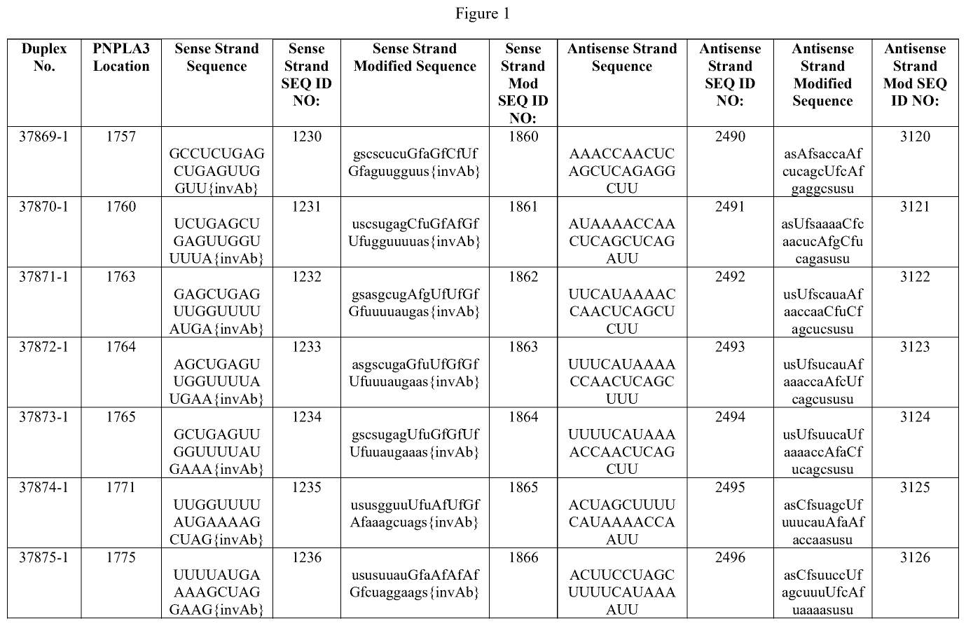

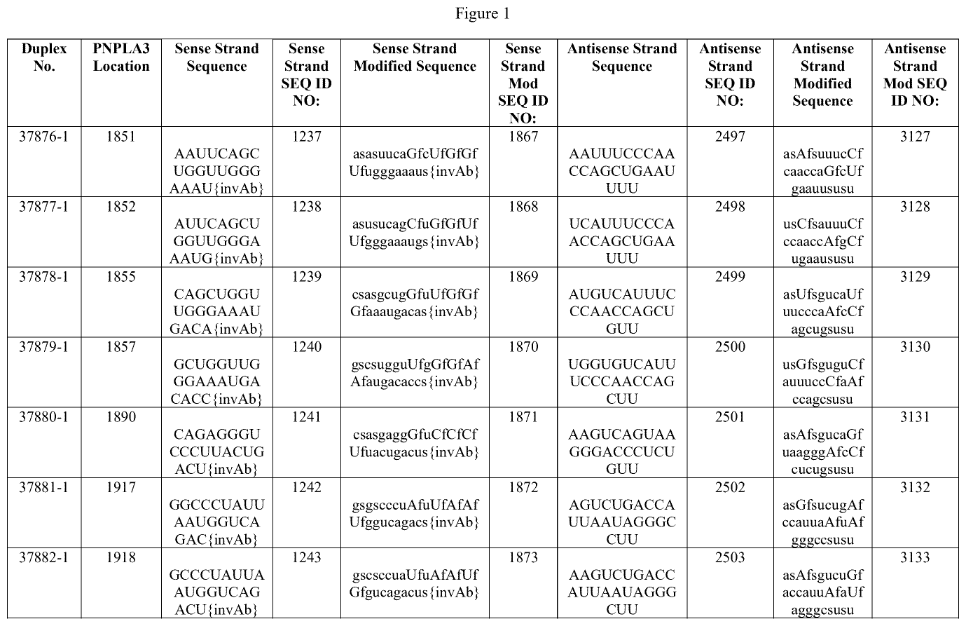

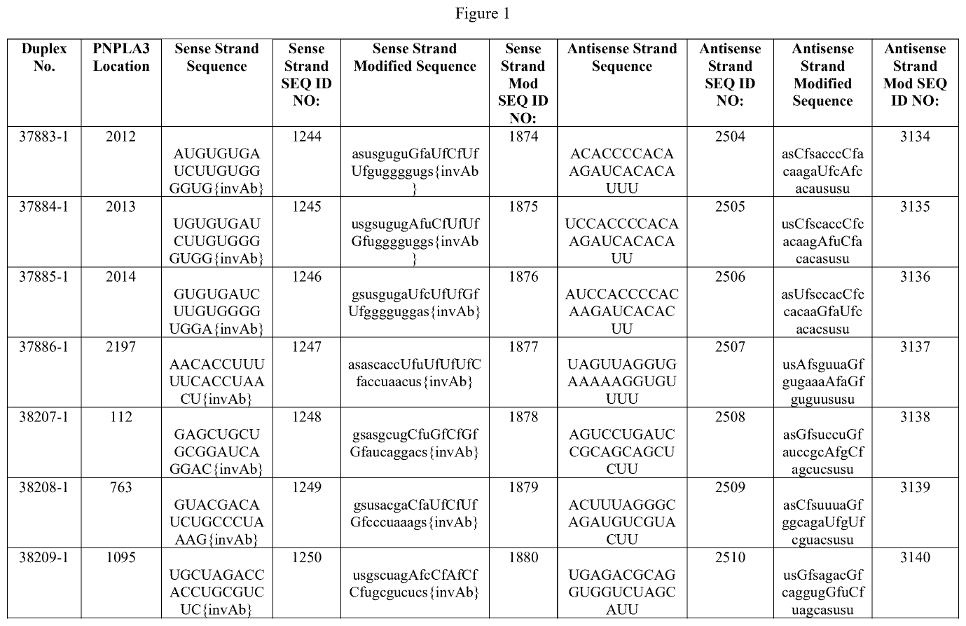

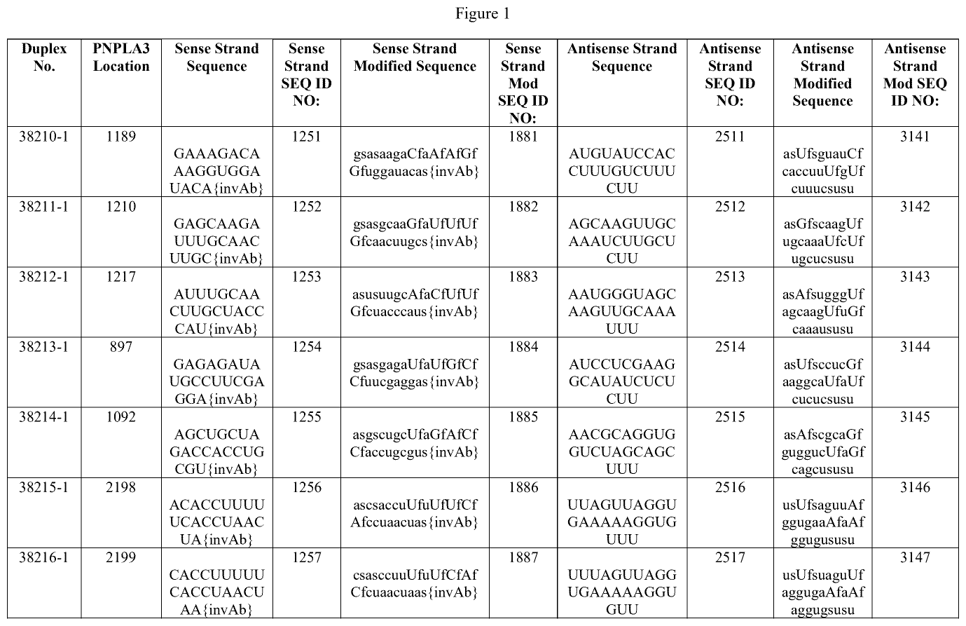

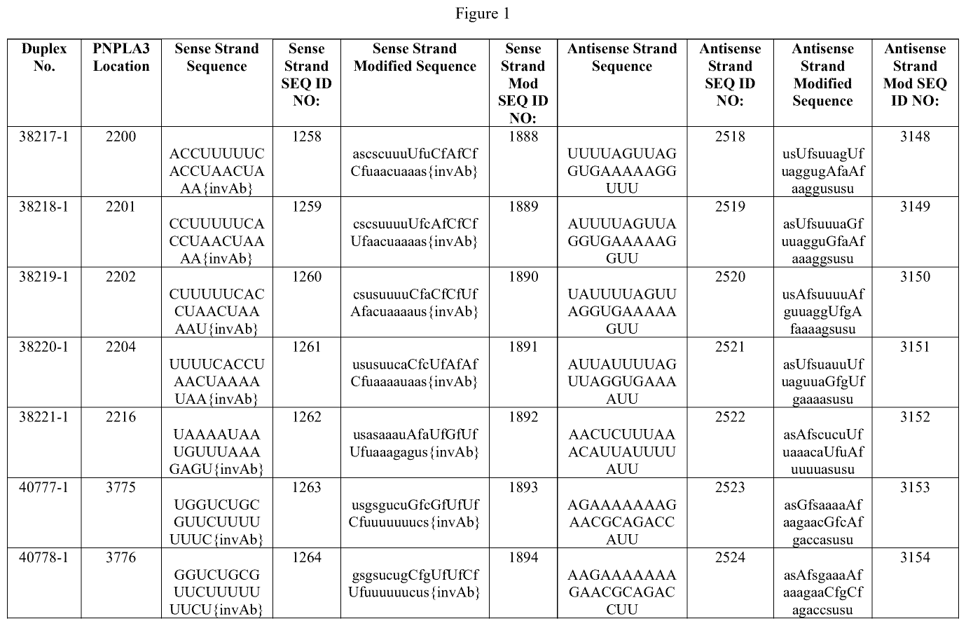

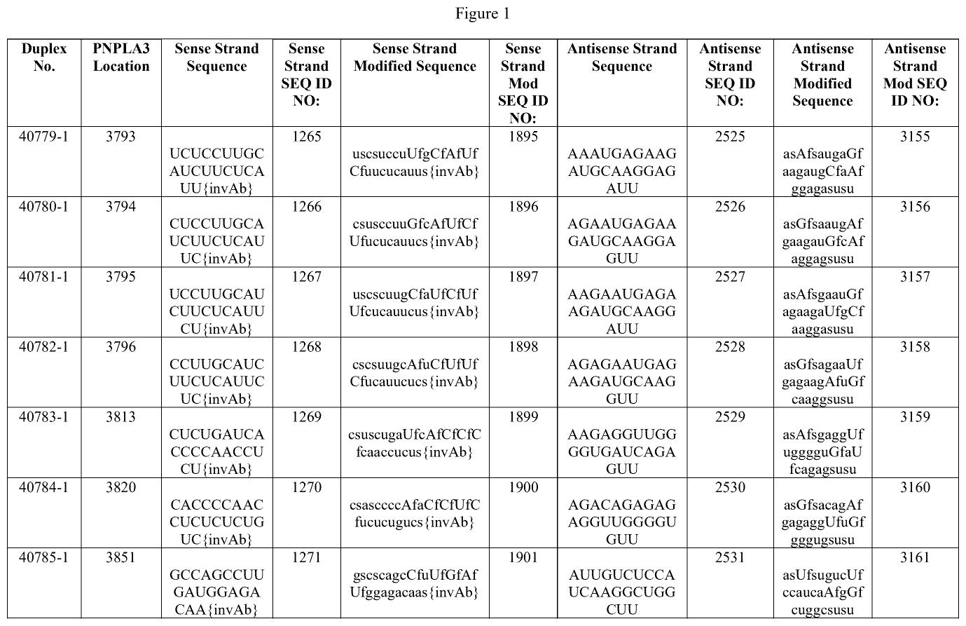

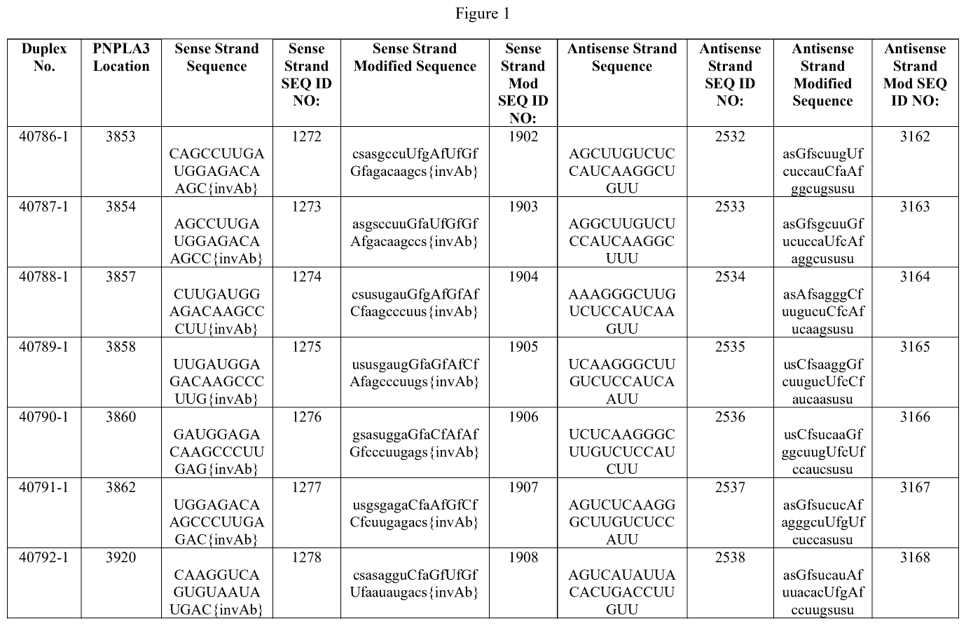

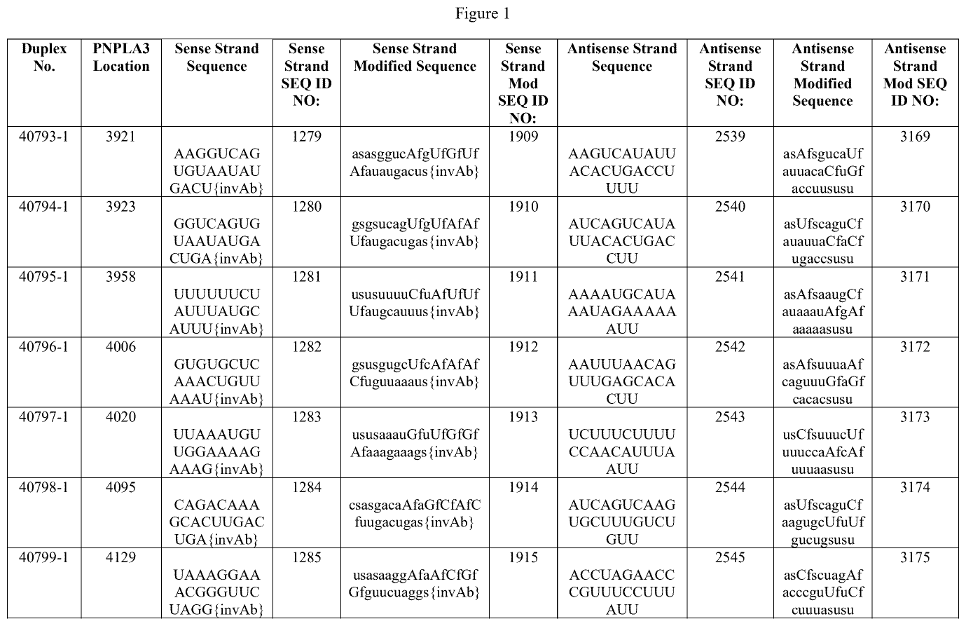

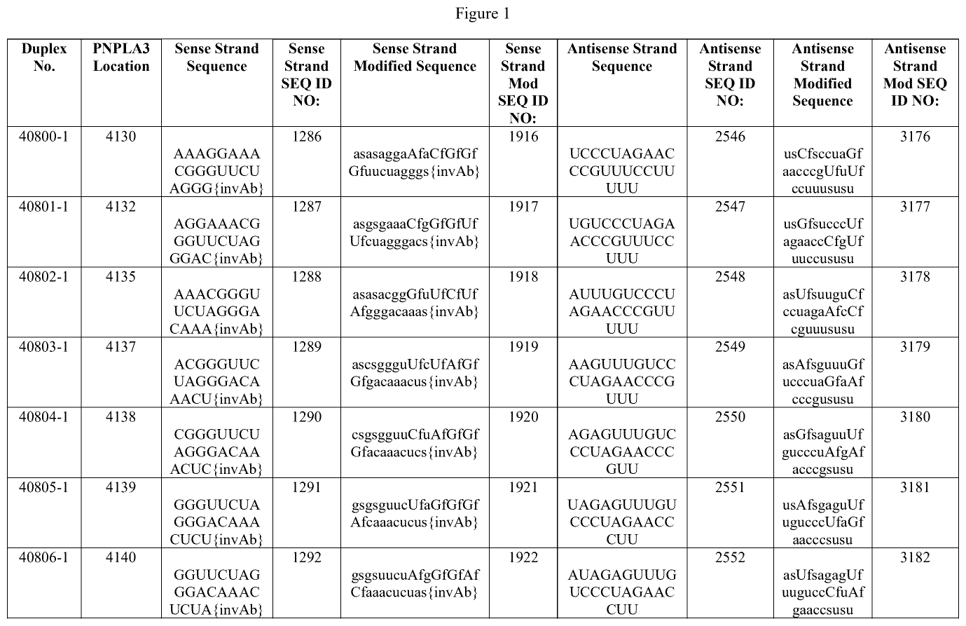

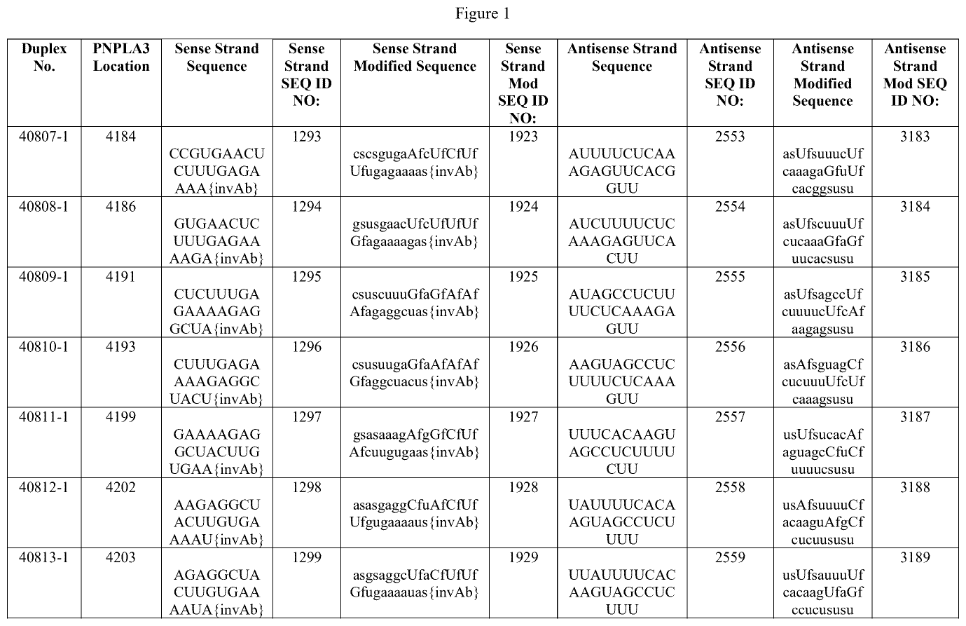

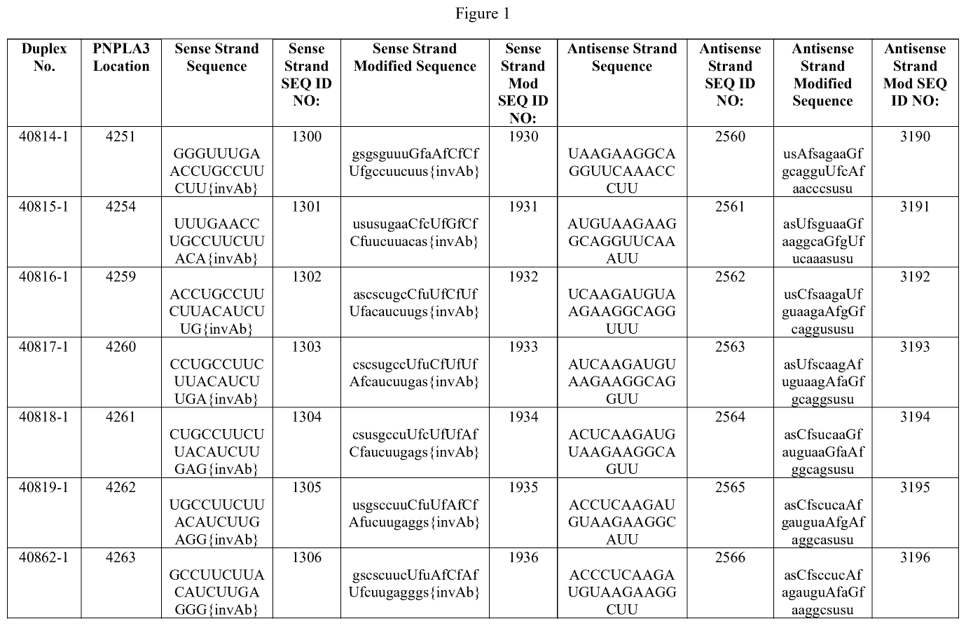

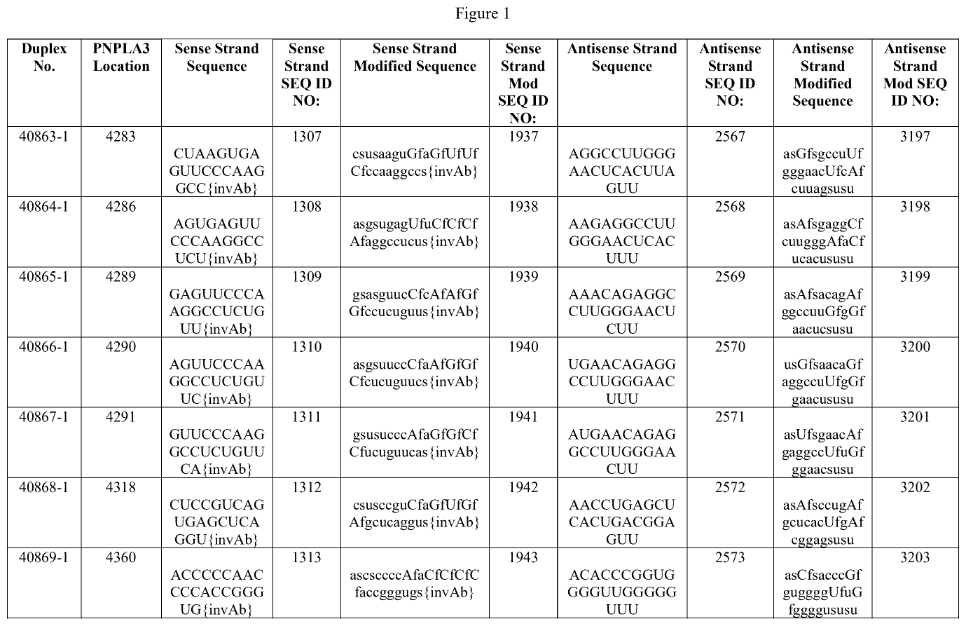

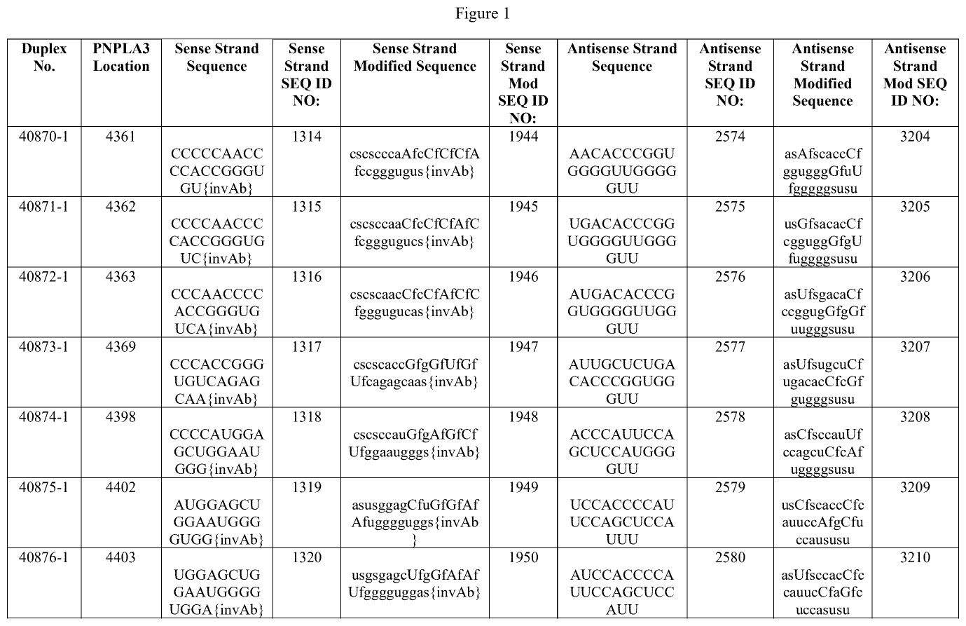

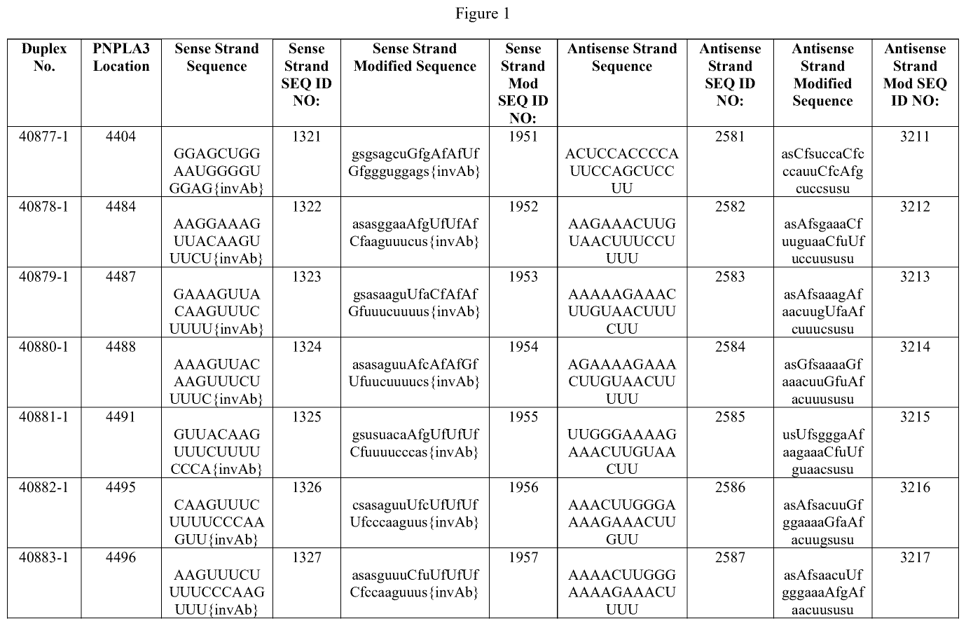

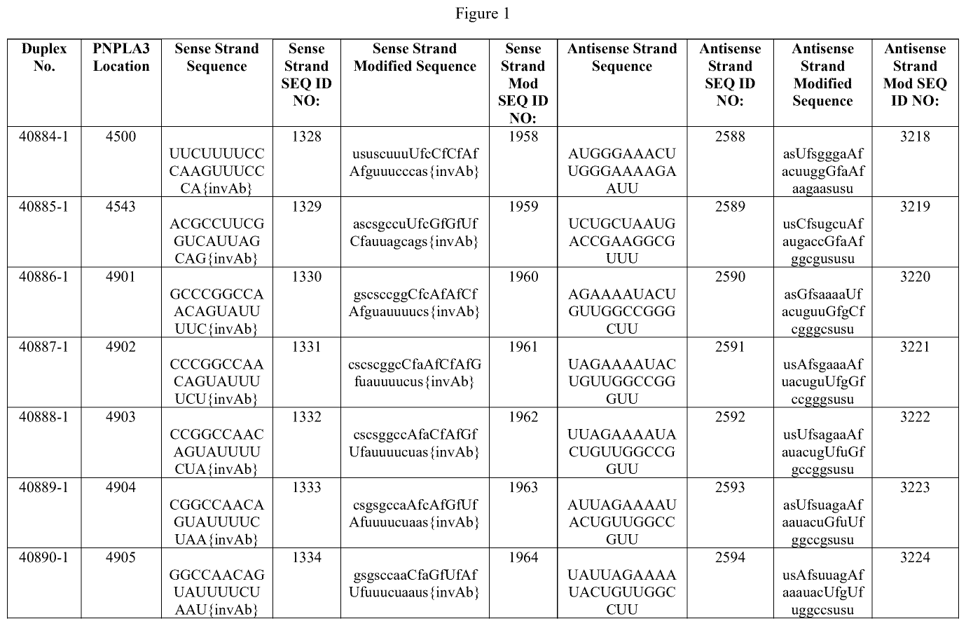

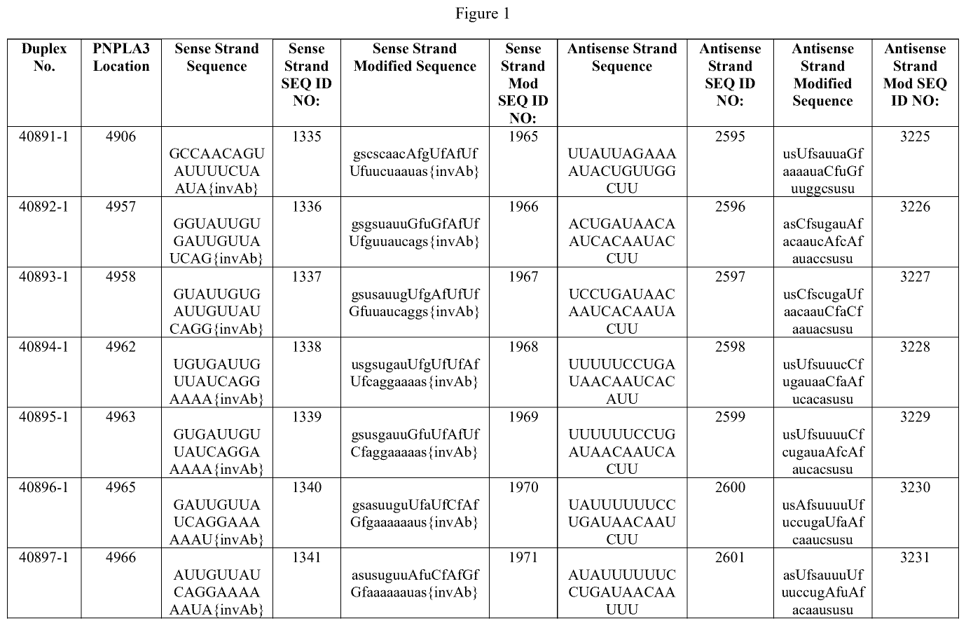

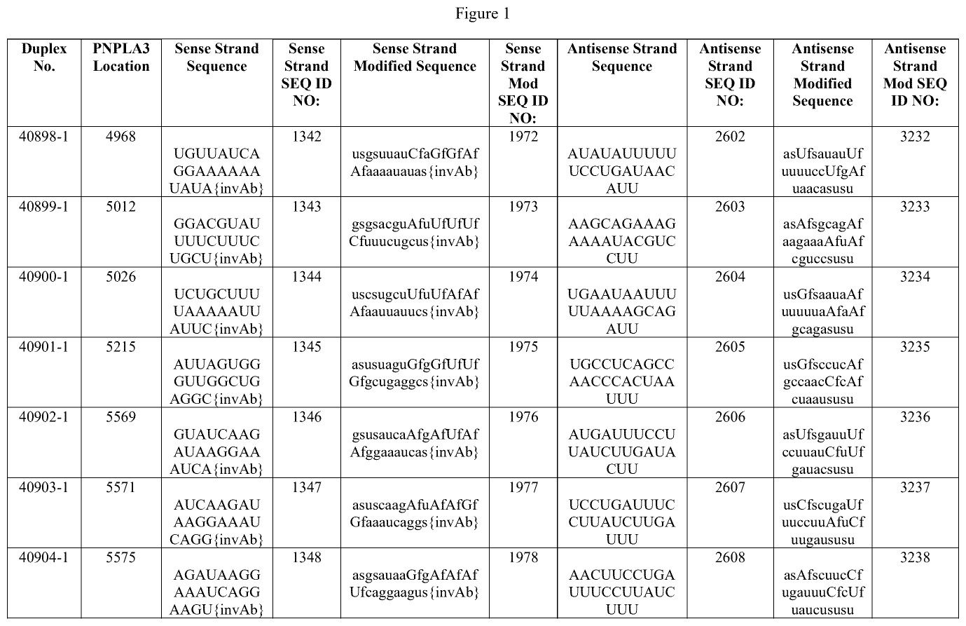

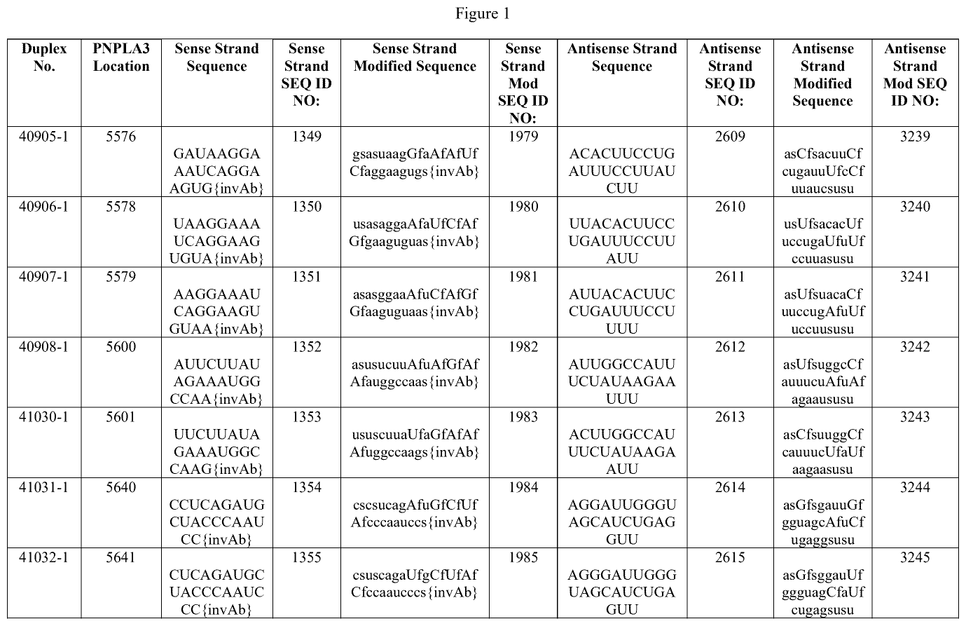

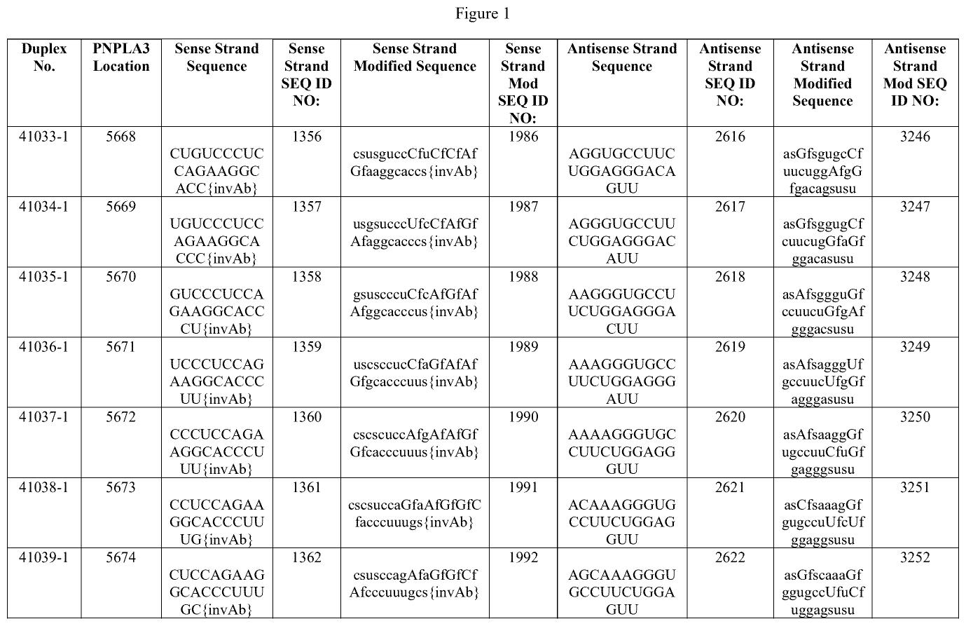

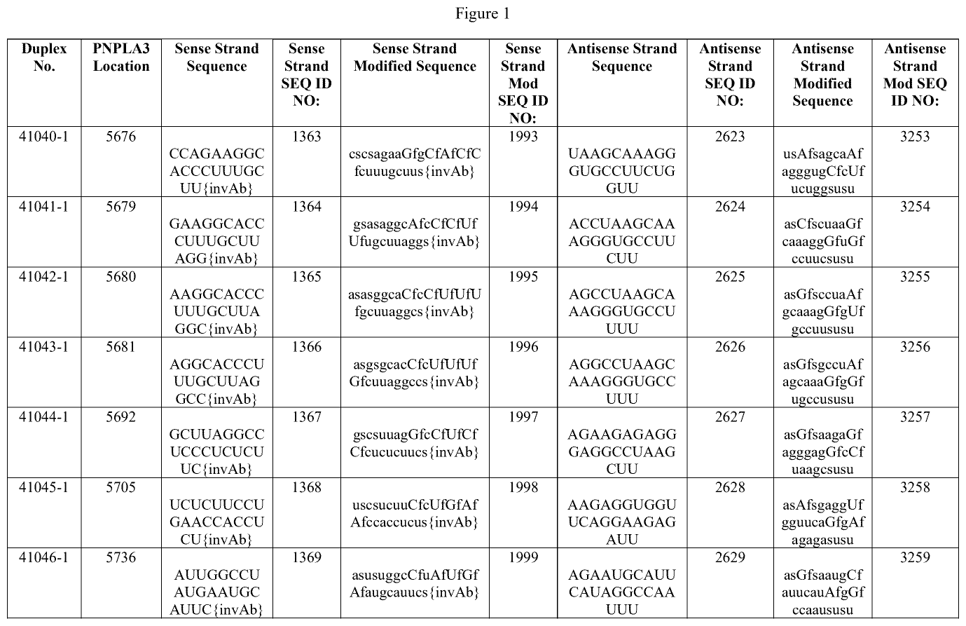

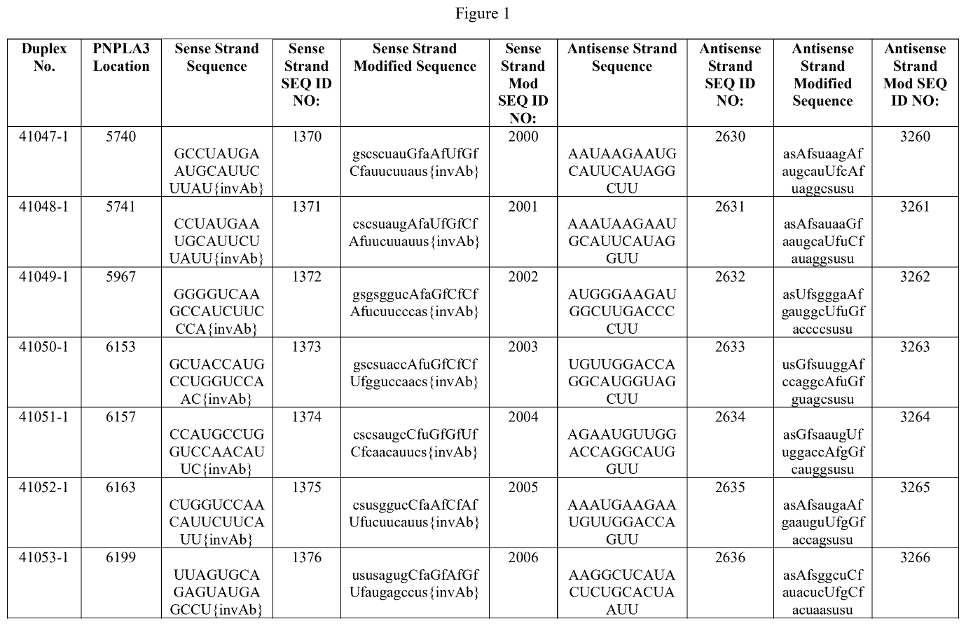

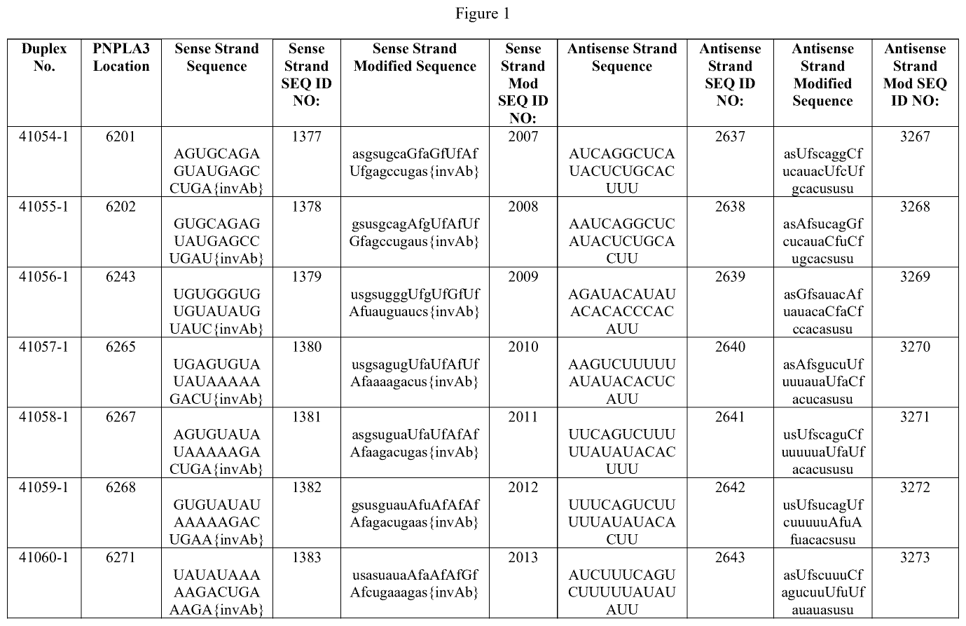

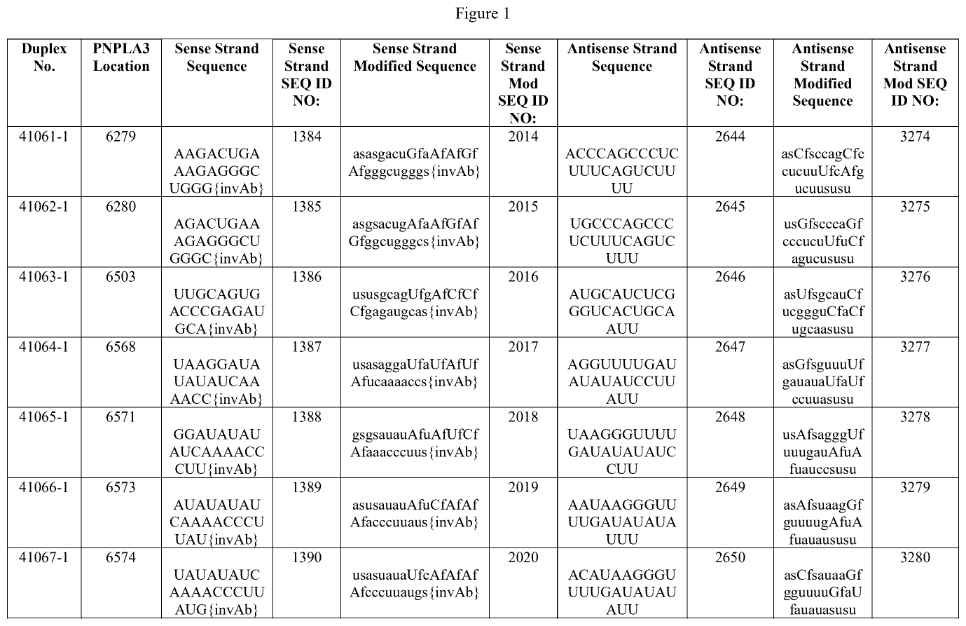

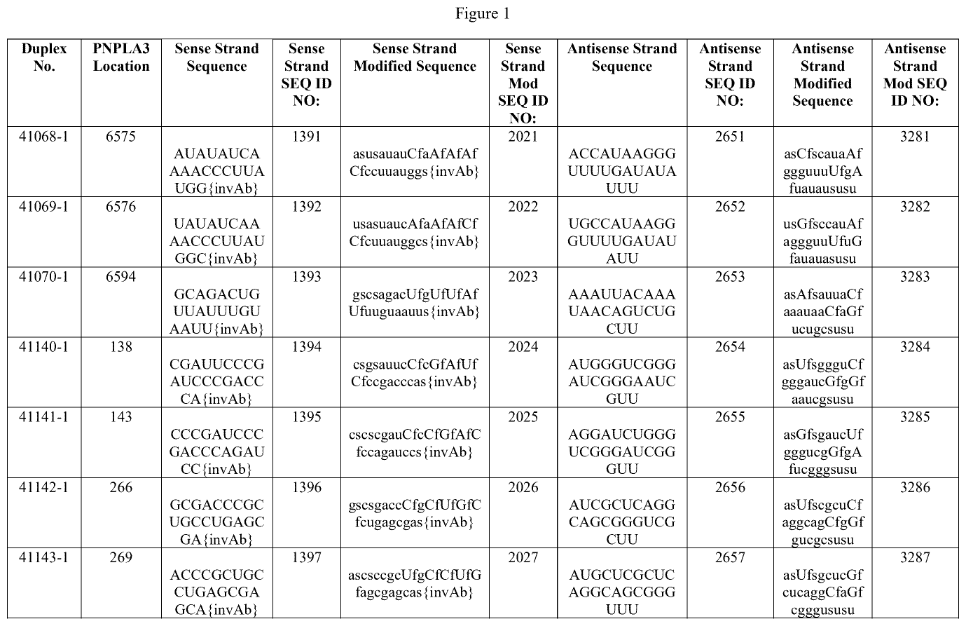

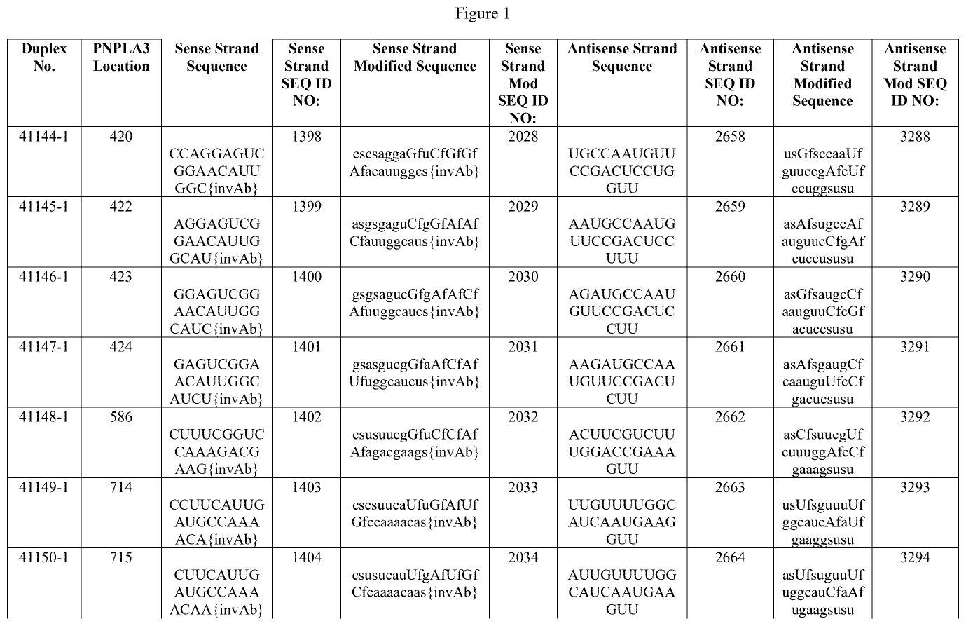

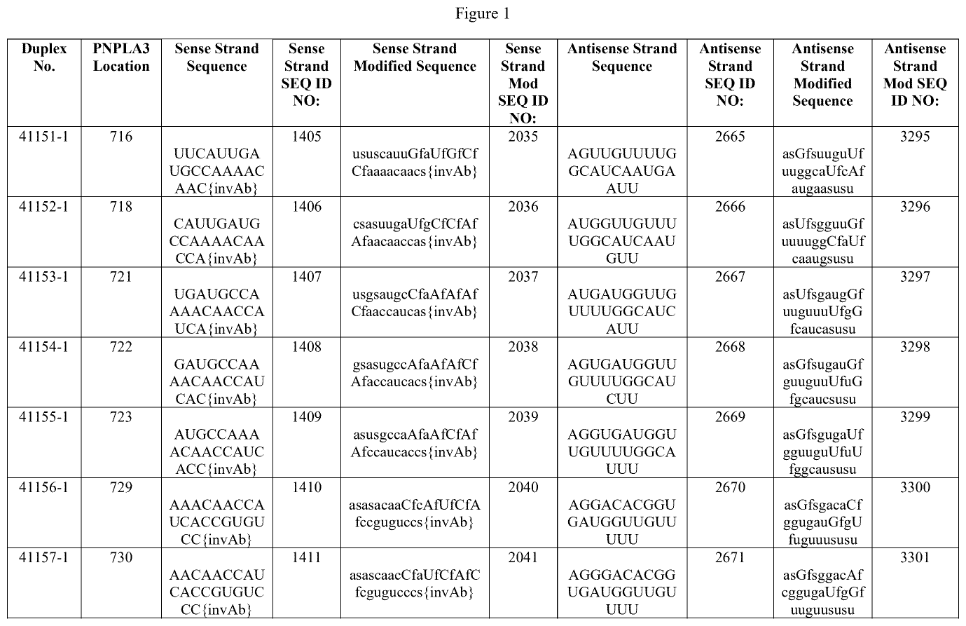

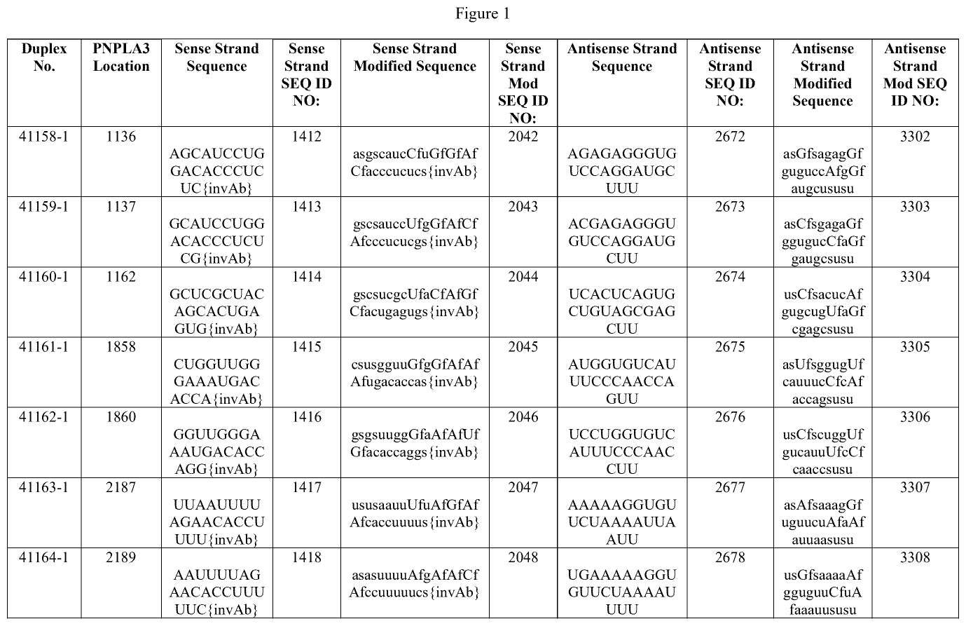

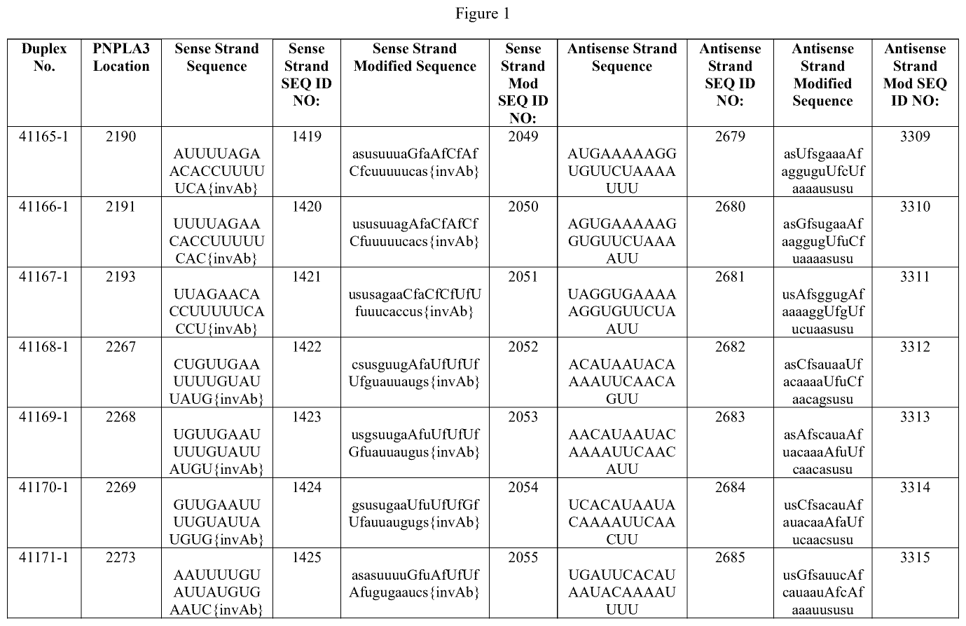

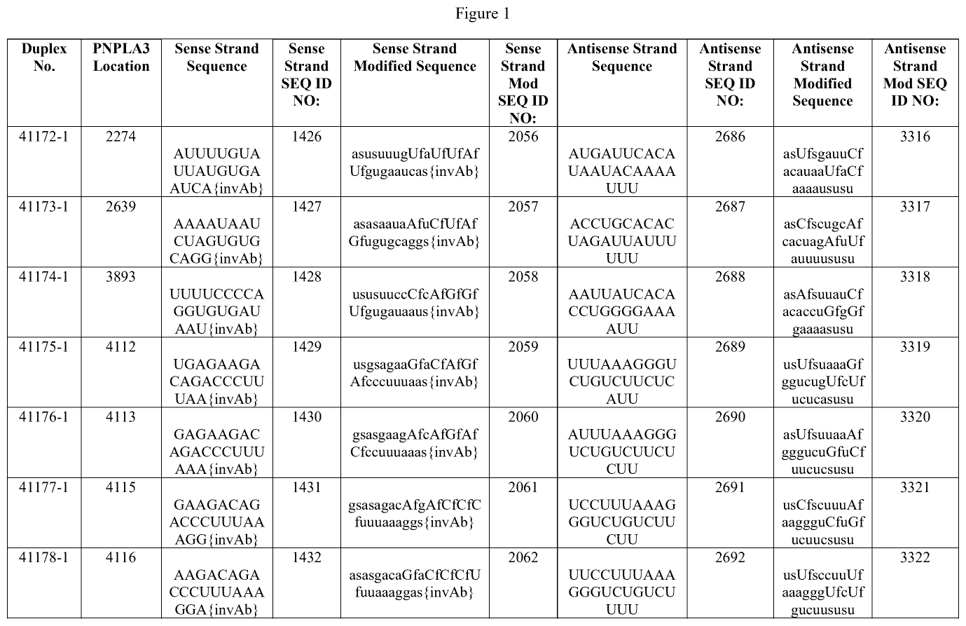

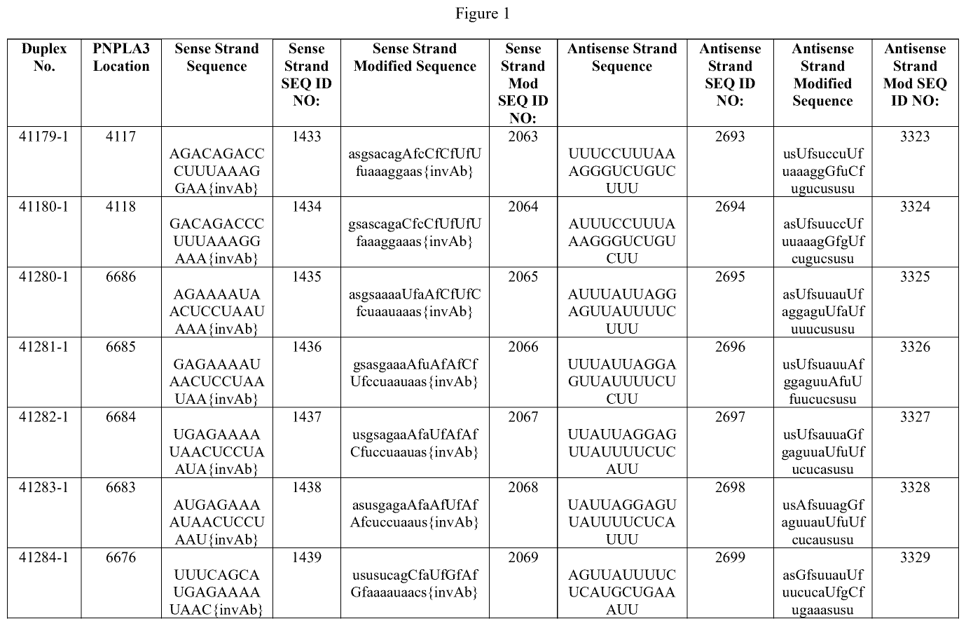

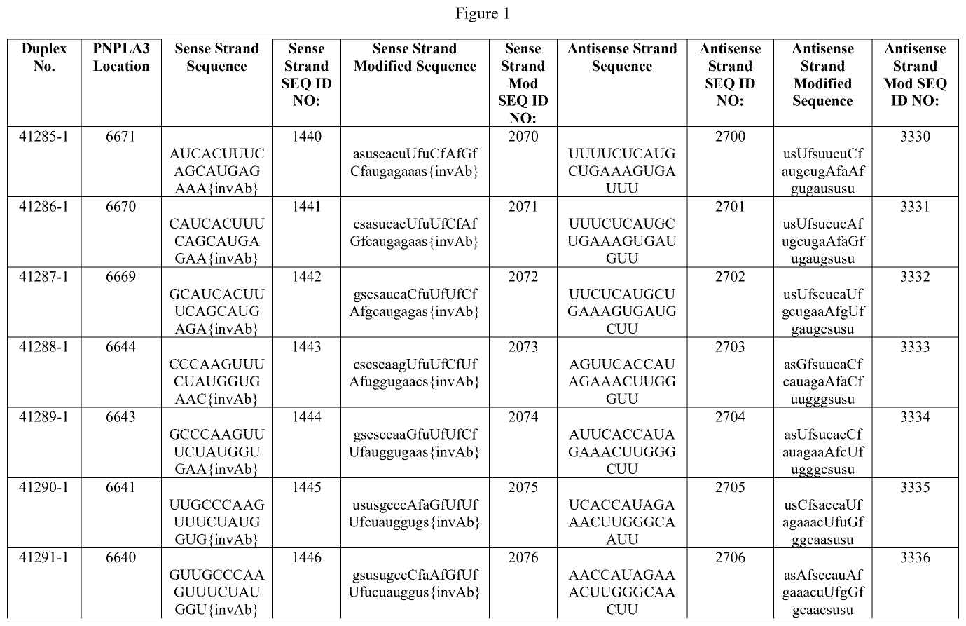

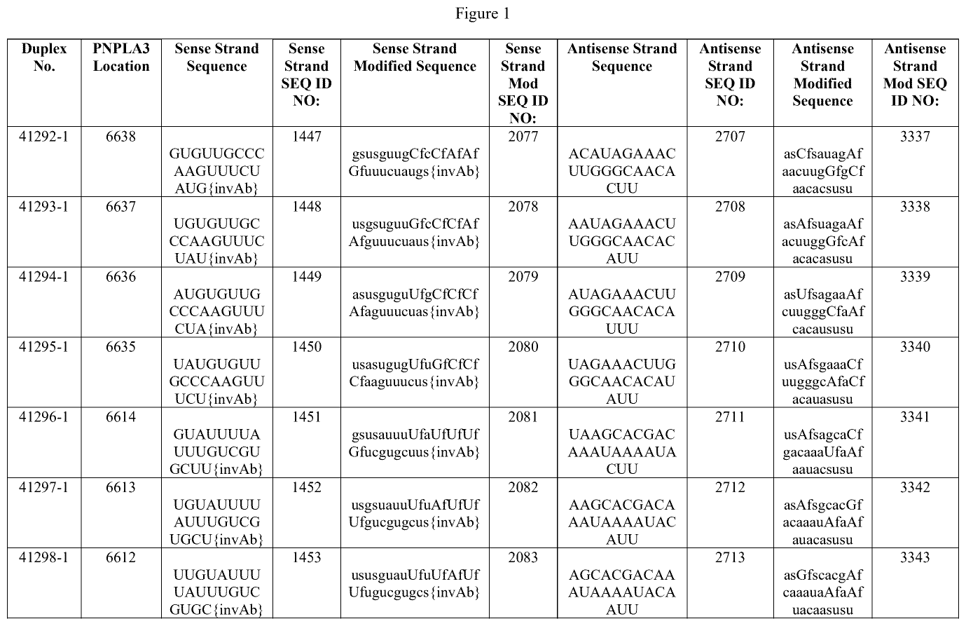

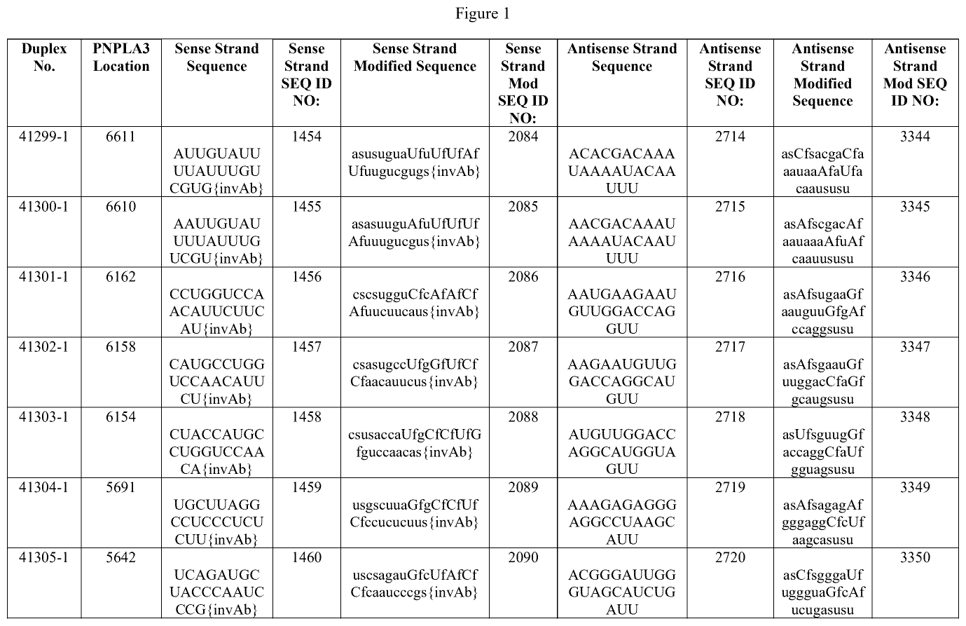

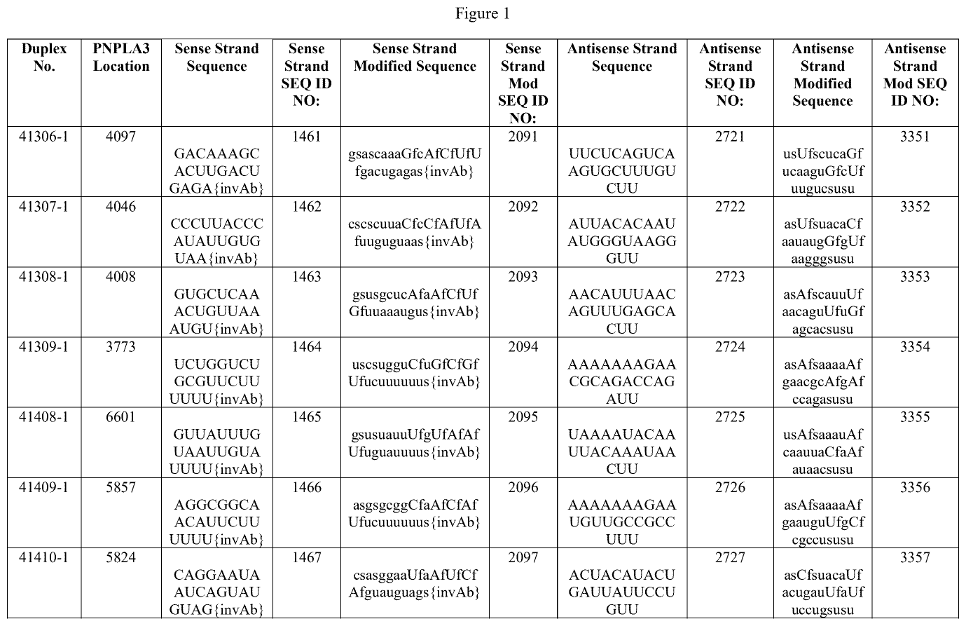

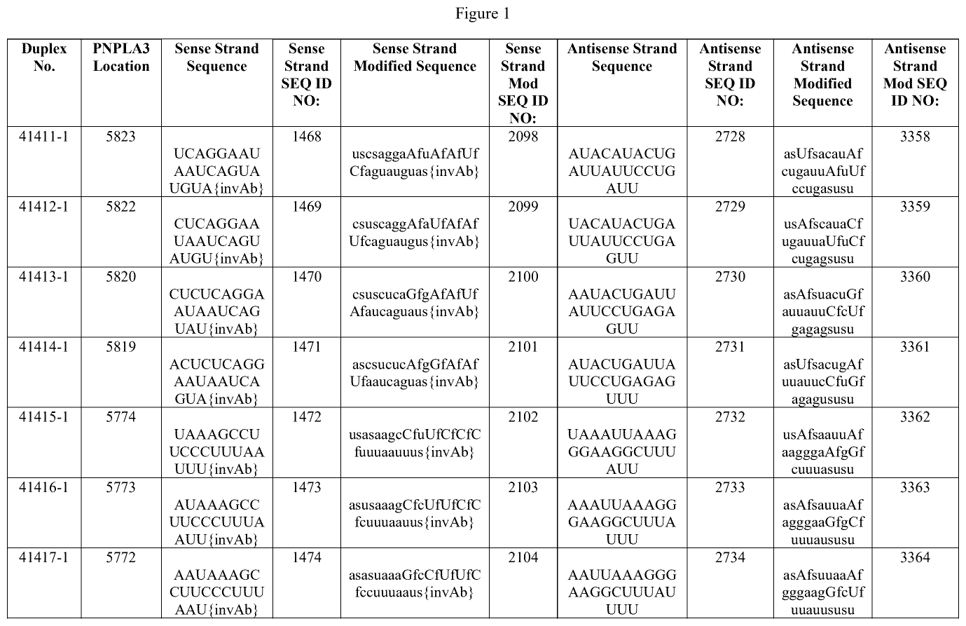

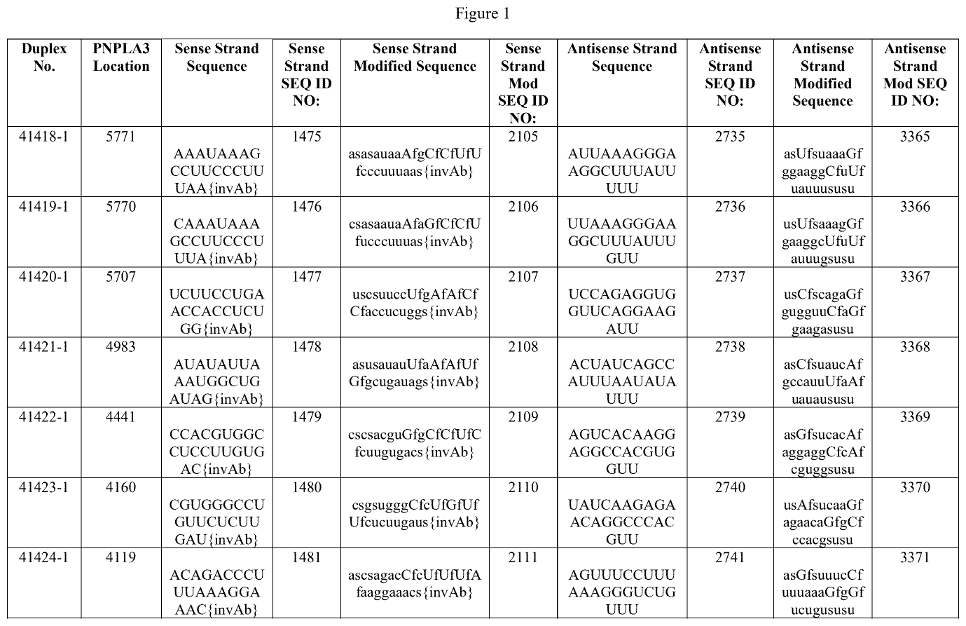

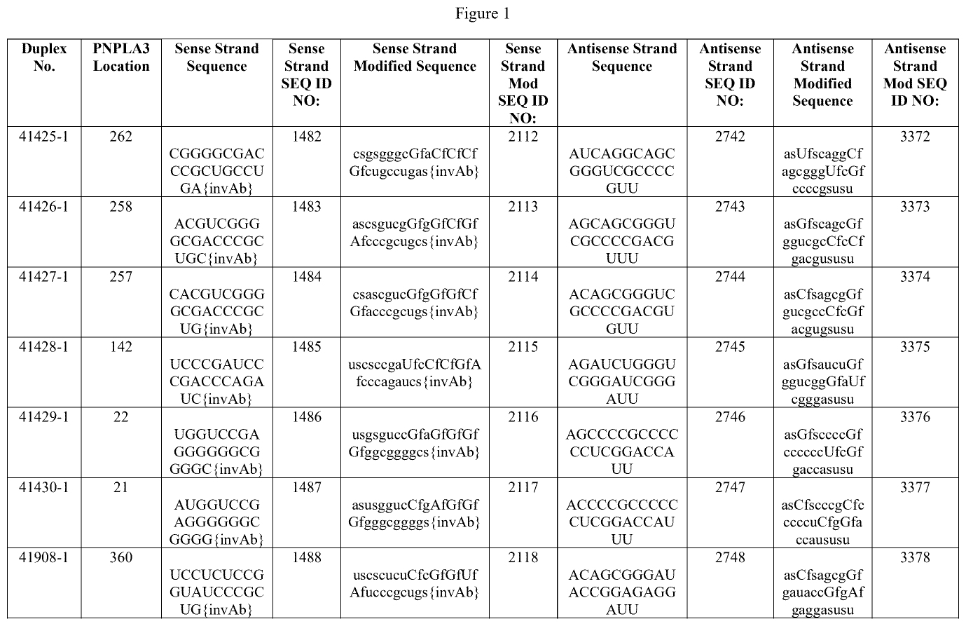

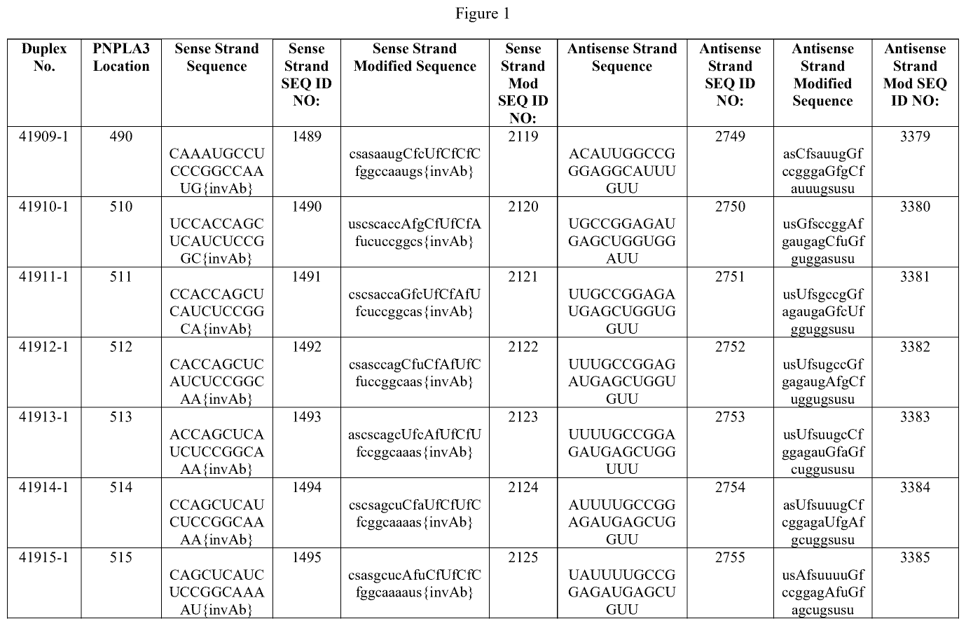

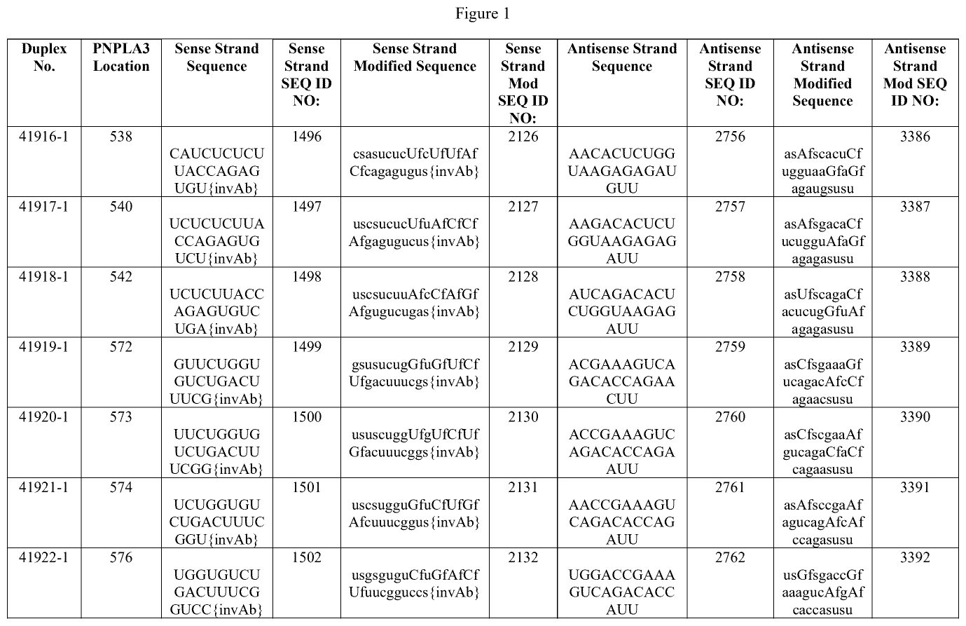

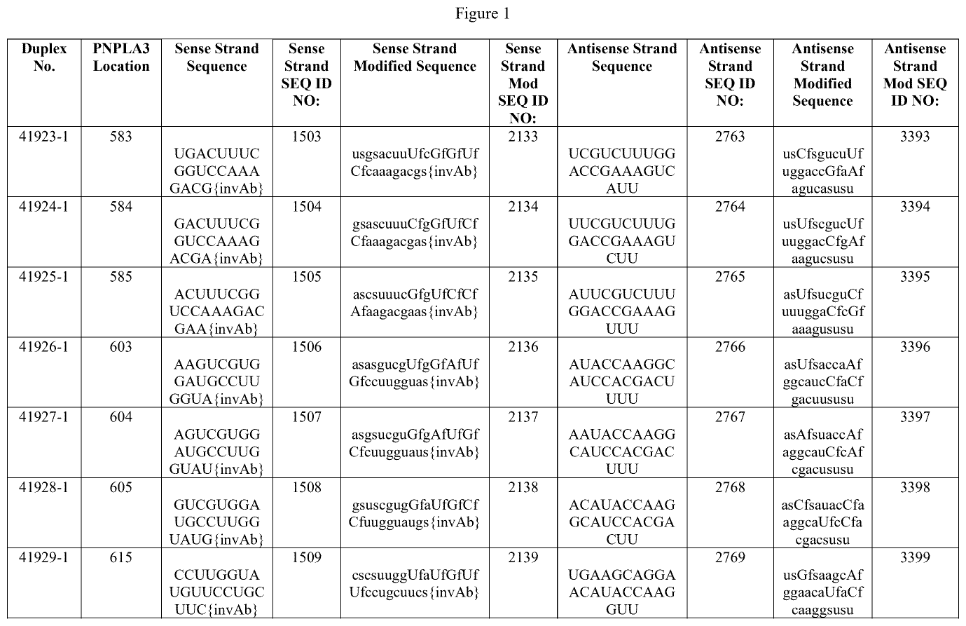

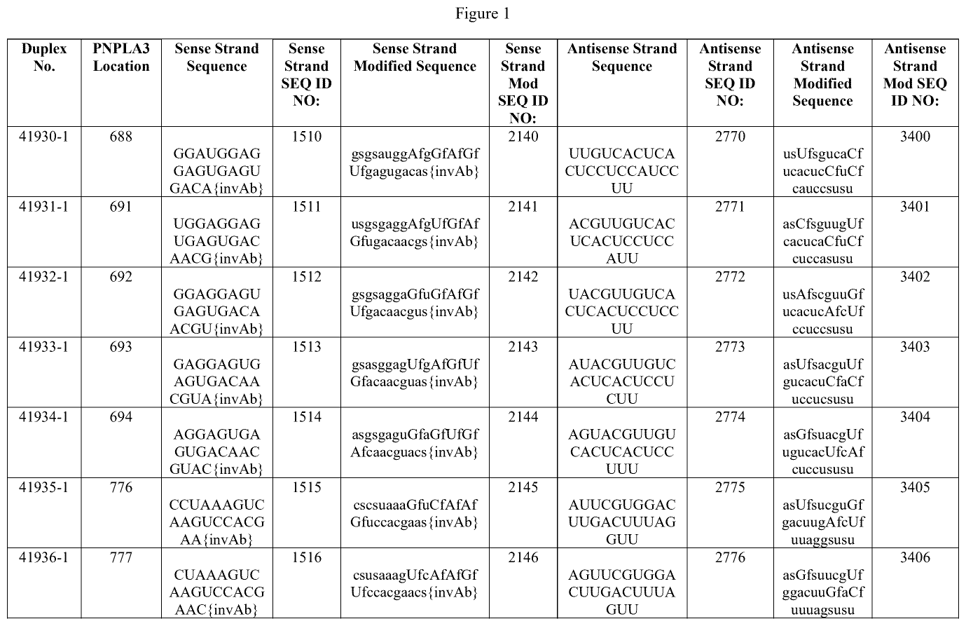

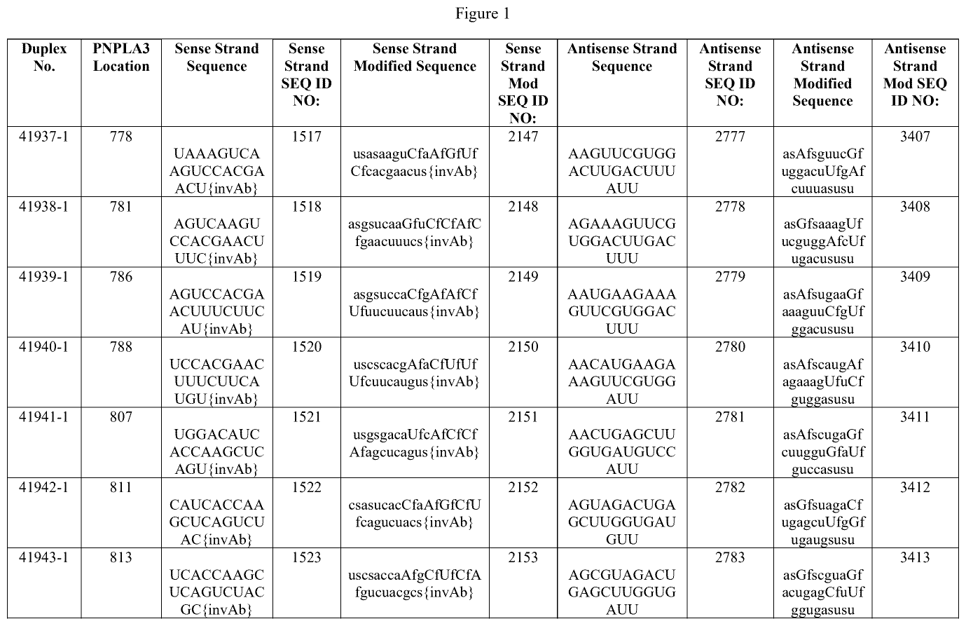

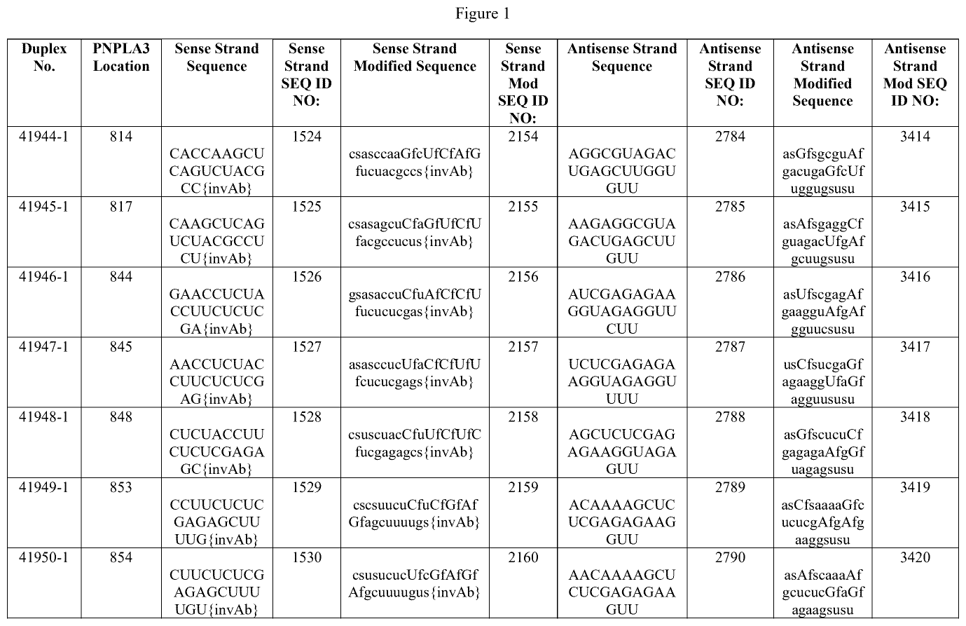

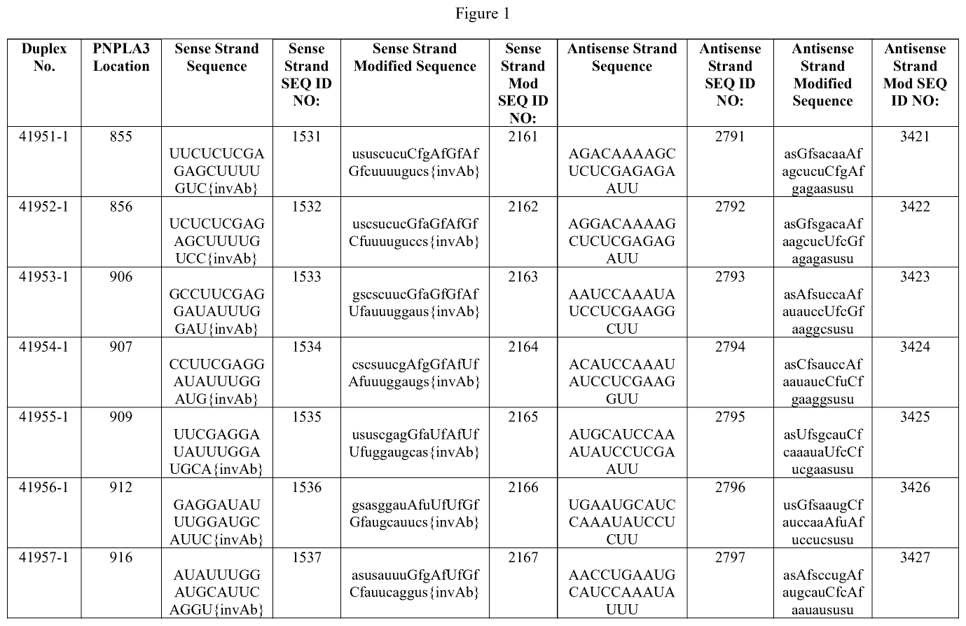

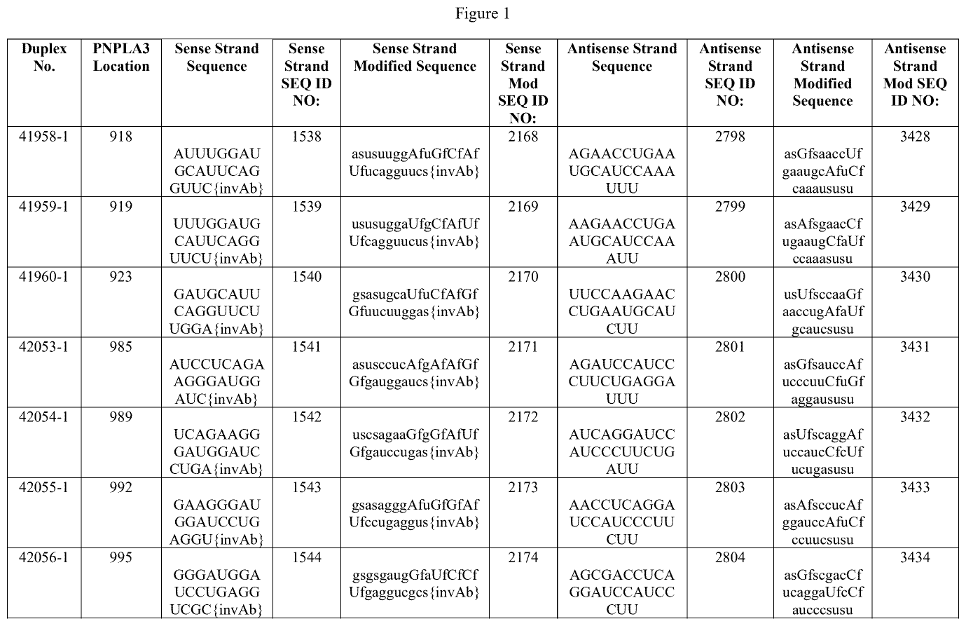

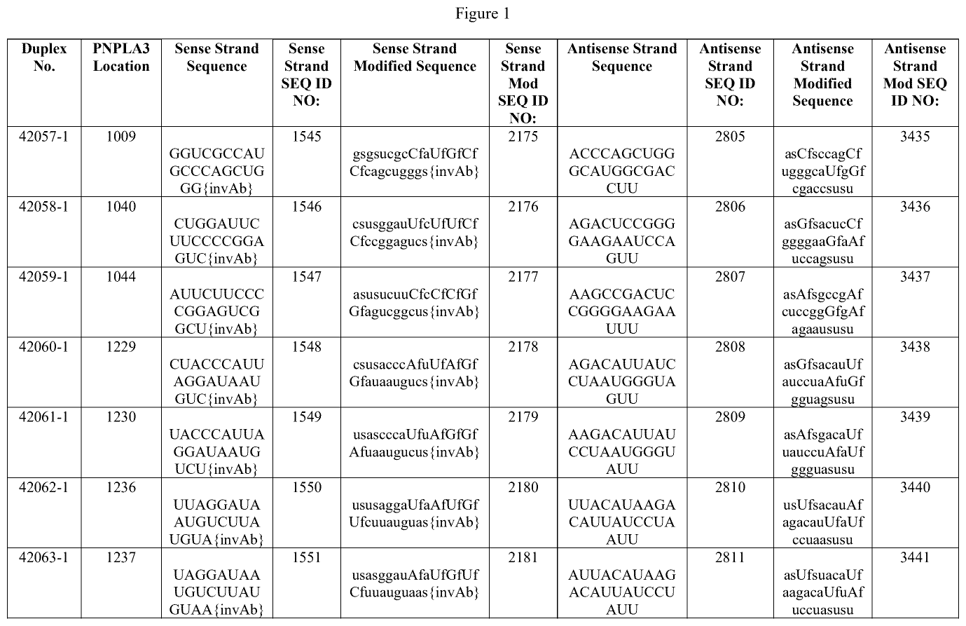

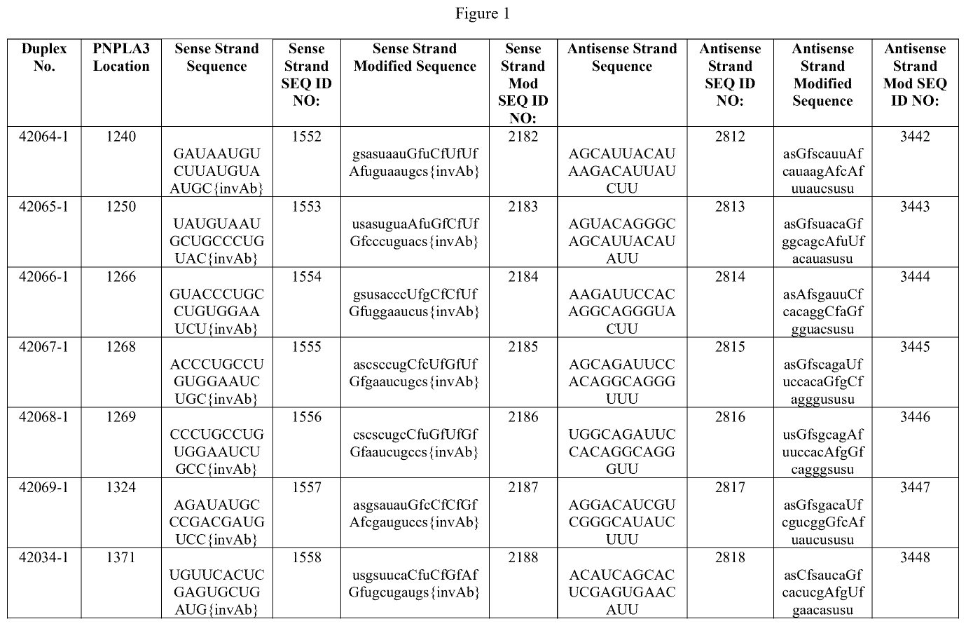

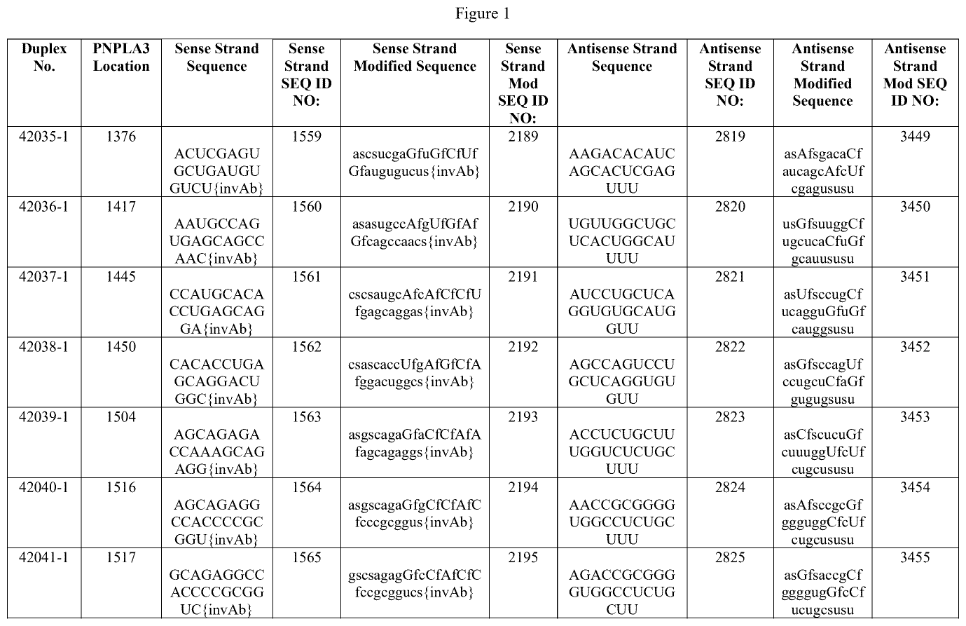

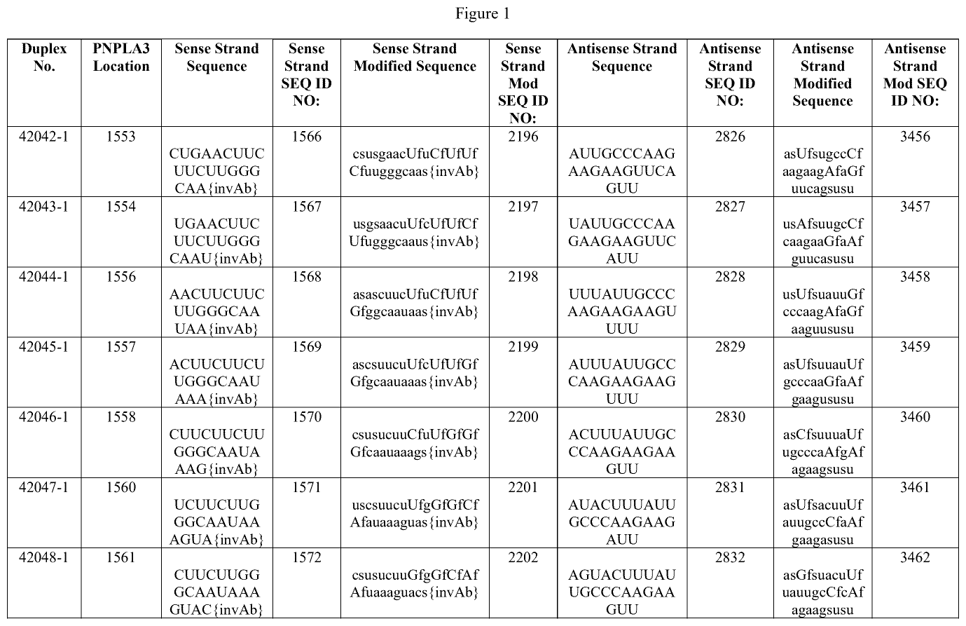

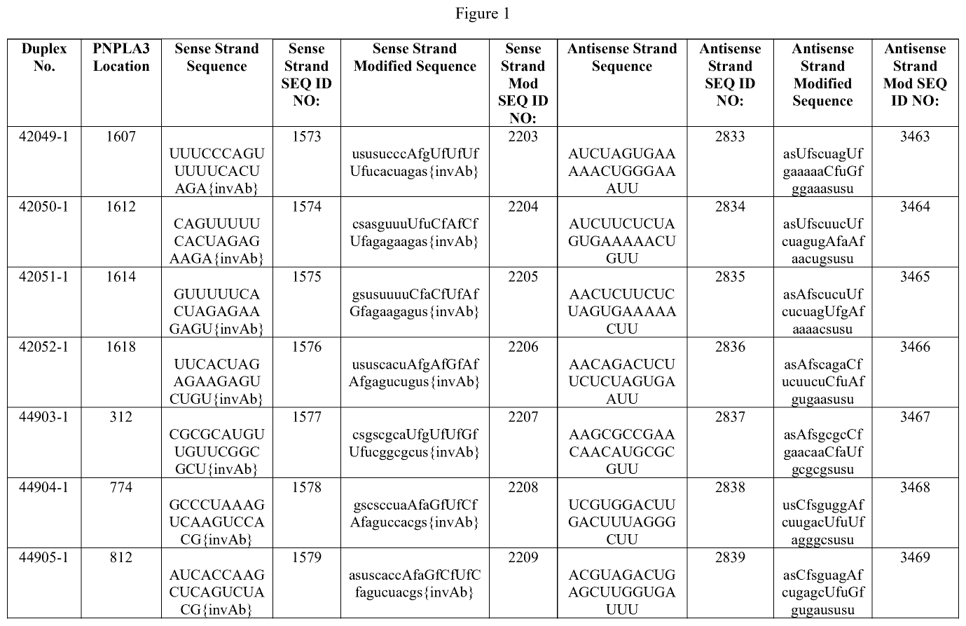

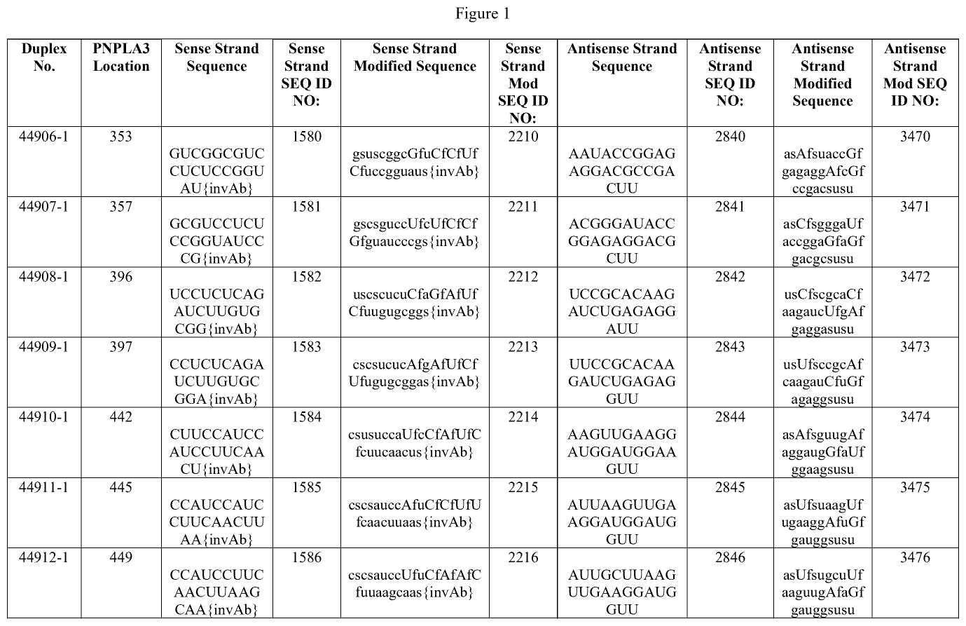

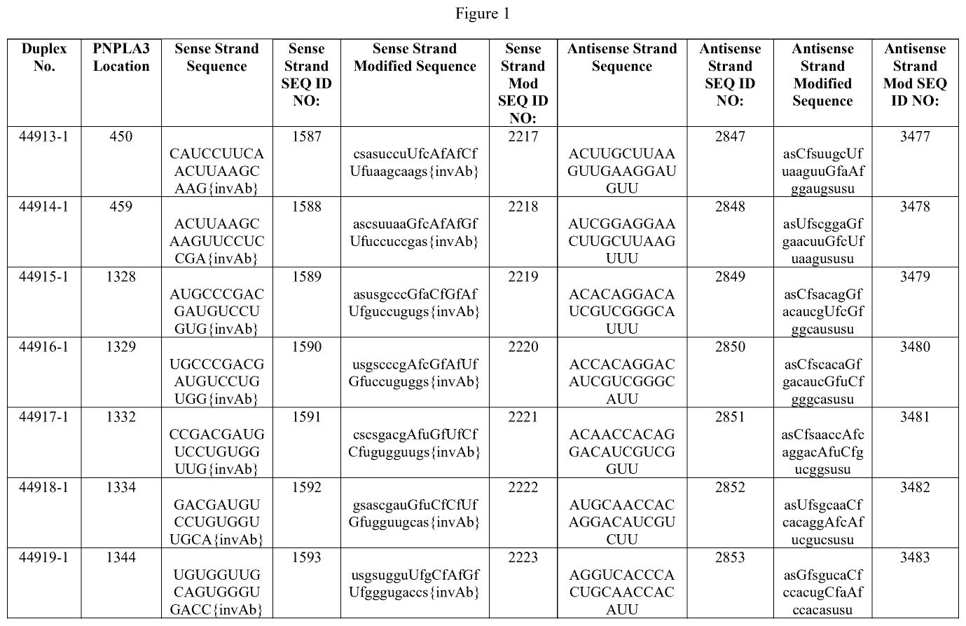

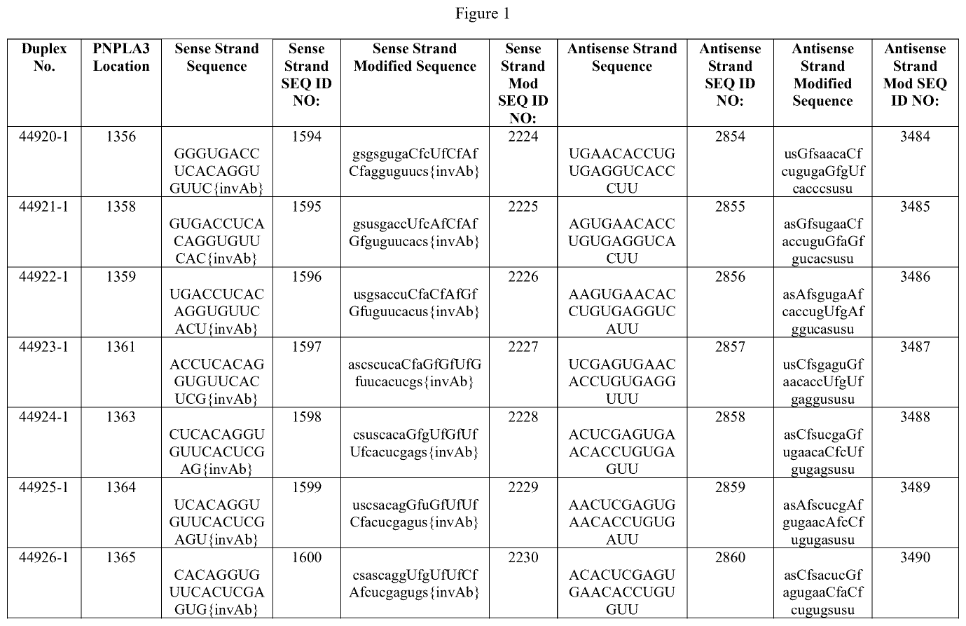

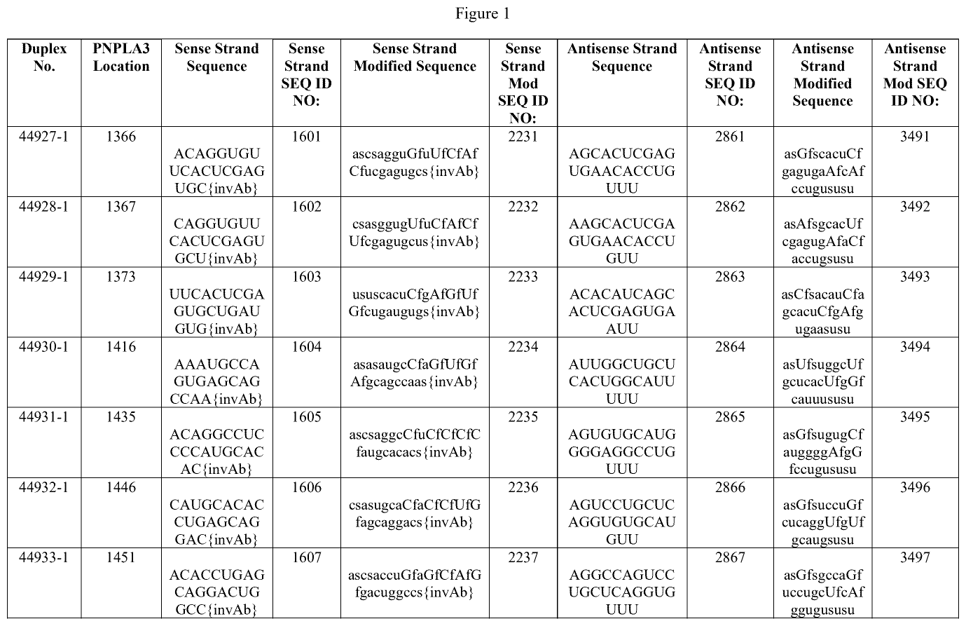

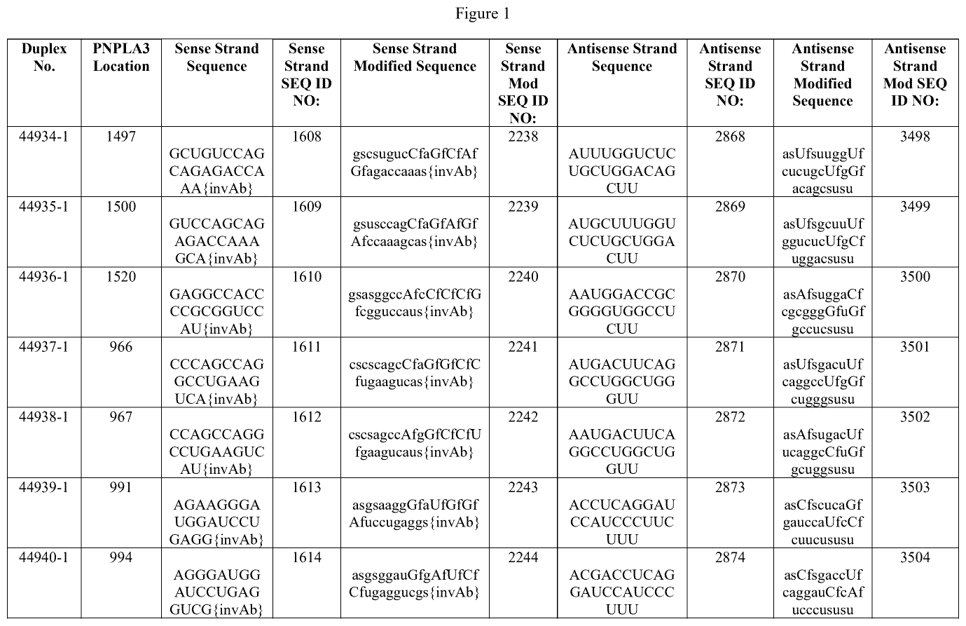

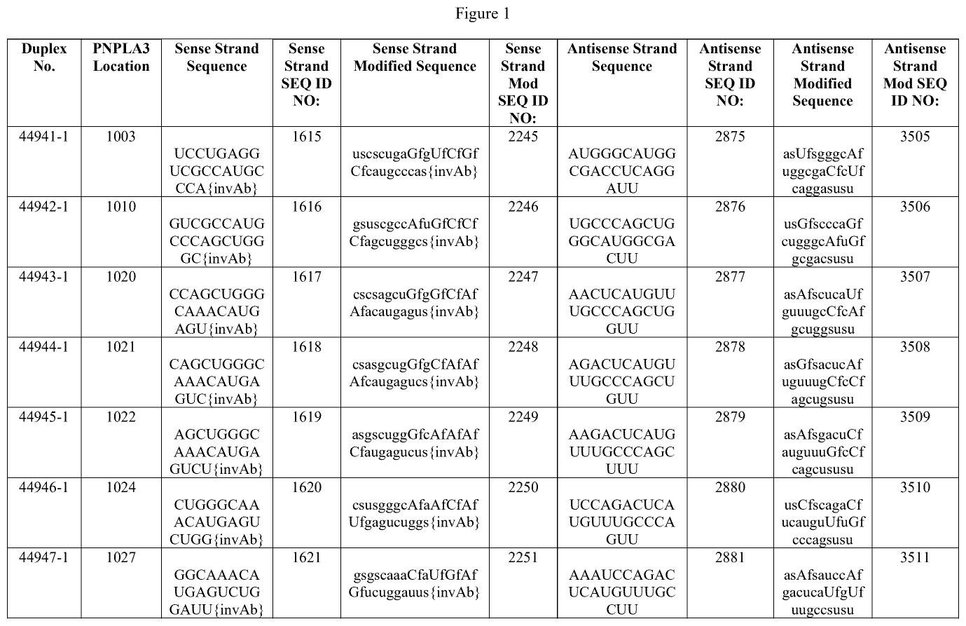

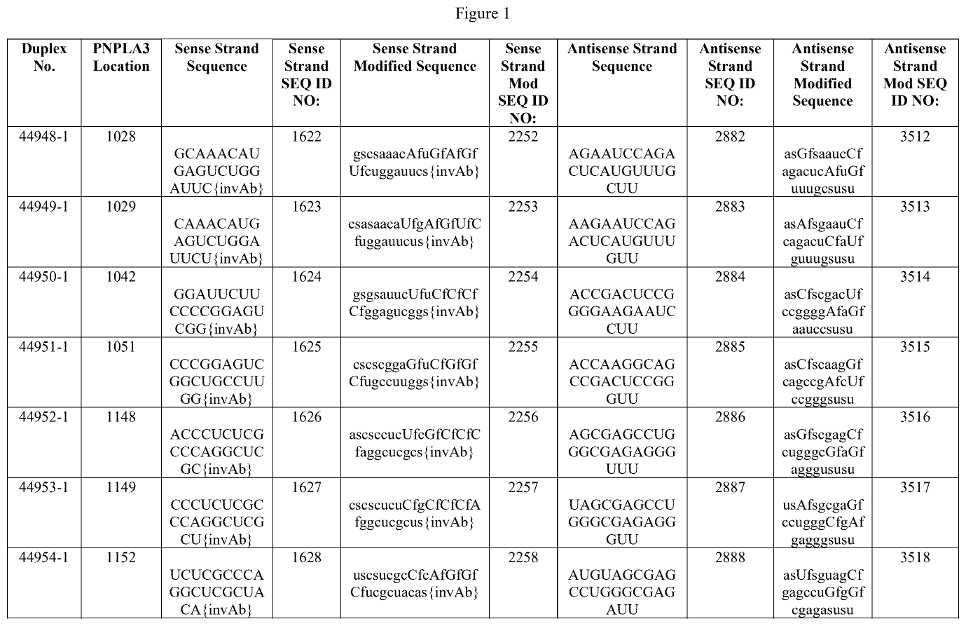

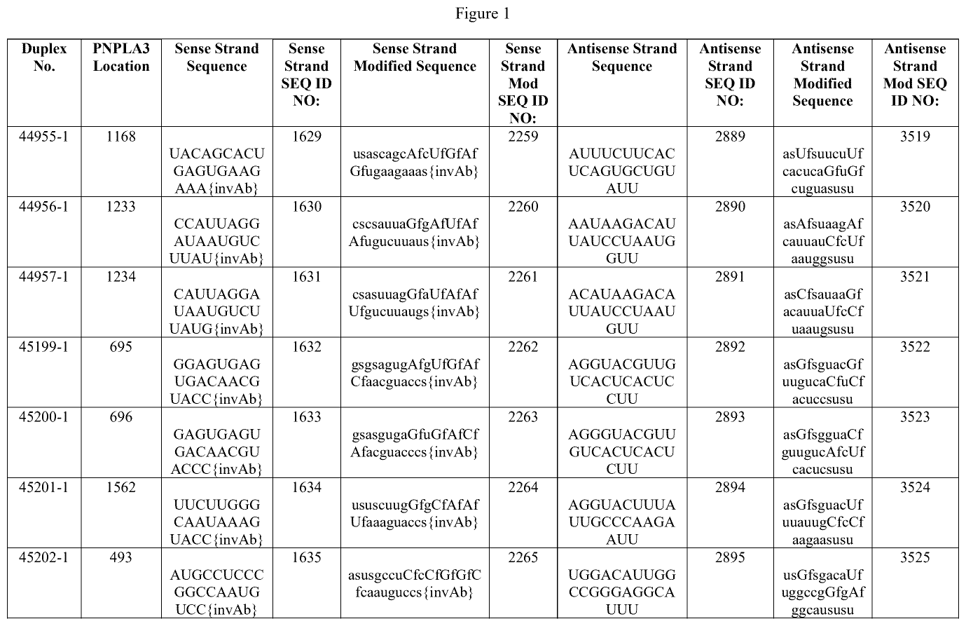

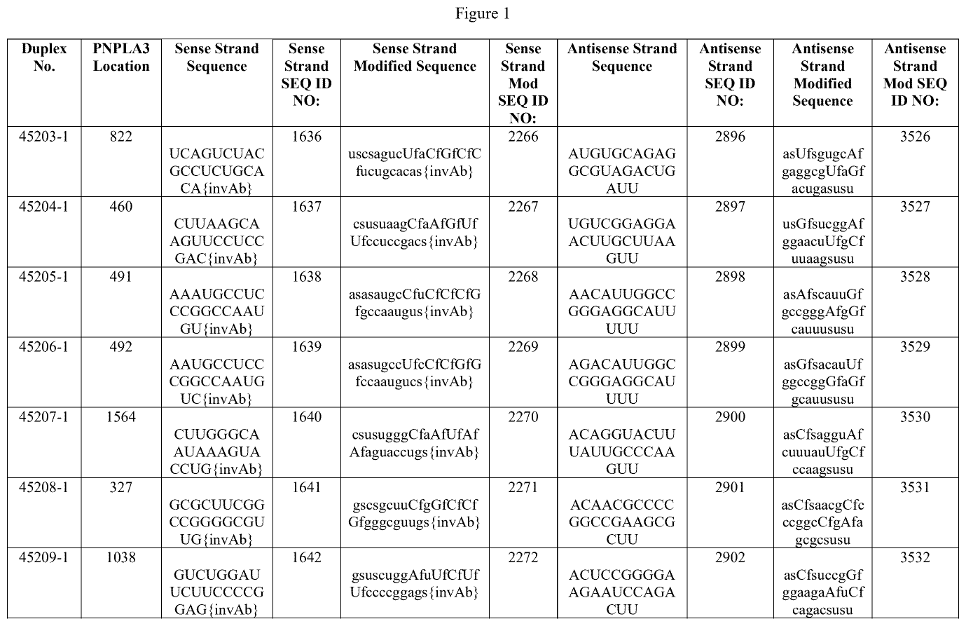

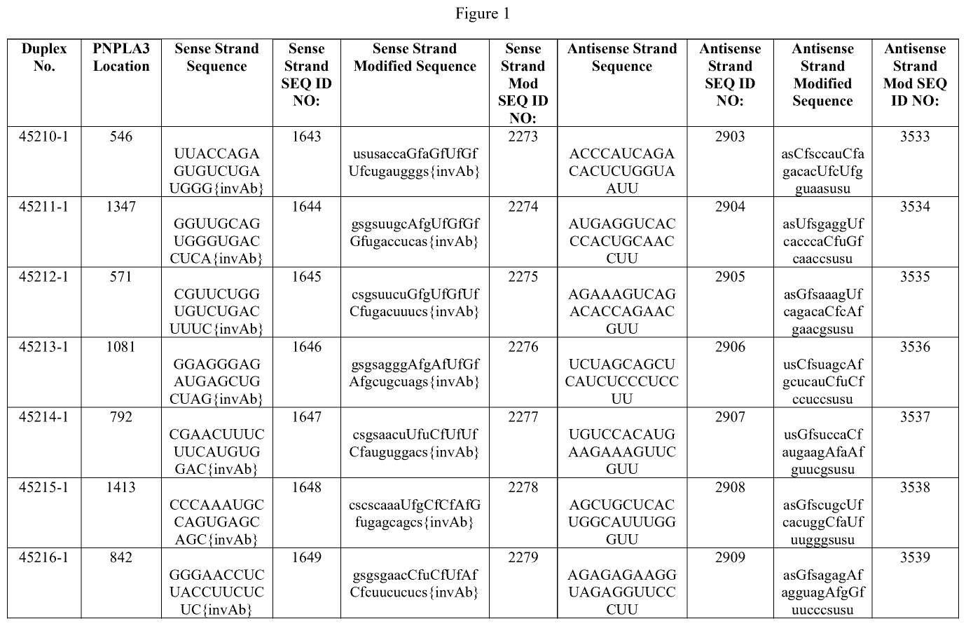

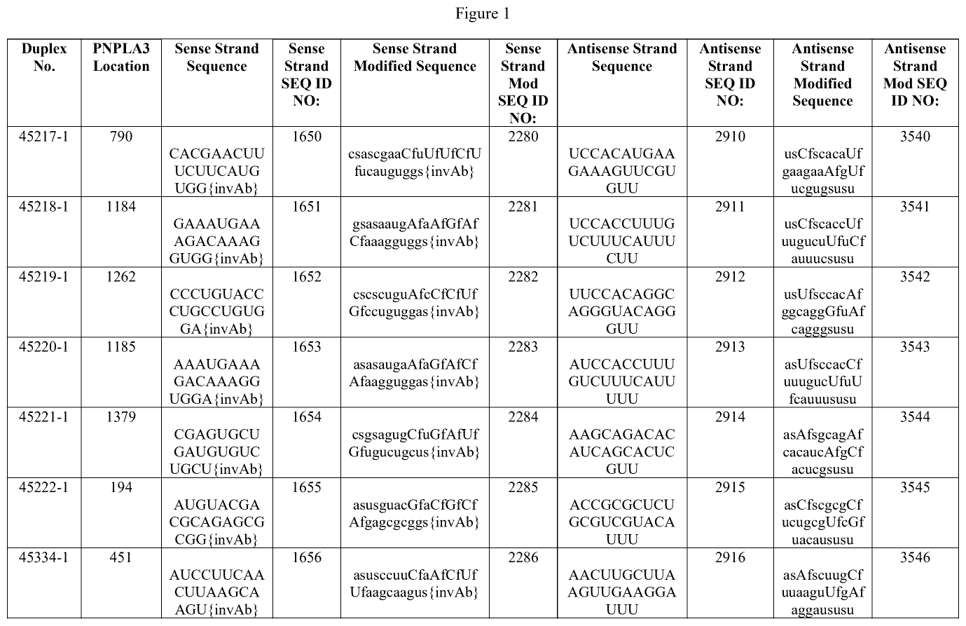

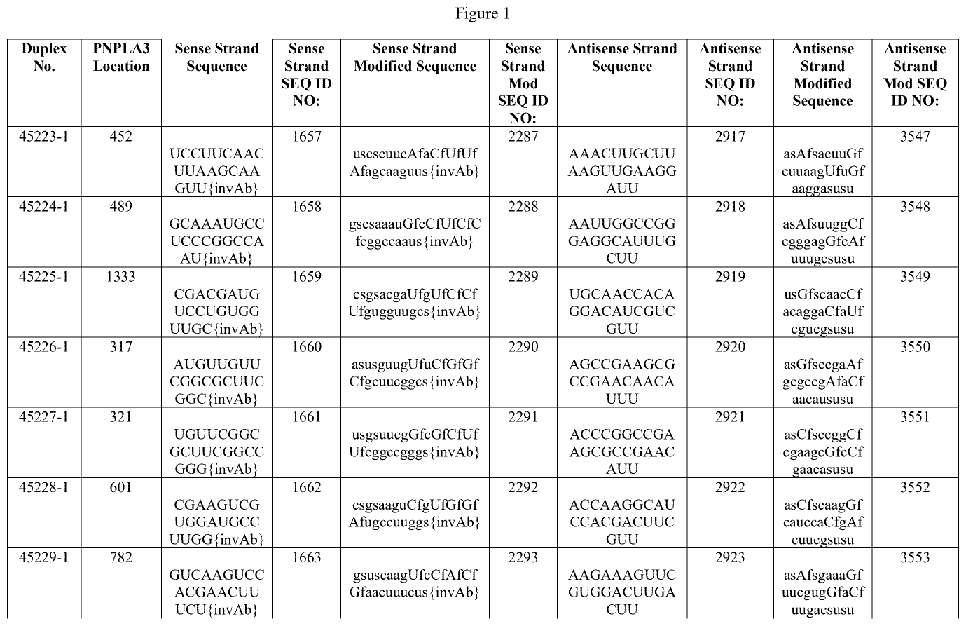

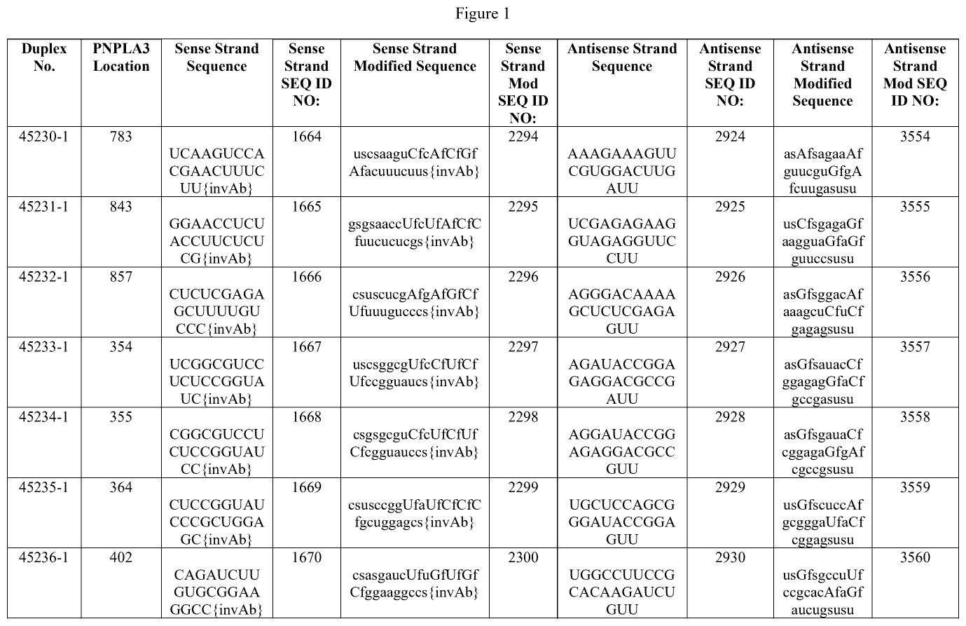

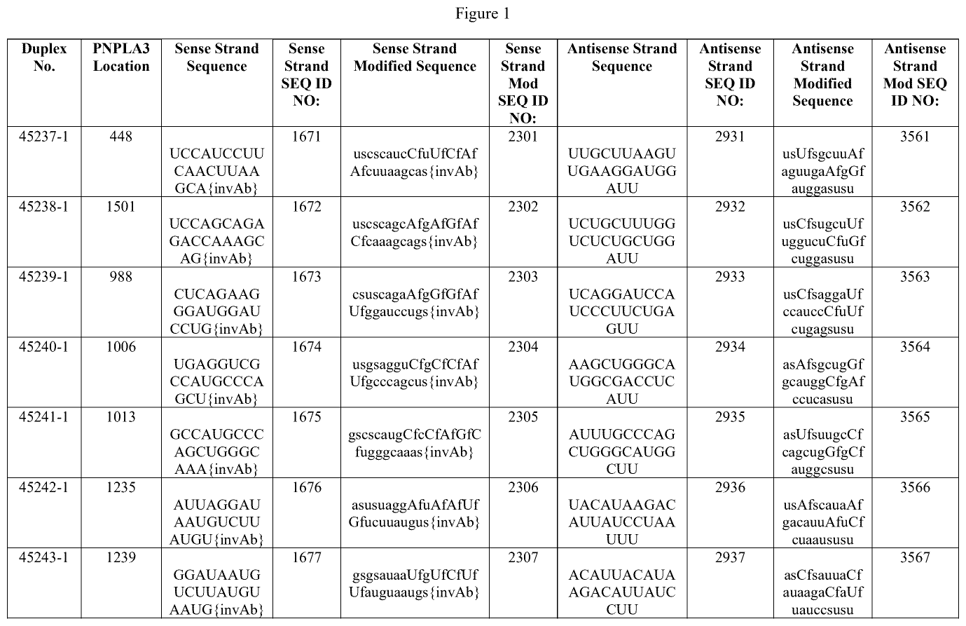

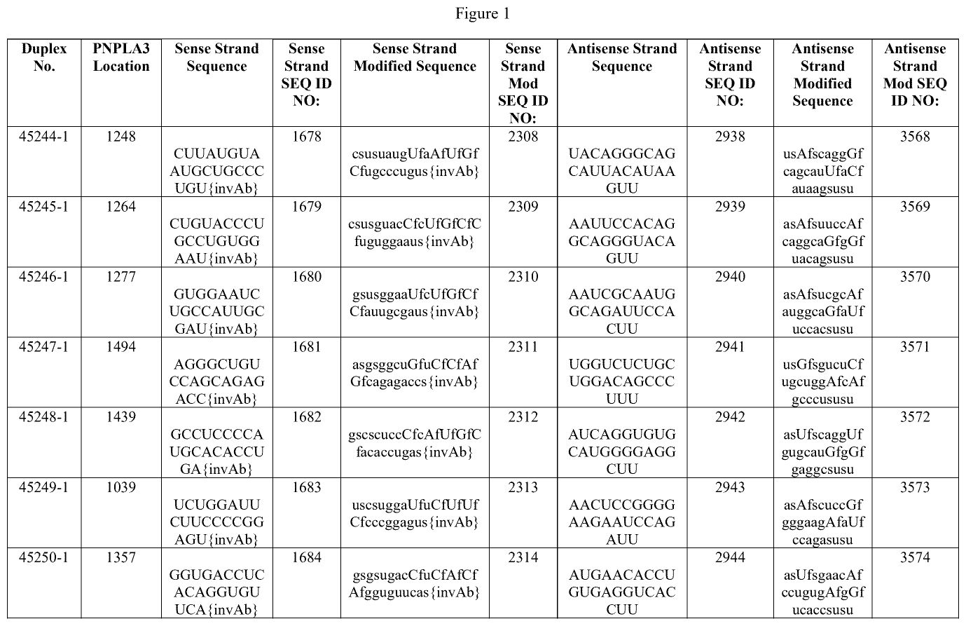

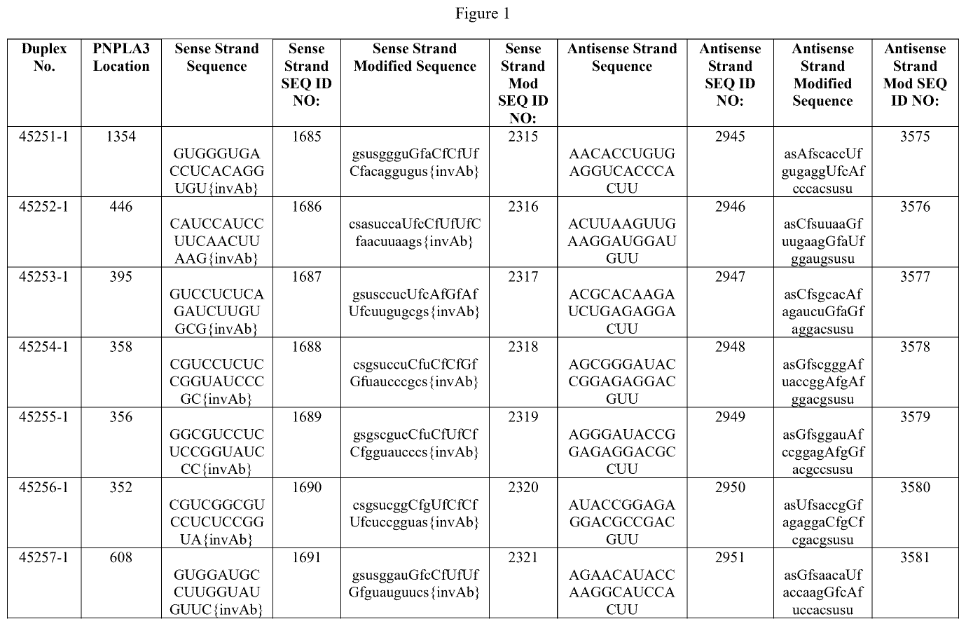

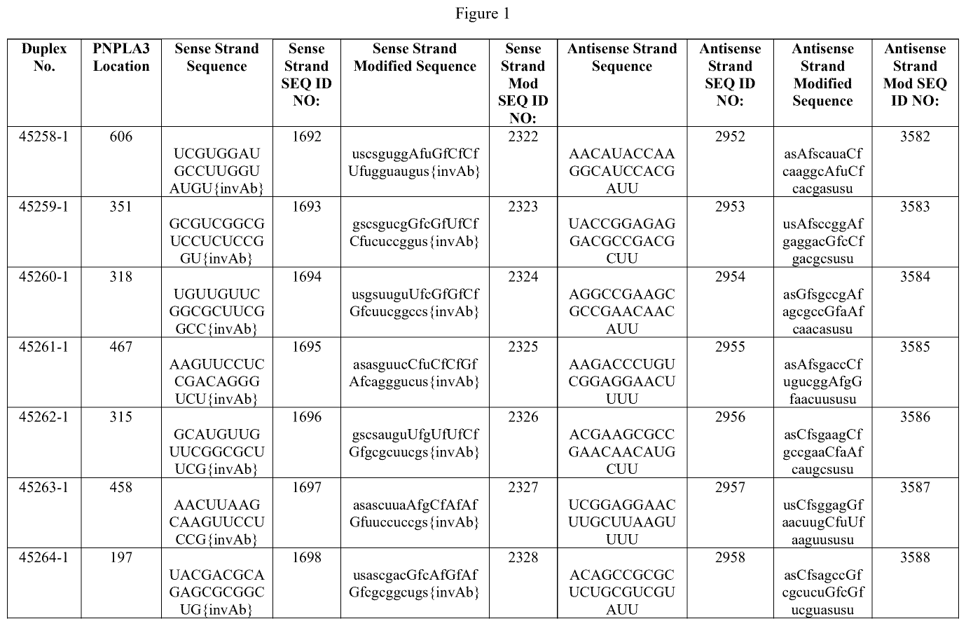

The nucleotide sequences in FIGS. 1-5 and other parts of the application are listed according to the following notations: A, U, G, and C=corresponding ribonucleotide; dT=deoxythymidine; dA=deoxyadenosine; dC=deoxycytidine; dG=deoxyguanosine; invAb=inverted A basic; invDT=inverted deoxythymidine; invDA=inverted deoxyadenosine; invDC=inverted deoxycytidine; invDG=inverted deoxyguanosin; a, u, g, and c=corresponding 2′-O-methyl ribonucleotide; Af, Uf, Gf, and Cf=corresponding 2′-deoxy-2′-fluoro (“2′-fluoro”) ribonucleotide; Ab=Abasic; MeO-I=2′ methoxy inosine; GNA=glycol nucleic acid; sGNA=glycol nucleic acid with 3′ phosphorothioate; LNA=locked nucleic acid. Insertion of an “s” in the sequence indicates that the two adjacent nucleotides are connected by a phosphorothiodiester group (e.g. a phosphorothioate internucleotide linkage). Unless indicated otherwise, all other nucleotides are connected by 3′-5′ phosphodiester groups.

### DETAILED DESCRIPTION

The present disclosure is based, in part, on the design and generation of RNAi constructs that target the PNPLA3 gene and reduce expression of PNPLA3 in liver cells in a non-sequence specific manner. The non-sequence specific inhibition of PNPLA3 expression is useful for treating or preventing conditions associated with PNPLA3 expression, including liver-related diseases, such as, for example, simple fatty liver (steatosis), nonalcoholic steatohepatitis (NASH), cirrhosis (irreversible, advanced scarring of the liver), or PNPLA3-related obesity.

The disclosure provides compositions and methods for regulating the expression of the Patatin-Like Phospholipase Domain Containing 3 (PNPLA3) gene. In some embodiments, the gene may be within a cell or subject, such as a mammal (e.g., a human). In some embodiments, compositions of the disclosure comprise RNAi constructs that target a PNPLA3 mRNA and reduce PNPLA3 expression in a cell or mammal. Such RNAi constructs are useful for treating or preventing various forms of liver-related diseases, such as, for example, simple fatty liver (steatosis), nonalcoholic fatty liver disease (NAFLD), nonalcoholic steatohepatitis (NASH), cirrhosis (irreversible, advanced scarring of the liver), or PNPLA3-related obesity.

In 2008, a genome wide association study (GWAS) exploring nonsynonymous sequence variations, or single nucleotide polymorphisms (SNPs), associated with NAFLD identified a variant in PNPLA3, (rs738409[G], encoding I148M; which can be referred to as PNPLA3-rs738409, PNPLA3-ma, or PNPLA3-minor allele), as significantly associated with hepatic fat content. Since this initial report, subsequent GWAS confirmed PNPLA3 rs738409 as the major genetic determinant of NAFLD, significantly associated with 1) increased levels of the serum biomarker for liver damage, alanine transaminase (ALT), 2) NAFLD incidence, progression, and severity, 3) both obese and lean individuals, and 4) the only known SNP shown to be significantly associated with all stages of NAFLD: steatosis, NASH, cirrhosis and hepatic cell carcinoma. The consensus among numerous GWAS indicates that the association of PNPLA3 rs738409 with NAFLD is independent of age, gender, ethnicity, metabolic syndrome, body mass index, insulin resistance, and serum lipids. Furthermore, statistical analyses from multiple sources estimate approximately 50% of NAFLD patients carry the PNPLA3 rs738409 mutation. Patients can be homozygous or heterozygous for the PNPLA3 rs738409 mutation. Additionally, it has been discovered that patients having the PNPLA3 rs738409 mutation often also carry an rs738408 mutation 3 base pairs away (Tian et al (2010) Nature Genetics 42:21-23). Thus, a patient can have a PNPLA3-rs738409 minor allele, a PNPLA3-rs738408 minor allele or a PNPLA3-rs738409-rs738408 double minor allele mutation (PNPLA3-dma).

Investigators have developed mouse models for exploring PNPLA3 function in vivo. To date, no detectable metabolic phenotype has been identified as the result of PNPLA3-deficiency or PNPLA3 over-expression. In contrast, expression of PNPLA3I148M in both transgenic mice and knock-in mice led to increased hepatic triglyceride levels akin to NAFLD. Thus, the combined in vivo mouse model data points to expression of the mutant PNPLA3I148M protein, and not over-expression of the wild-type protein, as the driver of the disease phenotype. These findings, in addition to the high frequency of the minor allele in NAFLD-affected individuals and prevailing association with the disease, underline PNPLA3 rs738409 as a prime therapeutic target for NAFLD.

RNA interference (RNAi) is the process of introducing exogeneous RNA into a cell leading to specific degradation of the mRNA encoding the targeted protein with a resultant decrease in protein expression. Advances in both the RNAi technology and hepatic delivery, as well as growing positive outcomes with other RNAi-based therapies, suggest RNAi as a compelling means to therapeutically treat NAFLD by directly targeting PNPLA3.

As used herein, the term “RNAi construct” refers to an agent comprising an RNA molecule that is capable of downregulating expression of a target gene (e.g. PNPLA3) via an RNA interference mechanism when introduced into a cell. “RNA interference” is the process by which a nucleic acid molecule induces the cleavage and degradation of a target RNA molecule (e.g. messenger RNA or mRNA molecule) in a sequence-specific manner, e.g., through an RNA induced silencing complex (RISC) pathway. In some embodiments, the RNAi construct comprises a double-stranded RNA (dsRNA) molecule comprising two antiparallel strands of contiguous nucleotides that are sufficiently complementary to each other to hybridize to form a duplex region. A double-stranded RNAi construct also may be referred to as an RNAi “trigger.” The terms “hybridize” or “hybridization” refer to the pairing of complementary polynucleotides, typically via hydrogen bonding (e.g., Watson-Crick, Hoogsteen or reversed Hoogsteen hydrogen bonding) between complementary bases in the two polynucleotides. The strand comprising a region having a sequence that is substantially complementary to a target sequence (e.g., target mRNA) is referred to as the “antisense strand.” The “sense strand” refers to the strand that includes a region that is substantially complementary to a region of the antisense strand. In some embodiments, the sense strand may comprise a region that has a sequence that is substantially identical to the target sequence.

In certain embodiments, the sense strand and antisense strand of the double-stranded RNA may be two separate molecules that hybridize to form a duplex region but are otherwise unconnected. Such double-stranded RNA molecules formed from two separate strands are referred to as “small interfering RNAs” or “short interfering RNAs” (siRNAs). siRNAs are a class of non-coding, double-stranded RNA molecules that are typically about 20-27 base pairs and are central to RNAi. Thus, in some embodiments, the RNAi constructs of the disclosure comprise an siRNA. In other embodiments, the RNAi construct may be a microRNA (also known as “miRNA” or “mature miRNA”). miRNAs are small (approximately 18-24 nucleotides in length), non-coding RNA molecules present in plants, animals, and some viruses. miRNAs resemble siRNA, but miRNAs originate from hairpin mRNA structures. miRNAs regulate gene expression by base-pairing to complementary regions of target mRNAs.

In some embodiments, the disclosure provides an RNAi construct directed to PNPLA3. In some embodiments, the RNAi construct is an siRNA that comprises a sense strand and an antisense strand, wherein the antisense strand comprises a region that is complementary to PNPLA3 mRNA sequence. The region of the RNAi antisense strand may be complementary to any suitable region of a PNPLA3 mRNA sequence. For example, the antisense strand may comprise a region that is complementary to the coding region or the 3′ untranslated region (UTR) of a PNPLA3 mRNA sequence. Exemplary PNPLA3 target sequences within the coding region (reference sequence GenBank Accession No. NM_025225.2) and 3′ UTR are set forth in Table 1. The antisense strand of the RNAi construct desirably hybridizes to a PNPLA3 mRNA sequence listed in Table 1.

TABLE 1
PNPLA3 siRNA Target Sites

| | | PNPLA3 Location | |
|---|---|---|---|
| PNPLA3 | | (reference sequence | SEQ ID |
| Target Region | 23 mer Target Site | NM_025225.2) | NO: |
| Coding Region | CTGCAGGTCCTCTCAGATCTTGT | 391 | 1 |
| Coding Region | GAGAGATATGCCTTCGAGGATAT | 899 | 2 |
| Coding Region | CGCGGCTGGAGCTTGTCCTTCGC | 211 | 3 |
| Coding Region | AGATATGCCTTCGAGGATATTTG | 902 | 4 |
| Coding Region | TGCAGGTCCTCTCAGATCTTGTG | 392 | 5 |
| Coding Region | GGTCCAAAGACGAAGTCGTGGAT | 593 | 6 |
| Coding Region | CCTTCTACAGTGGCCTTATCCCT | 641 | 7 |
| Coding Region | TTTGCAACTTGCTACCCATTAGG | 1220 | 8 |
| Coding Region | GTGACAACGTACCCTTCATTGAT | 704 | 9 |
| Coding Region | CGACGCGCGCATGTTGTTCGGCG | 309 | 10 |
| Coding Region | AGATTTGCAACTTGCTACCCATT | 1217 | 11 |
| Coding Region | GGGAGAGATATGCCTTCGAGGAT | 897 | 12 |
| Coding Region | CTCCTTCCTTCAGAGGCGTGCGA | 662 | 13 |
| Coding Region | TGACAACGTACCCTTCATTGATG | 705 | 14 |
| Coding Region | GACAACGTACCCTTCATTGATGC | 706 | 15 |
| Coding Region | TACGACATCTGCCCTAAAGTCAA | 766 | 16 |
| Coding Region | ACTTGCTACCCATTAGGATAATG | 1226 | 17 |
| Coding Region | CTATGGGGAGTACGACATCTGCC | 756 | 18 |
| Coding Region | TGGAATCTGCCATTGCGATTGTC | 1280 | 19 |
| Coding Region | ACGACATCTGCCCTAAAGTCAAG | 767 | 20 |
| Coding Region | TATGCCTTCGAGGATATTTGGAT | 905 | 21 |
| Coding Region | TGAGCTGCTAGACCACCTGCGTC | 1092 | 22 |
| Coding Region | GCAACTTGCTACCCATTAGGATA | 1223 | 23 |
| Coding Region | CAACTTGCTACCCATTAGGATAA | 1224 | 24 |
| Coding Region | TGCACTGCGTCGGCGTCCTCTCC | 347 | 25 |
| Coding Region | AGCTTGTCCTTCGCGGGCTGCGG | 220 | 26 |
| Coding Region | TTCCAGATATGCCCGACGATGTC | 1322 | 27 |
| Coding Region | GATGGGGAAAACGTTCTGGTGTC | 562 | 28 |
| Coding Region | ATGGGGAAAACGTTCTGGTGTCT | 563 | 29 |
| Coding Region | CCCCGGATCTCAAGGTGCTGGGA | 878 | 30 |
| Coding Region | TCAGAGGCGTGCGATATGTGGAT | 671 | 31 |
| Coding Region | CGCCTCTGCACAGGGAACCTCTA | 832 | 32 |
| Coding Region | CGACATCTGCCCTAAAGTCAAGT | 768 | 33 |
| Coding Region | GGTGGATACATGAGCAAGATTTG | 1201 | 34 |
| Coding Region | AGCAAGATTTGCAACTTGCTACC | 1213 | 35 |
| Coding Region | TGCTGGGAGAGATATGCCTTCGA | 893 | 36 |
| Coding Region | GTCCAGAGACTGGTGACATGGCT | 1300 | 37 |
| Coding Region | GAAAACGTTCTGGTGTCTGACTT | 568 | 38 |
| Coding Region | TTCGGTCCAAAGACGAAGTCGTG | 590 | 39 |
| Coding Region | ATTTGCAACTTGCTACCCATTAG | 1219 | 40 |
| Coding Region | GAGTGAGTGACAACGTACCCTTC | 698 | 41 |
| Coding Region | GCTGGGAGAGATATGCCTTCGAG | 894 | 42 |
| Coding Region | CTGGGAGAGATATGCCTTCGAGG | 895 | 43 |
| Coding Region | ATGAAAGACAAAGGTGGATACAT | 1189 | 44 |
| Coding Region | TGAGTGACAACGTACCCTTCATT | 701 | 45 |
| Coding Region | CTCCACCTTTCCCAGTTTTTCAC | 1602 | 46 |
| Coding Region | TCCACCTTTCCCAGTTTTTCACT | 1603 | 47 |
| Coding Region | AGTACGACATCTGCCCTAAAGTC | 764 | 48 |
| 3′UTR | AGAACACCTTTTTCACCTAACTA | 2197 | 49 |
| 3′UTR | CCTCTGAGCTGAGTTGGTTTTAT | 1760 | 50 |
| 3′UTR | CTGAGCTGAGTTGGTTTTATGAA | 1763 | 51 |
| 3′UTR | TGAGCTGAGTTGGTTTTATGAAA | 1764 | 52 |
| 3′UTR | AGTTGGTTTTATGAAAAGCTAGG | 1771 | 53 |
| 3′UTR | TGCAGAGGGTCCCTTACTGACTG | 1890 | 54 |
| 3′UTR | GAACACCTTTTTCACCTAACTAA | 2198 | 55 |
| 3′UTR | AACACCTTTTTCACCTAACTAAA | 2199 | 56 |
| 3′UTR | ACACCTTTTTCACCTAACTAAAA | 2200 | 57 |
| 3′UTR | CACCTTTTTCACCTAACTAAAAT | 2201 | 58 |
| 3′UTR | ACCTTTTTCACCTAACTAAAATA | 2202 | 59 |
| 3′UTR | CTTTTTCACCTAACTAAAATAAT | 2204 | 60 |

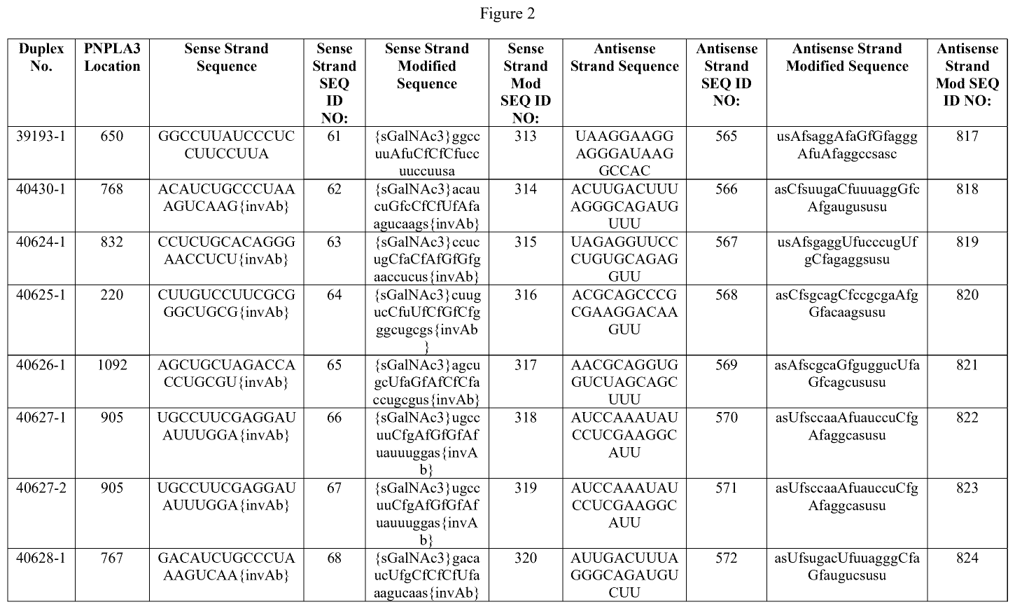

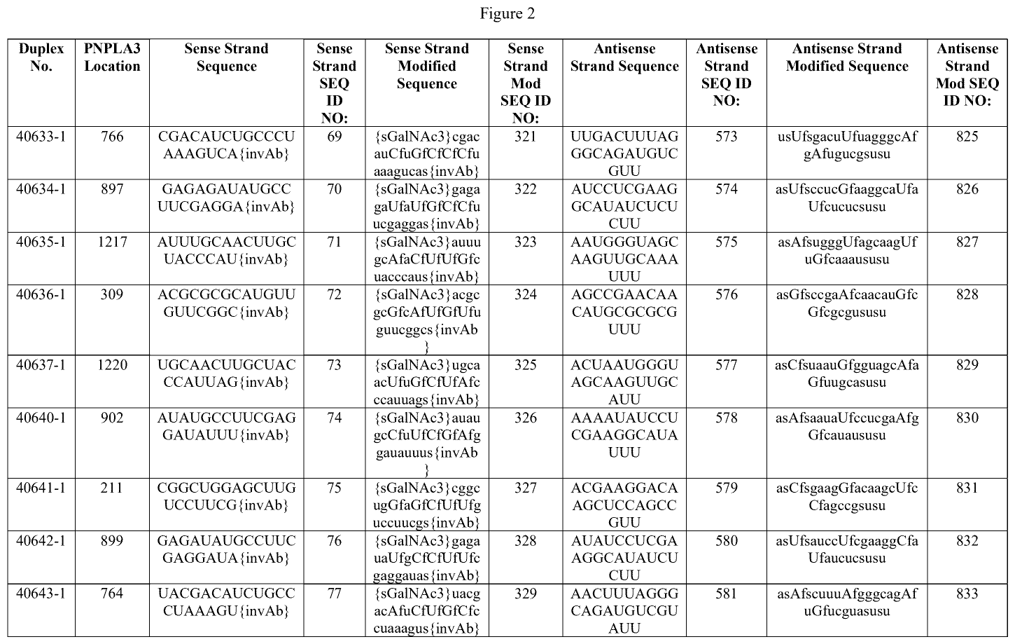

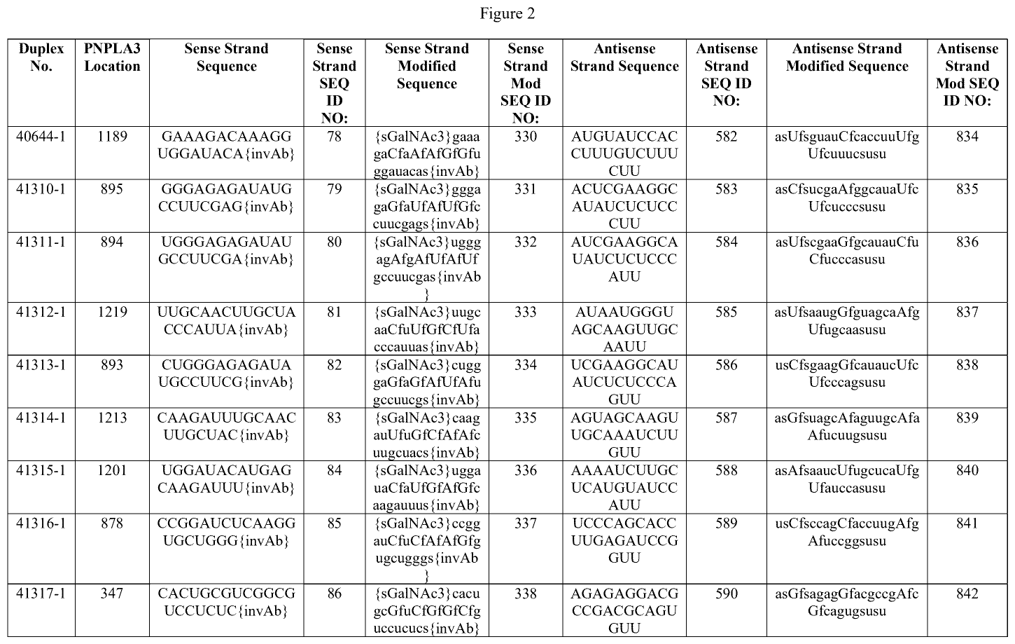

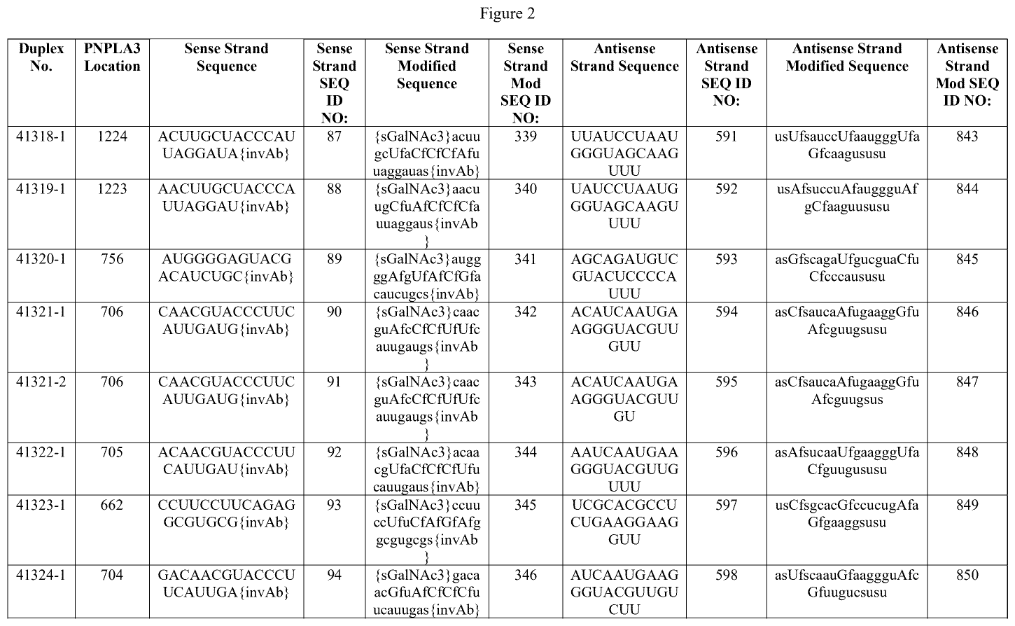

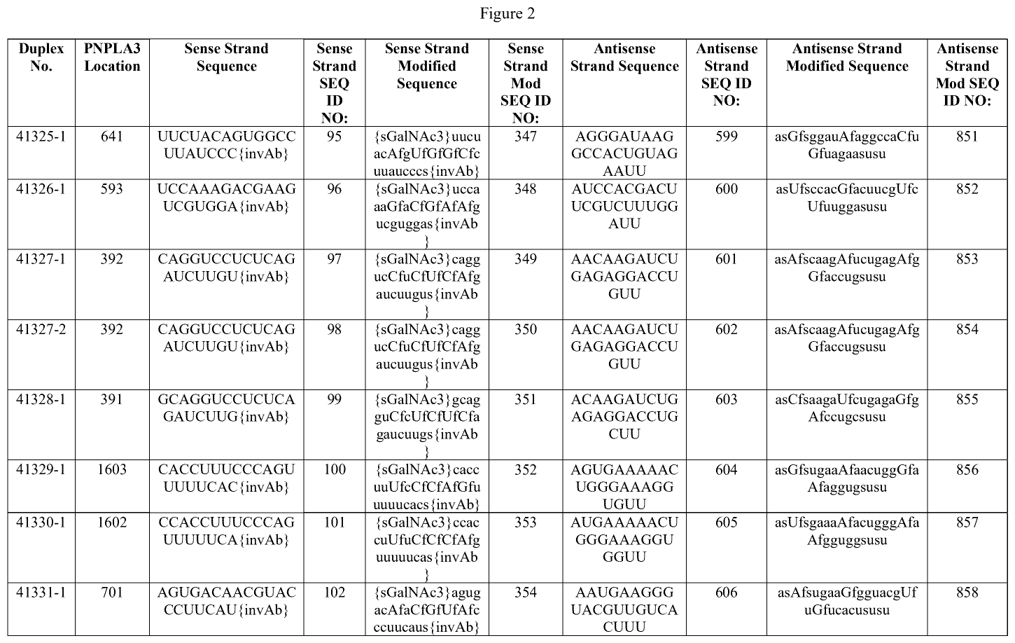

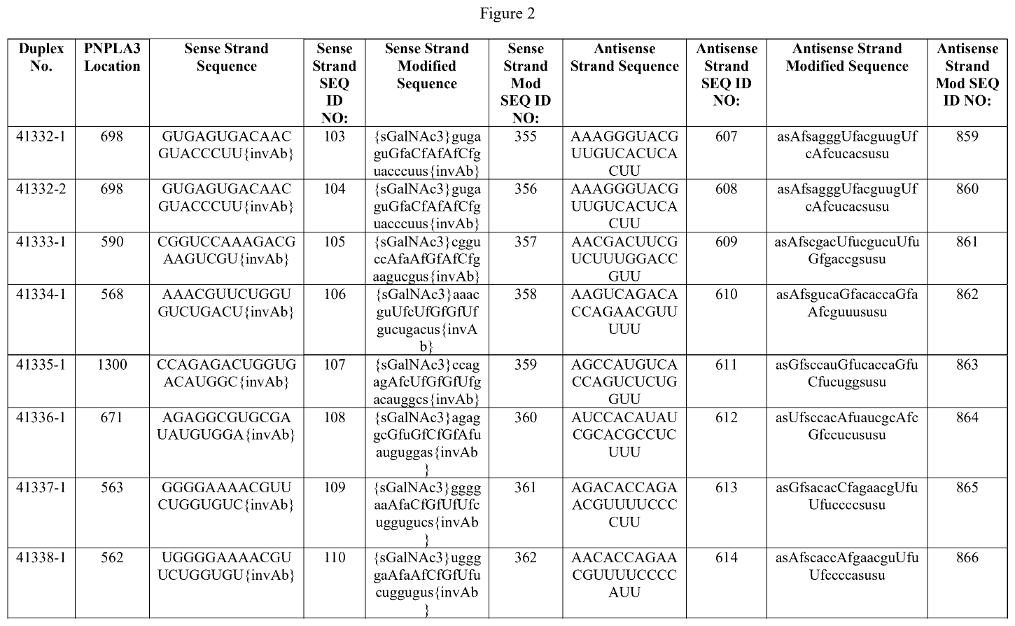

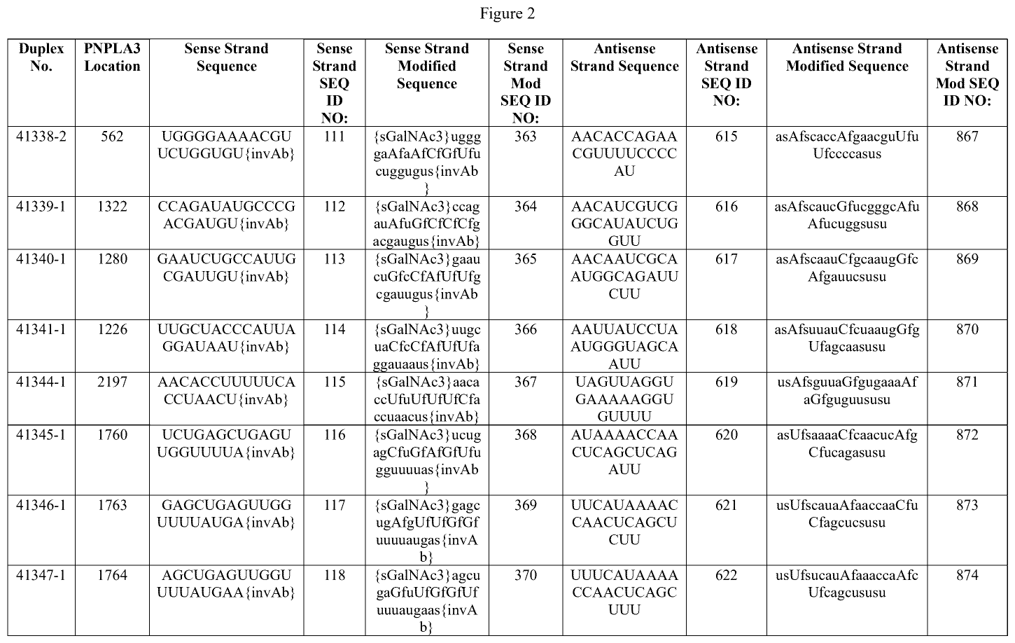

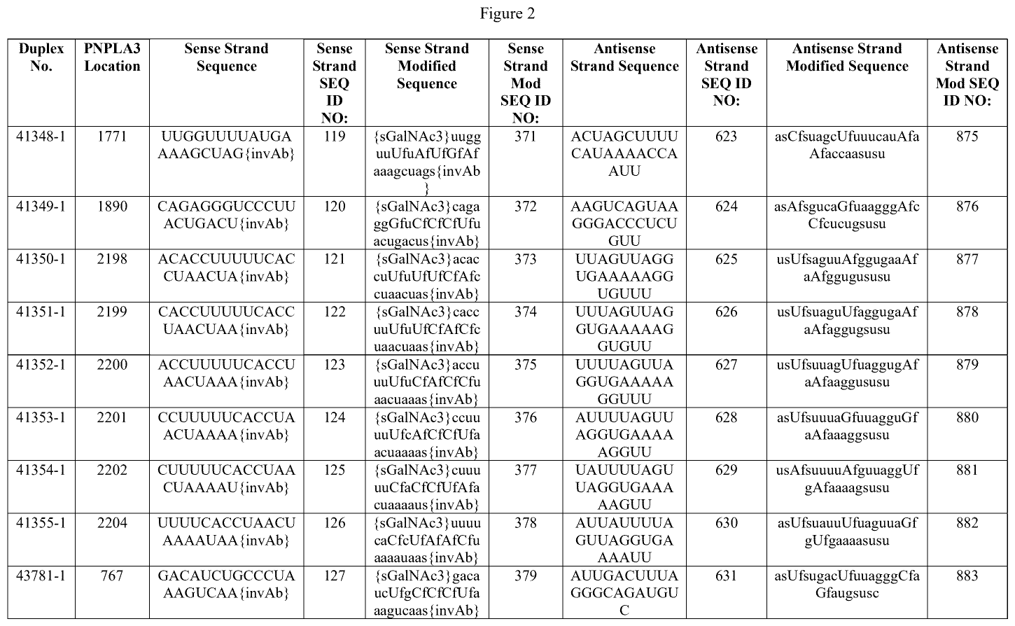

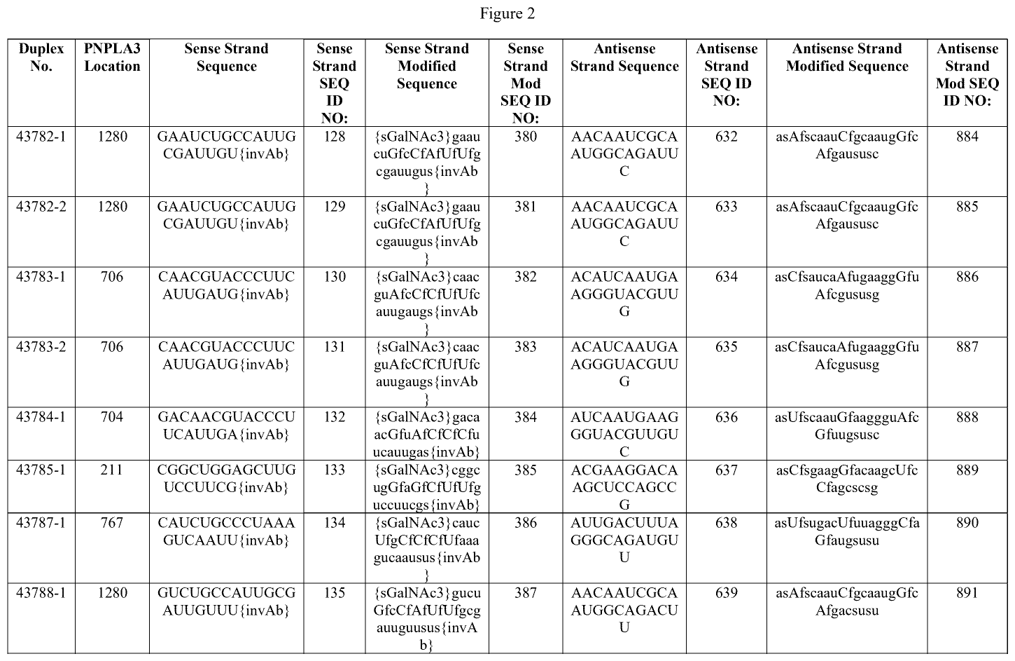

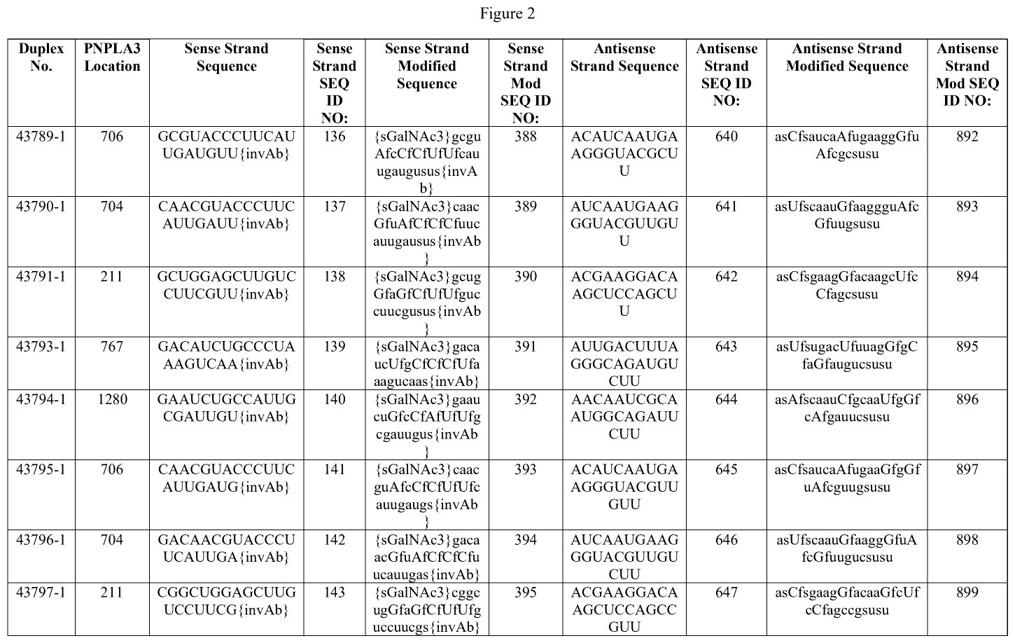

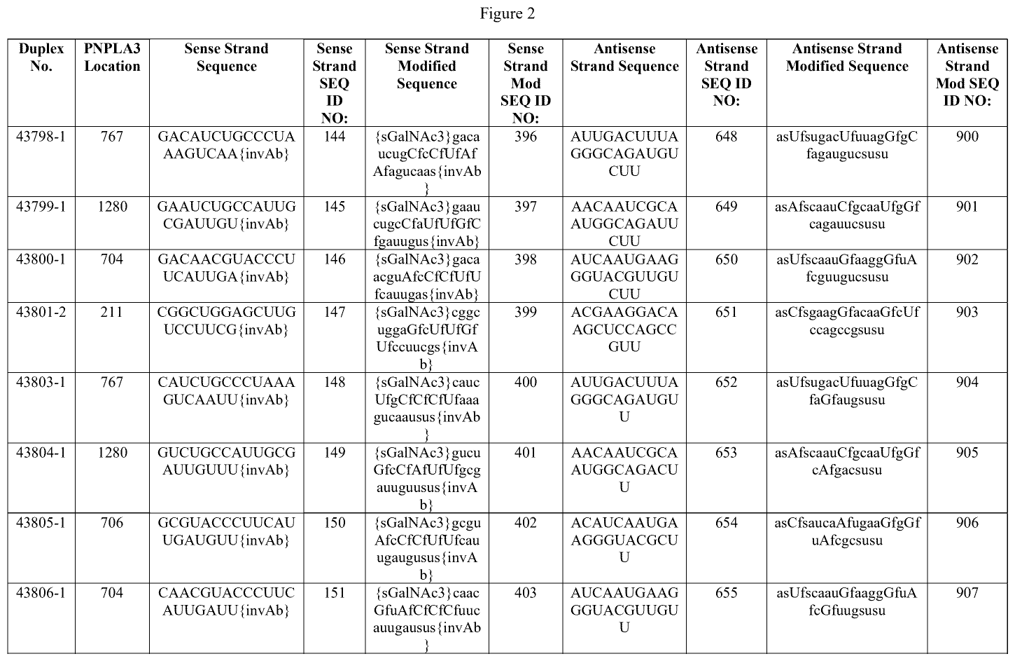

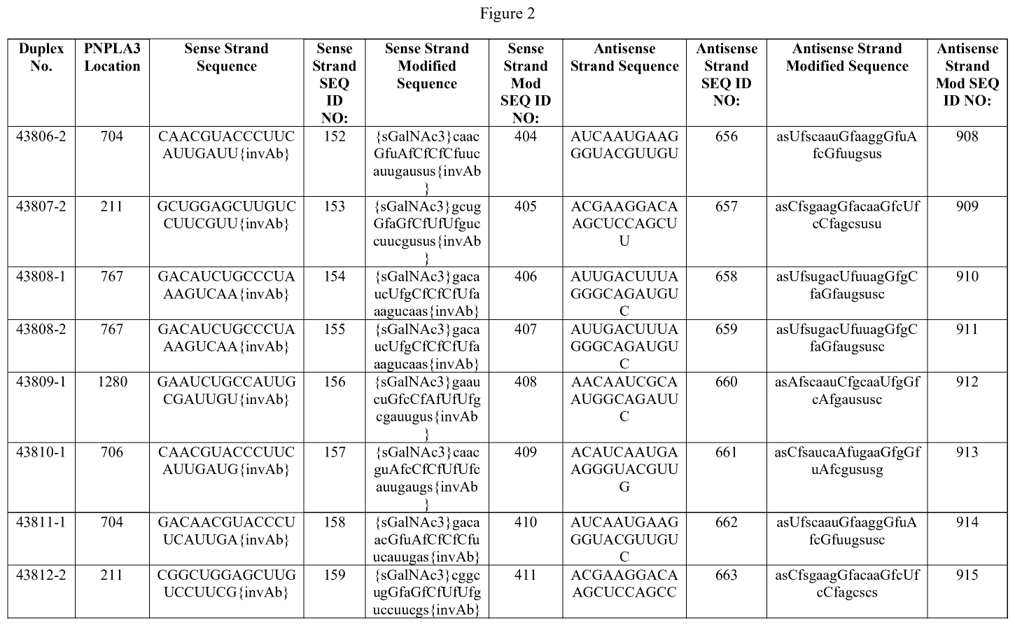

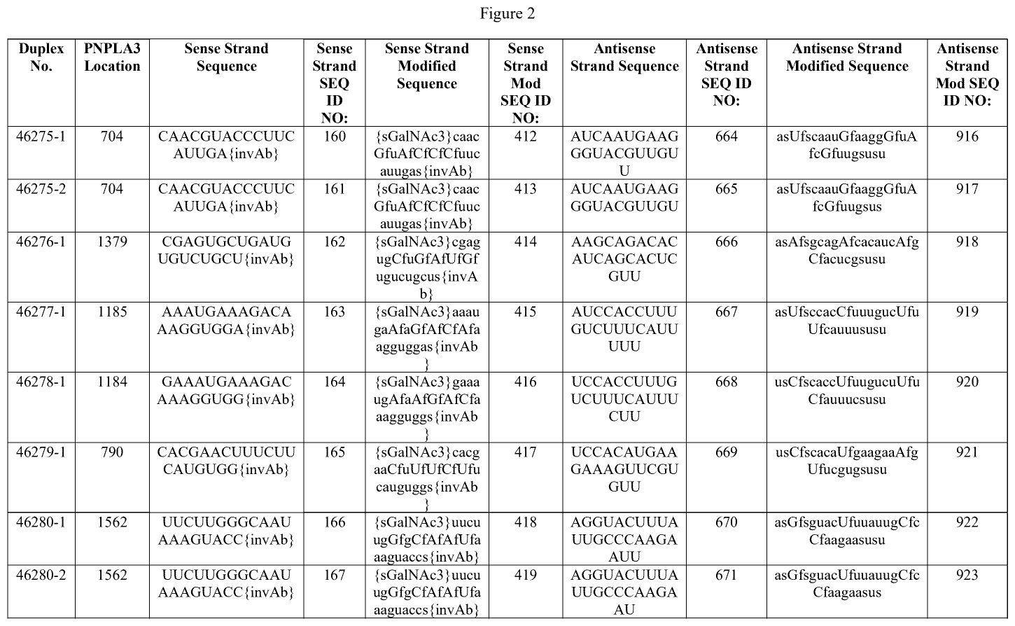

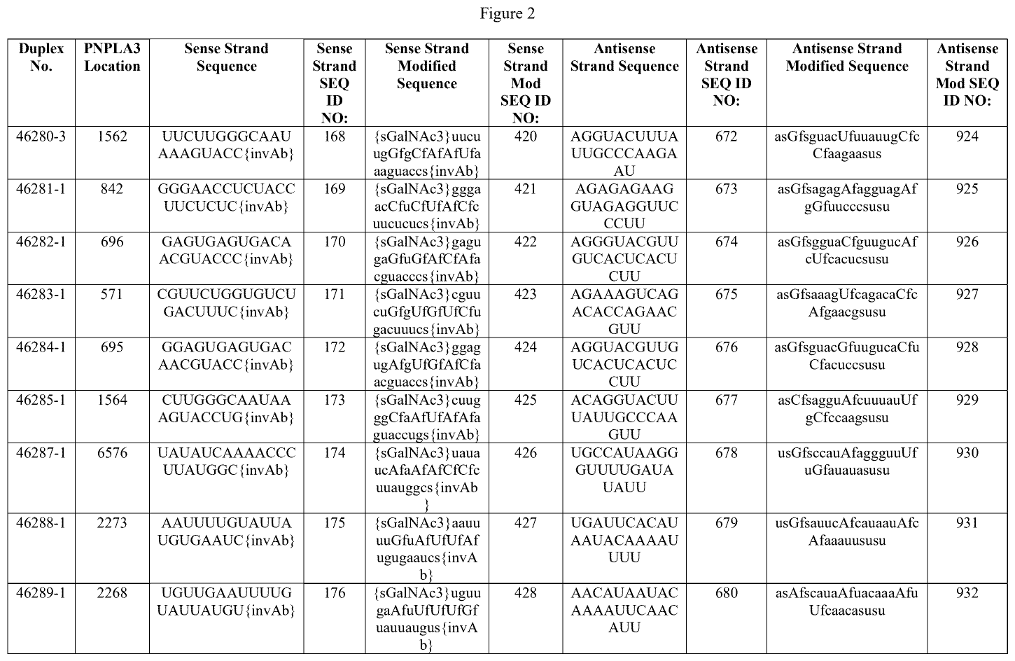

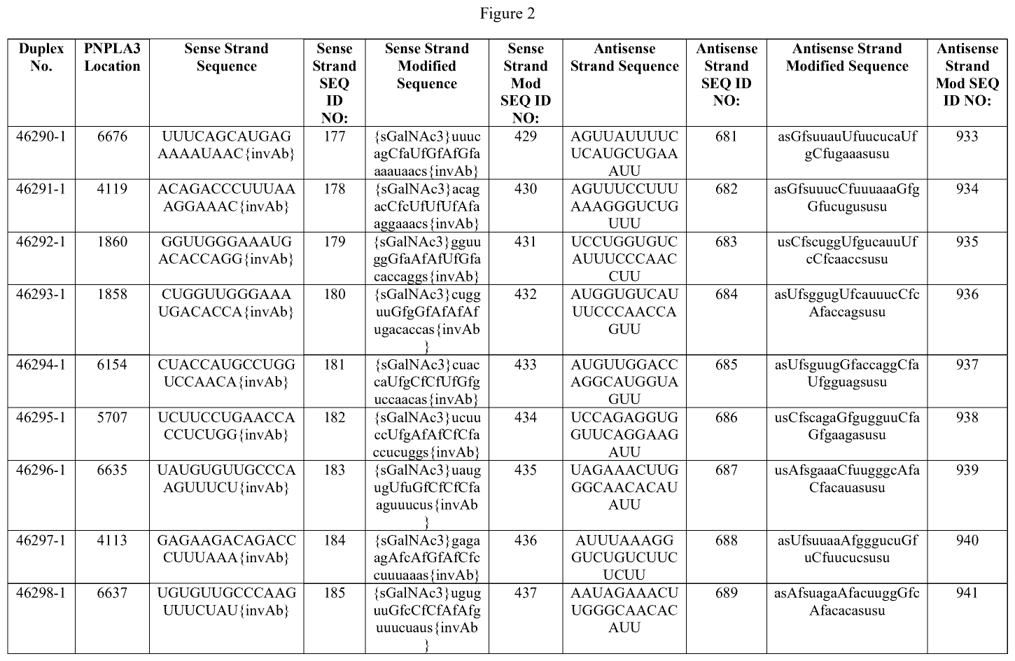

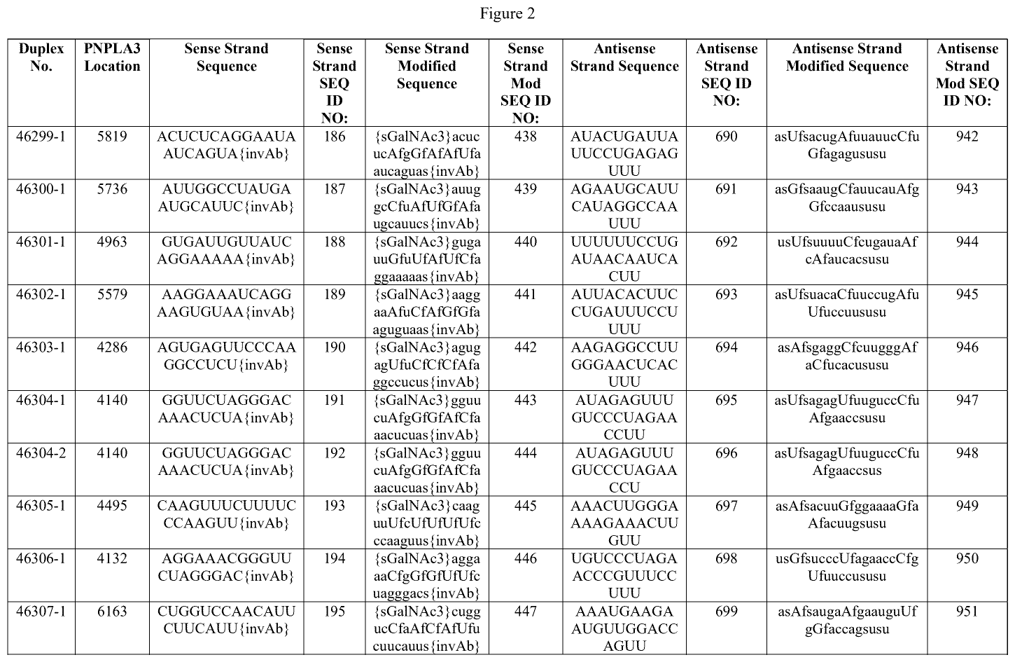

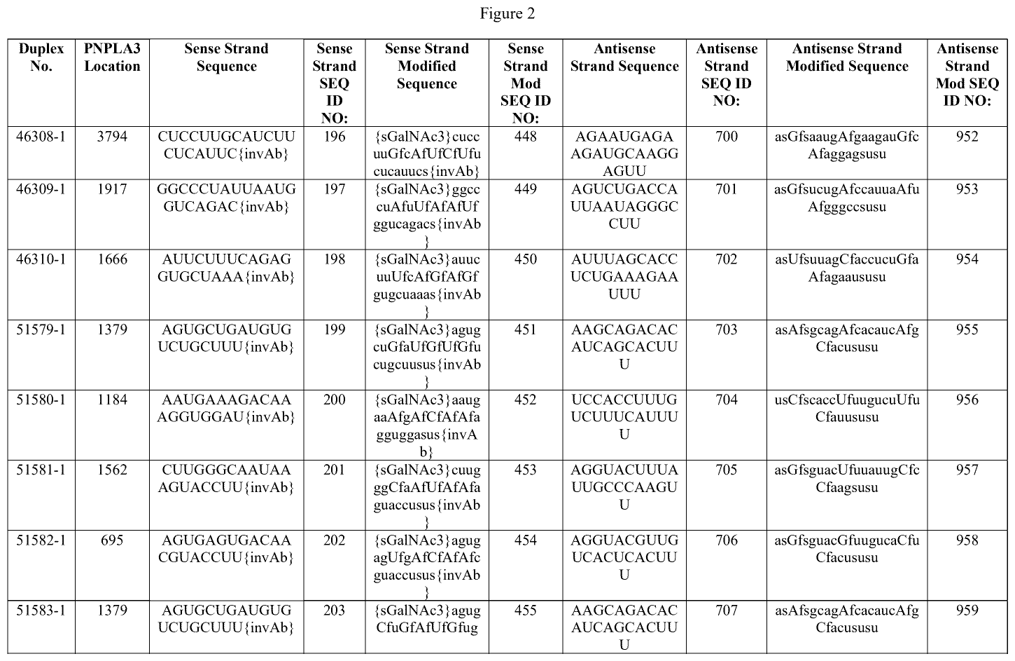

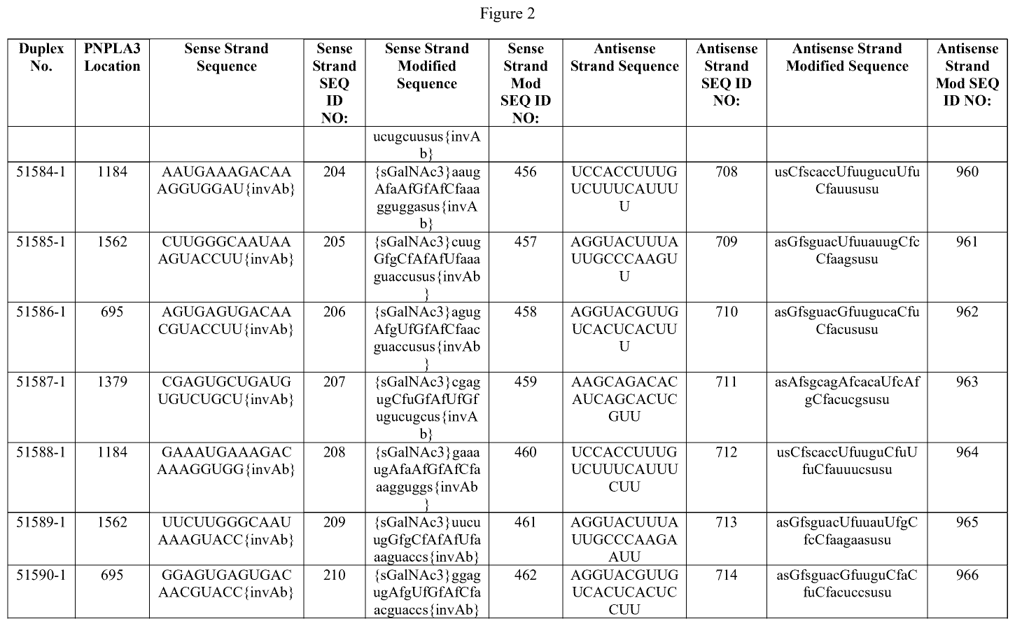

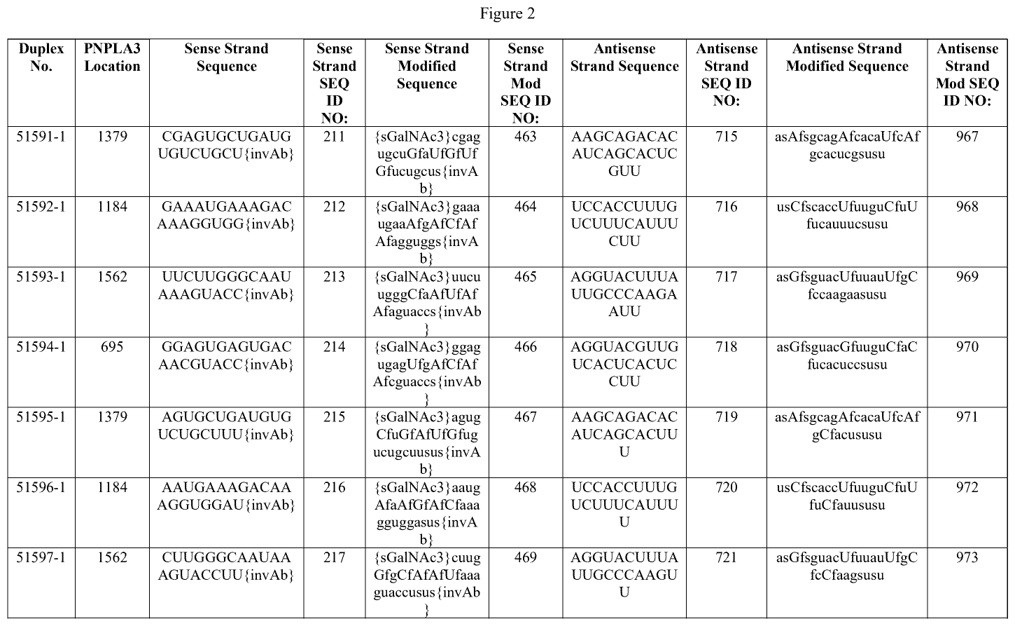

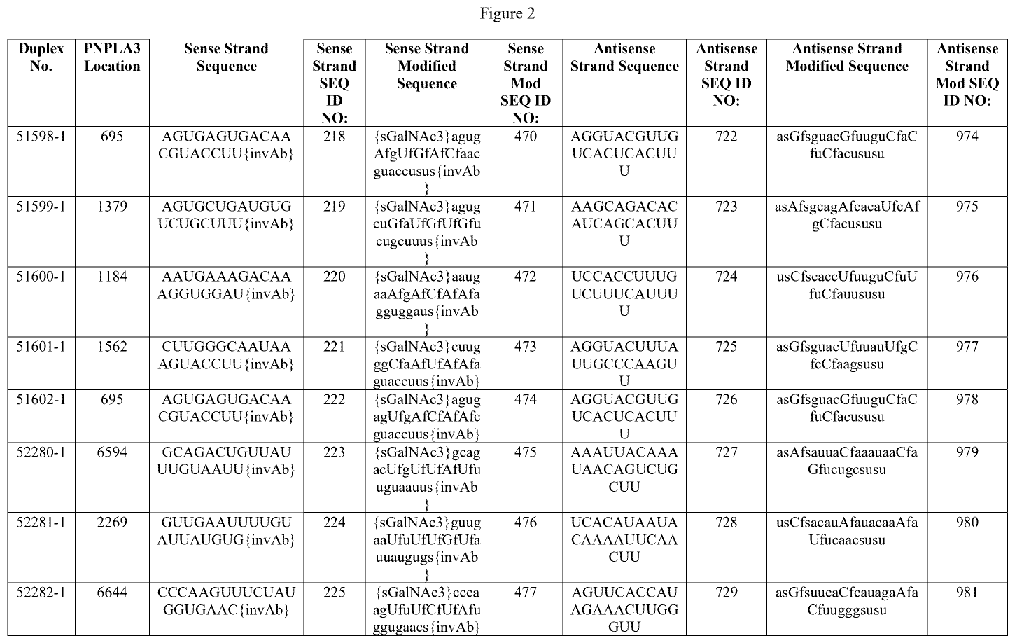

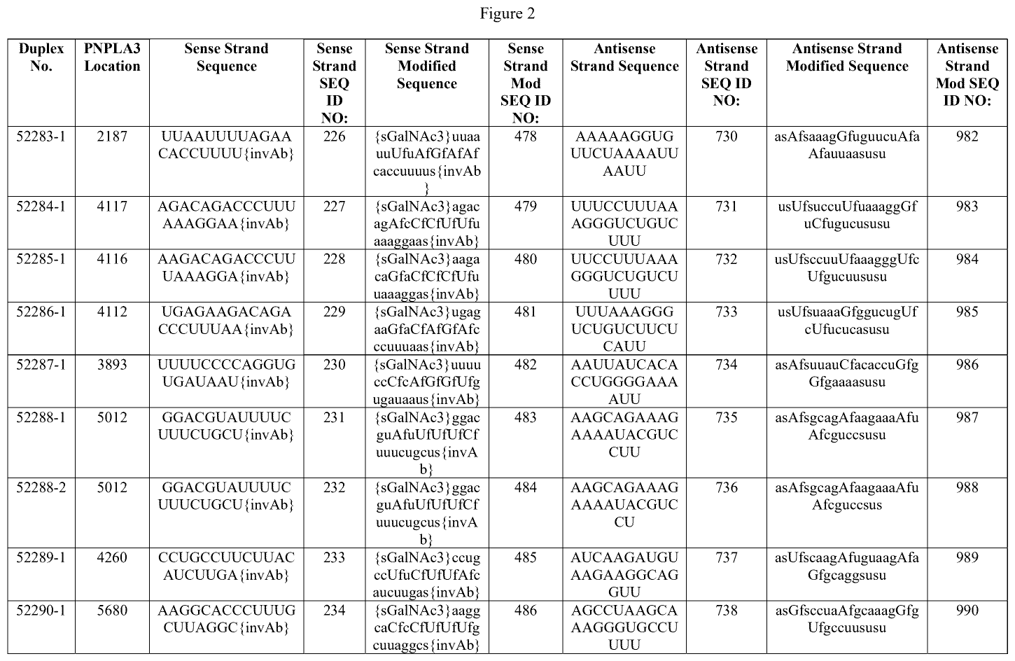

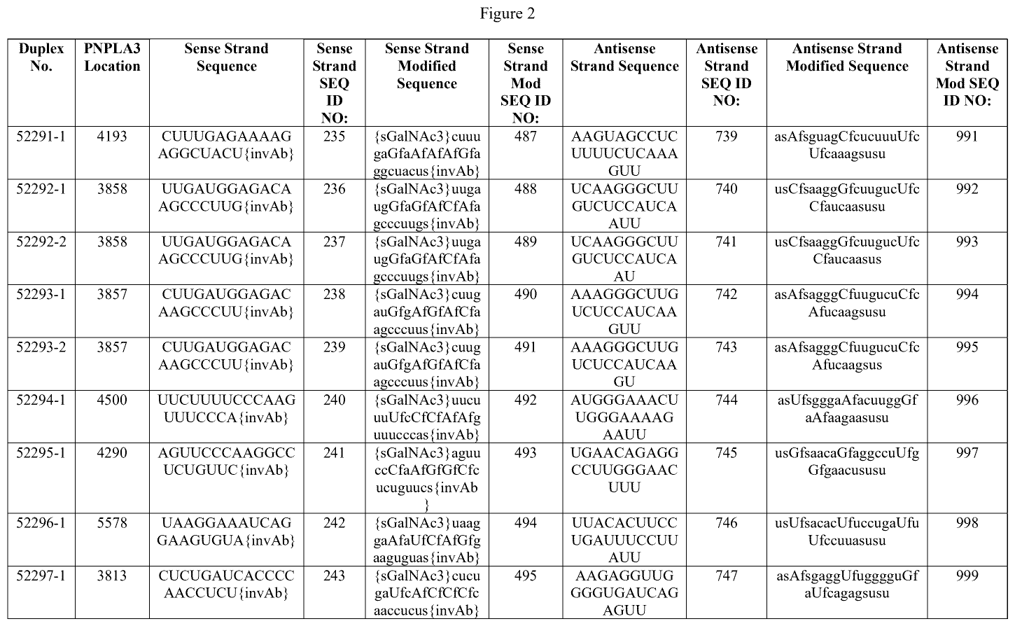

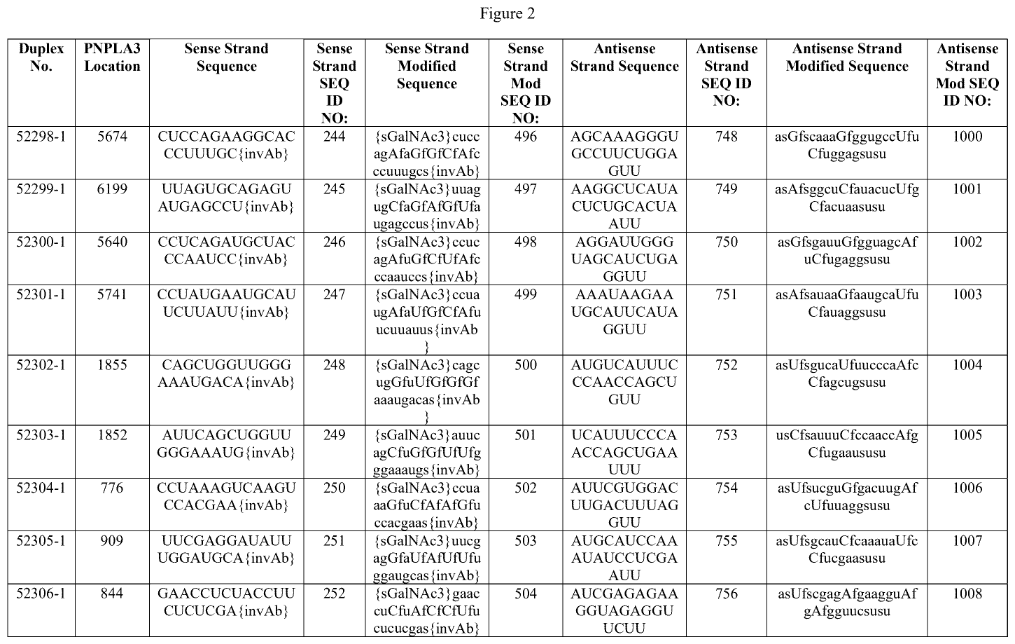

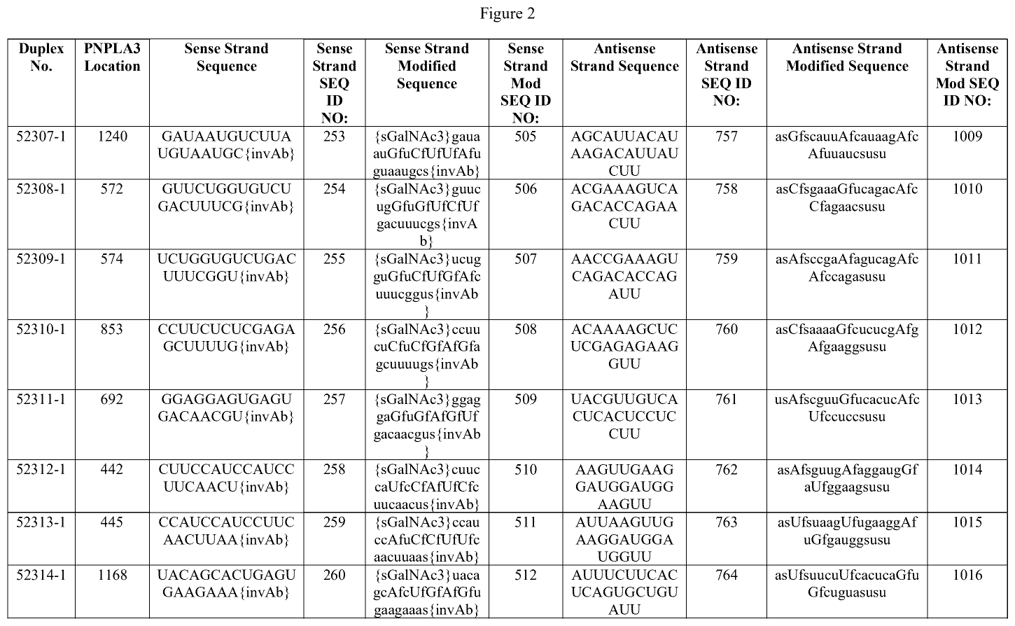

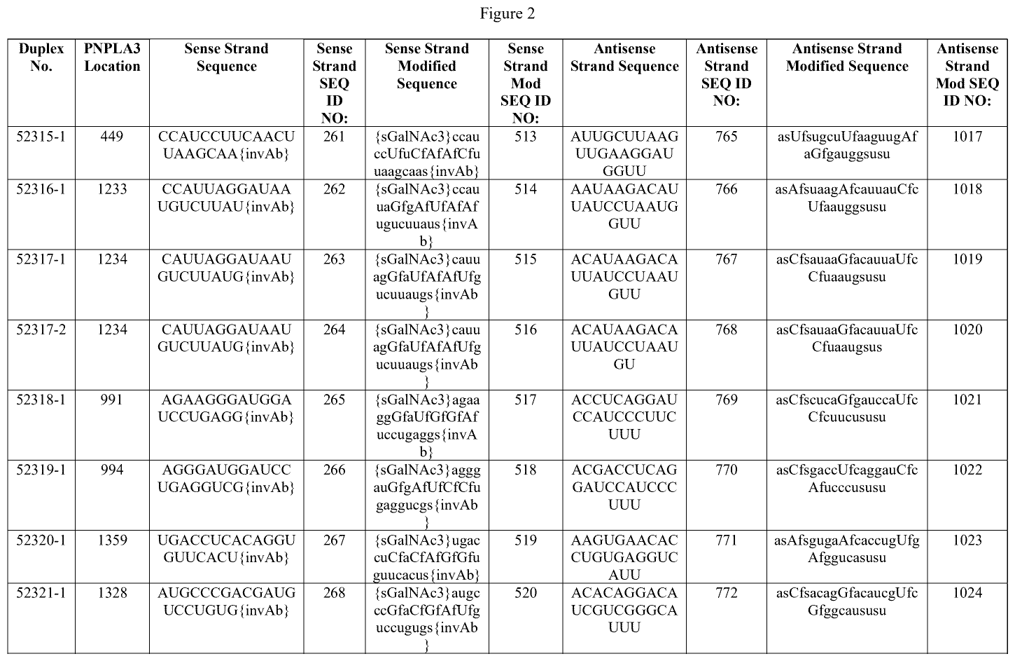

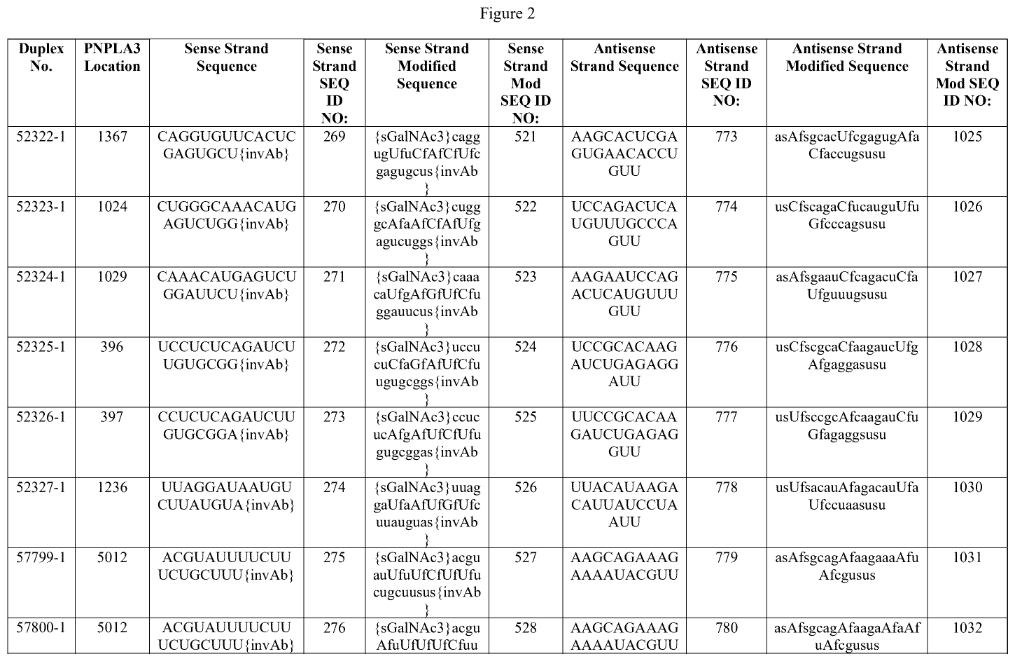

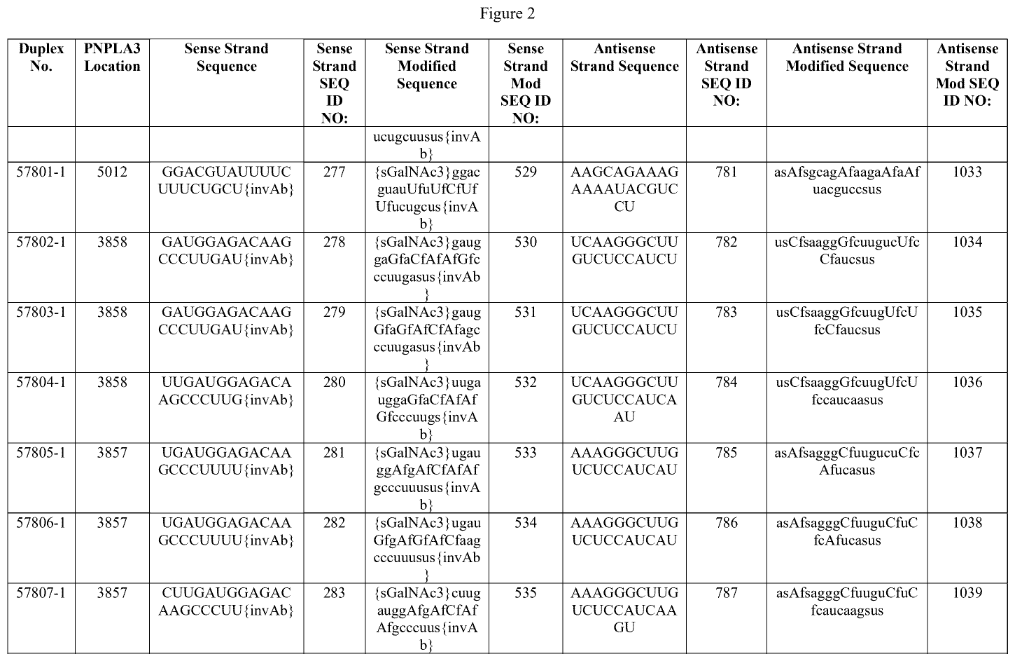

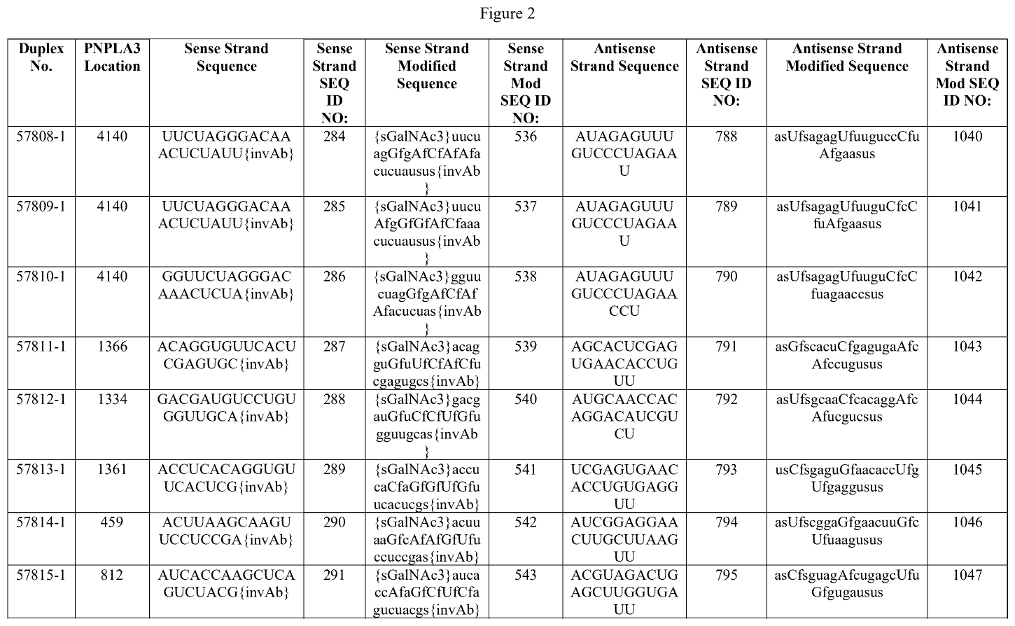

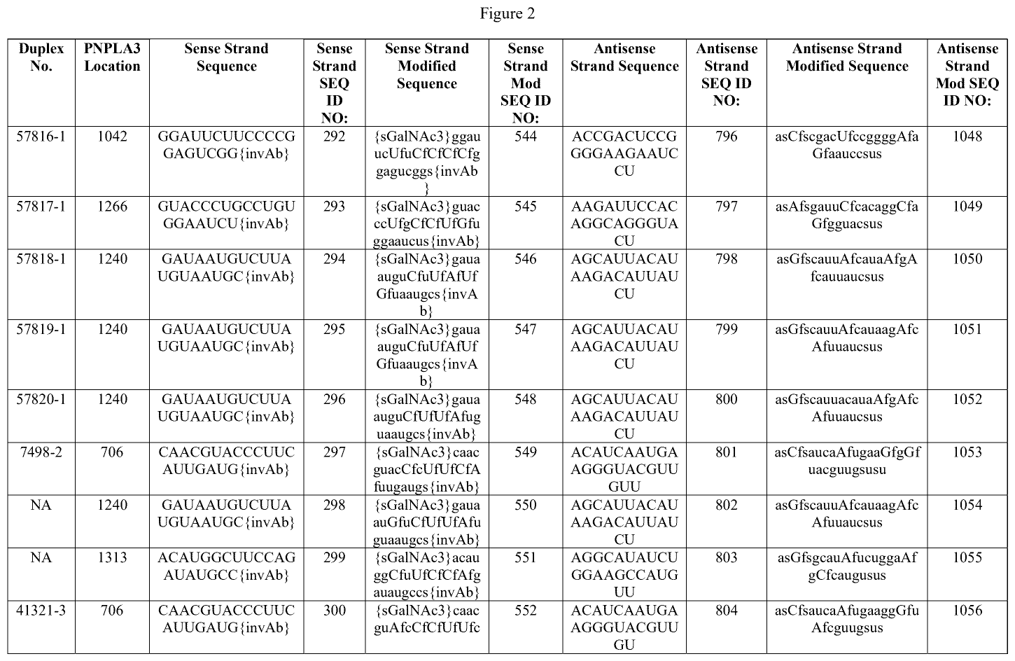

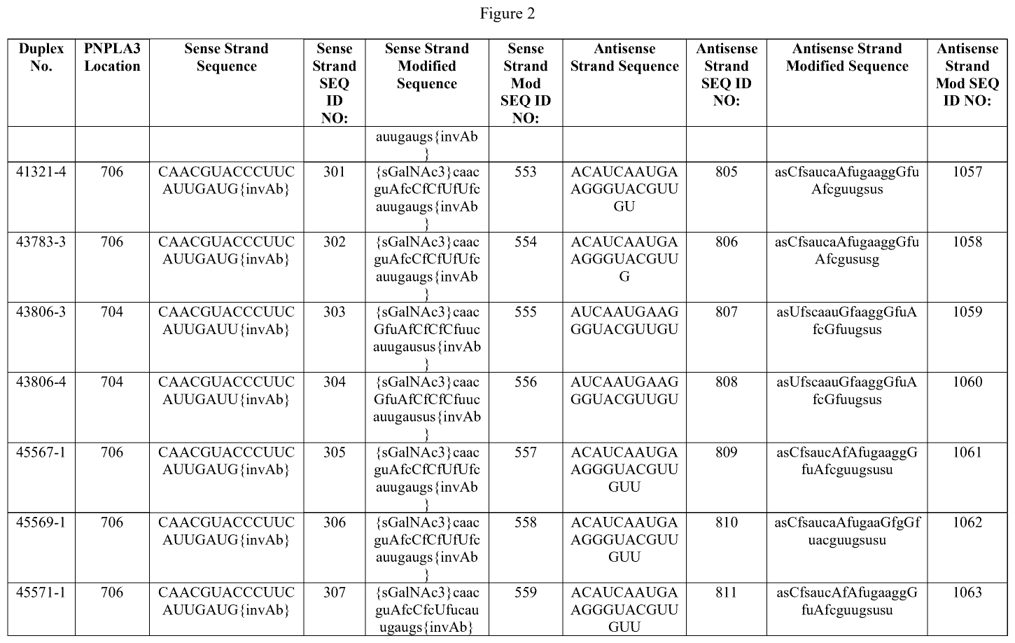

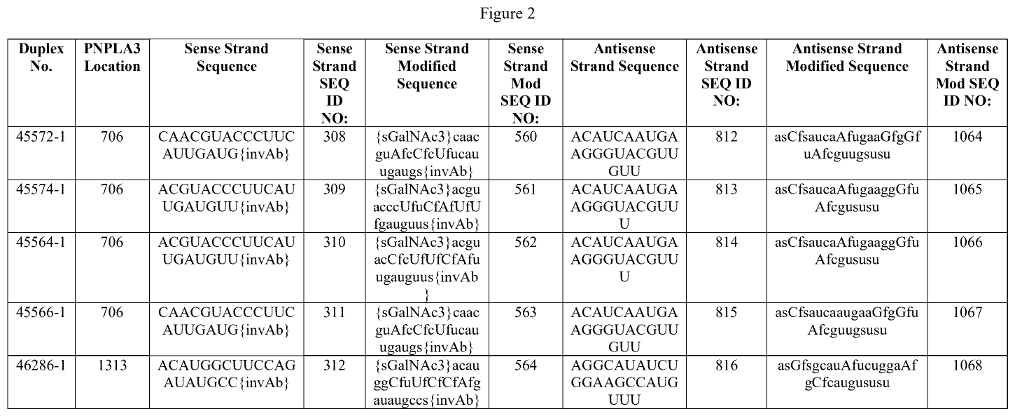

The disclosed RNAi construct is not required to hybridize to a particular PNPLA3 SNP. In such aspects, the RNAi construct may bind to the 3′ UTR of PNPLA3. As discussed above, non-sequence specific PNPLA3 RNAi may have broader therapeutic applications for diseases associated with PNPLA3 expression, including for example, simple fatty liver (steatosis), nonalcoholic steatohepatitis (NASH), cirrhosis (irreversible, advanced scarring of the liver), or PNPLA3-related obesity. In some embodiments, the RNAi construct is an siRNA molecule that contains any of the sequences set forth in FIG. 1 or 2 (e.g., an antisense strand comprising a sequence selected from SEQ ID NOs: 565-1068 and SEQ ID NOs: 2329-3588 and/or a sense strand comprising a sequence selected from SEQ ID NOs: 61-564 and SEQ ID NOs: 1069-2328).

A double-stranded RNAi molecule may include chemical modifications to ribonucleotides, including modifications to the ribose sugar, base, or backbone components of the ribonucleotides, such as those described herein or known in the art. Any such modifications, as used in a double-stranded RNA molecule (e.g. siRNA, shRNA, or the like), are encompassed by the term “double-stranded RNA” for the purposes of this disclosure.

As used herein, a first sequence is “complementary” to a second sequence if a polynucleotide comprising the first sequence can hybridize to a polynucleotide comprising the second sequence to form a duplex region under certain conditions, such as physiological conditions. Other such conditions can include moderate or stringent hybridization conditions, which are known to those of skill in the art. A first sequence is considered to be fully complementary (100% complementary) to a second sequence if a polynucleotide comprising the first sequence base pairs with a polynucleotide comprising the second sequence over the entire length of one or both nucleotide sequences without any mismatches. A sequence is “substantially complementary” to a target sequence if the sequence is at least about 80%, 85%, 90%, 95%, 96%, 97%, 98%, 99% or 100% complementary to a target sequence. Percent complementarity can be calculated by dividing the number of bases in a first sequence that are complementary to bases at corresponding positions in a second or target sequence by the total length of the first sequence. A sequence may also be said to be substantially complementary to another sequence if there are no more than 5, 4, 3, 2, or 1 mismatch over a 30 base pair duplex region when the two sequences are hybridized. Generally, if any nucleotide overhangs, as defined herein, are present, the sequence of such overhangs is not considered in determining the degree of complementarity between two sequences. By way of example, a sense strand of 21 nucleotides in length and an antisense strand of 21 nucleotides in length that hybridize to form a 19 base pair duplex region with a 2-nucleotide overhang at the 3′ end of each strand would be considered to be fully complementary as the term is used herein.

In some embodiments, a region of the antisense strand comprises a sequence that is fully complementary to a region of the target RNA sequence (e.g. PNPLA3 mRNA). In such embodiments, the sense strand may comprise a sequence that is fully complementary to the sequence of the antisense strand. In other such embodiments, the sense strand may comprise a sequence that is substantially complementary to the sequence of the antisense strand, e.g., having 1, 2, 3, 4, or 5 mismatches in the duplex region formed by the sense and antisense strands. In certain embodiments, it is preferred that any mismatches occur within the terminal regions (e.g. within 6, 5, 4, 3, 2, or 1 nucleotides of the 5′ and/or 3′ ends of the strands). In one embodiment, any mismatches in the duplex region formed from the sense and antisense strands desirably occur within 6, 5, 4, 3, 2, or 1 nucleotides of the 5′ end of the antisense strand.

Where the two substantially complementary strands of a dsRNA are comprised of separate RNA molecules, those molecules need not, but can be, covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs in the duplex is the number of nucleotides in the shortest strand of the dsRNA minus any overhangs that are present in the duplex. In addition to the duplex structure, an RNAi may comprise one or more nucleotide overhangs.

In other embodiments, the sense strand and the antisense strand that hybridize to form a duplex region may be part of a single RNA molecule, i.e., the sense and antisense strands are part of a self-complementary region of a single RNA molecule. In such cases, a single RNA molecule comprises a duplex region (also referred to as a stem region) and a loop region. The 3′ end of the sense strand is connected to the 5′ end of the antisense strand by a contiguous sequence of unpaired nucleotides, which will form the loop region. The loop region is typically of a sufficient length to allow the RNA molecule to fold back on itself such that the antisense strand can base pair with the sense strand to form the duplex or stem region. The loop region can comprise from about 3 to about 25, from about 5 to about 15, or from about 8 to about 12 unpaired nucleotides. As noted herein, such RNA molecules with at least partially self-complementary regions are referred to as “short hairpin RNAs” (shRNAs). In some embodiments, the loop region can comprise at least 1, 2, 3, 4, 5, 10, 20, or 25 unpaired nucleotides. In other embodiments, the loop region can have 10, 9, 8, 7, 6, 5, 4, 3, 2, or fewer unpaired nucleotides. In certain embodiments, the RNAi constructs of the disclosure comprise an shRNA. The length of a single, at least partially self-complementary RNA molecule can be from about 35 nucleotides to about 100 nucleotides, from about 45 nucleotides to about 85 nucleotides, or from about 50 to about 60 nucleotides and comprise a duplex region and loop region each having the lengths recited herein.

In some embodiments, the RNAi constructs of the disclosure comprise a sense strand and an antisense strand, wherein the antisense strand comprises a region having a sequence that is substantially or fully complementary to a PNPLA3 messenger RNA (mRNA) sequence. As used herein, a “PNPLA3 mRNA sequence” refers to any messenger RNA sequence, including splice variants, encoding a PNPLA3 protein, including PNPLA3 protein variants or isoforms from any species (e.g. mouse, rat, non-human primate, human). PNPLA3 protein is also known as adiponutrin (ADPN) and calcium-independent phospholipase A2-epsilon (iPLA(2)ε)).

A PNPLA3 mRNA sequence also includes the transcript sequence expressed as its complementary DNA (cDNA) sequence. A cDNA sequence refers to the sequence of an mRNA transcript expressed as DNA bases (e.g. guanine, adenine, thymine, and cytosine) rather than RNA bases (e.g. guanine, adenine, uracil, and cytosine). Thus, the antisense strand of the RNAi constructs of the disclosure may comprise a region having a sequence that is substantially or fully complementary to a target PNPLA3 mRNA sequence or PNPLA3 cDNA sequence. A PNPLA3 mRNA or cDNA sequence can include, but is not limited to, any PNPLA3 mRNA or cDNA sequence such as can be derived from the NCBI Reference sequence NM_025225.2.

A region of the antisense strand can be substantially complementary or fully complementary to at least 15 consecutive nucleotides of the PNPLA3 mRNA sequence. In some embodiments, the target region of the PNPLA3 mRNA sequence to which the antisense strand comprises a region of complementarity can range from about 15 to about 30 consecutive nucleotides, from about 16 to about 28 consecutive nucleotides, from about 18 to about 26 consecutive nucleotides, from about 17 to about 24 consecutive nucleotides, from about 19 to about 25 consecutive nucleotides, from about 19 to about 23 consecutive nucleotides, or from about 19 to about 21 consecutive nucleotides. In certain embodiments, the region of the antisense strand comprising a sequence that is substantially or fully complementary to a PNPLA3 mRNA sequence may, in some embodiments, comprise at least 15 contiguous nucleotides from an antisense sequence selected from SEQ ID NOs: 565-1068 and SEQ ID NOs: 2329-3588 (see FIG. 1 or 2). In other embodiments, the antisense sequence comprises at least 16, at least 17, at least 18, or at least 19 contiguous nucleotides from an antisense sequence selected from SEQ ID NOs: 565-1068 and SEQ ID NOs: 2329-3588. In some embodiments, the sense and/or antisense sequence comprises at least 15 nucleotides from a sequence listed in FIG. 1 or 2 with no more than 1, 2, or 3 nucleotide mismatches.

The sense strand of the RNAi construct typically comprises a sequence that is sufficiently complementary to the sequence of the antisense strand such that the two strands hybridize under physiological conditions to form a duplex region. A “duplex region” refers to the region in two complementary or substantially complementary polynucleotides that form base pairs with one another, either by Watson-Crick base pairing or other hydrogen bonding interaction, to create a duplex between the two polynucleotides. The duplex region of the RNAi construct should be of sufficient length to allow the RNAi construct to enter the RNA interference pathway, e.g. by engaging the Dicer enzyme and/or the RISC complex (described below). For instance, in some embodiments, the duplex region is about 15 to about 30 base pairs in length. Other lengths for the duplex region within this range are also suitable, such as about 15 to about 28 base pairs, about 15 to about 26 base pairs, about 15 to about 24 base pairs, about 15 to about 22 base pairs, about 17 to about 28 base pairs, about 17 to about 26 base pairs, about 17 to about 24 base pairs, about 17 to about 23 base pairs, about 17 to about 21 base pairs, about 19 to about 25 base pairs, about 19 to about 23 base pairs, about 20 to about 25 base pairs, or about 19 to about 21 base pairs. In one embodiment, the duplex region is about 17 to about 24 base pairs in length. In another embodiment, the duplex region is about 19 to about 21 base pairs in length.

In some embodiments, an siRNA construct of the disclosure contains a duplex region of about 18 to about 30 nucleotides that interacts with a target RNA sequence, e.g., a PNPLA3 target mRNA sequence, to direct the cleavage of the target RNA. For example, the siRNA may comprise a duplex region of about 19, 20, 21, 22, 23, 24, 25, 26, 27, 28, or 29 nucleotides that interacts with a PNPLA3 target mRNA sequence. Without wishing to be bound by theory, long double-stranded RNA introduced into cells can be broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15: 188).

For embodiments in which the sense strand and antisense strand are two separate molecules (e.g., an siRNA RNAi construct), the sense strand and antisense strand need not be the same length as the length of the duplex region. For instance, one or both strands maybe longer than the duplex region and have one or more unpaired nucleotides or mismatches flanking the duplex region. Thus, in some embodiments, the RNAi construct comprises at least one nucleotide overhang. As used herein, a “nucleotide overhang” refers to the unpaired nucleotide or nucleotides that extend beyond the duplex region at the terminal ends of the strands. Nucleotide overhangs are typically created when the 3′ end of one strand extends beyond the 5′ end of the other strand or when the 5′ end of one strand extends beyond the 3′ end of the other strand. The length of a nucleotide overhang is generally between 1 and 6 nucleotides, 1 and 5 nucleotides, 1 and 4 nucleotides, 1 and 3 nucleotides, 2 and 6 nucleotides, 2 and 5 nucleotides, or 2 and 4 nucleotides. In some embodiments, the nucleotide overhang comprises 1, 2, 3, 4, 5, or 6 nucleotides. In one particular embodiment, the nucleotide overhang comprises 1 to 4 nucleotides. In certain embodiments, the nucleotide overhang comprises 2 nucleotides. The nucleotides in the overhang can be ribonucleotides, deoxyribonucleotides, or modified nucleotides as described herein. In some embodiments, the overhang comprises a 5′-uridineuridine-3′ (5′-UU-3′) dinucleotide. In such embodiments, the UU dinucleotide may comprise ribonucleotides or modified nucleotides, e.g., 2′-modified nucleotides. In other embodiments, the overhang comprises a 5′-deoxythymidine-deoxythymidine-3′ (5′-dTdT-3′) dinucleotide.

The nucleotide overhang can be at the 5′ end or 3′ end of one or both strands. For example, in one embodiment, the RNAi construct comprises a nucleotide overhang at the 5′ end and the 3′ end of the antisense strand. In another embodiment, the RNAi construct comprises a nucleotide overhang at the 5′ end and the 3′ end of the sense strand. In some embodiments, the RNAi construct comprises a nucleotide overhang at the 5′ end of the sense strand and the 5′ end of the antisense strand. In other embodiments, the RNAi construct comprises a nucleotide overhang at the 3′ end of the sense strand and the 3′ end of the antisense strand.

The RNAi constructs may comprise a single nucleotide overhang at one end of the double-stranded RNA molecule and a blunt end at the other. A “blunt end” means that the sense strand and antisense strand are fully base-paired at the end of the molecule and there are no unpaired nucleotides that extend beyond the duplex region. In some embodiments, the RNAi construct comprises a nucleotide overhang at the 3′ end of the sense strand and a blunt end at the 5′ end of the sense strand and 3′ end of the antisense strand. In other embodiments, the RNAi construct comprises a nucleotide overhang at the 3′ end of the antisense strand and a blunt end at the 5′ end of the antisense strand and the 3′ end of the sense strand. In certain embodiments, the RNAi construct comprises a blunt end at both ends of the double-stranded RNA molecule. In such embodiments, the sense strand and antisense strand have the same length and the duplex region is the same length as the sense and antisense strands (i.e., the molecule is double-stranded over its entire length).

The sense strand and antisense strand can each independently be any suitable length, such as about 15 to about 30 nucleotides in length, about 18 to about 28 nucleotides in length, about 19 to about 27 nucleotides in length, about 19 to about 25 nucleotides in length, about 19 to about 23 nucleotides in length, about 20 to about 25 nucleotides in length, or about 21 to about 23 nucleotides in length. In certain embodiments, the sense strand and antisense strand are each about 18, about 19, about 20, about 21, about 22, about 23, about 24, or about 25 nucleotides in length. In some embodiments, the sense strand and antisense strand are of the same length but form a duplex region that is shorter than the strands such that the RNAi construct has two nucleotide overhangs. For instance, in one embodiment, the RNAi construct comprises (i) a sense strand and an antisense strand that are each 21 nucleotides in length, (ii) a duplex region that is 19 base pairs in length, and (iii) nucleotide overhangs of 2 unpaired nucleotides at both the 3′ end of the sense strand and the 3′ end of the antisense strand. In another embodiment, the RNAi construct comprises (i) a sense strand and an antisense strand that are each 23 nucleotides in length, (ii) a duplex region that is 21 base pairs in length, and (iii) nucleotide overhangs of 2 unpaired nucleotides at both the 3′ end of the sense strand and the 3′ end of the antisense strand. In other embodiments, the sense strand and antisense strand have the same length and form a duplex region over their entire length such that there are no nucleotide overhangs on either end of the double-stranded molecule. In one such embodiment, the RNAi construct is blunt ended and comprises (i) a sense strand and an antisense strand, each of which is 21 nucleotides in length, and (ii) a duplex region that is 21 base pairs in length. In another embodiment, the RNAi construct is blunt ended and comprises (i) a sense strand and an antisense strand, each of which is 23 nucleotides in length, and (ii) a duplex region that is 23 base pairs in length.

In other embodiments, the sense strand or the antisense strand is longer than the other strand and the two strands form a duplex region having a length equal to that of the shorter strand such that the RNAi construct comprises at least one nucleotide overhang. For example, in one embodiment, the RNAi construct comprises (i) a sense strand that is 19 nucleotides in length, (ii) an antisense strand that is 21 nucleotides in length, (iii) a duplex region of 19 base pairs in length, and (iv) a single nucleotide overhang of 2 unpaired nucleotides at the 3′ end of the antisense strand. In another embodiment, the RNAi construct comprises (i) a sense strand that is 21 nucleotides in length, (ii) an antisense strand that is 23 nucleotides in length, (iii) a duplex region of 21 base pairs in length, and (iv) a single nucleotide overhang of 2 unpaired nucleotides at the 3′ end of the antisense strand.

The antisense strand of the RNAi constructs of the disclosure can comprise the sequence of any one of the antisense sequences listed in FIG. 1 or FIG. 2 (e.g., any one of SEQ ID NOs: 565-816 or SEQ ID NOs: 2329-2958), or the sequence of nucleotides 1-21 of any of these antisense sequences. Each of the antisense sequences of SEQ ID NOs: 565-816 and SEQ ID NOs: 2329-2958 comprises a sequence of 20 or 21 consecutive nucleotides (first 20 or 21 nucleotides counting from the 5′ end) that is complementary to a PNPLA3 mRNA sequence, plus an optional two-nucleotide overhang sequence. Thus, in some embodiments, the antisense strand comprises a sequence of nucleotides 1-20 or 1-21 of any one of SEQ ID NOs: 565-816 or SEQ ID NOs: 2329-2958.

### Modified Nucleotides

The RNAi constructs of the disclosure may comprise one or more modified nucleotides. A “modified nucleotide” refers to a nucleotide that has one or more chemical modifications to the nucleoside, nucleobase, pentose ring, or phosphate group. As used herein, modified nucleotides do not encompass ribonucleotides containing adenosine monophosphate, guanosine monophosphate, uridine monophosphate, and cytidine monophosphate, and deoxyribonucleotides containing deoxyadenosine monophosphate, deoxyguanosine monophosphate, deoxythymidine monophosphate, and deoxycytidine monophosphate. However, the RNAi constructs may comprise combinations of modified nucleotides, ribonucleotides, and deoxyribonucleotides. Incorporation of modified nucleotides into one or both strands of double-stranded RNA molecules can improve the in vivo stability of the RNA molecules, e.g., by reducing the molecules' susceptibility to nucleases and other degradation processes. The potency of RNAi constructs for reducing expression of the target gene can also be enhanced by incorporation of modified nucleotides.

In certain embodiments, the modified nucleotides have a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than H or OH. Such 2′ modifications include, but are not limited to, 2′-O-alkyl (e.g. O—C1-C10 or O—C1-C10 substituted alkyl), 2′-O-allyl (O—CH2CH═CH2), 2′-C-allyl, 2′-fluoro, 2′-O-methyl (OCH3), 2′-O-methoxyethyl (O—(CH2)2OCH3), 2′-OCF3, 2′-O(CH2)2SCH3, 2′-O-aminoalkyl, 2′-amino (e.g., NH2), 2′-O-ethylamine, and 2′-azido. Modifications at the 5′ position of the pentose ring include, but are not limited to, 5′-methyl (R or S); 5′-vinyl, and 5′-methoxy.

A “bicyclic sugar modification” refers to a modification of the pentose ring where a bridge connects two atoms of the ring to form a second ring resulting in a bicyclic sugar structure. In some embodiments, the bicyclic sugar modification comprises a bridge between the 4′ and 2′ carbons of the pentose ring. Nucleotides comprising a sugar moiety with a bicyclic sugar modification are referred to herein as “bicyclic nucleic acids” or “BNAs.” Exemplary bicyclic sugar modifications include, but are not limited to, α-L-Methyleneoxy (4′-CH₂—O-2′) bicyclicnucleic acid (BNA); s-D-Methyleneoxy (4′-CH₂—O-2′) BNA (also referred to as a locked nucleic acid or LNA); Ethyleneoxy (4′-(CH₂)2-O-2′) BNA; Aminooxy (4′-CH₂—O—N(R)-2′)BNA; Oxyamino (4′-CH₂—N(R)—O-2′) BNA; Methyl(methyleneoxy) (4′-CH(CH₃)—O-2′) BNA (also referred to as constrained ethyl or cEt); methylene-thio (4′-CH₂—S-2′) BNA; methylene-amino (4′-CH₂—N(R)-2′) BNA; methyl carbocyclic (4′-CH₂—CH(CH₃)-2′) BNA; propylene carbocyclic (4′-(CH₂)3-2′) BNA; and Methoxy(ethyleneoxy) (4′-CH(CH₂OMe)-O-2′)BNA (also referred to as constrained MOE or cMOE). These and other sugar-modified nucleotides that can be incorporated into the RNAi constructs of the disclosure are described in, e.g., U.S. Pat. No. 9,181,551, U.S. Patent Publication No. 2016/0122761, and Deleavey and Damha, Chemistry and Biology, 19: 937-954 (2012).

In some embodiments, the RNAi constructs comprise one or more 2′-fluoro modified nucleotides, 2′-O-methyl modified nucleotides, 2′-O-methoxyethyl modified nucleotides, 2′-O-allyl modified nucleotides, bicyclic nucleic acids (BNAs), or combinations thereof. In certain embodiments, the RNAi constructs comprise one or more 2′-fluoro modified nucleotides, 2′-O-methyl modified nucleotides, 2′-O-methoxyethyl modified nucleotides, or combinations thereof. In one particular embodiment, the RNAi constructs comprise one or more 2′-fluoro modified nucleotides, 2′-O-methyl modified nucleotides, or combinations thereof.

Both the sense and antisense strands of the RNAi constructs can comprise one or multiple modified nucleotides. For instance, in some embodiments, the sense strand comprises 1, 2, 3, 4, 5, 6, 7, 8, 9, 10 or more modified nucleotides. In certain embodiments, all nucleotides in the sense strand are modified nucleotides. In some embodiments, the antisense strand comprises 1, 2, 3, 4, 5, 6, 7, 8, 9, 10 or more modified nucleotides. In other embodiments, all nucleotides in the antisense strand are modified nucleotides. In certain other embodiments, all nucleotides in the sense strand and all nucleotides in the antisense strand are modified nucleotides. In these and other embodiments, the modified nucleotides can be 2′-fluoro modified nucleotides, 2′-O-methyl modified nucleotides, or combinations thereof.

In some embodiments, all pyrimidine nucleotides preceding an adenosine nucleotide in the sense strand and/or in the antisense strand are modified nucleotides. For example, where the sequence 5′-CA-3′ or 5′-UA-3′ appears in either strand, the cytidine and uridine nucleotides are modified nucleotides, preferably 2′-O-methyl modified nucleotides. In certain embodiments, all pyrimidine nucleotides in the sense strand are modified nucleotides (e.g. 2′-O-methyl modified nucleotides), and the 5′ nucleotide in all occurrences of the sequence 5′-CA-3′ or 5′-UA-3′ in the antisense strand are modified nucleotides (e.g. 2′-O-methyl modified nucleotides). In other embodiments, all nucleotides in the duplex region are modified nucleotides. In such embodiments, the modified nucleotides are preferably 2′-O-methyl modified nucleotides, 2′-fluoro modified nucleotides, or combinations thereof.

In embodiments in which the RNAi construct comprises a nucleotide overhang, the nucleotides in the overhang can be ribonucleotides, deoxyribonucleotides, or modified nucleotides. In one embodiment, the nucleotides in the overhang are deoxyribonucleotides, e.g., deoxythymidine. In another embodiment, the nucleotides in the overhang are modified nucleotides. For instance, in some embodiments, the nucleotides in the overhang are 2′-O-methyl modified nucleotides, 2′-fluoro modified nucleotides, 2′-methoxyethyl modified nucleotides, or combinations thereof.

The RNAi constructs of the disclosure may also comprise one or more modified internucleotide linkages. As used herein, the term “modified internucleotide linkage” refers to an internucleotide linkage other than the natural 3′ to 5′ phosphodiester linkage. In some embodiments, the modified internucleotide linkage is a phosphorous-containing internucleotide linkage, such as a phosphotriester, an aminoalkyl phosphotriester, an alkylphosphonate (e.g., methylphosphonate, 3′-alkylene phosphonate), a phosphinate, a phosphoramidate (e.g., 3′-aminophosphoramidate and aminoalkylphosphoramidate), a phosphorothioate (P═S), a chiralphosphorothioate, a phosphorodithioate, a thionophosphoramidate, a thionoalkylphosphonate, athionoalkylphosphotriester, and a boranophosphate. In one embodiment, a modified internucleotide linkage is a 2′ to 5′ phosphodiester linkage. In other embodiments, the modified internucleotide linkage is a non-phosphorous-containing internucleotide linkage and thus can be referred to as a modified internucleoside linkage. Such non-phosphorous-containing linkages include, but are not limited to, morpholino linkages (formed in part from the sugar portion of a nucleoside); siloxane linkages (—O—Si(H)₂—O—); sulfide, sulfoxide and sulfone linkages; formacetyl and thioformacetyl linkages; alkene containing backbones; sulfamate backbones; methylenemethylimino (—CH₂—N(CH₃)—O—CH₂—) and methylenehydrazino linkages; sulfonate and sulfonamide linkages; amide linkages; and others having mixed N, O, S and CH₂ component parts. In one embodiment, the modified internucleoside linkage is a peptide-based linkage (e.g., aminoethylglycine) to create a peptide nucleic acid or PNA, such as those described in U.S. Pat. Nos. 5,539,082; 5,714,331; and 5,719,262. Other suitable modified internucleotide and internucleoside linkages that may be employed in the disclosed RNAi constructs are described in U.S. Pat. Nos. 6,693,187 and 9,181,551, U.S. Patent Publication No. 2016/0122761, and Deleavey and Damha, supra.

In certain embodiments, the RNAi constructs comprise one or more phosphorothioate internucleotide linkages. The phosphorothioate internucleotide linkages may be present in the sense strand, antisense strand, or both strands of the RNAi constructs. For instance, in some embodiments, the sense strand comprises 1, 2, 3, 4, 5, 6, 7, 8, or more phosphorothioate internucleotide linkages. In other embodiments, the antisense strand comprises 1, 2, 3, 4, 5, 6, 7, 8, or more phosphorothioate internucleotide linkages. In still other embodiments, both strands comprise 1, 2, 3, 4, 5, 6, 7, 8, or more phosphorothioate internucleotide linkages. The RNAi constructs can comprise one or more phosphorothioate internucleotide linkages at the 3′-end, the 5′-end, or both the 3′- and 5′-ends of the sense strand, the antisense strand, or both strands. For instance, in certain embodiments, the RNAi construct comprises about 1 to about 6 or more (e.g., about 1, 2, 3, 4, 5, 6 or more) consecutive phosphorothioate internucleotide linkages at the 3′-end of the sense strand, the antisense strand, or both strands. In other embodiments, the RNAi construct comprises about 1 to about 6 or more (e.g., about 1, 2, 3, 4, 5, 6 or more) consecutive phosphorothioate internucleotide linkages at the 5′-end of the sense strand, the antisense strand, or both strands. In one embodiment, the RNAi construct comprises a single phosphorothioate internucleotide linkage at the 3′ end of the sense strand and a single phosphorothioate internucleotide linkage at the 3′ end of the antisense strand. In another embodiment, the RNAi construct comprises two consecutive phosphorothioate internucleotide linkages at the 3′ end of the antisense strand (i.e., a phosphorothioate internucleotide linkage at the first and second internucleotide linkages at the 3′ end of the antisense strand). In another embodiment, the RNAi construct comprises two consecutive phosphorothioate internucleotide linkages at both the 3′ and 5′ ends of the antisense strand. In yet another embodiment, the RNAi construct comprises two consecutive phosphorothioate internucleotide linkages at both the 3′ and 5′ ends of the antisense strand and two consecutive phosphorothioate internucleotide linkages at the 5′ end of the sense strand. In still another embodiment, the RNAi construct comprises two consecutive phosphorothioate internucleotide linkages at both the 3′ and 5′ ends of the antisense strand and two consecutive phosphorothioate internucleotide linkages at both the 3′ and 5′ ends of the sense strand (i.e. a phosphorothioate internucleotide linkage at the first and second internucleotide linkages at both the 5′ and 3′ ends of the antisense strand and a phosphorothioate internucleotide linkage at the first and second internucleotide linkages at both the 5′ and 3′ ends of the sense strand). In any of the embodiments in which one or both strands comprise one or more phosphorothioate internucleotide linkages, the remaining internucleotide linkages within the strands can be the natural 3′ to 5′ phosphodiester linkages. For instance, in some embodiments, each internucleotide linkage of the sense and antisense strands is selected from phosphodiester and phosphorothioate, wherein at least one internucleotide linkage is a phosphorothioate.

In embodiments in which the RNAi construct comprises a nucleotide overhang, two or more of the unpaired nucleotides in the overhang can be connected by a phosphorothioate internucleotide linkage. In certain embodiments, all the unpaired nucleotides in a nucleotide overhang at the 3′ end of the antisense strand and/or the sense strand are connected by phosphorothioate internucleotide linkages. In other embodiments, all the unpaired nucleotides in a nucleotide overhang at the 5′ end of the antisense strand and/or the sense strand are connected by phosphorothioate internucleotide linkages. In still other embodiments, all the unpaired nucleotides in any nucleotide overhang are connected by phosphorothioate internucleotide linkages.

In certain embodiments, the modified nucleotides incorporated into one or both of the strands of the RNAi constructs of the disclosure have a modification of the nucleobase (also referred to herein as “base”). A “modified nucleobase” or “modified base” refers to a base other than the naturally occurring purine bases adenine (A) and guanine (G) and pyrimidine bases thymine (T), cytosine (C), and uracil (U). Modified nucleobases can be synthetic or naturally occurring modifications and include, but are not limited to, universal bases, 5-methylcytosine (5-me-C), 5-hydroxymethyl cytosine, xanthine (X), hypoxanthine (I), 2-aminoadenine, 6-methyladenine, 6-methylguanine, and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine, and 3-deazaadenine.

In some embodiments, the modified base is a universal base. A “universal base” refers to a base analog that indiscriminately forms base pairs with all of the natural bases in RNA and DNA without altering the double helical structure of the resulting duplex region. Universal bases are known to those of skill in the art and include, but are not limited to, inosine, C-phenyl, C-naphthyl and other aromatic derivatives, azole carboxamides, and nitroazole derivatives, such as 3-nitropyrrole, 4-nitroindole, 5-nitroindole, and 6-nitroindole.

Other suitable modified bases that can be incorporated into the disclosed RNAi constructs include those described in, for example, Herdewijn, Antisense Nucleic Acid Drug Dev., 10: 297-310 (2000) and Peacock et al., J. Org. Chem., 76: 7295-7300 (2011). The skilled person is well aware that guanine, cytosine, adenine, thymine, and uracil may be replaced by other nucleobases, such as the modified nucleobases described above, without substantially altering the base pairing properties of a polynucleotide comprising a nucleotide bearing such replacement nucleobase.

In some embodiments, the 5′ end of the sense strand, antisense strand, or both the antisense and sense strands of the disclosed RNAi constructs comprises a phosphate moiety. As used herein, the term “phosphate moiety” refers to a terminal phosphate group that includes unmodified phosphates (—O—P═O)(OH)OH) as well as modified phosphates. Modified phosphates include phosphates in which one or more of the O and OH groups are replaced with H, O, S, N(R) or alkyl where R is H, an amino protecting group or unsubstituted or substituted alkyl. Exemplary phosphate moieties include, but are not limited to, 5′-monophosphate; 5′diphosphate; 5′-triphosphate; 5′-guanosine cap (7-methylated or non-methylated); 5′-adenosinecap or any other modified or unmodified nucleotide cap structure; 5′-monothiophosphate (phosphorothioate); 5′-monodithiophosphate (phosphorodithioate); 5′-alpha-thiotriphosphate; 5′-gamma-thiotriphosphate, 5′-phosphoramidates; 5′-vinylphosphates; 5′-alkylphosphonates (wherein “alkyl” can be methyl, ethyl, isopropyl, propyl, etc.); and 5′-alkyletherphosphonates (wherein “alkylether” can be methoxymethyl, ethoxymethyl, etc.).

The modified nucleotides that can be incorporated into the RNAi constructs of the disclosure may have more than one chemical modification described herein. For instance, the modified nucleotide may have a modification to the ribose sugar as well as a modification to the nucleobase. By way of example, a modified nucleotide may comprise a 2′ sugar modification (e.g., 2′-fluoro or 2′-methyl) and comprise a modified base (e.g., 5-methyl cytosine or pseudouracil). In other embodiments, the modified nucleotide may comprise a sugar modification in combination with a modification to the 5′ phosphate that would create a modified internucleotide or internucleoside linkage when the modified nucleotide was incorporated into a polynucleotide. For instance, in some embodiments, the modified nucleotide may comprise a sugar modification, such as a 2′-fluoro modification, a 2′-O-methyl modification, or a bicyclic sugar modification, as well as a 5′ phosphorothioate group. Accordingly, in some embodiments, one or both strands of the RNAi constructs of the disclosure comprise a combination of 2′ modified nucleotides or BNAs and phosphorothioate internucleotide linkages. In certain embodiments, both the sense and antisense strands of the RNAi constructs of the disclosure comprise a combination of 2′-fluoro modified nucleotides, 2′-O-methyl modified nucleotides, and phosphorothioate internucleotide linkages. Exemplary siRNA constructs comprising modified nucleotides and internucleotide linkages are shown in FIG. 1 and FIG. 2.

The antisense strand of the RNAi constructs of the disclosure can comprise the sequence of any one of the modified antisense sequences listed in FIG. 1 or FIG. 2 (e.g., any one of SEQ ID NOs: 817-1068 or SEQ ID NOs: 2959-3588), or the sequence of nucleotides 1-21 of any of these antisense sequences. In some embodiments, the antisense strand comprises a sequence of nucleotides 1-20 or 1-21 of any one of SEQ ID NOs: 817-1068 or SEQ ID NOs: 2959-3588.

### Function of RNAi Constructs

The disclosed RNAi constructs desirably reduce or inhibit the expression of PNPLA3 in cells, particularly liver cells. Accordingly, in one embodiment, the present disclosure provides a method of reducing PNPLA3 expression in a cell by contacting the cell with any RNAi construct described herein. The cell may be in vitro or in vivo. PNPLA3 expression can be assessed by measuring the amount or level of PNPLA3 mRNA, PNPLA3 protein, or another biomarker linked to PNPLA3 expression. The reduction of PNPLA3 expression in cells or animals treated with an RNAi construct of the disclosure can be determined relative to the PNPLA3 expression in cells or animals not treated with the RNAi construct or treated with a control RNAi construct. For instance, in some embodiments, reduction of PNPLA3 expression is assessed by (a) measuring the amount or level of PNPLA3 mRNA in liver cells treated with a RNAi construct of the disclosure, (b) measuring the amount or level of PNPLA3 mRNA in liver cells treated with a control RNAi construct (e.g., RNAi construct directed to a RNA molecule not expressed in liver cells or a RNAi construct having a nonsense or scrambled sequence) or no construct, and (c) comparing the measured PNPLA3 mRNA levels from treated cells in (a) to the measured PNPLA3 mRNA levels from control cells in (b). The PNPLA3 mRNA levels in the treated cells and controls cells can be normalized to RNA levels for a control gene (e.g., 18S ribosomal RNA) prior to comparison. PNPLA3 mRNA levels can be measured by a variety of methods, including Northern blot analysis, nuclease protection assays, fluorescence in situ hybridization (FISH), reverse-transcriptase (RT)-PCR, real-time RT-PCR, quantitative PCR, and the like.

In other embodiments, reduction of PNPLA3 expression is assessed by (a) measuring the amount or level of PNPLA3 protein in liver cells treated with a RNAi construct described herein, (b) measuring the amount or level of PNPLA3 protein in liver cells treated with a control RNAi construct (e.g., RNAi construct directed to a RNA molecule not expressed in liver cells or a RNAi construct having a nonsense or scrambled sequence) or no construct, and (c) comparing the measured PNPLA3 protein levels from treated cells in (a) to the measured PNPLA3 protein levels from control cells in (b). PNPLA3 protein levels can be measured using any suitable method known to those of skill in the art, including but not limited to, western blots, immunoassays (e.g., ELISA), and flow cytometry. Any suitable method of measuring PNPLA3 mRNA or protein can be used to assess the efficacy of the RNAi constructs described herein.

In some embodiments, the methods to assess PNPLA3 expression levels are performed in vitro in cells that natively express PNPLA3 (e.g., liver cells) or cells that have been engineered to express PNPLA3. In certain embodiments, the methods are performed in vitro in liver cells. Suitable liver cells include, but are not limited to, primary hepatocytes (e.g. human, non-human primate, or rodent hepatocytes), HepAD38 cells, HuH-6 cells, HuH-7 cells, HuH-5-2 cells, BNLCL2 cells, Hep3B cells, or HepG2 cells. In one embodiment, the liver cells are Hep3B cells. In another embodiment, the liver cells are HepG2 cells.

In other embodiments, the methods to assess PNPLA3 expression levels are performed in vivo. For example, the RNAi constructs and any control RNAi constructs can be administered to an animal (e.g., rodent or non-human primate), and PNPLA3 mRNA or protein levels may be assessed in liver tissue harvested from the animal following treatment. Alternatively or additionally, a biomarker or functional phenotype associated with PNPLA3 expression can be assessed in the treated animals.

In certain embodiments, expression of PNPLA3 is reduced in liver cells by at least 10%, at least 15%, at least 20%, at least 25%, at least 30%, at least 35%, at least 40%, at least 45%, or at least 50% by an RNAi construct described herein. In some embodiments, expression of PNPLA3 is reduced in liver cells by at least 60%, at least 65%, at least 70%, at least 75%, at least 80%, or at least 85% by an RNAi construct described herein. In other embodiments, the expression of PNPLA3 is reduced in liver cells by about 90% or more, e.g., 91%, 92%, 93%, 94%, 95%, 96%, 97%, 98%, 99%, or more by an RNAi construct described herein. The percent reduction of PNPLA3 expression can be measured by any of the methods described herein or otherwise known in the art. For instance, in certain embodiments, the RNAi constructs described herein inhibit at least 60% of PNPLA3 expression at 5 nM in Hep3B cells (contains wild type PNPLA3) in vitro. In related embodiments, the RNAi constructs described herein inhibit at least 65%, at least 70%, at least 75%, at least 80%, at least 85%, or at least 90% of PNPLA3 expression at 5 nM in Hep3B cells in vitro. In other embodiments, the RNAi constructs described herein inhibit at least 91%, at least 92%, at least 93%, at least 94%, at least 95%, at least 96%, at least 97%, at least 98%, at least 99%, or 100% of PNPLA3 expression at 5 nM in Hep3B cells in vitro. In certain embodiments, the RNAi constructs described herein inhibit at least 60% of PNPLA3 expression at 5 nM in HepG2 cells (contains wild type PNPLA3) in vitro. In related embodiments, the RNAi constructs described herein inhibit at least 65%, at least 70%, at least 75%, at least 80%, at least 85%, or at least 90% of PNPLA3 expression at 5 nM in HepG2 cells in vitro. In other embodiments, the RNAi constructs described herein inhibit at least 91%, at least 92%, at least 93%, at least 94%, at least 95%, at least 96%, at least 97%, at least 98%, at least 99%, or 100% of PNPLA3 expression at 5 nM in HepG2 cells in vitro. Reduction of PNPLA3 can be measured using a variety of techniques including, for example, RNA FISH or droplet digital PCR (see, e.g., Kamitaki et al., Digital PCR. Methods in Molecular Biology, 1768: 401-422 (2018). doi:10.1007/978-1-4939-7778-9_23).

In some embodiments, an IC₅₀ value is calculated to assess the potency of an RNAi construct described herein for inhibiting PNPLA3 expression in liver cells. An “IC₅₀ value” is the dose/concentration required to achieve 50% inhibition of a biological or biochemical function. The IC₅₀ value of any substance or antagonist can be determined by constructing a dose-response curve and examining the effect of different concentrations of the substance or antagonist on expression levels or functional activity in any assay. IC₅₀ values can be calculated for a given antagonist or substance by determining the concentration needed to inhibit half of the maximum biological response or native expression levels. Thus, the IC₅₀ value for any RNAi construct can be calculated by determining the concentration of the RNAi construct needed to inhibit half of the native PNPLA3 expression level in liver cells (e.g., PNPLA3 expression level in control liver cells) in any assay, such as an immunoassay, RNA FISH assay, or a droplet digital PCR assay. The RNAi constructs described herein may inhibit PNPLA3 expression in liver cells (e.g. Hep3B cells or HepG2 cells) with an IC₅₀ of less than about 40 nM (e.g., less than about 35, nM, 30 nM, 25 nM, 20 nM, 15 nM, 10 nM, 5 nM, or 1 nM). For example, the disclosed RNAi constructs may inhibit PNPLA3 expression in liver cells with an IC₅₀ of about 0.001 nM to about 40 nM, about 0.001 nM to about 30 nM, about 0.001 nM to about 20 nM, about 0.001 nM to about 10 nM, about 0.001 nM to about 5 nM, about 0.001 nM to about 1 nM, about 0.1 nM to about 10 nM, about 0.1 nM to about 5 nM, or about 0.1 nM to about 1 nM.

The RNAi constructs described herein can readily be made using techniques known in the art, such as, for example, conventional nucleic acid solid phase synthesis. The polynucleotides of the RNAi constructs can be assembled on a suitable nucleic acid synthesizer utilizing standard nucleotide or nucleoside precursors (e.g., phosphoramidites). Automated nucleic acid synthesizers are sold commercially by several vendors, including DNA/RNA synthesizers from Applied Biosystems (Foster City, CA), MerMade synthesizers from BioAutomation (Irving, TX), and OligoPilot synthesizers from GE Healthcare Life Sciences (Pittsburgh, PA).

The 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides.

The 2′-O-silyl group can be removed via exposure to fluoride ions, which can include any source of fluoride ion, e.g., those salts containing fluoride ion paired with inorganic counterions, e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Exemplary fluoride ion sources include, but are not limited to, tetrabutylammonium fluoride or aminohydrofluorides (e.g., combining aqueous HF with triethylamine in a dipolar aprotic solvent, e.g., dimethylformamide).

The choice of protecting groups for use on the phosphite triesters and phosphotriesters can alter the stability of the triesters towards fluoride. Methyl protection of the phosphotriester or phosphitetriester can stabilize the linkage against fluoride ions and improve process yields.

Since ribonucleosides have a reactive 2′ hydroxyl substituent, it may be desirable to protect the reactive 2′ position in RNA with a protecting group that is orthogonal to a 5′-O-dimethoxytrityl protecting group, e.g., one stable to treatment with acid. Silyl protecting groups meet this criterion and can be readily removed in a final fluoride deprotection step that can result in minimal RNA degradation.

Tetrazole catalysts can be used in the standard phosphoramidite coupling reaction. Exemplary catalysts include, e.g., tetrazole, S-ethyl-tetrazole, benzylthiotetrazole, and pnitrophenyltetrazole.

Additional methods of synthesizing the RNAi constructs described herein will be evident to those of ordinary skill in the art. Additionally, the various synthetic steps may be performed in an alternate sequence or order to give the desired compounds. Other synthetic chemistry transformations, protecting groups (e.g., for hydroxyl, amino, etc., present on the bases) and protecting group methodologies (protection and deprotection) useful in synthesizing the RNAi constructs described herein are known in the art and include, for example, those described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Custom synthesis of RNAi constructs is also available from several commercial vendors, including Dharmacon, Inc. (Lafayette, CO), AxoLabs GmbH (Kulmbach, Germany), and Ambion, Inc. (Foster City, CA).

The RNAi constructs described herein may comprise a ligand. As used herein, a “ligand” refers to any compound or molecule that can interact with another compound or molecule, either directly or indirectly. The interaction of a ligand with another compound or molecule may elicit a biological response (e.g., initiate a signal transduction cascade, induce receptor mediated endocytosis) or may just be a physical association. The ligand can modify one or more properties of the double-stranded RNA molecule to which is attached, such as the pharmacodynamic, pharmacokinetic, binding, absorption, cellular distribution, cellular uptake, charge and/or clearance properties of the RNA molecule.

The ligand may comprise a serum protein (e.g., human serum albumin, low-density lipoprotein, globulin), a cholesterol moiety, a vitamin (e.g., biotin, vitamin E, vitamin B12), a folate moiety, a steroid, a bile acid (e.g., cholic acid), a fatty acid (e.g., palmitic acid, myristic acid), a carbohydrate (e.g., a dextran, pullulan, chitin, chitosan, inulin, cyclodextrin or hyaluronic acid), a glycoside, a phospholipid, or an antibody or binding fragment thereof (e.g., a whole antibody or binding fragment that targets the RNAi construct to a specific cell type, such as liver cells). Other examples of ligands include dyes, intercalating agents (e.g., acridines), cross-linkers (e.g., psoralene, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g., adamantane acetic acid, 1-pyrene butyric acid, dihydrotestosterone, 1,3-BisO(hexadecyl)glycerol, geranyloxyhexyl group, hexadecylglycerol, borneol, menthol, 1,3-propanediol, heptadecyl group, 03-(oleoyl)lithocholic acid, 03-(oleoyl)cholenic acid, dimethoxytrityl, or phenoxazine), peptides (e.g., antennapedia peptide, Tat peptide, RGD peptides), alkylating agents, polymers (e.g., polyethylene glycol (PEG), PEG-40K), poly amino acids, and polyamines (e.g., spermine, spermidine).

In certain embodiments, the ligands have endosomolytic properties. The endosomolytic ligands promote the lysis of the endosome and/or transport of the RNAi construct described herein, or its components, from the endosome to the cytoplasm of the cell. The endosomolytic ligand may be a polycationic peptide or peptidomimetic which shows pH-dependent membrane activity and fusogenicity. In one embodiment, the endosomolytic ligand assumes its active conformation at endosomal pH. The “active” conformation is that conformation in which the endosomolytic ligand promotes lysis of the endosome and/or transport of the RNAi construct described herein, or its components, from the endosome to the cytoplasm of the cell. Exemplary endosomolytic ligands include the GALA peptide (Subbarao et al., Biochemistry, Vol. 26: 2964-2972, 1987), the EALA peptide (Vogel et al., J. Am. Chem. Soc., Vol. 118: 1581-1586, 1996), and their derivatives (Turk et al., Biochem. Biophys. Acta, Vol. 1559: 56-68, 2002). In one embodiment, the endosomolytic component may contain a chemical group (e.g., an amino acid) which will undergo a change in charge or protonation in response to a change in pH. The endosomolytic component may be linear or branched.

In some embodiments, the ligand comprises a lipid or other hydrophobic molecule. In one embodiment, the ligand comprises a cholesterol moiety or other steroid. Cholesterol conjugated oligonucleotides have been reported to be more active than their unconjugated counterparts (Manoharan, Antisense Nucleic Acid Drug Development, Vol. 12: 103-228, 2002). Ligands comprising cholesterol moieties and other lipids for conjugation to nucleic acid molecules have also been described in U.S. Pat. Nos. 7,851,615; 7,745,608; and 7,833,992. In another embodiment, the ligand may comprise a folate moiety. Polynucleotides conjugated to folate moieties can be taken up by cells via a receptor-mediated endocytosis pathway. Such folate-polynucleotide conjugates are described in, e.g., U.S. Pat. No. 8,188,247.

Given that PNPLA3 is expressed in liver cells (e.g., hepatocytes), in certain embodiments, it is desirable to specifically deliver the RNAi construct to liver cells. In some embodiments, RNAi constructs can be specifically targeted to the liver by employing ligands that bind to or interact with proteins expressed on the surface of liver cells. For example, in certain embodiments, a ligand may comprise one or more antigen binding proteins (e.g. antibodies or binding fragments thereof (e.g. Fab, scFv)) that specifically bind to a receptor expressed on hepatocytes.

In certain embodiments, the ligand comprises a carbohydrate. A “carbohydrate” refers to a compound made up of one or more monosaccharide units having at least 6 carbon atoms (which can be linear, branched, or cyclic) with an oxygen, nitrogen or sulfur atom bonded to each carbon atom. Carbohydrates include, but are not limited to, sugars (e.g., monosaccharides, disaccharides, trisaccharides, tetrasaccharides, and oligosaccharides containing from about 4, 5, 6, 7, 8, or 9 monosaccharide units), and polysaccharides, such as starches, glycogen, cellulose, and polysaccharide gums. In some embodiments, the carbohydrate incorporated into the ligand is a monosaccharide selected from a pentose, hexose, or heptose and di- and tri-saccharides including such monosaccharide units. In other embodiments, the carbohydrate incorporated into the ligand is an amino sugar, such as galactosamine, glucosamine, N-acetylgalactosamine, and N-acetylglucosamine.

In some embodiments, the ligand comprises a hexose or hexosamine. The hexose may be selected from glucose, galactose, mannose, fucose, or fructose. The hexosamine may be selected from fructosamine, galactosamine, glucosamine, or mannosamine. In certain embodiments, the ligand comprises glucose, galactose, galactosamine, or glucosamine. In one embodiment, the ligand comprises glucose, glucosamine, or N-acetylglucosamine. In another embodiment, the ligand comprises galactose, galactosamine, or N-acetyl-galactosamine. In particular embodiments, the ligand comprises N-acetyl-galactosamine. Ligands comprising glucose, galactose, and N-acetyl-galactosamine (GalNAc) are particularly effective in targeting compounds to liver cells (see, e.g., D'Souza and Devarajan, J. Control Release, Vol. 203: 126-139, 2015). Examples of GalNAc- or galactose-containing ligands that can be incorporated into the RNAi constructs described herein are described in U.S. Pat. Nos. 7,491,805; 8,106,022; and 8,877,917; U.S. Patent Publication No. 2003/0130186; and WIPO Publication No. WO 2013/166155.

In certain embodiments, the ligand comprises a multivalent carbohydrate moiety. As used herein, a “multivalent carbohydrate moiety” refers to a moiety comprising two or more carbohydrate units capable of independently binding or interacting with other molecules. For example, a multivalent carbohydrate moiety comprises two or more binding domains comprised of carbohydrates that can bind to two or more different molecules or two or more different sites on the same molecule. The valency of the carbohydrate moiety denotes the number of individual binding domains within the carbohydrate moiety. For instance, the terms “monovalent,” “bivalent,” “trivalent,” and “tetravalent” with reference to the carbohydrate moiety refer to carbohydrate moieties with one, two, three, and four binding domains, respectively. The multivalent carbohydrate moiety may comprise a multivalent lactose moiety, a multivalent galactose moiety, a multivalent glucose moiety, a multivalent N-acetyl-galactosamine moiety, a multivalent N-acetyl-glucosamine moiety, a multivalent mannose moiety, or a multivalent fucose moiety. In some embodiments, the ligand comprises a multivalent galactose moiety. In other embodiments, the ligand comprises a multivalent N-acetyl-galactosamine moiety. In these and other embodiments, the multivalent carbohydrate moiety is bivalent, trivalent, or tetravalent. In such embodiments, the multivalent carbohydrate moiety can be bi-antennary or tri-antennary. In one particular embodiment, the multivalent N-acetyl-galactosamine moiety is trivalent or tetravalent. In another particular embodiment, the multivalent galactose moiety is trivalent or tetravalent. Exemplary trivalent and tetravalent GalNAc-containing ligands for incorporation into the RNAi constructs are described in detail below.

The ligand can be attached or conjugated to the RNA molecule of the RNAi construct directly or indirectly. For instance, in some embodiments, the ligand is covalently attached directly to the sense or antisense strand of the RNAi construct. In other embodiments, the ligand is covalently attached via a linker to the sense or antisense strand of the RNAi construct. The ligand can be attached to nucleobases, sugar moieties, or internucleotide linkages of polynucleotides (e.g., sense strand or antisense strand) of the RNAi constructs described herein. Conjugation or attachment to purine nucleobases or derivatives thereof can occur at any position including, endocyclic and exocyclic atoms. In certain embodiments, the 2-, 6-, 7-, or 8-positions of a purine nucleobase are attached to a ligand. Conjugation or attachment to pyrimidine nucleobases or derivatives thereof can also occur at any position. In some embodiments, the 2, 5-, and 6-positions of a pyrimidine nucleobase can be attached to a ligand. Conjugation or attachment to sugar moieties of nucleotides can occur at any carbon atom. Example carbon atoms of a sugar moiety that can be attached to a ligand include the 2′, 3′, and 5′ carbon atoms. The 1′ position can also be attached to a ligand, such as in a basic residue. Internucleotide linkages can also support ligand attachments. For phosphorus-containing linkages (e.g., phosphodiester, phosphorothioate, phosphorodithiotate, phosphoroamidate, and the like), the ligand can be attached directly to the phosphorus atom or to an O, N, or S atom bound to the phosphorus atom. For amine- or amide-containing internucleoside linkages (e.g., PNA), the ligand can be attached to the nitrogen atom of the amine or amide or to an adjacent carbon atom.

In certain embodiments, the ligand may be attached to the 3′ or 5′ end of either the sense or antisense strand. In certain embodiments, the ligand is covalently attached to the 5′ end of the sense strand. In other embodiments, the ligand is covalently attached to the 3′ end of the sense strand. For example, in some embodiments, the ligand is attached to the 3′-terminal nucleotide of the sense strand. In certain such embodiments, the ligand is attached at the 3′-position of the 3′-terminal nucleotide of the sense strand. In alternative embodiments, the ligand is attached near the 3′ end of the sense strand, but before one or more terminal nucleotides (i.e. before 1, 2, 3, or 4 terminal nucleotides). In some embodiments, the ligand is attached at the 2′-position of the sugar of the 3′-terminal nucleotide of the sense strand.

In certain embodiments, the ligand is attached to the sense or antisense strand via a linker. A “linker” is an atom or group of atoms that covalently joins a ligand to a polynucleotide component of the RNAi construct. The linker may be from about 1 to about 30 atoms in length, from about 2 to about 28 atoms in length, from about 3 to about 26 atoms in length, from about 4 to about 24 atoms in length, from about 6 to about 20 atoms in length, from about 7 to about 20 atoms in length, from about 8 to about 20 atoms in length, from about 8 to about 18 atoms in length, from about 10 to about 18 atoms in length, and from about 12 to about 18 atoms in length. In some embodiments, the linker may comprise a bifunctional linking moiety, which generally comprises an alkyl moiety with two functional groups. One of the functional groups is selected to bind to the compound of interest (e.g., sense or antisense strand of the RNAi construct) and the other is selected to bind essentially any selected group, such as a ligand as described herein. In certain embodiments, the linker comprises a chain structure or an oligomer of repeating units, such as ethylene glycol or amino acid units. Examples of functional groups that are typically employed in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Linkers that may be used to attach a ligand to the sense or antisense strand in the RNAi constructs described herein include, but are not limited to, pyrrolidine, 8-amino-3,6-di oxaoctanoic acid, succinimidyl 4-(N-maleimidomethyl)cyclohexane-1-carboxylate, 6-aminohexanoic acid, substituted C₁-C₁₀ alkyl, substituted or unsubstituted C₂-C₁₀ alkenyl or substituted or unsubstituted C₂-C₁₀ alkynyl. Preferred substituent groups for such linkers include, but are not limited to, hydroxyl, amino, alkoxy, carboxy, benzyl, phenyl, nitro, thiol, thioalkoxy, halogen, alkyl, aryl, alkenyl, and alkynyl.

In certain embodiments, the linkers are cleavable. A cleavable linker is one which is sufficiently stable outside the cell, but which upon entry into a target cell is cleaved to release the two parts the linker is holding together. In some embodiments, the cleavable linker is cleaved at least 10 times, 20 times, 30 times, 40 times, 50 times, 60 times, 70 times, 80 times, 90 times, or more, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linkers are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linker by reduction; esterases; endosomes or agents that can create an acidic environment, e.g., those that result in a pH of five or lower; enzymes that can hydrolyze or degrade an acid cleavable linker by acting as a general acid, peptidases (which can be substrate specific), and phosphatases.

A cleavable linker may comprise a moiety that is susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable group that is cleaved at a preferred pH, thereby releasing the RNA molecule from the ligand inside the cell, or into the desired compartment of the cell.

A linker can include a cleavable group that is cleavable by a particular enzyme. The type of cleavable group incorporated into a linker can depend on the cell to be targeted. For example, liver-targeting ligands can be linked to RNA molecules through a linker that includes an ester group. Liver cells are rich in esterases, and therefore the linker will be cleaved more efficiently in liver cells than in cell types that are not esterase-rich. Other types of cells rich in esterases include cells of the lung, renal cortex, and testis. Linkers that contain peptide bonds can be used when targeting cells rich in peptidases, such as liver cells and synoviocytes.

In general, the suitability of a candidate cleavable linker can be evaluated by testing the ability of a degradative agent (or condition) to cleave the candidate linker. It will also be desirable to also test the candidate cleavable linker for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In some embodiments, useful candidate linkers are cleaved at least 2, 4, 10, 20, 50, 70, or 100 times faster in the cell (or under in vitro conditions selected to mimic intracellular conditions) as compared to blood or serum (or under in vitro conditions selected to mimic extracellular conditions).

In other embodiments, redox cleavable linkers are utilized. Redox cleavable linkers are cleaved upon reduction or oxidation. An example of reductively cleavable group is a disulfide linking group (—S—S—). To determine if a candidate cleavable linker is a suitable “reductively cleavable linker,” or, for example, is suitable for use with a particular RNAi construct and particular ligand, one or more methods described herein can be used. For example, a candidate linker can be evaluated by incubation with dithiothreitol (DTT), or other reducing agent known in the art, which mimics the rate of cleavage that would be observed in a cell, e.g., a target cell. The candidate linkers can also be evaluated under conditions which are selected to mimic blood or serum conditions. In a specific embodiment, candidate linkers are cleaved by at most 10% in the blood. In other embodiments, useful candidate linkers are degraded at least 2, 4, 10, 20, 50, 70, or 100 times faster in the cell (or under in vitro conditions selected to mimic intracellular conditions) as compared to blood (or under in vitro conditions selected to mimic extracellular conditions).

In yet other embodiments, phosphate-based cleavable linkers are cleaved by agents that degrade or hydrolyze the phosphate group. An example of an agent that hydrolyzes phosphate groups in cells are enzymes, such as phosphatases in cells. Examples of phosphate-based cleavable groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Specific embodiments include —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —SP(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. Another specific embodiment is —O—P(O)(OH)—O—. These candidate linkers can be evaluated using methods analogous to those described above.

In other embodiments, the linkers may comprise acid cleavable groups, which are groups that are cleaved under acidic conditions. In some embodiments, acid cleavable groups are cleaved in an acidic environment with a pH of about 6.5 or lower (e.g., about 6.0, 5.5, 5.0, or lower), or by agents, such as enzymes that can act as a general acid. In a cell, specific low pH organelles, such as endosomes and lysosomes, can provide a cleaving environment for acid cleavable groups. Examples of acid cleavable linking groups include, but are not limited to, hydrazones, esters, and esters of amino acids. Acid cleavable groups can have the general formula —C═NN—, C(O)O, or —OC(O). A specific embodiment is when the carbon attached to the oxygen of the ester (the alkoxy group) is an aryl group, substituted alkyl group, or tertiaryalkyl group such as dimethyl, pentyl or t-butyl. These candidates can be evaluated using methods analogous to those described above.

In other embodiments, the linkers may comprise ester-based cleavable groups, which are cleaved by enzymes, such as esterases and amidases in cells. Examples of ester-based cleavable groups include, but are not limited to, esters of alkylene, alkenylene and alkynylene groups. Ester cleavable groups have the general formula —C(O)O—, or —OC(O)—. These candidate linkers can be evaluated using methods analogous to those described above.

In further embodiments, the linkers may comprise peptide-based cleavable groups, which are cleaved by enzymes, such as peptidases and proteases in cells. Peptide-based cleavable groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide-based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

Other types of linkers suitable for attaching ligands to the sense or antisense strands in the RNAi constructs described herein are known in the art and can include the linkers described in, e.g., U.S. Pat. Nos. 7,723,509; 8,017,762; 8,828,956; 8,877,917; and 9,181,551.

In certain embodiments, the ligand covalently attached to the sense or antisense strand of the RNAi constructs described herein comprises a GalNAc moiety, e.g, a multivalent GalNAc moiety. In some embodiments, the multivalent GalNAc moiety is a trivalent GalNAc moiety and is attached to the 3′ end of the sense strand. In other embodiments, the multivalent GalNAc moiety is a trivalent GalNAc moiety and is attached to the 5′ end of the sense strand. In yet other embodiments, the multivalent GalNAc moiety is a tetravalent GalNAc moiety and is attached to the 3′ end of the sense strand. In still other embodiments, the multivalent GalNAc moiety is a tetravalent GalNAc moiety and is attached to the 5′ end of the sense strand.

In some embodiments, the RNAi constructs described herein may be delivered to a cell or tissue of interest by administering a vector that encodes and controls the intracellular expression of the RNAi construct. A “vector” (also referred to herein as an “expression vector”) is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated viral vectors, retroviral vectors, and the like. A vector can be replicated in a living cell, or it can be made synthetically.

Generally, a vector for expressing an RNAi construct described herein will comprise one or more promoters operably linked to sequences encoding the RNAi construct. The phrases “operably linked,” “operatively linked,” or “under transcriptional control” may be used interchangeably herein to indicate when a promoter is in the correct location and orientation in relation to a polynucleotide sequence to control the initiation of transcription by RNA polymerase and expression of the polynucleotide sequence. A “promoter” refers to a sequence recognized by the synthetic machinery of the cell, or introduced synthetic machinery, required to initiate the specific transcription of a gene sequence. Suitable promoters include, but are not limited to, RNA pol I, pol II, HI or U6 RNA pol III, and viral promoters (e.g., human cytomegalovirus (CMV) immediate early gene promoter, the SV40 early promoter, and the Rous sarcoma virus long terminal repeat). In some embodiments, an HI or U6RNA pol III promoter is employed. The promoter can be a tissue-specific or inducible promoter. Of particular interest are liver-specific promoters, such as promoter sequences from the human alpha-1 antitrypsin gene, albumin gene, hemopexin gene, and hepatic lipase gene. Inducible promoters include, for example, promoters regulated by ecdysone, estrogen, progesterone, tetracycline, and isopropyl-PD1-thiogalactopyranoside (IPTG).

When the RNAi construct comprises an siRNA, the two separate strands (sense and antisense strand) can be expressed from a single vector or two separate vectors. For example, in some embodiments, the sequence encoding the sense strand is operably linked to a promoter on a first vector and the sequence encoding the antisense strand is operably linked to a promoter on a second vector. In such an embodiment, the first and second vectors are co-introduced, e.g., by infection or transfection, into a target cell, such that the sense and antisense strands, once transcribed, will hybridize intracellularly to form the siRNA molecule. In another embodiment, the sense and antisense strands are transcribed from two separate promoters located in a single vector. In such embodiments, the sequence encoding the sense strand may be operably linked to a first promoter and the sequence encoding the antisense strand may be operably linked to a second promoter, wherein the first and second promoters are located in a single vector. In one embodiment, the vector comprises a first promoter operably linked to a sequence encoding the siRNA molecule, and a second promoter operably linked to the same sequence in the opposite direction, such that transcription of the sequence from the first promoter results in the synthesis of the sense strand of the siRNA molecule and transcription of the sequence from the second promoter results in synthesis of the antisense strand of the siRNA molecule.

When the RNAi construct comprises a shRNA, a sequence encoding the single, at least partially self-complementary RNA molecule is operably linked to a promoter to produce a single transcript. In some embodiments, the sequence encoding the shRNA comprises an inverted repeat joined by a linker polynucleotide sequence to produce the stem and loop structure of the shRNA following transcription.

In some embodiments, the vector encoding an RNAi construct described herein is a viral vector. Various viral vector systems that are suitable to express the RNAi constructs described herein include, but are not limited to, adenoviral vectors, retroviral vectors (e.g., lentiviral vectors, maloney murine leukemia virus), adeno-associated viral vectors; herpes simplex viral vectors; SV40 vectors; polyoma viral vectors; papilloma viral vectors; picomaviral vectors; and pox viral vectors (e.g., vaccinia virus). In certain embodiments, the viral vector is a retroviral vector (e.g., lentiviral vector).

Various vectors suitable for use in the present disclosure, methods for inserting nucleic acid sequences encoding siRNA or shRNA molecules into vectors, and methods of delivering the vectors to the cells of interest are known in the art (see, e.g., Dornburg, Gene Therap., Vol. 2: 301-310, 1995; Eglitis, Biotechniques, Vol. 6: 608-614, 1988; Miller, HumGene Therap., Vol. 1: 5-14, 1990; Anderson, Nature, Vol. 392: 25-30, 1998; Rubinson D A et al., Nat. Genet., Vol. 33: 401-406, 2003; Brummelkamp et al., Science, Vol. 296: 550-553, 2002; Brummelkamp et al., Cancer Cell, Vol. 2: 243-247, 2002; Lee et al., Nat Biotechnol, Vol. 20: 500-505, 2002; Miyagishi et al., Nat Biotechnol, Vol. 20: 497-500, 2002; Paddison et al., GenesDev, Vol. 16: 948-958, 2002; Paul et al., Nat Biotechnol, Vol. 20: 505-508, 2002; Sui et al., Proc Natl Acad Sci USA, Vol. 99: 5515-5520, 2002; and Yu et al., Proc Natl Acad Sci USA, Vol. 99: 6047-6052, 2002).

### Compositions

The disclosure also provides compositions and formulations comprising the RNAi constructs described herein and pharmaceutically acceptable carriers, excipients, or diluents. Such compositions and formulations are useful for reducing expression of PNPLA3 in a subject in need thereof. Where clinical applications are contemplated, pharmaceutical compositions and formulations will be prepared in a form appropriate for the intended application. Generally, this will entail preparing compositions that are essentially free of pyrogens, as well as other impurities that could be harmful to humans or animals.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier, excipient, or diluent” includes solvents, buffers, solutions, dispersion media, coatings, antibacterial and antifungal agents, isotonic and absorption delaying agents, etc., acceptable for use in formulating pharmaceuticals, such as pharmaceuticals suitable for administration to humans. The use of such media and agents for pharmaceutically active substances is well known in the art. Except insofar as any conventional media or agent is incompatible with the RNAi constructs described herein, its use in therapeutic compositions is contemplated. Supplementary active ingredients also can be incorporated into the compositions, provided they do not inactivate the vectors or RNAi constructs of the compositions.

Compositions and methods for the formulation of pharmaceutical compositions depend on several criteria, including, but not limited to, route of administration, type and extent of disease or disorder to be treated, and dose to be administered. In some embodiments, the pharmaceutical compositions are formulated based on the intended route of delivery. For instance, in certain embodiments, the pharmaceutical compositions are formulated for parenteral delivery. Parenteral forms of delivery include intravenous, intraarterial, subcutaneous, intrathecal, intraperitoneal, and intramuscular injection or infusion. In one embodiment, the pharmaceutical composition is formulated for intravenous delivery. In such an embodiment, the pharmaceutical composition may include a lipid-based delivery vehicle. In another embodiment, the pharmaceutical composition is formulated for subcutaneous delivery. In such an embodiment, the pharmaceutical composition may include a targeting ligand (e.g., GalNAc-containing ligands described herein).

In some embodiments, the pharmaceutical compositions comprise an effective amount of an RNAi construct described herein. An “effective amount” is an amount sufficient to produce a beneficial or desired clinical result. In some embodiments, an effective amount is an amount sufficient to reduce PNPLA3 expression in hepatocytes of a subject. In some embodiments, an effective amount may be an amount sufficient to only partially reduce PNPLA3 expression, for example, to a level comparable to expression of the wild-type PNPLA3 allele in human heterozygotes. Human heterozygous carriers of loss of function PNPLA3 variant alleles were reported to have lower serum levels of non-HDL cholesterol and a lower risk of coronary artery disease and myocardial infarction as compared to non-carriers (Nioi et al., New England Journal of Medicine, Vol. 374(22): 2131-2141, 2016). Thus, without being bound by theory, it is believed that partial reduction of PNPLA3 expression may be sufficient to achieve the beneficial reduction of serum non-HDL cholesterol and reduction of risk of coronary artery disease and myocardial infarction.

An effective amount of an RNAi construct may be from about 0.01 mg/kg body weight to about 100 mg/kg body weight, about 0.05 mg/kg body weight to about 75 mg/kg body weight, about 0.1 mg/kg body weight to about 50 mg/kg body weight, about 1 mg/kg to about 30 mg/kg body weight, about 2.5 mg/kg of body weight to about 20 mg/kg bodyweight, or about 5 mg/kg body weight to about 15 mg/kg body weight. In certain embodiments, a single effective dose of an RNAi construct may be about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. The pharmaceutical composition comprising an effective amount of RNAi construct can be administered weekly, biweekly, monthly, quarterly, or biannually. The precise determination of what would be considered an effective amount and frequency of administration may be based on several factors, including a patient's size, age, gender, type of disorder to be treated (e.g., myocardial infarction, heart failure, coronary artery disease, hypercholesterolemia), particular RNAi construct employed, and route of administration. Estimates of effective dosages and in vivo half-lives for any particular RNAi construct described herein can be ascertained using conventional methods and/or testing in appropriate animal models.

Colloidal dispersion systems, such as macromolecule complexes, nanocapsules, microspheres, beads, and lipid-based systems, including oil-in-water emulsions, micelles, mixed micelles, and liposomes, may be used as delivery vehicles for the RNAi constructs described herein or vectors encoding such constructs. Commercially available fat emulsions that are suitable for delivering the nucleic acids described herein include INTRALIPID®, LIPOSYN®, LIPOSYN® II, LIPOSYN® III, NUTRILIPID, and other similar lipid emulsions. A preferred colloidal system for use as a delivery vehicle in vivo is a liposome (i.e., an artificial membrane vesicle). The RNAi constructs described herein may be encapsulated within liposomes, such as cationic liposomes. Alternatively, RNAi constructs may be complexed to lipids, such as cationic lipids. Suitable lipids and liposomes include neutral (e.g., dioleoylphosphatidyl ethanolamine (DOPE), dimyristoylphosphatidyl choline (DMPC), and dipalmitoyl phosphatidylcholine (DPPC)), distearolyphosphatidyl choline), negative (e.g., dimyristoylphosphatidyl glycerol (DMPG)), and cationic (e.g., dioleoyltetramethylaminopropyl (DOTAP) and dioleoylphosphatidyl ethanolamine (DOTMA)). The preparation and use of such colloidal dispersion systems is well known in the art. Exemplary formulations also are disclosed in, e.g., U.S. Pat. Nos. 5,783,565; 5,837,533; 5,981,505; 6,127,170; 6,217,900; 6,379,965; 6,383,512; 6,747,014; 7,202,227; and WO 03/093449.

In some embodiments, the RNAi constructs are fully encapsulated in a lipid formulation, e.g., to form a SPLP, pSPLP, SNALP, or other nucleic acid-lipid particle. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a noncationic lipid, and a lipid that prevents aggregation of the particle (e.g., a PEG-lipid conjugate). SNALPs and SPLPs are exceptionally useful for systemic applications, as they exhibit extended circulation lifetimes following intravenous injection and accumulate at distal sites (e.g., sites physically separated from the administration site). SPLPs include “pSPLP,” which include an encapsulated condensing agent-nucleic acid complex as set forth in PCT Publication No. WO 00/03683. The nucleic acid-lipid particles typically have a mean diameter of about 50 nm to about 150 nm, about 60 nm to about 130 nm, about 70 nm to about 110 nm, or about 70 nm to about 90 nm, and are substantially nontoxic. In addition, the nucleic acids present in the nucleic acid-lipid particles desirably are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; and 6,815,432; and PCT Publication No. WO 96/40964.

Pharmaceutical compositions suitable for injections include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, liquid polyethylene glycol, and the like), suitable mixtures thereof, and vegetable oils. The proper fluidity can be maintained, for example, by using a coating (such as lecithin), by maintaining the required particle size (in the case of dispersion), and/or by using surfactants. The prevention of the action of microorganisms can be brought about by various antibacterial and antifungal agents, such as, for example, parabens, chlorobutanol, phenol, sorbic acid, thimerosal, and the like. In many cases, isotonic agents (e.g., sugars or sodium chloride) may be included in the composition. Prolonged absorption of the injectable compositions can be brought about by including absorption-delaying agents, such as, for example, aluminum monostearate and gelatin.

Sterile injectable solutions may be prepared by incorporating an appropriate amount of the RNAi construct (alone or complexed with a ligand) into a solvent along with any other ingredients (such as described above) as desired, followed by filtered sterilization. Generally, dispersions are prepared by incorporating the various sterilized active ingredients into a sterile vehicle which contains the basic dispersion medium and the desired other ingredients. In the case of sterile powders for the preparation of sterile injectable solutions, suitable methods of preparation include vacuum-drying and freeze-drying techniques which yield a powder of the active ingredient(s) plus any additional desired ingredient from a previously sterile-filtered solution thereof.

The compositions provided herein may be formulated in a neutral or salt form. Pharmaceutically-acceptable salts include, for example, acid addition salts (formed with free amino groups) derived from inorganic acids (e.g., hydrochloric or phosphoric acids), or from organic acids (e.g., acetic, oxalic, tartaric, mandelic, and the like). Salts formed with free carboxyl groups can also be derived from inorganic bases (e.g., sodium, potassium, ammonium, calcium, or ferric hydroxides) or from organic bases (e.g., isopropylamine, trimethylamine, histidine, procaine, and the like).

For parenteral administration in an aqueous solution, for example, a solution generally is suitably buffered and a liquid diluent is first rendered isotonic with, e.g., sufficient saline or glucose. Such aqueous solutions may be used, for example, for intravenous, intramuscular, subcutaneous, and intraperitoneal administration. Sterile aqueous media desirably are employed as is known to those of skill in the art. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). For human administration, preparations should meet sterility, pyrogenicity, general safety and purity standards as required by FDA standards. In certain embodiments, a pharmaceutical composition described herein comprises or consists of a sterile saline solution and an RNAi construct described herein. In other embodiments, a pharmaceutical composition described herein comprises or consists of an RNAi construct described herein and sterile water (e.g. water for injection, WFI). In still other embodiments, a pharmaceutical composition described herein comprises or consists of an RNAi construct described herein and phosphate-buffered saline (PBS).

In some embodiments, the pharmaceutical compositions are packaged with or stored within a device for administration. Devices for injectable formulations include, but are not limited to, injection ports, pre-filled syringes, auto injectors, injection pumps, on-body injectors, and injection pens. Devices for aerosolized or powder formulations include, but are not limited to, inhalers, insufflators, aspirators, and the like. Thus, the present disclosure includes administration devices comprising a pharmaceutical composition described herein for treating or preventing one or more of the disorders described herein.

### Methods for Inhibiting PNPLA3 Expression

The present disclosure also provides methods of inhibiting expression of a PNPLA3 gene in a cell. The methods include contacting a cell with an RNAi construct, e.g., double-stranded RNAi construct, in an amount effective to inhibit expression of PNPLA3 in the cell, thereby inhibiting expression of PNPLA3 in the cell. Contacting a cell with an RNAi construct, e.g., a double-stranded RNAi construct, may be done in vitro or in vivo. Contacting a cell in vivo with the RNAi construct includes contacting a cell or group of cells within a subject, e.g., a human subject, with the RNAi construct. Combinations of in vitro and in vivo methods of contacting a cell also are within the scope of the present disclosure.

The present disclosure provides methods for reducing or inhibiting expression of PNPLA3 in a subject in need thereof as well as methods of treating or preventing conditions, diseases, or disorders associated with PNPLA3 expression or activity. A “condition, disease, or disorder associated with PNPLA3 expression” refers to conditions, diseases, or disorders in which PNPLA3 expression levels are altered or where elevated expression levels of PNPLA3 are associated with an increased risk of developing the condition, disease, or disorder.

Contacting a cell may be direct or indirect, as discussed above. Furthermore, contacting a cell may be accomplished via a targeting ligand, including any ligand described herein or known in the art. In preferred embodiments, the targeting ligand is a carbohydrate moiety, e.g., a GalNAc3 ligand, or any other ligand that directs the RNAi construct to a site of interest.

In one embodiment, contacting a cell with an RNAi includes “introducing” or “delivering the RNAi into the cell” by facilitating or effecting uptake or absorption into the cell. Absorption or uptake of an RNAi can occur through unaided diffusive or active cellular processes, or by auxiliary agents or devices. For in vivo introduction, for example, RNAi can be injected into a tissue site or administered systemically. In vitro introduction into a cell may be accomplished using methods known in the art, such as electroporation and lipofection. Additional approaches are described herein below and/or are known in the art.

The term “inhibiting,” as used herein, is used interchangeably with “reducing,” “silencing,” “downregulating”, “suppressing”, and other similar terms, and includes any level of inhibition.

The phrase “inhibiting expression of a PNPLA3” is intended to refer to inhibition of expression of any PNPLA3 gene (such as, e.g., a mouse PNPLA3 gene, a rat PNPLA3 gene, a monkey PNPLA3 gene, or a human PNPLA3 gene) as well as variants or mutants of a PNPLA3 gene. Thus, the PNPLA3 gene may be a wild-type PNPLA3 gene, a mutant PNPLA3 gene (such as a mutant PNPLA3 gene giving rise to amyloid deposition), or a transgenic PNPLA3 gene in the context of a genetically manipulated cell, group of cells, or organism.

“Inhibiting expression of a PNPLA3 gene” includes any level of inhibition of a PNPLA3 gene, e.g., at least partial suppression of the expression of a PNPLA3 gene. The expression of the PNPLA3 gene may be assessed based on the level, or the change in the level, of any variable associated with PNPLA3 gene expression, e.g., PNPLA3 mRNA level, PNPLA3 protein level, or the number or extent of amyloid deposits. This level may be assessed in an individual cell or in a group of cells, including, for example, a sample derived from a subject.

Inhibition may be assessed by a decrease in an absolute or relative level of one or more variables that are associated with PNPLA3 expression compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control). In some embodiments, expression of a PNPLA3 gene is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

Inhibition of the expression of a PNPLA3 gene may be manifested by a reduction of the amount of mRNA expressed by a first cell or group of cells (such cells may be present, for example, in a sample derived from a subject) in which a PNPLA3 gene is transcribed and which has or have been treated (e.g., by contacting the cell or cells with an RNAi construct described herein, or by administering an RNAi construct described herein to a subject in which the cells are or were present), such that the expression of a PNPLA3 gene is inhibited, as compared to a second cell or group of cells substantially identical to the first cell or group of cells but which has not or have not been so treated (control cell(s)). Inhibition may be assessed by expressing the level of mRNA in treated cells as a percentage of the level of mRNA in control cells, using the following formula:

$$\frac{( m R N A in control cells ) - ( m R N A in treated cells )}{m R N A in control cells} \times 100 \%$$

Alternatively, inhibition of the expression of a PNPLA3 gene may be assessed in terms of a reduction of a parameter that is functionally linked to PNPLA3 gene expression, e.g., PNPLA3 protein expression or Hedgehog pathway protein activities. PNPLA3 gene silencing may be determined in any cell expressing PNPLA3, either endogenously or recombinantly, by any assay known in the art.

Inhibition of the expression of a PNPLA3 protein may be manifested by a reduction in the level of the PNPLA3 protein that is expressed by a cell or group of cells (e.g., the level of protein expressed in a sample obtained from a subject). As explained above, for the assessment of mRNA suppression, the inhibition of protein expression levels in a treated cell or group of cells may similarly be expressed as a percentage of the level of protein in a control cell or group of cells.

A control cell or group of cells that may be used to assess the inhibition of the expression of a PNPLA3 gene includes a cell or group of cells that has not yet been contacted with an RNAi construct described herein. For example, the control cell or group of cells may be derived from an individual subject (e.g., a human or animal subject) prior to treatment of the subject with an RNAi construct.

The level of PNPLA3 mRNA that is expressed by a cell or group of cells, or the level of circulating PNPLA3 mRNA, may be determined using any method known in the art for assessing mRNA expression, such as those mentioned above. In some embodiments, the level of expression of PNPLA3 in a sample is determined by detecting a transcribed polynucleotide, or portion thereof, e.g., mRNA of the PNPLA3 gene. In this regard, for example, RNA may be extracted from cells using RNA extraction techniques including, for example, acid phenol/guanidine isothiocyanate extraction (RNAzol B; Biogenesis), RNeasy RNA preparation kits (Qiagen), or PAXgene (PreAnalytix, Switzerland). Typical assay formats utilizing ribonucleic acid hybridization include nuclear run-on assays, RT-PCR, RNase protection assays (Melton et al., Nuc. Acids Res., 12:7035), northern blotting, in situ hybridization, and microarray analysis. Circulating PNPLA3 mRNA may be detected using methods described in WO 2012/177906.

In one embodiment, the level of expression of PNPLA3 is determined using a nucleic acid probe. The term “probe,” as used herein, refers to any molecule that is capable of selectively binding to a specific PNPLA3 sequence. Probes can be synthesized by one of skill in the art or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting isolated mRNA with a nucleic acid molecule (probe) that can hybridize to PNPLA3 mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in determining the level of PNPLA3 mRNA.

An alternative method for determining the level of expression of PNPLA3 in a sample involves the process of nucleic acid amplification and/or reverse transcriptase (to prepare cDNA) of for example mRNA in the sample, e.g., by RT-PCR (see, e.g., U.S. Pat. No. 4,683,202), ligase chain reaction (Barany (1991) Proc. Natl. Acad. Sci. USA 88: 189-193), self-sustained sequence replication (Guatelli et al. (1990) Proc. Natl. Acad. Sci. USA 87: 1874-1878), transcriptional amplification system (Kwoh et al. (1989) Proc. Natl. Acad. Sci. USA 86: 1173-1177), Q-Beta Replicase (Lizardi et al. (1988) Bio/Technology 6: 1197), rolling circle replication (Lizardi et al., supra; and U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In some aspects of the disclosure, the level of expression of PNPLA3 may be determined by quantitative fluorogenic RT-PCR {i.e., the TAQMAN™ System). The expression levels of PNPLA3 mRNA may be monitored using a membrane blot (such as used in hybridization analysis such as northern, Southern, dot, and the like), or microwells, sample tubes, gels, beads or fibers (or any solid support comprising bound nucleic acids) (see, e.g., U.S. Pat. Nos. 5,445,934; 5,677,195; 5,770,722; 5,744,305; and 5,874,219). The determination of PNPLA3 expression level may also comprise using nucleic acid probes in solution. In certain embodiments, the level of mRNA expression is assessed using branched DNA (bDNA) assays or real time PCR (qPCR).

The level of PNPLA3 protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, colorimetric assays, spectrophotometric assays, flow cytometry, immunodiffusion (single or double), immunoelectrophoresis, Western blotting, radioimmunoassay (RIA), enzyme-linked immunosorbent assays (ELISAs), immunofluorescent assays, electrochemiluminescence assays, etc.

In some embodiments, the efficacy of the methods described herein can be monitored by detecting or monitoring a reduction in a symptom of a PNPLA3 disease, such as reduction in edema swelling of the extremities, face, larynx, upper respiratory tract, abdomen, trunk, and genitals, prodrome; laryngeal swelling; nonpruritic rash; nausea; vomiting; or abdominal pain. These symptoms may be assessed in vitro or in vivo using any method known in the art.

In some embodiments, the RNAi construct or a composition comprising the RNAi construct is administered to a subject such that the RNAi construct is delivered to a specific site within the subject. The inhibition of expression of PNPLA3 may be assessed using measurements of the level or change in the level of PNPLA3 mRNA or PNPLA3 protein in a sample derived from fluid or tissue from the specific site within the subject. In some embodiments, the RNAi construct may be delivered to a site such as the liver, choroid plexus, retina, and pancreas. The site may also be a subsection or subgroup of cells from any one of the aforementioned sites. The site may also include cells that express a particular type of receptor.

### Methods of Treating or Preventing PNPLA3-Associated Diseases

The present disclosure provides therapeutic and prophylactic methods which include administering to a subject with a PNPLA3-associated disease, disorder, and/or condition, or prone to developing, a PNPLA3-associated disease, disorder, and/or condition, an RNAi construct, compositions (e.g., pharmaceutical compositions) comprising an RNAi construct, or vectors comprising an RNAi construct as described herein. Non-limiting examples of PNPLA3-associated diseases include, for example, fatty liver (steatosis), nonalcoholic steatohepatitis (NASH), cirrhosis of the liver, accumulation of fat in the liver, inflammation of the liver, hepatocellular necrosis, liver fibrosis, obesity, and nonalcoholic fatty liver disease (NAFLD). In one embodiment, the PNPLA3-associated disease is NAFLD. In another embodiment, the PNPLA3-associated disease is NASH. In another embodiment, the PNPLA3-associated disease is fatty liver (steatosis). In another embodiment, the PNPLA3-associated disease is insulin resistance. In another embodiment, the PNPLA3-associated disease is not insulin resistance.

In certain embodiments, the present disclosure provides a method for reducing the expression of PNPLA3 in a patient in need thereof comprising administering to the patient any of the RNAi constructs described herein. The term “patient,” as used herein, refers to a mammal, including humans, and can be used interchangeably with the term “subject.” The expression level of PNPLA3 in hepatocytes in the patient desirably is reduced following administration of the RNAi construct as compared to the PNPLA3 expression level in a patient not receiving the RNAi construct.

The methods described herein are useful for treating a subject having a PNPLA3-associated disease, e.g., a subject that would benefit from reduction in PNPLA3 gene expression and/or PNPLA3 protein production. In one aspect, the present disclosure provides methods of reducing the level of Patatin-Like Phospholipase Domain Containing 3 (PNPLA3) gene expression in a subject having nonalcoholic fatty liver disease (NAFLD). In another aspect, the present disclosure provides methods of reducing the level of PNPLA3 protein in a subject with NAFLD. The present disclosure also provides methods of reducing the level of activity of the hedgehog pathway in a subject with NAFLD.

The treatment methods (and uses) described herein include administering to the subject, e.g., a human, a therapeutically effective amount of the disclosed RNAi construct targeting a PNPLA3 gene, a pharmaceutical composition comprising the RNAi construct, or a vector comprising the RNAi construct.

In one aspect, the disclosure provides methods of preventing at least one symptom in a subject having NAFLD, e.g., the presence of elevated hedgehog signaling pathways, fatigue, weakness, weight loss, loss of apetite, nausea, abdominal pain, spider-like blood vessels, yellowing of the skin and eyes (jaundice), itching, fluid buildup and swelling of the legs (edema), abdomen swelling (ascites), and mental confusion. The methods include administering to the subject a prophylactically effective amount of the RNAi construct, e.g., dsRNA, pharmaceutical compositions comprising the RNAi construct, or vectors encoding the RNAi construct, thereby preventing at least one symptom in the subject having a disorder that would benefit from reduction in PNPLA3 gene expression. A “prophylactically effective amount” refers to an amount effective, at dosages and for periods of time necessary, to achieve a desired prophylactic result (e.g., prevention of disease onset).

In another aspect, the present disclosure provides uses of a therapeutically effective amount of an RNAi construct described herein for treating a subject, e.g., a subject that would benefit from a reduction and/or inhibition of PNPLA3 gene expression. In a further aspect, the present disclosure provides uses of an RNAi construct, e.g., a dsRNA, targeting an PNPLA3 gene or pharmaceutical composition comprising an RNAi construct targeting an PNPLA3 gene in the manufacture of a medicament for treating a subject, e.g., a subject that would benefit from a reduction and/or inhibition of PNPLA3 gene expression and/or PNPLA3 protein production, such as a subject having a disorder that would benefit from reduction in PNPLA3 gene expression, e.g., a PNPLA3-associated disease.

The disclosure provides uses of an RNAi construct, e.g., a dsRNA, for preventing at least one symptom in a subject suffering from a disorder that would benefit from a reduction and/or inhibition of PNPLA3 gene expression and/or PNPLA3 protein production. For example, the disclosure provides uses of the RNAi construct described herein, compositions comprising same, and vectors comprising same, in the treatment of NAFLD.

In a further aspect, the present invention provides uses of the disclosed RNAi construct, compositions comprising same, or a vector comprising same, in the manufacture of a medicament for preventing at least one symptom in a subject suffering from a disorder that would benefit from a reduction and/or inhibition of PNPLA3 gene expression and/or PNPLA3 protein production, such as a PNPLA3-associated disease.

In one embodiment, an RNAi construct targeting PNPLA3 is administered to a subject having a PNPLA3-associated disease, e.g., nonalcoholic fatty liver disease (NAFLD), such that the expression of a PNPLA3 gene, e.g., in a cell, tissue, blood or other tissue or fluid of the subject are reduced by at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 62%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more when the RNAi construct is administered to the subject.

The methods and uses provided herein include administering a composition described herein such that expression of the target PNPLA3 gene is decreased for any suitable amount of time, such as for about 1, 2, 3, 4 5, 6, 7, 8, 12, 16, 18, 24, 28, 32, 36, 40, 44, 48, 52, 56, 60, 64, 68, 72, 76, or about 80 hours. In one embodiment, expression of the target PNPLA3 gene is decreased for an extended duration, e.g., at least about two, three, four, five, six, seven days or more, e.g., about one week, two weeks, three weeks, or about four weeks or longer.

Administration of the RNAi construct according to the methods and uses of the described herein may result in a reduction of the severity, signs, symptoms, and/or markers of such diseases or disorders in a patient with a PNPLA3-associated disease, e.g., NAFLD. By “reduction” in this context is meant a statistically significant decrease in such level. The reduction can be, for example, at least about 5%, 1%, 15%, 20%, 25%, 30%, 35%, 40%, 45%, 50%, 55%, 60%, 65%, 70%, 75%, 80%, 85%, 90%, 95%, or about 100%. Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of NAFLD may be assessed, for example, by periodic monitoring of NAFLD symptoms, liver fat levels, or expression of downstream genes. Comparison of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an RNAi targeting PNPLA3 or pharmaceutical composition thereof, “effective against” an PNPLA3-associated disease indicates that administration in a clinically appropriate manner results in a beneficial effect for at least a statistically significant fraction of patients, such as improvement of symptoms, a cure, a reduction in disease, extension of life, improvement in quality of life, or other effect generally recognized as positive by medical doctors familiar with treating NAFLD and/or an PNPLA3-associated disease and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given RNAi drug or formulation of that drug can also be judged using an experimental animal model for the given disease as known in the art. When using an experimental animal model, efficacy of treatment is evidenced when a statistically significant reduction in a marker or symptom is observed.

Subjects can be administered any therapeutically effective amount of the RNAi construct. Exemplary therapeutically effective amounts of the RNAi construct include, but are not limited to, 0.01 mg/kg, 0.02 mg/kg, 0.03 mg/kg, 0.04 mg/kg, 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg dsRNA, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 9.0 mg/kg, 10 mg/kg, 15 mg/kg, 20 mg/kg, 25 mg/kg, 30 mg/kg, 35 mg/kg, 40 mg/kg, 45 mg/kg, or about 50 mg/kg. In one embodiment, subjects can be administered 0.5 mg/kg of the RNAi construct. Values and ranges intermediate to the recited values also are encompassed by the present disclosure.

Administration of the RNAi construct, or a composition comprising same, can reduce the presence of PNPLA3 protein levels, e.g., in a cell, tissue, blood, urine or other compartment of the patient by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more.

Before administration of a full dose of the RNAi, patients can be administered a smaller dose, such as a 5% infusion, and monitored for adverse effects, such as an allergic reaction. In another example, the patient can be monitored for unwanted immunostimulatory effects, such as increased cytokine (e.g., TNF-alpha or INF-alpha) levels.

Owing to the inhibitory effects on PNPLA3 expression, a composition according to the disclosure or a pharmaceutical composition prepared therefrom can enhance the quality of life.

An RNAi described herein may be administered in “naked” form, where the modified or unmodified RNAi construct is directly suspended in aqueous or suitable buffer solvent, as a “free RNAi.” A free RNAi is administered in the absence of a pharmaceutical composition. The free RNAi may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolality of the buffer solution containing the RNAi can be adjusted such that it is suitable for administering to a subject.

Alternatively, an RNAi described herein may be administered as a pharmaceutical composition, such as a dsRNA liposomal formulation.

Subjects that would benefit from a reduction and/or inhibition of PNPLA3 gene expression are those having nonalcoholic fatty liver disease (NAFLD) and/or an PNPLA3-associated disease or disorder as described herein.

Treatment of a subject that would benefit from a reduction and/or inhibition of PNPLA3 gene expression includes therapeutic and prophylactic treatment.

The disclosure further provides methods and uses of an RNAi construct or a pharmaceutical composition thereof for treating a subject that would benefit from reduction and/or inhibition of PNPLA3 gene expression, e.g., a subject having a PNPLA3-associated disease, in combination with other pharmaceuticals and/or other therapeutic methods, e.g., with known pharmaceuticals and/or known therapeutic methods, such as, for example, those which are currently employed for treating these disorders.

For example, in certain embodiments, an RNAi targeting a PNPLA3 gene is administered in combination with, e.g., an agent useful in treating an PNPLA3-associated disease. For example, additional therapeutics and therapeutic methods suitable for treating a subject that would benefit from reduction in PNPLA3 expression, e.g., a subject having a PNPLA3-associated disease, include an RNAi construct targeting a different portion of the PNPLA3 gene, a therapeutic agent, and/or procedures for treating a PNPLA3-associated disease or a combination of any of the foregoing.

In certain embodiments, a first RNAi construct targeting a PNPLA3 gene is administered in combination with a second RNAi construct targeting a different portion of the PNPLA3 gene. For example, the first RNAi construct may comprise a first sense strand and a first antisense strand forming a double stranded region, wherein substantially all of the nucleotides of said first sense strand and substantially all of the nucleotides of the first antisense strand are modified nucleotides, wherein said first sense strand is conjugated to a ligand attached at the 3′-terminus, and wherein the ligand is one or more GalNAc derivatives attached through a bivalent or trivalent branched linker; and the second RNAi construct may comprise a second sense strand and a second antisense strand forming a double stranded region, wherein substantially all of the nucleotides of the second sense strand and substantially all of the nucleotides of the second antisense strand are modified nucleotides, wherein the second sense strand is conjugated to a ligand attached at the 3′-terminus, and wherein the ligand is one or more GalNAc derivatives attached through a bivalent or trivalent branched linker. In one embodiment, all of the nucleotides of the first and second sense strand and/or all of the nucleotides of the first and second antisense strand comprise a modification. The modified nucleotides may be any one or combination of the modified nucleotides described herein.

In other embodiments, a first RNAi construct targeting a PNPLA3 gene is administered in combination with a second RNAi construct targeting a gene that is different from the PNPLA3 gene. For example, the RNAi construct targeting the PNPLA3 gene may be administered in combination with an RNAi construct targeting the SCAP gene. SCAP (SREBP Cleavage Activating Protein) is the only known regulator of the transcription factors of the SREBP family. The SREBP (Sterol Response Element Binding Protein) family play important roles in regulating de novo lipogenesis and triglyceride (TG) accumulation within the liver. The first RNAi construct targeting a PNPLA3 gene and the second RNAi construct targeting a different gene, e.g., the SCAP gene, may be administered as parts of the same pharmaceutical composition. Alternatively, the first RNAi construct targeting a PNPLA3 gene and the second RNAi construct targeting a different gene, e.g., the SCAP gene, may be administered as parts of different pharmaceutical compositions.

The RNAi construct and an additional therapeutic agent and/or treatment may be administered at the same time and/or in the same combination, e.g., parenterally, or the additional therapeutic agent can be administered as part of a separate composition or at separate times and/or by another method known in the art or described herein.

The present disclosure also provides methods of using an RNAi construct and/or a composition containing an RNAi construct to reduce and/or inhibit PNPLA3 expression (gene or protein expression) in a cell. In yet other aspects, use of an RNAi construct and/or a composition comprising an RNAi construct for the manufacture of a medicament for reducing and/or inhibiting PNPLA3 gene expression in a cell are provided. In still other aspects, the present disclosure provides an RNAi described herein and/or a composition comprising an RNAi construct described herein for use in reducing and/or inhibiting PNPLA3 protein production in a cell. In yet other aspects, use of an RNAi construct and/or a composition comprising an RNAi construct for the manufacture of a medicament for reducing and/or inhibiting PNPLA3 protein production in a cell are provided. The methods and uses include contacting the cell with an RNAi construct, e.g., a dsRNA, and maintaining the cell for a time sufficient to obtain degradation of the mRNA transcript of a PNPLA3 gene, thereby inhibiting expression of the PNPLA3 gene or inhibiting PNPLA3 protein production in the cell. Reduction in gene expression can be assessed by any methods known in the art or described herein for determining mRNA or protein levels.

In the methods and uses described herein, the cell may be contacted in vitro or in vivo, i.e., the cell may be outside (e.g., in cell culture) or within a subject. A cell suitable for treatment using the methods described herein may be any cell that expresses an PNPLA3 gene, e.g., a cell from a subject having NAFLD or a cell comprising an expression vector comprising a PNPLA3 gene or portion of a PNPLA3 gene. A suitable cell for use in the disclosed methods includes, for example, a mammalian cell, e.g., a primate cell (such as a human cell or a non-human primate cell, e.g., a monkey cell or a chimpanzee cell), a non-primate cell (such as a cow cell, a pig cell, a camel cell, a llama cell, a horse cell, a goat cell, a rabbit cell, a sheep cell, a hamster, a guinea pig cell, a cat cell, a dog cell, a rat cell, a mouse cell, a lion cell, a tiger cell, a bear cell, or a buffalo cell), a bird cell (e.g., a duck cell or a goose cell), or a whale cell. In one embodiment, the cell is a human cell.

PNPLA3 gene expression may be inhibited in the cell by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%.

PNPLA3 protein production may be inhibited in the cell by at least about 5%, 6%, 7%, 8%, 9%, 10%, 11%, 12%, 13%, 14%, 15, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%.

The in vivo methods and uses described herein may include administering to a subject a composition containing an RNAi construct, where the RNAi construct includes a nucleotide sequence that is complementary to at least a part of an RNA transcript of the PNPLA3 gene of the subject. When the organism to be treated is a human, the composition can be administered by any means known in the art including, but not limited to subcutaneous, intravenous, oral, intraperitoneal, or parenteral routes, including intracranial (e.g., intraventricular, intraparenchymal, and intrathecal), intramuscular, transdermal, airway (aerosol), nasal, rectal, and topical (including buccal and sublingual) administration. In certain embodiments, the compositions are administered by subcutaneous or intravenous infusion or injection. In one embodiment, the compositions are administered by subcutaneous injection.

In some embodiments, the administration is via a depot injection. A depot injection may release the RNAi construct in a consistent way over a prolonged time period. Thus, a depot injection may reduce the frequency of dosing needed to obtain a desired effect, e.g., a desired inhibition of PNPLA3, or a therapeutic or prophylactic effect. A depot injection may also provide more consistent serum concentrations. Depot injections may include subcutaneous injections or intramuscular injections. In some embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In preferred embodiments, the infusion pump is a subcutaneous infusion pump. In other embodiments, the pump is a surgically implanted pump that delivers the RNAi to the subject.

The mode of administration may be chosen based upon whether local or systemic treatment is desired and based upon the area to be treated. The route and site of administration may be chosen to enhance targeting.

The methods and uses include administering to the mammal, e.g., a human, a composition comprising an RNAi construct, e.g., an siRNA, that targets an PNPLA3 gene in a cell of the mammal and maintaining the mammal for a time sufficient to obtain degradation of the mRNA transcript of the PNPLA3 gene, thereby inhibiting expression of the PNPLA3 gene in the mammal. Reduction in gene expression and/or protein expression can be assessed in a sample obtained from the RNAi construct-administered subject by any method known in the art or described herein. In one embodiment, a tissue sample serves as the tissue material for monitoring the reduction in PNPLA3 gene and/or protein expression. In another embodiment, a blood sample serves as the tissue material for monitoring the reduction in PNPLA3 gene and/or protein expression.

In some embodiments, verification of RISC-mediated cleavage of a target mRNA (e.g., PNPLA3 mRNA) in vivo following administration of an RNAi construct may be assessed by performing 5′-RACE or modifications of the protocol as known in the art (Lasham A et al., (2010) Nucleic Acid Res., 38 (3) p-el9; and Zimmermann et al. (2006) Nature 441: 111-4).

It is understood that all ribonucleic acid sequences disclosed herein can be converted to deoxyribonucleic acid sequences by substituting a thymine base for a uracil base in the sequence. Likewise, all deoxyribonucleic acid sequences disclosed herein can be converted to ribonucleic acid sequences by substituting a uracil base for a thymine base in the sequence. Deoxyribonucleic acid sequences, ribonucleic acid sequences, and sequences containing mixtures of deoxyribonucleotides and ribonucleotides of all sequences disclosed herein are encompassed by the present disclosure.

Additionally, any nucleic acid sequences disclosed herein may be modified with any combination of chemical modifications. One of skill in the art will readily appreciate that such designation as “RNA” or “DNA” to describe modified polynucleotides is, in certain instances, arbitrary. For example, a polynucleotide comprising a nucleotide having a 2′-OH substituent on the ribose sugar and a thymine base could be described as a DNA molecule having a modified sugar (2′-OH for the natural 2′-H of DNA) or as an RNA molecule having a modified base (thymine (methylated uracil) for natural uracil of RNA).

Accordingly, nucleic acid sequences provided herein, including but not limited to those set forth in the sequence listing, are intended to encompass nucleic acids containing any combination of natural or modified RNA and/or DNA, including, but not limited to, such nucleic acids having modified nucleobases. By way of a further example and without limitation, a polynucleotide having the sequence “ATCGATCG” (encompasses any polynucleotides having such a sequence, whether modified or unmodified, including, but not limited to, such compounds comprising RNA bases, such as those having sequence “AUCGAUCG” and those having some DNA bases and some RNA bases such as “AUCGATCG,” and polynucleotides having other modified bases, such as “ATmeCGAUCG,” wherein meC indicates a cytosine base comprising a methyl group at the 5-position.

The following examples, including the experiments conducted and the results achieved, are provided for illustrative purposes only and are not to be construed as limiting the scope of the appended claims.

### EXAMPLES

All animal experiments described herein were approved by the Institutional Animal Care and Use Committee (IACUC) of Amgen and cared for in accordance to the Guide for the Care and Use of Laboratory Animals, 8th Edition (National Research Council (U.S.)). Committee for the Update of the Guide for the Care and Use of Laboratory Animals, Institute for Laboratory Animal Research (U.S.), and National Academies Press (U.S.) (2011) Guide for the care and use of laboratory animals. 8th Ed., National Academies Press, Washington, D.C. Mice were single-housed in an air-conditioned room at 22±2° C. with a twelve-hour light; twelve-hour darkness cycle (0600-1800 hours). Animals had ad libitum access to a regular chow diet (Envigo, 2920X, or a diet as stated otherwise) and to water (reverse osmosis-purified) via automatic watering system, unless otherwise indicated. At termination, blood was collected by cardiac puncture under deep anesthesia, and then, following Association for Assessment and Accreditation of Laboratory Animal Care (AAALAC) guidelines, euthanized by a secondary physical method.

### Example 1: Selection, Design and Synthesis of Modified PNPLA3 siRNA Molecules

The identification and selection of optimal sequences for therapeutic siRNA molecules targeting patatin-like phospholipase domain-containing 3 (PNPLA3) were identified using bioinformatics analysis of a human PNPLA3 transcript (GenBank Accession No. NM_025225.2). The bioinformatics analysis identified more than 450 suitable human/cynomolgus triggers across the entire PNPLA3 transcript and revealed an extended 3′UTR of approximately 4,500 bp that has yet to be explored. Table 1 lists PNPLA3 mRNA target sequences identified as having therapeutic properties.

More than 600 siRNA molecules directed against the sequences listed in Table 1 were synthesized, and these sequences were modified to include a single chemical modification to improve the potency and in vivo stability of PNPLA3 siRNA sequences. The sense and antisense sequences for each of the unmodified and modified PNPLA3 siRNAs are shown in FIG. 1 and FIG. 2.

A panel of 194 siRNA molecules (or “triggers”) selective for human and cynomolgus (human/cyno) PNPLA3 spanning the coding region (CDR) and annotated 3′UTR were generated, along with an additional 162 human/cyno triggers targeting the extended 3′UTR and 64 human/marmoset triggers. Human-only triggers targeting the coding region also were prepared.

### Example 2: Efficacy of Select PNPLA3 siRNA Molecules in RNA FISH Assay

siRNA molecules synthesized in Example 1 with a single modification pattern (shown in FIG. 1) were screened in an in vitro siRNA transfection assay followed by a fluorescent in situ hybridization assay targeting ribonucleic acid molecules (RNA FISH) to determine IC₅₀ and maximum activity values. HepG2 cells (purchased from ATCC) were cultured in minimal essential medium EMEM from ATCC supplemented with 10% fetal bovine serum (FBS, Sigma) and 1% penicillin-streptomycin (P-S, Corning).

The siRNA transfection was performed as follows: 1 μL of test siRNAs and 4 μL of plain EMEM were added to PDL-coated CellCarrier-384 Ultra assay plates (PerkinElmer) by Bravo Automated Liquid Handling (Agilent). 5 μL of Lipofectamine RNAiMAX (Thermo Fisher Scientific), pre-diluted in EMEM (specifically 0.06 μL of RNAiMAX in 5 μL EMEM), was then dispensed into the assay plates by Multidrop Combi Reagent Dispenser (Thermo Fisher Scientific). After a 20 minute incubation of the siRNA/RNAiMAX mixture at room temperature (RT), 30 μL of HepG2 cells (2000 cells per well) in EMEM supplemented with 10% FBS and 1% P-S were added to the transfection complex using Multidrop Combi Reagent Dispenser. The assay plates were incubated for 20 minutes at RT prior to being placed in an incubator. Cells were then incubated for 72 hours at 37° C. and 5% CO2.

The RNA FISH assay was performed using the Affymetrix QUANTIGENE® View RNA HC Screening Assay kit (QVP0011), the Affymetrix View HC Signal Amplification Kit 3-plex (QVP0213), and Affymetrix gene specific probes: Human PNPLA3 (VA6-20279-01) and Human PPIB (VA1-10148-01), following the manufacturer's protocol using an in-house assembled automated FISH assay platform for liquid handling.

In brief, cells were fixed in 4% formaldehyde (Thermo Fisher Scientific) for 15 minutes at RT, permeabilized with detergent for 3 minutes at RT and then treated with protease solution for 10 minutes at RT. Incubation of PNPLA3 and PPIB probe pairs was done for 3 hours, while for Preamplifiers, Amplifiers and Label Probes were for 1 hr each. All hybridization steps were carried out at 40° C. in a Cytomat 2 C-LIN automated incubator (Thermo Fisher Scientific). After hybridization reactions, cells were stained for 30 minutes with Hoechst and CellMask Blue (Thermo Fisher Scientific).

All plates were imaged on an Opera Phenix High Content Screening System (PerkinElmer), using the UV Channel for Hoechst 33342 and Cell Mask Blue, the 488 Channel for PPIB probes, and the 647 Channel for PNPLA3 probes.

The images were analyzed using Columbus Image Data Storage and Analysis System (PerkinElmer) to obtain mean spot counts per cell. The spot counts were normalized using the high (containing phosphate-buffered saline, Corning) and low (without target probe pairs) control wells. The normalized values against the total siRNA concentrations were plotted and the data were fit to a four-parameter sigmoidal model in Genedata Screener (Genedata) to obtain IC₅₀ and maximum activity. The results for all siRNAs generated are shown in Table 2. Results for the panel of 194 human/cyno siRNA molecules spanning the PNPLA3 CDR and annotated 3′UTR are shown in Table 3. Results for the panel of 162 human/cyno siRNA molecules targeting the extended 3′UTR are shown in Table 4, and results for the panel 64 human/marmoset siRNA molecules are shown in Table 5.

PNPLA3 knockdown provides a percentage of knockdown compared to control. Negative values indicate a decrease in PNPLA3 levels. 22 siRNA triggers achieved >75% mRNA knockdown. In comparison, a minor allele-specific siRNA directed to PNPLA3 I148M (described in WO 2020/123508) reduces the expression of PNPLA3 mRNA by approximately 81%. The top 48 triggers were synthesized as GalNAc-siRNA conjugates.

TABLE 2

| | PNPLA3 | % Avg Max | |
|---|---|---|---|
| Duplex No. | Location | Activity | Avg IC₅₀ (nM) |
| 44956-1 | 1233 | −97.0 | 1.61 |
| 45212-1 | 571 | −90.8 | 16.80 |
| 44928-1 | 1367 | −90.5 | 1.39 |
| 44912-1 | 449 | −88.2 | 1.99 |
| 45207-1 | 1564 | −87.8 | 9.98 |
| 45221-1 | 1379 | −86.7 | 38.80 |
| 44911-1 | 445 | −85.8 | 1.37 |
| 41946-1 | 844 | −85.8 | 1.59 |
| 44957-1 | 1234 | −84.3 | 0.33 |
| 42048-1 | 1561 | −83.6 | 1.92 |
| 41955-1 | 909 | −82.8 | 3.84 |
| 37781-1 | 698 | −82.7 | 2.69 |
| 44939-1 | 991 | −82.5 | 17.70 |
| 42064-1 | 1240 | −82.3 | 1.21 |
| 44908-1 | 396 | −82.3 | 2.88 |
| 41921-1 | 574 | −81.9 | 2.43 |
| 37893-1 | 768 | −81.8 | 5.28 |
| 41932-1 | 692 | −81.1 | 5.52 |
| 37600-1 | 347 | −80.9 | 1.77 |
| 37930-1 | 1219 | −80.9 | 2.11 |
| 38215-1 | 2198 | −80.2 | 1.78 |
| 45218-1 | 1184 | −79.8 | 19.00 |
| 37785-1 | 704 | −79.6 | 1.85 |
| 37744-1 | 391 | −79.6 | 2.68 |
| 45220-1 | 1185 | −79.5 | 19.30 |
| 38217-1 | 2200 | −79.4 | 1.93 |
| 41942-1 | 811 | −79.4 | 0.53 |
| 38216-1 | 2199 | −79.4 | 2.63 |
| 37745-1 | 392 | −79.3 | 2.49 |
| 44922-1 | 1359 | −79.0 | 4.64 |
| 44955-1 | 1168 | −79.0 | 4.93 |
| 41949-1 | 853 | −79.0 | 3.37 |
| 45201-1 | 1562 | −78.8 | 4.34 |
| 44905-1 | 812 | −78.8 | 2.95 |
| 45216-1 | 842 | −78.7 | 10.80 |
| 44910-1 | 442 | −78.7 | 13.40 |
| 41935-1 | 776 | −78.6 | 4.31 |
| 44909-1 | 397 | −78.6 | 2.14 |
| 44915-1 | 1328 | −78.6 | 4.53 |
| 41919-1 | 572 | −78.6 | 4.54 |
| 44946-1 | 1024 | −78.3 | 1.31 |
| 44949-1 | 1029 | −78.2 | 9.80 |
| 44940-1 | 994 | −78.0 | 4.38 |
| 42062-1 | 1236 | −78.0 | 2.04 |
| 45200-1 | 696 | −77.9 | 9.55 |
| 41950-1 | 854 | −77.9 | 11.75 |
| 44923-1 | 1361 | −77.9 | 14.60 |
| 37889-1 | 764 | −77.7 | 2.12 |
| 44914-1 | 459 | −76.9 | 3.20 |
| 42066-1 | 1266 | −76.7 | 14.70 |
| 37789-1 | 756 | −76.5 | 1.88 |
| 44950-1 | 1042 | −76.5 | 4.28 |
| 37588-1 | 309 | −76.4 | 1.50 |
| 37786-1 | 705 | −76.4 | 1.56 |
| 37864-1 | 1602 | −76.3 | 2.46 |
| 45199-1 | 695 | −76.2 | 6.84 |
| 37907-1 | 893 | −75.8 | 1.58 |
| 41954-1 | 907 | −75.6 | 5.08 |
| 41960-1 | 923 | −75.4 | 5.48 |
| 41953-1 | 906 | −75.4 | 4.68 |
| 37763-1 | 590 | −75.3 | 2.50 |
| 44927-1 | 1366 | −75.1 | 0.91 |
| 44918-1 | 1334 | −75.0 | 5.36 |
| 37915-1 | 905 | −75.0 | 11.08 |
| 41917-1 | 540 | −74.9 | 2.83 |
| 37843-1 | 1224 | −74.8 | 4.86 |
| 41920-1 | 573 | −74.8 | 4.89 |
| 37563-1 | 211 | −74.7 | 5.64 |
| 45217-1 | 790 | −74.6 | 22.90 |
| 45219-1 | 1262 | −74.3 | 29.80 |
| 37787-1 | 706 | −74.2 | 1.70 |
| 37846-1 | 1280 | −74.1 | 2.70 |
| 44951-1 | 1051 | −73.9 | 2.67 |
| 37765-1 | 593 | −73.7 | 2.61 |
| 42047-1 | 1560 | −73.7 | 15.82 |
| 42060-1 | 1229 | −73.7 | 1.96 |
| 37886-1 | 2197 | −73.6 | 2.11 |
| 44925-1 | 1364 | −73.6 | 5.41 |
| 37842-1 | 1223 | −73.3 | 2.30 |
| 44906-1 | 353 | −73.3 | 47.70 |
| 44920-1 | 1356 | −73.3 | 10.60 |
| 41922-1 | 576 | −73.3 | 1.17 |
| 45210-1 | 546 | −73.2 | 9.79 |
| 44917-1 | 1332 | −73.2 | 2.06 |
| 37567-1 | 220 | −73.0 | 2.02 |
| 37932-1 | 1220 | −72.9 | 2.69 |
| 38218-1 | 2201 | −72.6 | 1.64 |
| 37892-1 | 767 | −72.3 | 1.06 |
| 37908-1 | 894 | −72.3 | 2.04 |
| 41951-1 | 855 | −72.2 | 4.09 |
| 41947-1 | 845 | −72.2 | 1.21 |
| 42063-1 | 1237 | −72.1 | 0.52 |
| 38214-1 | 1092 | −71.3 | 4.71 |
| 37770-1 | 662 | −71.3 | 3.87 |
| 37865-1 | 1603 | −71.2 | 1.45 |
| 38210-1 | 1189 | −71.2 | 3.68 |
| 37758-1 | 562 | −71.2 | 2.61 |
| 37759-1 | 563 | −71.0 | 1.80 |
| 37913-1 | 902 | −70.8 | 2.32 |
| 37880-1 | 1890 | −70.5 | 3.03 |
| 44924-1 | 1363 | −70.5 | 3.45 |
| 41939-1 | 786 | −70.3 | 7.09 |
| 37858-1 | 1322 | −70.1 | 2.11 |
| 41945-1 | 817 | −70.1 | 1.36 |
| 44921-1 | 1358 | −69.8 | 6.09 |
| 44926-1 | 1365 | −69.8 | 4.52 |
| 45241-1 | 1013 | −69.6 | 26.60 |
| 37782-1 | 701 | −69.3 | 2.64 |
| 42054-1 | 989 | −69.3 | 14.45 |
| 37845-1 | 1226 | −69.2 | 5.66 |
| 37927-1 | 1201 | −69.1 | 2.08 |
| 42043-1 | 1554 | −69.1 | 2.53 |
| 37762-1 | 568 | −69.1 | 5.37 |
| 44947-1 | 1027 | −69.0 | 7.08 |
| 41952-1 | 856 | −68.7 | 2.23 |
| 42067-1 | 1268 | −68.5 | 3.03 |
| 42052-1 | 1618 | −68.3 | 2.86 |
| 42046-1 | 1558 | −68.0 | 9.21 |
| 42035-1 | 1376 | −68.0 | 3.19 |
| 42049-1 | 1607 | −67.9 | 5.67 |
| 40810-1 | 4193 | −67.9 | 1.81 |
| 37849-1 | 1300 | −67.9 | 1.60 |
| 38213-1 | 897 | −67.8 | 2.38 |
| 37897-1 | 832 | −67.6 | 1.64 |
| 37929-1 | 1213 | −67.6 | 1.10 |
| 37777-1 | 671 | −67.5 | 3.47 |
| 42034-1 | 1371 | −67.4 | 3.19 |
| 41910-1 | 510 | −67.2 | 7.79 |
| 42056-1 | 995 | −67.0 | 1.19 |
| 37874-1 | 1771 | −67.0 | 2.17 |
| 38220-1 | 2204 | −66.9 | 1.73 |
| 37903-1 | 878 | −66.8 | 1.84 |
| 37767-1 | 641 | −66.7 | 4.83 |
| 37912-1 | 899 | −66.7 | 9.32 |
| 42042-1 | 1553 | −66.7 | 7.24 |
| 41927-1 | 604 | −66.6 | 10.83 |
| 38212-1 | 1217 | −66.6 | 3.50 |
| 37871-1 | 1763 | −66.6 | 2.21 |
| 45263-1 | 458 | −66.4 | 38.60 |
| 44943-1 | 1020 | −66.4 | 16.40 |
| 44932-1 | 1446 | −66.2 | 3.83 |
| 42065-1 | 1250 | −66.2 | 3.16 |
| 38219-1 | 2202 | −66.2 | 1.75 |
| 37909-1 | 895 | −66.1 | 2.49 |
| 42061-1 | 1230 | −65.9 | 2.15 |
| 44937-1 | 966 | −65.9 | 11.50 |
| 40789-1 | 3858 | −65.9 | 1.60 |
| 44948-1 | 1028 | −65.8 | 5.49 |
| 37870-1 | 1760 | −65.6 | 1.27 |
| 45203-1 | 822 | −65.5 | 9.72 |
| 45334-1 | 451 | −65.4 | 0.78 |
| 40788-1 | 3857 | −65.2 | 1.00 |
| 41163-1 | 2187 | −65.2 | 0.94 |
| 40817-1 | 4260 | −65.2 | 2.35 |
| 37757-1 | 560 | −65.1 | 2.46 |
| 37872-1 | 1764 | −65.1 | 2.17 |
| 37756-1 | 557 | −65.0 | 5.74 |
| 37888-1 | 762 | −65.0 | 1.55 |
| 41031-1 | 5640 | −65.0 | 20.16 |
| 45215-1 | 1413 | −64.9 | 3.96 |
| 37753-1 | 497 | −64.9 | 5.10 |
| 41179-1 | 4117 | −64.8 | 1.68 |
| 41175-1 | 4112 | −64.7 | 1.08 |
| 41150-1 | 715 | −64.6 | 1.28 |
| 41959-1 | 919 | −64.6 | 3.24 |
| 37877-1 | 1852 | −64.5 | 3.61 |
| 41178-1 | 4116 | −64.5 | 0.25 |
| 45225-1 | 1333 | −64.2 | Undefined |
| 42050-1 | 1612 | −64.0 | 1.76 |
| 41914-1 | 514 | −63.9 | 6.33 |
| 37905-1 | 887 | −63.8 | 6.37 |
| 41174-1 | 3893 | −63.7 | 1.02 |
| 37866-1 | 1604 | −63.6 | 2.05 |
| 44944-1 | 1021 | −63.6 | 2.68 |
| 37920-1 | 1101 | −63.5 | 5.96 |
| 37760-1 | 564 | −63.4 | 11.50 |
| 42044-1 | 1556 | −63.3 | 17.39 |
| 37890-1 | 765 | −63.3 | 1.34 |
| 37921-1 | 1104 | −63.1 | 1.88 |
| 41943-1 | 813 | −63.1 | 3.87 |
| 41149-1 | 714 | −63.0 | 1.68 |
| 44929-1 | 1373 | −63.0 | 9.61 |
| 41908-1 | 360 | −62.6 | 13.30 |
| 37764-1 | 591 | −62.3 | 21.50 |
| 41145-1 | 422 | −62.3 | 0.78 |
| 37561-1 | 206 | −62.2 | 3.42 |
| 42055-1 | 992 | −62.1 | 2.82 |
| 37754-1 | 555 | −62.1 | 4.56 |
| 37570-1 | 245 | −61.7 | 3.21 |
| 37595-1 | 339 | −61.5 | 1.78 |
| 45250-1 | 1357 | −61.4 | Undefined |
| 37878-1 | 1855 | −61.3 | 2.78 |
| 40884-1 | 4500 | −61.2 | 5.64 |
| 44935-1 | 1500 | −61.0 | 24.20 |
| 44903-1 | 312 | −60.8 | 12.60 |
| 37583-1 | 302 | −60.6 | 3.39 |
| 38208-1 | 763 | −60.4 | 3.37 |
| 37585-1 | 305 | −60.3 | 22.60 |
| 37587-1 | 307 | −60.3 | 3.42 |
| 40866-1 | 4290 | −60.1 | 2.00 |
| 41152-1 | 718 | −60.1 | 0.35 |
| 37779-1 | 675 | −60.1 | 13.70 |
| 37761-1 | 567 | −60.0 | 2.81 |
| 45232-1 | 857 | −60.0 | 33.20 |
| 41928-1 | 605 | −60.0 | 2.81 |
| 41170-1 | 2269 | −59.9 | 2.02 |
| 37896-1 | 805 | −59.8 | 4.08 |
| 45257-1 | 608 | −59.8 | Undefined |
| 37891-1 | 766 | −59.8 | 1.09 |
| 37569-1 | 244 | −59.8 | 2.19 |
| 37910-1 | 896 | −59.7 | 8.15 |
| 44938-1 | 967 | −59.6 | Undefined |
| 41070-1 | 6594 | −59.6 | 1.76 |
| 37852-1 | 1312 | −59.4 | 4.67 |
| 37586-1 | 306 | −59.3 | 1.80 |
| 37580-1 | 291 | −59.1 | 7.76 |
| 37911-1 | 898 | −59.1 | 1.82 |
| 44952-1 | 1148 | −59.0 | 27.70 |
| 37581-1 | 293 | −58.8 | 3.74 |
| 42040-1 | 1516 | −58.6 | 14.84 |
| 37850-1 | 1310 | −58.4 | 3.73 |
| 41146-1 | 423 | −58.4 | 2.01 |
| 37565-1 | 213 | −58.1 | 13.90 |
| 37844-1 | 1225 | −58.0 | 3.70 |
| 37568-1 | 243 | −57.9 | 2.01 |
| 41941-1 | 807 | −57.8 | 1.86 |
| 41039-1 | 5674 | −57.7 | 1.12 |
| 37594-1 | 338 | −57.6 | 8.13 |
| 41142-1 | 266 | −57.6 | 2.01 |
| 37788-1 | 708 | −57.5 | 2.82 |
| 44904-1 | 774 | −57.5 | 5.83 |
| 37750-1 | 415 | −57.5 | 22.70 |
| 41934-1 | 694 | −57.3 | 1.26 |
| 45247-1 | 1494 | −57.3 | Undefined |
| 41915-1 | 515 | −57.3 | 8.83 |
| 40906-1 | 5578 | −57.3 | 2.78 |
| 41053-1 | 6199 | −57.2 | 0.25 |
| 41042-1 | 5680 | −57.0 | 1.20 |
| 40783-1 | 3813 | −56.9 | 3.54 |
| 37906-1 | 889 | −56.8 | Undefined |
| 40899-1 | 5012 | −56.8 | 3.59 |
| 41288-1 | 6644 | −56.7 | 1.74 |
| 41171-1 | 2273 | −56.7 | 2.83 |
| 37848-1 | 1296 | −56.2 | 2.86 |
| 37885-1 | 2014 | −56.1 | 2.87 |
| 37584-1 | 303 | −56.1 | 5.70 |
| 37873-1 | 1765 | −55.8 | 2.29 |
| 40907-1 | 5579 | −55.6 | 1.52 |
| 38221-1 | 2216 | −55.6 | 2.60 |
| 44919-1 | 1344 | −55.6 | 6.36 |
| 41052-1 | 6163 | −55.5 | 0.39 |
| 41155-1 | 723 | −55.5 | 0.23 |
| 37869-1 | 1757 | −55.4 | 2.63 |
| 41048-1 | 5741 | −55.2 | 1.72 |
| 37863-1 | 1596 | −55.1 | 2.53 |
| 44916-1 | 1329 | −55.1 | 15.50 |
| 41424-1 | 4119 | −55.0 | 1.83 |
| 37593-1 | 337 | −54.8 | 7.08 |
| 41050-1 | 6153 | −54.5 | 1.62 |
| 37578-1 | 272 | −54.5 | 4.98 |
| 37851-1 | 1311 | −54.0 | 2.55 |
| 44942-1 | 1010 | −53.8 | 22.60 |
| 37881-1 | 1917 | −53.7 | 6.86 |
| 37776-1 | 669 | −53.7 | 20.80 |
| 41162-1 | 1860 | −53.7 | 0.95 |
| 41944-1 | 814 | −53.6 | 0.76 |
| 41176-1 | 4113 | −53.4 | 1.27 |
| 41161-1 | 1858 | −53.3 | 0.68 |
| 37895-1 | 770 | −53.3 | 1.92 |
| 41911-1 | 511 | −53.1 | 2.80 |
| 41066-1 | 6573 | −53.0 | 0.23 |
| 40806-1 | 4140 | −52.9 | 2.26 |
| 37774-1 | 666 | −52.6 | 2.97 |
| 41153-1 | 721 | −52.5 | 1.43 |
| 42039-1 | 1504 | −52.3 | 3.54 |
| 41956-1 | 912 | −52.1 | 4.01 |
| 45246-1 | 1277 | −51.6 | Undefined |
| 40882-1 | 4495 | −51.3 | 3.01 |
| 44930-1 | 1416 | −51.2 | Undefined |
| 41916-1 | 538 | −51.2 | 2.64 |
| 37868-1 | 1754 | −51.1 | 2.21 |
| 41295-1 | 6635 | −51.1 | 0.62 |
| 37847-1 | 1289 | −51.0 | Undefined |
| 37597-1 | 341 | −51.0 | 3.81 |
| 38209-1 | 1095 | −51.0 | 3.54 |
| 37867-1 | 1666 | −50.9 | 2.42 |
| 44945-1 | 1022 | −50.9 | 16.00 |
| 44954-1 | 1152 | −50.8 | Undefined |
| 37876-1 | 1851 | −50.7 | Undefined |
| 41413-1 | 5820 | −50.6 | 1.46 |
| 41414-1 | 5819 | −50.6 | 1.19 |
| 37755-1 | 556 | −50.5 | 2.73 |
| 37572-1 | 248 | −50.5 | 10.30 |
| 37571-1 | 246 | −50.5 | 6.47 |
| 45239-1 | 988 | −50.5 | Undefined |
| 45211-1 | 1347 | −50.3 | Undefined |
| 41418-1 | 5771 | −50.3 | 1.73 |
| 37784-1 | 703 | −50.3 | 13.30 |
| 37775-1 | 667 | −50.3 | 7.64 |
| 41069-1 | 6576 | −50.3 | 1.26 |
| 45202-1 | 493 | −50.3 | Undefined |
| 37574-1 | 251 | −50.2 | 7.09 |
| 37861-1 | 1402 | −50.1 | 3.16 |
| 41299-1 | 6611 | −49.9 | 3.01 |
| 37562-1 | 207 | −49.8 | 3.77 |
| 37853-1 | 1317 | −49.8 | Undefined |
| 41057-1 | 6265 | −49.7 | 0.76 |
| 37747-1 | 412 | −49.6 | 2.21 |
| 40893-1 | 4958 | −49.6 | 7.06 |
| 37919-1 | 1094 | −49.6 | 8.21 |
| 41925-1 | 585 | −49.3 | 8.51 |
| 40782-1 | 3796 | −49.3 | 3.35 |
| 37768-1 | 660 | −49.3 | 1.70 |
| 37859-1 | 1352 | −49.0 | 4.78 |
| 44953-1 | 1149 | −49.0 | Undefined |
| 37766-1 | 595 | −48.8 | 2.62 |
| 41065-1 | 6571 | −48.7 | 1.96 |
| 41931-1 | 691 | −48.6 | 1.18 |
| 41177-1 | 4115 | −48.4 | 2.23 |
| 37899-1 | 867 | −48.3 | 5.69 |
| 42041-1 | 1517 | −48.3 | 6.18 |
| 41924-1 | 584 | −48.2 | 6.92 |
| 41957-1 | 916 | −47.9 | 0.73 |
| 41294-1 | 6636 | −47.9 | 1.72 |
| 45259-1 | 351 | −47.8 | 40.30 |
| 42045-1 | 1557 | −47.8 | 5.43 |
| 37884-1 | 2013 | −47.3 | 2.45 |
| 37769-1 | 661 | −47.3 | Undefined |
| 41289-1 | 6643 | −47.2 | 0.78 |
| 45233-1 | 354 | −47.2 | Undefined |
| 40885-1 | 4543 | −47.2 | 3.40 |
| 45258-1 | 606 | −47.1 | Undefined |
| 37557-1 | 121 | −47.0 | 3.90 |
| 45204-1 | 460 | −46.8 | 12.60 |
| 40865-1 | 4289 | −46.7 | 2.97 |
| 37875-1 | 1775 | −46.7 | 2.43 |
| 37841-1 | 1221 | −46.5 | 4.28 |
| 41307-1 | 4046 | −46.2 | 3.56 |
| 37882-1 | 1918 | −46.2 | 3.15 |
| 41426-1 | 258 | −46.1 | 2.53 |
| 37749-1 | 414 | −45.9 | 4.85 |
| 37559-1 | 204 | −45.8 | 3.19 |
| 40898-1 | 4968 | −45.8 | 1.01 |
| 40900-1 | 5026 | −45.7 | 0.91 |
| 37783-1 | 702 | −45.7 | 5.25 |
| 37560-1 | 205 | −45.4 | 3.29 |
| 42038-1 | 1450 | −45.4 | 1.28 |
| 41410-1 | 5824 | −45.4 | 2.16 |
| 37772-1 | 664 | −45.3 | 1.66 |
| 37599-1 | 344 | −45.2 | 12.00 |
| 40892-1 | 4957 | −45.2 | 3.78 |
| 41151-1 | 716 | −45.1 | 3.55 |
| 37917-1 | 939 | −45.1 | 1.76 |
| 41408-1 | 6601 | −45.0 | 6.07 |
| 37926-1 | 1200 | −45.0 | Undefined |
| 37752-1 | 419 | −44.8 | 26.80 |
| 41293-1 | 6637 | −44.7 | 0.72 |
| 41948-1 | 848 | −44.6 | 1.07 |
| 40777-1 | 3775 | −44.6 | 8.56 |
| 40784-1 | 3820 | −44.5 | 10.52 |
| 41308-1 | 4008 | −44.5 | 4.12 |
| 42069-1 | 1324 | −44.5 | 0.51 |
| 41933-1 | 693 | −44.2 | 1.02 |
| 44907-1 | 357 | −44.1 | 13.10 |
| 41169-1 | 2268 | −43.9 | 0.39 |
| 41030-1 | 5601 | −43.4 | 1.14 |
| 37748-1 | 413 | −43.2 | 3.55 |
| 37901-1 | 869 | −42.7 | Undefined |
| 37856-1 | 1320 | −42.6 | 3.47 |
| 45264-1 | 197 | −42.5 | Undefined |
| 41157-1 | 730 | −42.5 | 1.26 |
| 41290-1 | 6641 | −42.5 | 0.78 |
| 41912-1 | 512 | −42.2 | 1.75 |
| 45229-1 | 782 | −42.0 | Undefined |
| 41280-1 | 6686 | −41.8 | 0.92 |
| 41936-1 | 777 | −41.6 | 0.72 |
| 37883-1 | 2012 | −41.5 | 13.70 |
| 40791-1 | 3862 | −41.3 | 2.68 |
| 40779-1 | 3793 | −41.1 | 1.67 |
| 41937-1 | 778 | −41.0 | 1.49 |
| 41300-1 | 6610 | −41.0 | 12.53 |
| 37582-1 | 294 | −40.9 | Undefined |
| 37904-1 | 886 | −40.8 | 8.22 |
| 37857-1 | 1321 | −40.7 | 1.62 |
| 41420-1 | 5707 | −40.5 | 2.86 |
| 41419-1 | 5770 | −40.5 | 1.77 |
| 38211-1 | 1210 | −39.9 | Undefined |
| 45248-1 | 1439 | −39.7 | 45.30 |
| 45236-1 | 402 | −39.7 | Undefined |
| 42053-1 | 985 | −39.6 | Undefined |
| 37879-1 | 1857 | −39.6 | Undefined |
| 41040-1 | 5676 | −39.3 | 9.14 |
| 37746-1 | 411 | −39.2 | 32.60 |
| 37743-1 | 369 | −39.2 | 7.55 |
| 37924-1 | 1125 | −39.0 | 14.50 |
| 45213-1 | 1081 | −38.8 | Undefined |
| 37751-1 | 416 | −38.8 | 21.10 |
| 42059-1 | 1044 | −38.6 | 10.70 |
| 37564-1 | 212 | −38.1 | Undefined |
| 45249-1 | 1039 | −38.0 | Undefined |
| 41298-1 | 6612 | −38.0 | 0.25 |
| 40903-1 | 5571 | −38.0 | 3.19 |
| 40818-1 | 4261 | −37.9 | Undefined |
| 41923-1 | 583 | −37.8 | 1.98 |
| 41909-1 | 490 | −37.4 | 2.42 |
| 42037-1 | 1445 | −37.3 | Undefined |
| 37773-1 | 665 | −37.2 | >100 |
| 40864-1 | 4286 | −37.1 | 1.59 |
| 41291-1 | 6640 | −36.8 | 0.58 |
| 41281-1 | 6685 | −36.8 | 0.70 |
| 41287-1 | 6669 | −36.8 | 1.42 |
| 37566-1 | 219 | −36.7 | Undefined |
| 41958-1 | 918 | −36.7 | 0.18 |
| 45256-1 | 352 | −36.5 | Undefined |
| 37771-1 | 663 | −36.4 | >100 |
| 41049-1 | 5967 | −36.4 | 1.18 |
| 40795-1 | 3958 | −36.1 | 2.49 |
| 41284-1 | 6676 | −36.1 | 0.18 |
| 41305-1 | 5642 | −36.0 | Undefined |
| 40790-1 | 3860 | −36.0 | 1.85 |
| 41301-1 | 6162 | −35.8 | Undefined |
| 41166-1 | 2191 | −35.8 | 0.31 |
| 40796-1 | 4006 | −35.8 | 2.18 |
| 41309-1 | 3773 | −35.7 | 2.48 |
| 41285-1 | 6671 | −35.5 | 1.14 |
| 41158-1 | 1136 | −35.3 | 3.51 |
| 40888-1 | 4903 | −35.2 | Undefined |
| 41282-1 | 6684 | −35.0 | 0.22 |
| 40792-1 | 3920 | −34.9 | 2.40 |
| 41046-1 | 5736 | −34.2 | 0.60 |
| 41913-1 | 513 | −34.2 | 4.74 |
| 40891-1 | 4906 | −34.1 | 1.86 |
| 41156-1 | 729 | −34.1 | 0.26 |
| 41421-1 | 4983 | −33.6 | Undefined |
| 41286-1 | 6670 | −33.6 | 0.38 |
| 40808-1 | 4186 | −33.5 | Undefined |
| 40780-1 | 3794 | −33.1 | 1.49 |
| 40878-1 | 4484 | −33.1 | 7.66 |
| 40785-1 | 3851 | −33.0 | 3.36 |
| 41297-1 | 6613 | −32.9 | 0.39 |
| 45209-1 | 1038 | −32.6 | Undefined |
| 40875-1 | 4402 | −32.2 | 3.47 |
| 41173-1 | 2639 | −32.2 | 4.98 |
| 41051-1 | 6157 | −32.1 | 1.39 |
| 41147-1 | 424 | −32.0 | 1.80 |
| 41415-1 | 5774 | −31.6 | 0.93 |
| 41154-1 | 722 | −31.4 | 1.72 |
| 40805-1 | 4139 | −31.3 | 2.89 |
| 41044-1 | 5692 | −31.3 | 0.33 |
| 37780-1 | 679 | −31.1 | 40.40 |
| 41306-1 | 4097 | −31.0 | 1.16 |
| 41159-1 | 1137 | −30.9 | 0.30 |
| 37855-1 | 1319 | −30.8 | >100 |
| 40797-1 | 4020 | −30.7 | 1.24 |
| 41160-1 | 1162 | −30.6 | 2.11 |
| 41045-1 | 5705 | −30.5 | Undefined |
| 41068-1 | 6575 | −30.4 | 1.82 |
| 44941-1 | 1003 | −30.3 | 18.20 |
| 40902-1 | 5569 | −30.1 | 1.05 |
| 41180-1 | 4118 | −30.0 | 0.73 |
| 41283-1 | 6683 | −30.0 | 0.34 |
| 40887-1 | 4902 | −29.9 | 22.40 |
| 40896-1 | 4965 | −29.9 | Undefined |
| 41054-1 | 6201 | −29.3 | 0.76 |
| 41168-1 | 2267 | −29.3 | Undefined |
| 41033-1 | 5668 | −29.0 | 2.21 |
| 37590-1 | 334 | −28.3 | >100 |
| 41296-1 | 6614 | −28.3 | 1.30 |
| 41918-1 | 542 | −28.2 | Undefined |
| 37862-1 | 1431 | −28.2 | Undefined |
| 45237-1 | 448 | −28.2 | Undefined |
| 41292-1 | 6638 | −28.0 | 0.44 |
| 45240-1 | 1006 | −27.9 | Undefined |
| 37898-1 | 866 | −27.7 | >100 |
| 41411-1 | 5823 | −27.5 | Undefined |
| 45222-1 | 194 | −27.3 | Undefined |
| 40803-1 | 4137 | −27.2 | 2.73 |
| 41043-1 | 5681 | −26.7 | 0.51 |
| 37558-1 | 122 | −26.7 | >100 |
| 41059-1 | 6268 | −26.6 | 0.24 |
| 40868-1 | 4318 | −26.4 | 2.72 |
| 41422-1 | 4441 | −26.4 | 1.72 |
| 40880-1 | 4488 | −26.2 | 1.92 |
| 40816-1 | 4259 | −25.9 | 4.50 |
| 41302-1 | 6158 | −25.8 | 1.97 |
| 37596-1 | 340 | −25.5 | >100 |
| 37914-1 | 904 | −25.2 | Undefined |
| 41032-1 | 5641 | −25.1 | 2.63 |
| 40881-1 | 4491 | −25.1 | 1.40 |
| 37598-1 | 343 | −24.8 | >100 |
| 41409-1 | 5857 | −24.8 | >33.3 |
| 41416-1 | 5773 | −24.5 | >100 |
| 41038-1 | 5673 | −23.7 | >33.3 |
| 40786-1 | 3853 | −23.4 | >33.3 |
| 41062-1 | 6280 | −23.2 | Undefined |
| 40811-1 | 4199 | −23.1 | >100 |
| 45235-1 | 364 | −23.0 | >100 |
| 37556-1 | 115 | −22.8 | >100 |
| 40883-1 | 4496 | −22.6 | >100 |
| 40793-1 | 3921 | −22.5 | >100 |
| 40781-1 | 3795 | −22.4 | >100 |
| 45226-1 | 317 | −22.2 | >100 |
| 44931-1 | 1435 | −21.9 | >100 |
| 40813-1 | 4203 | −21.9 | >100 |
| 45254-1 | 358 | −21.8 | >100 |
| 45255-1 | 356 | −21.5 | >100 |
| 45243-1 | 1239 | −21.4 | >100 |
| 40809-1 | 4191 | −20.7 | 3.46 |
| 41417-1 | 5772 | −20.6 | >100 |
| 40895-1 | 4963 | −20.5 | >100 |
| 40890-1 | 4905 | −20.1 | >100 |
| 45230-1 | 783 | −19.8 | >100 |
| 40799-1 | 4129 | −19.4 | >33.3 |
| 40801-1 | 4132 | −19.4 | >100 |
| 45214-1 | 792 | −19.4 | >100 |
| 41930-1 | 688 | −19.2 | >100 |
| 45234-1 | 355 | −18.7 | >100 |
| 40876-1 | 4403 | −18.7 | >100 |
| 37900-1 | 868 | −18.6 | >100 |
| 41058-1 | 6267 | −18.5 | >100 |
| 41055-1 | 6202 | −18.1 | >100 |
| 41063-1 | 6503 | −17.9 | 0.69 |
| 41165-1 | 2190 | −17.9 | >100 |
| 45206-1 | 492 | −17.8 | >100 |
| 41041-1 | 5679 | −17.5 | >100 |
| 42057-1 | 1009 | −16.9 | >100 |
| 40870-1 | 4361 | −16.8 | >100 |
| 37592-1 | 336 | −16.7 | >100 |
| 37589-1 | 329 | −16.5 | >100 |
| 37854-1 | 1318 | −16.4 | >100 |
| 40897-1 | 4966 | −16.4 | >100 |
| 45242-1 | 1235 | −16.3 | >100 |
| 42058-1 | 1040 | −16.2 | >100 |
| 45238-1 | 1501 | −16.1 | >100 |
| 38207-1 | 112 | −15.7 | >100 |
| 41143-1 | 269 | −15.7 | 0.43 |
| 41423-1 | 4160 | −15.6 | >100 |
| 40807-1 | 4184 | −15.2 | >100 |
| 40905-1 | 5576 | −15.0 | >100 |
| 41061-1 | 6279 | −14.6 | >100 |
| 41056-1 | 6243 | −14.3 | >100 |
| 40778-1 | 3776 | −14.2 | >100 |
| 37591-1 | 335 | −14.2 | >100 |
| 40889-1 | 4904 | −13.7 | >100 |
| 37887-1 | 757 | −13.4 | 1.18 |
| 40901-1 | 5215 | −12.9 | >100 |
| 40802-1 | 4135 | −12.8 | >100 |
| 45260-1 | 318 | −12.6 | >100 |
| 40879-1 | 4487 | −12.5 | >100 |
| 42051-1 | 1614 | −12.3 | >100 |
| 40863-1 | 4283 | −12.3 | >100 |
| 40877-1 | 4404 | −12.1 | >100 |
| 37923-1 | 1121 | −11.5 | >100 |
| 45253-1 | 395 | −11.3 | >100 |
| 40800-1 | 4130 | −11.0 | >100 |
| 41035-1 | 5670 | −10.6 | >100 |
| 45208-1 | 327 | −10.3 | >100 |
| 45251-1 | 1354 | −10.0 | >100 |
| 44934-1 | 1497 | −9.8 | >100 |
| 45245-1 | 1264 | −9.4 | >100 |
| 37577-1 | 254 | −8.7 | >100 |
| 45262-1 | 315 | −8.5 | >100 |
| 40794-1 | 3923 | −8.5 | >100 |
| 41060-1 | 6271 | −7.7 | >100 |
| 41167-1 | 2193 | −7.6 | >100 |
| 41940-1 | 788 | −7.6 | >100 |
| 40886-1 | 4901 | −7.4 | >100 |
| 45261-1 | 467 | −7.3 | >100 |
| 40814-1 | 4251 | −7.2 | >100 |
| 40869-1 | 4360 | −7.1 | >100 |
| 40867-1 | 4291 | −6.6 | >100 |
| 45228-1 | 601 | −6.4 | >100 |
| 37902-1 | 870 | −6.1 | >100 |
| 45227-1 | 32 | −6.1 | >100 |
| 37860-1 | 1399 | −6.0 | >100 |
| 41430-1 | 21 | −5.5 | >100 |
| 40871-1 | 4362 | −4.7 | >100 |
| 44936-1 | 1520 | −4.3 | >100 |
| 41172-1 | 2274 | −4.2 | >100 |
| 44913-1 | 450 | −3.2 | >100 |
| 40908-1 | 5600 | −3.0 | >100 |
| 41429-1 | 22 | −2.6 | >100 |
| 45252-1 | 446 | −2.1 | >100 |
| 37576-1 | 253 | −2.0 | >100 |
| 45223-1 | 452 | −1.8 | >100 |
| 41047-1 | 5740 | −0.9 | >100 |
| 40798-1 | 4095 | −0.8 | >100 |
| 41427-1 | 257 | −0.7 | >100 |
| 45231-1 | 843 | −0.6 | >100 |
| 40815-1 | 4254 | −0.6 | >100 |
| 45205-1 | 491 | −0.5 | >100 |
| 40812-1 | 4202 | −0.4 | >100 |
| 37778-1 | 673 | 0.3 | >100 |
| 37573-1 | 249 | 0.5 | >100 |
| 42036-1 | 1417 | 0.9 | >100 |
| 40904-1 | 5575 | 2.2 | >100 |
| 40894-1 | 4962 | 2.3 | >100 |
| 37555-1 | 113 | 3.6 | >100 |
| 37579-1 | 281 | 3.7 | >100 |
| 40872-1 | 4363 | 4.2 | >100 |
| 37894-1 | 769 | 5.0 | >100 |
| 40819-1 | 4262 | 5.4 | >100 |
| 41141-1 | 143 | 5.4 | >100 |
| 41938-1 | 781 | 5.4 | >100 |
| 41148-1 | 586 | 5.5 | >100 |
| 41064-1 | 6568 | 5.5 | >100 |
| 41144-1 | 420 | 6.9 | >100 |
| 41303-1 | 6154 | 8.5 | >100 |
| 45224-1 | 489 | 8.7 | >100 |
| 40874-1 | 4398 | 9.5 | >100 |
| 45244-1 | 1248 | 10.3 | >100 |
| 41140-1 | 138 | 10.5 | >100 |
| 40787-1 | 3854 | 11.5 | >100 |
| 41036-1 | 5671 | 11.6 | >100 |
| 42068-1 | 1269 | 12.4 | >100 |
| 44933-1 | 1451 | 13.6 | >100 |
| 37575-1 | 252 | 13.8 | >100 |
| 40862-1 | 4263 | 14.2 | >100 |
| 41929-1 | 615 | 15.9 | >100 |
| 41164-1 | 2189 | 20.4 | >100 |
| 41428-1 | 142 | 21.1 | >100 |
| 41304-1 | 5691 | 21.2 | >100 |
| 40804-1 | 4138 | 34.1 | >100 |
| 41412-1 | 5822 | 34.3 | >100 |
| 41067-1 | 6574 | 36.3 | >100 |
| 40873-1 | 4369 | 37.6 | >100 |
| 41037-1 | 5672 | 38.0 | >100 |
| 41425-1 | 262 | 39.7 | >100 |
| 41034-1 | 5669 | 40.4 | >100 |
| 41926-1 | 603 | 46.7 | >100 |

TABLE 3
RNA FISH Assay with Human/Cyno PNPLA3-Spanning siRNA

| | PNPLA3 | PNPLA3 |
|---|---|---|
| Duplex No. | Location | knockdown (%) |
| 38207-1 | 112 | −15.7 |
| 37555-1 | 113 | 3.6 |
| 37556-1 | 115 | −22.8 |
| 37557-1 | 121 | −47.0 |
| 37558-1 | 122 | −26.7 |
| 37559-1 | 204 | −45.8 |
| 37560-1 | 205 | −45.4 |
| 37561-1 | 206 | −62.2 |
| 37562-1 | 207 | −49.8 |
| 37563-1 | 211 | −74.7 |
| 37564-1 | 212 | −38.1 |
| 37565-1 | 213 | −58.1 |
| 37566-1 | 219 | −36.7 |
| 37567-1 | 220 | −73.0 |
| 37568-1 | 243 | −57.9 |
| 37569-1 | 244 | −59.8 |
| 37570-1 | 245 | −61.7 |
| 37571-1 | 246 | −50.5 |
| 37572-1 | 248 | −50.5 |
| 37573-1 | 249 | 0.5 |
| 37574-1 | 251 | −50.2 |
| 37575-1 | 252 | 13.8 |
| 37576-1 | 253 | −2.0 |
| 37577-1 | 254 | −4.4 |
| 37578-1 | 272 | −54.5 |
| 37579-1 | 281 | 3.7 |
| 37580-1 | 291 | −59.1 |
| 37581-1 | 293 | −58.8 |
| 37582-1 | 294 | −40.9 |
| 37583-1 | 302 | −60.6 |
| 37584-1 | 303 | −56.1 |
| 37585-1 | 305 | −60.3 |
| 37586-1 | 306 | −59.3 |
| 37587-1 | 307 | −60.3 |
| 37588-1 | 309 | −76.4 |
| 37589-1 | 329 | −17.8 |
| 37590-1 | 334 | −28.3 |
| 37591-1 | 335 | −14.2 |
| 37592-1 | 336 | −16.7 |
| 37593-1 | 337 | −54.8 |
| 37594-1 | 338 | −57.6 |
| 37595-1 | 339 | −61.5 |
| 37596-1 | 340 | −25.5 |
| 37597-1 | 341 | −51.0 |
| 37598-1 | 343 | −24.8 |
| 37599-1 | 344 | −45.2 |
| 37600-1 | 347 | −80.9 |
| 37743-1 | 369 | −32.7 |
| 37744-1 | 391 | −79.6 |
| 37745-1 | 392 | −77.0 |
| 37746-1 | 411 | −39.2 |
| 37747-1 | 412 | −49.6 |
| 37748-1 | 413 | −43.2 |
| 37749-1 | 414 | −45.9 |
| 37750-1 | 415 | −57.5 |
| 37751-1 | 416 | −38.8 |
| 37752-1 | 419 | −44.8 |
| 37753-1 | 497 | −64.9 |
| 37754-1 | 555 | −62.1 |
| 37755-1 | 556 | −50.5 |
| 37756-1 | 557 | −65.0 |
| 37757-1 | 560 | −65.1 |
| 37758-1 | 562 | −71.2 |
| 37759-1 | 563 | −71.0 |
| 37760-1 | 564 | −63.4 |
| 37761-1 | 567 | −60.0 |
| 37762-1 | 568 | −69.1 |
| 37763-1 | 590 | −75.3 |
| 37764-1 | 591 | −62.3 |
| 37765-1 | 593 | −73.7 |
| 37766-1 | 595 | −48.8 |
| 37767-1 | 641 | −66.7 |
| 37768-1 | 660 | −49.3 |
| 37769-1 | 661 | −47.3 |
| 37770-1 | 662 | −71.3 |
| 37771-1 | 663 | −36.4 |
| 37772-1 | 664 | −45.3 |
| 37773-1 | 665 | −37.2 |
| 37774-1 | 666 | −52.6 |
| 37775-1 | 667 | −50.3 |
| 37776-1 | 669 | −53.7 |
| 37777-1 | 671 | −67.5 |
| 37778-1 | 673 | 0.3 |
| 37779-1 | 675 | −60.1 |
| 37780-1 | 679 | −39.6 |
| 37781-1 | 698 | −76.0 |
| 37782-1 | 701 | −69.3 |
| 37783-1 | 702 | −45.7 |
| 37784-1 | 703 | −50.3 |
| 37785-1 | 704 | −79.6 |
| 37786-1 | 705 | −76.4 |
| 37787-1 | 706 | −74.2 |
| 37788-1 | 708 | −57.5 |
| 37789-1 | 756 | −76.5 |
| 37887-1 | 757 | −13.4 |
| 37888-1 | 762 | −65.0 |
| 38208-1 | 763 | −60.4 |
| 37889-1 | 764 | −77.7 |
| 37890-1 | 765 | −63.3 |
| 37891-1 | 766 | −59.8 |
| 37892-1 | 767 | −72.3 |
| 37893-1 | 768 | −81.8 |
| 37894-1 | 769 | 5.0 |
| 37895-1 | 770 | −53.3 |
| 37896-1 | 805 | −59.8 |
| 37897-1 | 832 | −67.6 |
| 37898-1 | 866 | −27.7 |
| 37899-1 | 867 | −48.3 |
| 37900-1 | 868 | −18.6 |
| 37901-1 | 869 | −42.7 |
| 37902-1 | 870 | −6.1 |
| 37903-1 | 878 | −66.8 |
| 37904-1 | 886 | −40.8 |
| 37905-1 | 887 | −63.8 |
| 37906-1 | 889 | −56.8 |
| 37907-1 | 893 | −75.8 |
| 37908-1 | 894 | −72.3 |
| 37909-1 | 895 | −66.1 |
| 37910-1 | 896 | −59.7 |
| 38213-1 | 897 | −67.8 |
| 37911-1 | 898 | −59.1 |
| 37912-1 | 899 | −66.7 |
| 37913-1 | 902 | −70.8 |
| 37914-1 | 904 | −25.2 |
| 37915-1 | 905 | −67.7 |
| 37917-1 | 939 | −45.1 |
| 38214-1 | 1092 | −71.3 |
| 37919-1 | 1094 | −49.6 |
| 38209-1 | 1095 | −51.0 |
| 37920-1 | 1101 | −63.5 |
| 37921-1 | 1104 | −63.1 |
| 37923-1 | 1121 | −11.5 |
| 37924-1 | 1125 | −39.0 |
| 38210-1 | 1189 | −71.2 |
| 37926-1 | 1200 | −45.0 |
| 37927-1 | 1201 | −69.1 |
| 38211-1 | 1210 | −39.9 |
| 37929-1 | 1213 | −67.6 |
| 38212-1 | 1217 | −66.6 |
| 37930-1 | 1219 | −80.9 |
| 37932-1 | 1220 | −72.9 |
| 37841-1 | 1221 | −46.5 |
| 37842-1 | 1223 | −73.3 |
| 37843-1 | 1224 | −74.8 |
| 37844-1 | 1225 | −58.0 |
| 37845-1 | 1226 | −73.0 |
| 37846-1 | 1280 | −74.1 |
| 37847-1 | 1289 | −51.0 |
| 37848-1 | 1296 | −56.2 |
| 37849-1 | 1300 | −67.9 |
| 37850-1 | 1310 | −58.4 |
| 37851-1 | 1311 | −54.0 |
| 37852-1 | 1312 | −59.4 |
| 37853-1 | 1317 | −49.8 |
| 37854-1 | 1318 | −16.4 |
| 37855-1 | 1319 | −30.8 |
| 37856-1 | 1320 | −42.6 |
| 37857-1 | 1321 | −40.7 |
| 37858-1 | 1322 | −70.1 |
| 37859-1 | 1352 | −62.5 |
| 37860-1 | 1399 | −20.3 |
| 37861-1 | 1402 | −50.1 |
| 37862-1 | 1431 | −28.2 |
| 37863-1 | 1596 | −55.1 |
| 37864-1 | 1602 | −76.3 |
| 37865-1 | 1603 | −71.2 |
| 37866-1 | 1604 | −63.6 |
| 37867-1 | 1666 | −50.9 |
| 37868-1 | 1754 | −51.1 |
| 37869-1 | 1757 | −55.4 |
| 37870-1 | 1760 | −65.6 |
| 37871-1 | 1763 | −66.6 |
| 37872-1 | 1764 | −65.1 |
| 37873-1 | 1765 | −55.8 |
| 37874-1 | 1771 | −67.0 |
| 37875-1 | 1775 | −46.7 |
| 37876-1 | 1851 | −50.7 |
| 37877-1 | 1852 | −64.5 |
| 37878-1 | 1855 | −61.3 |
| 37879-1 | 1857 | −39.6 |
| 37880-1 | 1890 | −70.5 |
| 37881-1 | 1917 | −53.7 |
| 37882-1 | 1918 | −46.2 |
| 37883-1 | 2012 | −41.5 |
| 37884-1 | 2013 | −47.3 |
| 37885-1 | 2014 | −56.1 |
| 37886-1 | 2197 | −73.6 |
| 38215-1 | 2198 | −80.2 |
| 38216-1 | 2199 | −79.4 |
| 38217-1 | 2200 | −79.4 |
| 38218-1 | 2201 | −72.6 |
| 38219-1 | 2202 | −66.2 |
| 38220-1 | 2204 | −66.9 |
| 38221-1 | 2216 | −55.6 |

TABLE 4
RNA FISH Assay with Human/Cyno
PNPLA3 Extended 3′ UTR siRNA

| | PNPLA3 | PNPLA3 |
|---|---|---|
| Duplex No. | Location | knockdown (%) |
| 41309-1 | 3773 | −35.7 |
| 40777-1 | 3775 | −51.4 |
| 40778-1 | 3776 | −14.2 |
| 40779-1 | 3793 | −45.5 |
| 40780-1 | 3794 | −33.1 |
| 40781-1 | 3795 | −22.4 |
| 40782-1 | 3796 | −43.5 |
| 40783-1 | 3813 | −49.7 |
| 40784-1 | 3820 | −41.5 |
| 40785-1 | 3851 | −33.0 |
| 40786-1 | 3853 | −23.4 |
| 40787-1 | 3854 | 11.5 |
| 40788-1 | 3857 | −60.2 |
| 40789-1 | 3858 | −66.3 |
| 40790-1 | 3860 | −36.0 |
| 40791-1 | 3862 | −38.3 |
| 40792-1 | 3920 | −34.9 |
| 40793-1 | 3921 | −22.5 |
| 40794-1 | 3923 | −8.5 |
| 40795-1 | 3958 | −42.7 |
| 40796-1 | 4006 | −35.8 |
| 41308-1 | 4008 | −41.7 |
| 40797-1 | 4020 | −30.7 |
| 41307-1 | 4046 | −48.3 |
| 40798-1 | 4095 | −0.8 |
| 41306-1 | 4097 | −31.0 |
| 40799-1 | 4129 | −19.4 |
| 40800-1 | 4130 | −11.0 |
| 40801-1 | 4132 | −19.4 |
| 40802-1 | 4135 | −12.8 |
| 40803-1 | 4137 | −27.2 |
| 40804-1 | 4138 | 34.1 |
| 40805-1 | 4139 | −31.3 |
| 40806-1 | 4140 | −47.1 |
| 40807-1 | 4184 | −15.2 |
| 40808-1 | 4186 | −33.5 |
| 40809-1 | 4191 | −47.8 |
| 40810-1 | 4193 | −67.3 |
| 40811-1 | 4199 | −23.1 |
| 40812-1 | 4202 | −0.4 |
| 40813-1 | 4203 | −21.9 |
| 40814-1 | 4251 | −7.2 |
| 40815-1 | 4254 | −0.6 |
| 40816-1 | 4259 | −25.9 |
| 40817-1 | 4260 | −50.9 |
| 40818-1 | 4261 | −37.9 |
| 40819-1 | 4262 | 5.4 |
| 40862-1 | 4263 | 14.2 |
| 40863-1 | 4283 | −12.3 |
| 40864-1 | 4286 | −37.1 |
| 40865-1 | 4289 | −42.4 |
| 40866-1 | 4290 | −61.5 |
| 40867-1 | 4291 | −6.6 |
| 40868-1 | 4318 | −26.4 |
| 40869-1 | 4360 | −7.1 |
| 40870-1 | 4361 | −16.8 |
| 40871-1 | 4362 | −4.7 |
| 40872-1 | 4363 | 4.2 |
| 40873-1 | 4369 | 37.6 |
| 40874-1 | 4398 | 9.5 |
| 40875-1 | 4402 | −42.4 |
| 40876-1 | 4403 | −18.7 |
| 40877-1 | 4404 | −12.1 |
| 40878-1 | 4484 | −33.1 |
| 40879-1 | 4487 | −12.5 |
| 40880-1 | 4488 | −26.2 |
| 40881-1 | 4491 | −25.1 |
| 40882-1 | 4495 | −52.1 |
| 40883-1 | 4496 | −22.6 |
| 40884-1 | 4500 | −57.0 |
| 40885-1 | 4543 | −47.2 |
| 40886-1 | 4901 | −7.4 |
| 40887-1 | 4902 | −29.9 |
| 40888-1 | 4903 | −35.2 |
| 40889-1 | 4904 | −13.7 |
| 40890-1 | 4905 | −20.1 |
| 40891-1 | 4906 | −34.1 |
| 40892-1 | 4957 | −38.1 |
| 40893-1 | 4958 | −41.3 |
| 40894-1 | 4962 | 2.3 |
| 40895-1 | 4963 | −20.5 |
| 40896-1 | 4965 | −29.9 |
| 40897-1 | 4966 | −16.4 |
| 40898-1 | 4968 | −42.4 |
| 40899-1 | 5012 | −46.0 |
| 40900-1 | 5026 | −42.2 |
| 40901-1 | 5215 | −12.9 |
| 40902-1 | 5569 | −30.1 |
| 40903-1 | 5571 | −54.7 |
| 40904-1 | 5575 | 2.2 |
| 40905-1 | 5576 | −15.0 |
| 40906-1 | 5578 | −49.9 |
| 40907-1 | 5579 | −57.2 |
| 40908-1 | 5600 | −3.0 |
| 41030-1 | 5601 | −40.8 |
| 41031-1 | 5640 | −44.1 |
| 41032-1 | 5641 | −25.1 |
| 41305-1 | 5642 | −36.0 |
| 41033-1 | 5668 | −29.0 |
| 41034-1 | 5669 | 40.4 |
| 41035-1 | 5670 | −10.6 |
| 41036-1 | 5671 | 11.6 |
| 41037-1 | 5672 | 38.0 |
| 41038-1 | 5673 | −23.7 |
| 41039-1 | 5674 | −54.9 |
| 41040-1 | 5676 | −46.4 |
| 41041-1 | 5679 | −17.5 |
| 41042-1 | 5680 | −55.6 |
| 41043-1 | 5681 | −26.7 |
| 41304-1 | 5691 | 21.2 |
| 41044-1 | 5692 | −31.3 |
| 41045-1 | 5705 | −30.5 |
| 41046-1 | 5736 | −49.3 |
| 41047-1 | 5740 | −0.9 |
| 41048-1 | 5741 | −49.7 |
| 41049-1 | 5967 | −36.4 |
| 41050-1 | 6153 | −56.7 |
| 41303-1 | 6154 | 8.5 |
| 41051-1 | 6157 | −32.1 |
| 41302-1 | 6158 | −25.8 |
| 41301-1 | 6162 | −35.8 |
| 41052-1 | 6163 | −52.9 |
| 41053-1 | 6199 | −57.4 |
| 41054-1 | 6201 | −29.3 |
| 41055-1 | 6202 | −18.1 |
| 41056-1 | 6243 | −14.3 |
| 41057-1 | 6265 | −43.7 |
| 41058-1 | 6267 | −18.5 |
| 41059-1 | 6268 | −26.6 |
| 41060-1 | 6271 | −7.7 |
| 41061-1 | 6279 | −14.6 |
| 41062-1 | 6280 | −23.2 |
| 41063-1 | 6503 | −39.8 |
| 41064-1 | 6568 | 5.5 |
| 41065-1 | 6571 | −45.0 |
| 41066-1 | 6573 | −56.1 |
| 41067-1 | 6574 | 36.3 |
| 41068-1 | 6575 | −30.4 |
| 41069-1 | 6576 | −44.4 |
| 41070-1 | 6594 | −44.0 |
| 41300-1 | 6610 | −43.8 |
| 41299-1 | 6611 | −43.6 |
| 41298-1 | 6612 | −38.0 |
| 41297-1 | 6613 | −32.9 |
| 41296-1 | 6614 | −28.3 |
| 41295-1 | 6635 | −54.2 |
| 41294-1 | 6636 | −52.8 |
| 41293-1 | 6637 | −42.4 |
| 41292-1 | 6638 | −28.0 |
| 41291-1 | 6640 | −36.8 |
| 41290-1 | 6641 | −44.6 |
| 41289-1 | 6643 | −45.9 |
| 41288-1 | 6644 | −52.3 |
| 41287-1 | 6669 | −36.8 |
| 41286-1 | 6670 | −33.6 |
| 41285-1 | 6671 | −35.5 |
| 41284-1 | 6676 | −36.1 |
| 41283-1 | 6683 | −30.0 |
| 41282-1 | 6684 | −35.0 |
| 41281-1 | 6685 | −36.8 |
| 41280-1 | 6686 | −38.6 |

TABLE 5
RNA FISH Assay with Human/Marmoset PNPLA3 siRNA

| | PNPLA3 | PNPLA3 |
|---|---|---|
| Duplex No. | Location | Knockdown (%) |
| 41430-1 | 21 | −5.5 |
| 41429-1 | 22 | −2.6 |
| 41140-1 | 138 | 10.5 |
| 41428-1 | 142 | 21.1 |
| 41141-1 | 143 | 5.4 |
| 41427-1 | 257 | 7.3 |
| 41426-1 | 258 | −40.1 |
| 41425-1 | 262 | 57.8 |
| 41142-1 | 266 | −56.7 |
| 41143-1 | 269 | −4.0 |
| 41144-1 | 420 | 19.6 |
| 41145-1 | 422 | −55.4 |
| 41146-1 | 423 | −52.7 |
| 41147-1 | 424 | −30.1 |
| 41148-1 | 586 | 1.7 |
| 41149-1 | 714 | −57.8 |
| 41150-1 | 715 | −58.6 |
| 41151-1 | 716 | −39.0 |
| 41152-1 | 718 | −57.2 |
| 41153-1 | 721 | −54.6 |
| 41154-1 | 722 | −33.4 |
| 41155-1 | 723 | −52.9 |
| 41156-1 | 729 | −33.9 |
| 41157-1 | 730 | −36.7 |
| 41158-1 | 1136 | −25.9 |
| 41159-1 | 1137 | −32.9 |
| 41160-1 | 1162 | −28.0 |
| 41161-1 | 1858 | −53.3 |
| 41162-1 | 1860 | −62.0 |
| 41163-1 | 2187 | −59.0 |
| 41164-1 | 2189 | 20.4 |
| 41165-1 | 2190 | −17.9 |
| 41166-1 | 2191 | −39.5 |
| 41167-1 | 2193 | −7.6 |
| 41168-1 | 2267 | −29.3 |
| 41169-1 | 2268 | −36.5 |
| 41170-1 | 2269 | −48.3 |
| 41171-1 | 2273 | −49.3 |
| 41172-1 | 2274 | −4.2 |
| 41173-1 | 2639 | −32.2 |
| 41174-1 | 3893 | −61.4 |
| 41175-1 | 4112 | −57.5 |
| 41176-1 | 4113 | −54.6 |
| 41177-1 | 4115 | −41.7 |
| 41178-1 | 4116 | −63.4 |
| 41179-1 | 4117 | −66.1 |
| 41180-1 | 4118 | −30.0 |
| 41424-1 | 4119 | −57.0 |
| 41423-1 | 4160 | −15.6 |
| 41422-1 | 4441 | −26.4 |
| 41421-1 | 4983 | −33.6 |
| 41420-1 | 5707 | −38.5 |
| 41419-1 | 5770 | −37.0 |
| 41418-1 | 5771 | −43.1 |
| 41417-1 | 5772 | −20.6 |
| 41416-1 | 5773 | −24.5 |
| 41415-1 | 5774 | −31.6 |
| 41414-1 | 5819 | −47.9 |
| 41413-1 | 5820 | −51.7 |
| 41412-1 | 5822 | 35.8 |
| 41411-1 | 5823 | −27.5 |
| 41410-1 | 5824 | −38.3 |
| 41409-1 | 5857 | −24.8 |
| 41408-1 | 6601 | −38.2 |

### Example 3: Efficacy Screening of Select PNPLA3 siRNA Molecules in AAV-Based Mouse Models Containing Human PNPLA3 Sequences

Adeno-associated adenovirus (AAV; serotype AAVDJ8; endotoxin-free, prepared internally by Amgen) diluted in phosphate buffered saline (Thermo Fisher Scientific, 14190-136) was administered at 1×10¹² viral particles per animal into the tail vein of C57BL/6NCrl male or female mice (Charles River Laboratories Inc.) to drive expression of human PNPLA3 sequences in the liver. Four AAV constructs were designed from the ENST00000216180.7 PNPLA3 transcript for in vivo screening; one containing the full-length coding sequence for PNPLA3^{rs738409-rs738408} (AAV-CDS), and three enhanced green fluorescence protein (eGFP) reporter constructs containing stretches of the 3′ untranslated region (nucleotides (nt) 1620-3410 (AAV-A), nt 3310-5100 (AAV-B), and nt 5000-6689 (AAV-C). Each AAV construct also contained a benchmark sequence to compare siRNA-mediated knockdown efficacy across AAVs and studies.

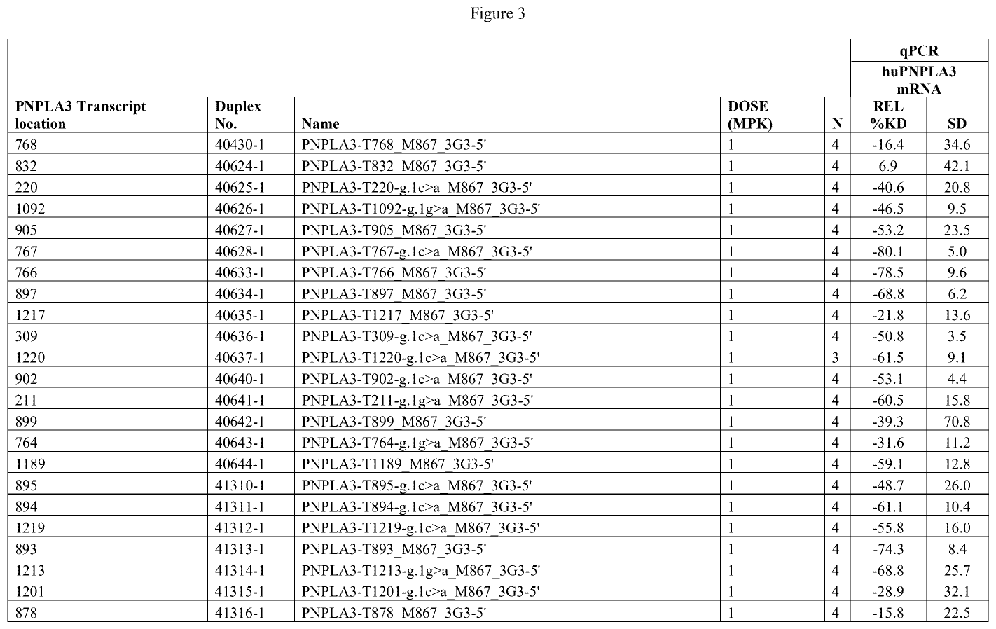

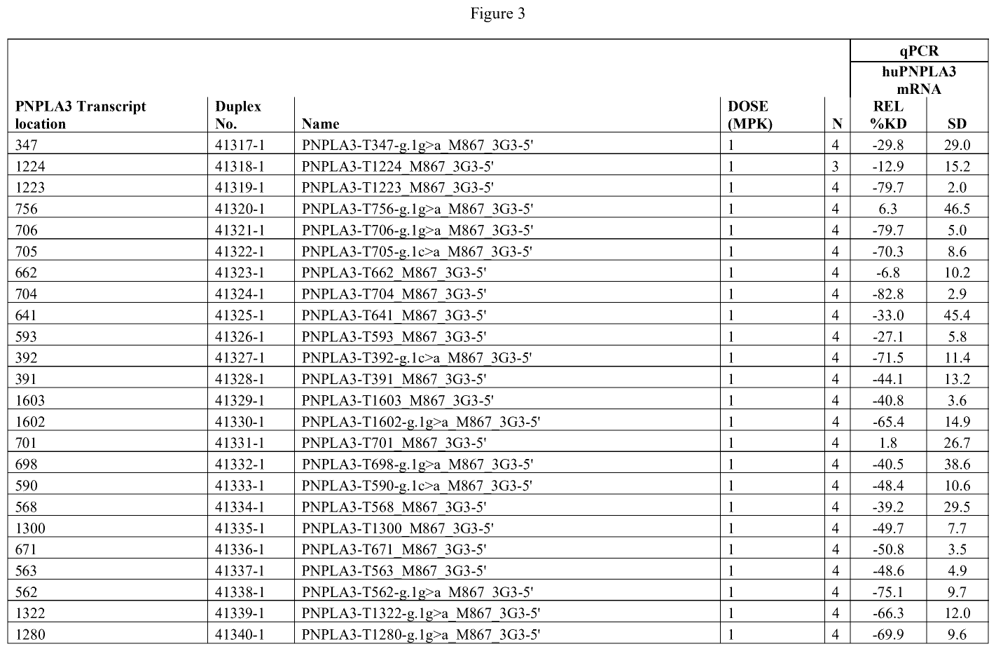

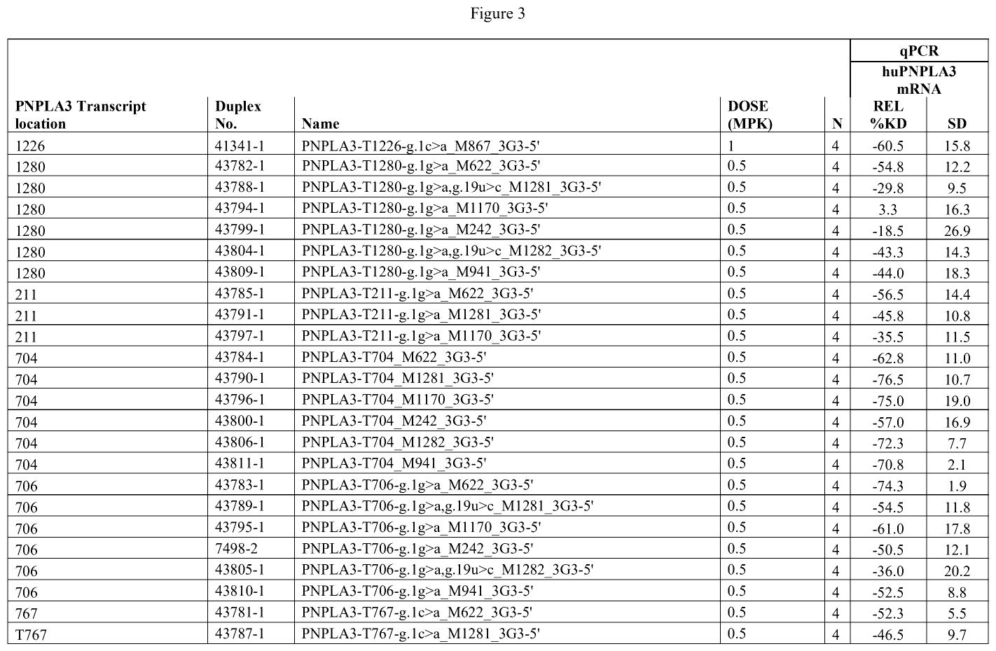

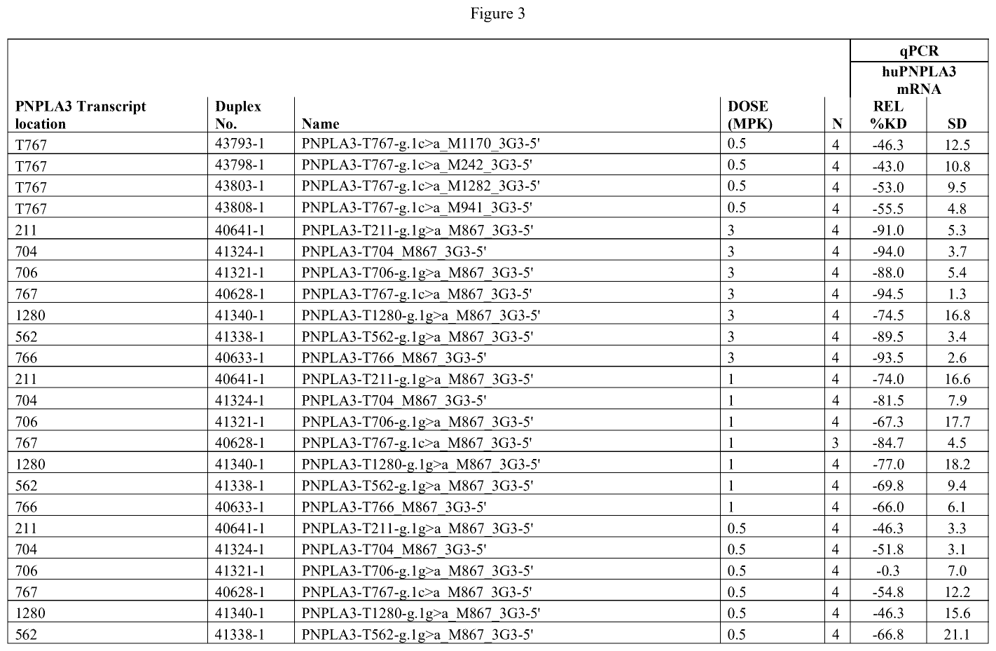

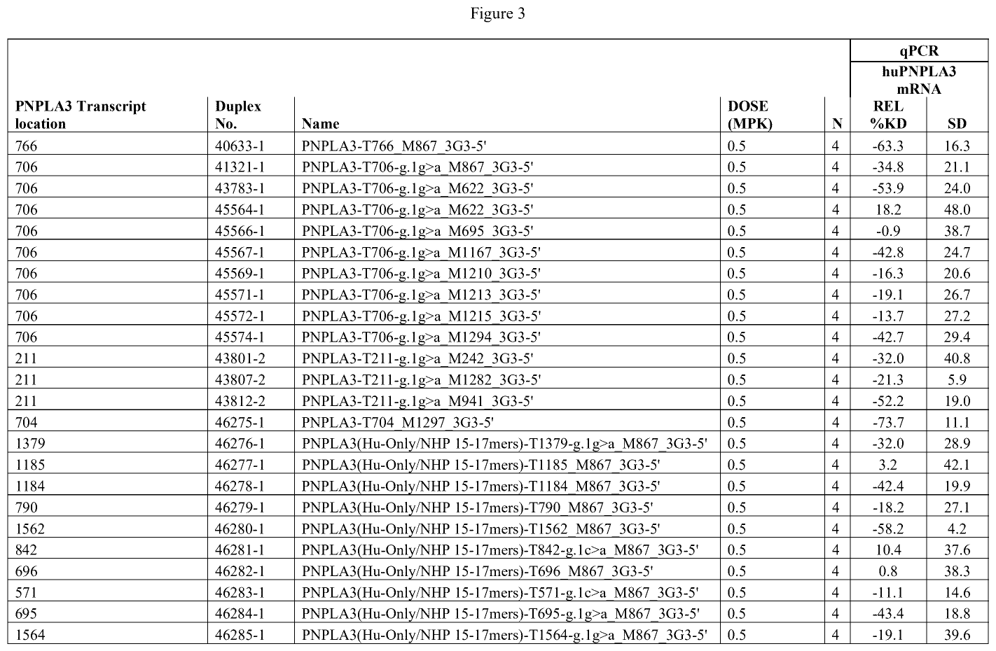

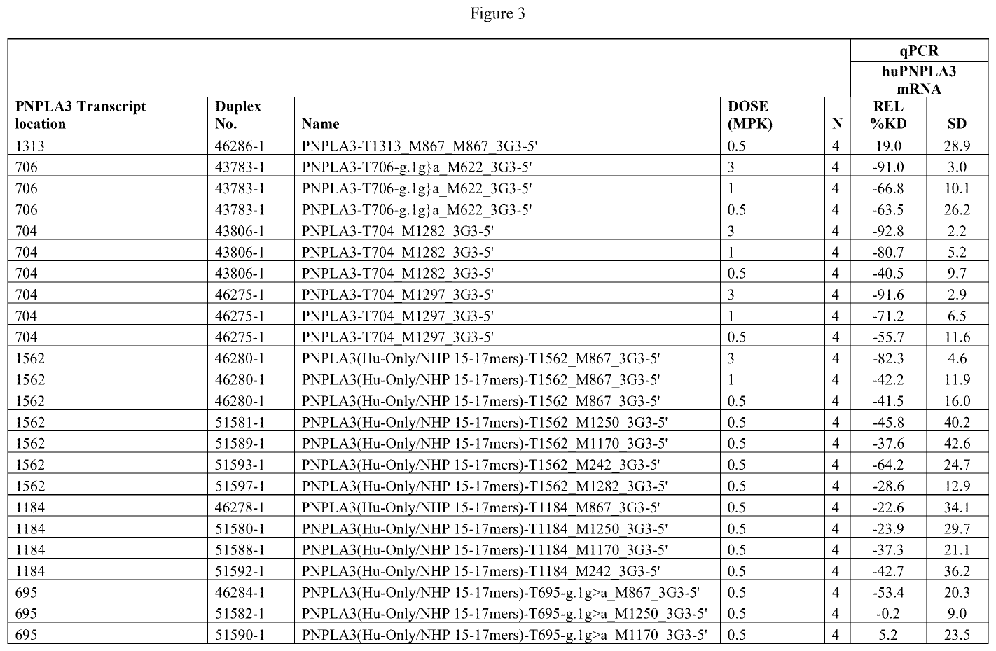

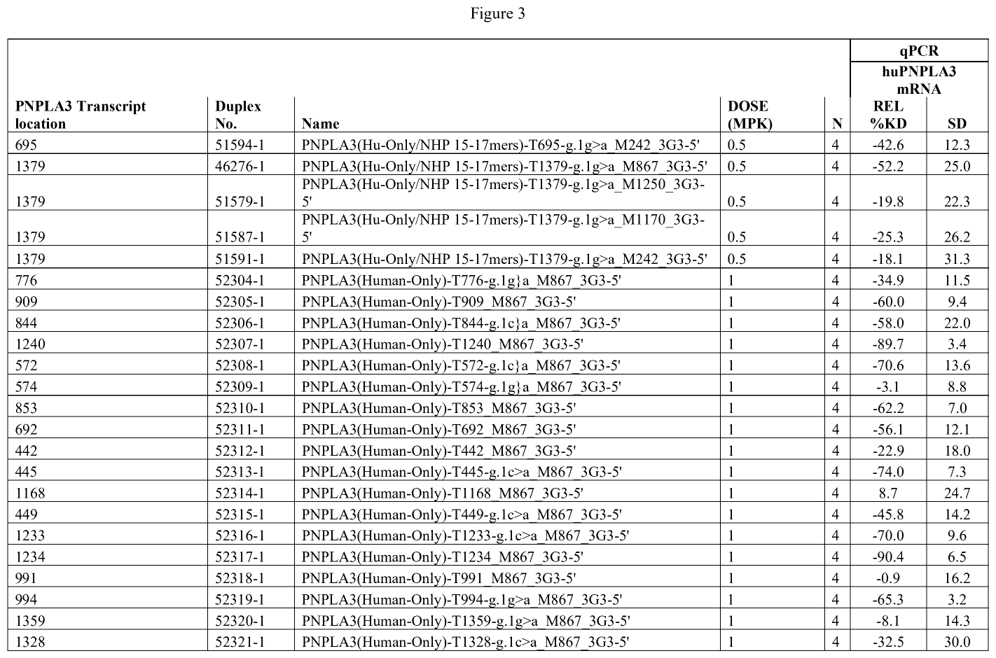

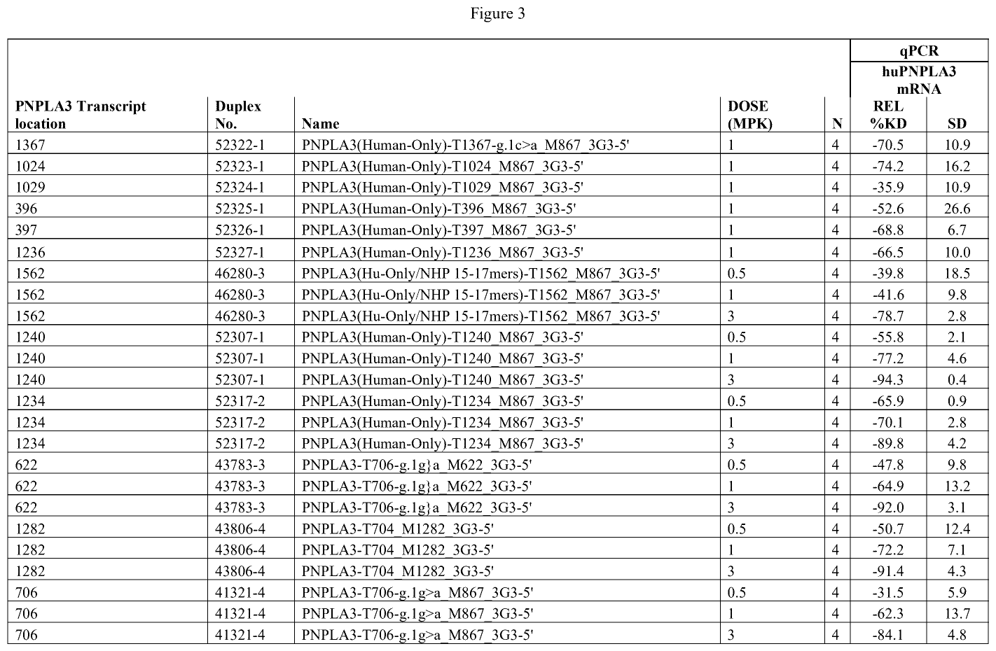

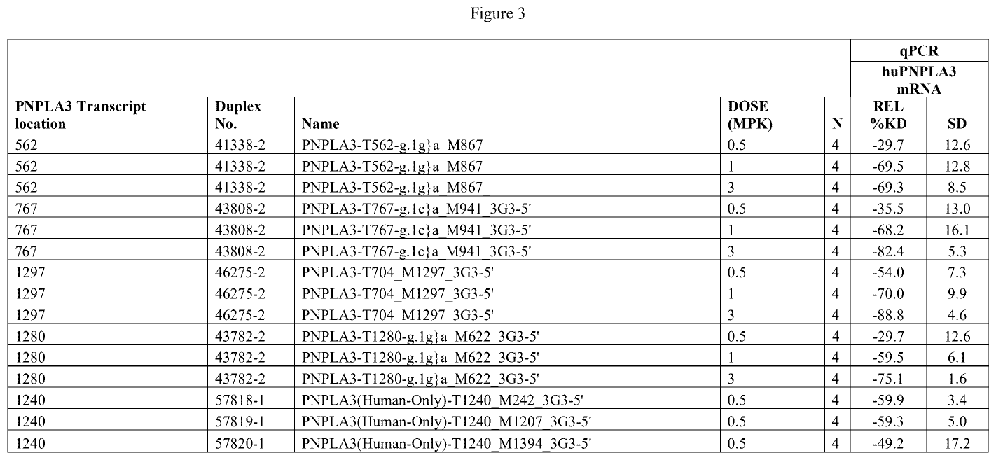

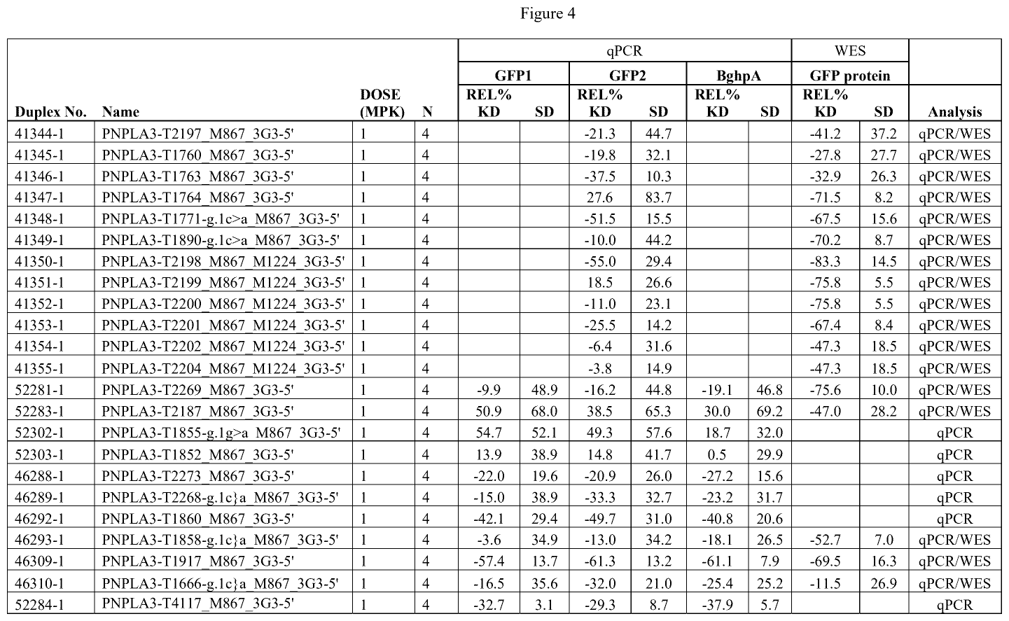

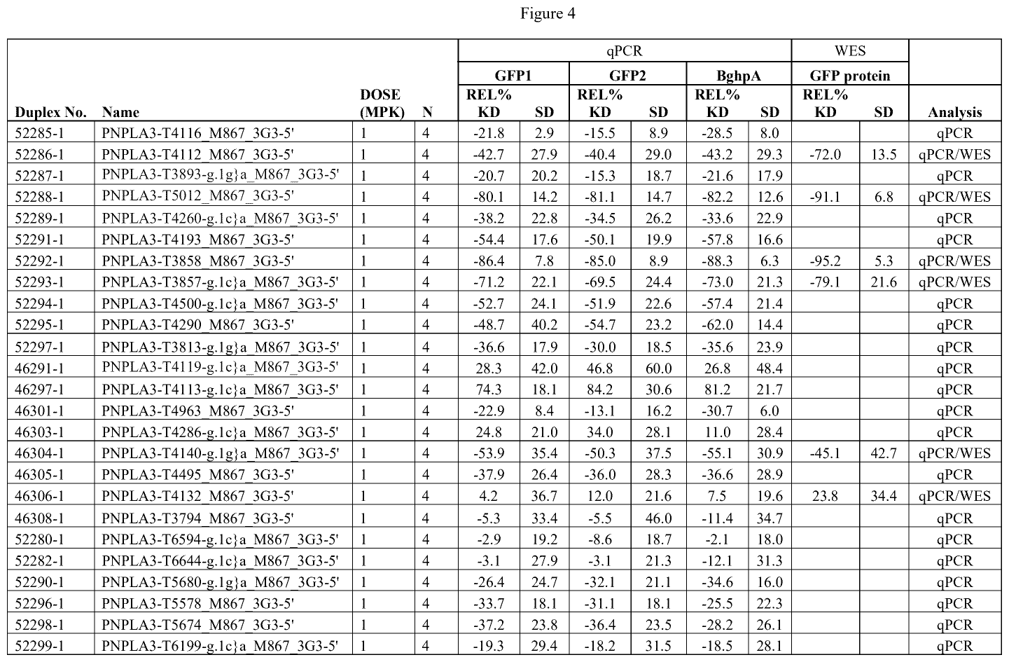

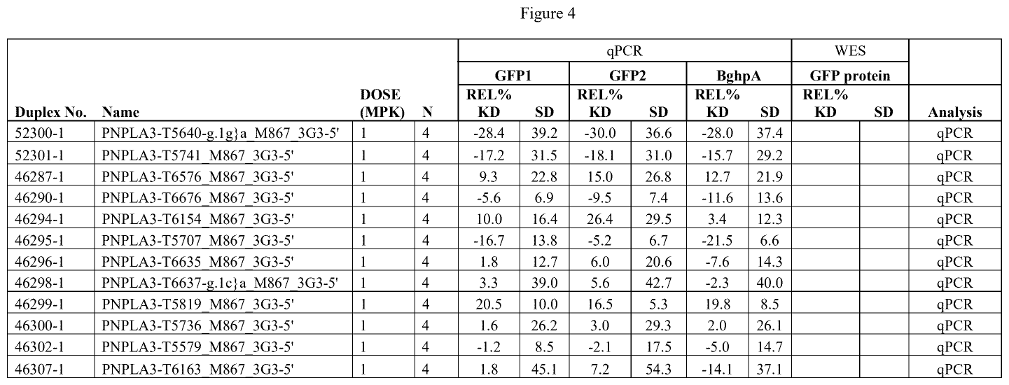

GalNAc-conjugated siRNAs shown in FIG. 2 were tested against PNPLA3^{rs738409-rs738408}, AAV-A, AAV-B or AAV-C. FIG. 2 also shows the unmodified versions of these siRNA sequences. Two weeks after AAV injection, mice (generally 8-12 weeks of age and an n=3-4 animals per group) were treated with a single dose of siRNA via subcutaneous injection, at 0.5, 1.0, or 3.0 milligrams per kilogram of animal, diluted in phosphate buffered saline (Thermo Fisher Scientific, 14190-136). After 28 days post-siRNA injection, animals were euthanized, and livers were collected from the animals and snap-frozen in liquid nitrogen. A portion of the liver was processed for purified RNA using a QIACube Classic instrument (Qiagen, 9001292) and RNeasy Mini kits (Qiagen, 74106) according to manufacturer's instructions. Samples were analyzed using a NanoDropp™ 8000 Spectrophotometer (Thermo Scientific, ND-8000-GL). RNA was treated with RQ1 RNase-Free DNase (Promega, M6101) and prepared for Real-Time qPCR using the TAQMAN™ RNA-to-CT™ 1-Step kit (Applied Biosystems, 4392656). Real-Time qPCR was run on a QuantStudio Real-Time PCR machine. Results were based on gene expression of human PNPLA3 (Invitrogen, hs00228747_m1), GFP1 (IDT custom assay: Forward primer: CTATGTGCAGGAGAGAACCATC (Sense; SEQ ID NO: 3589), Reverse primer: GCCCTTCAGCTCGATTCTATT (AntiSense; SEQ ID NO: 3590), Probe: 5′-6FAM-TACAAGACCCGCGCTGAAGTCAAG TAMRA-3′ (Sense; SEQ ID NO: 3591)), GFP2 (IDT custom assay: Forward primer: TCATCTGCACCACTGGAAAG (Sense; SEQ ID NO: 3592), Reverse primer: CTGCTTCATATGGTCTGGGTATC (AntiSense; SEQ ID NO: 3593), Probe: 5′-6FAM CCAACACTGGTCACTACCCTCACC TAMRA-3′ (Sense; SEQ ID NO: 3594), and/or bovine growth hormone polyadenylation (BghpA, which is included in each AAV construct; IDT custom assay: Forward: 5′-GCCAGCCATCTGTTGT-3′ (SEQ ID NO: 3595), Reverse: 5′-GGAGTGGCACCTTCCA-3′ (SEQ ID NO: 3596), Probe: 5′-6FAM-TCCCCCGTGCCTTCCTTGACC TAMRA-3′ (SEQ ID NO: 3597), as normalized to mouse TATA-binding protein (Tbp) (IDT, Hex Mm.PT.39a.22214839), and presented as the relative percent knockdown of human PNPLA3, GFP, and/or BghpA mRNA expression, normalized to mouse Tbp, compared to vehicle-treated control animals. For some groups, another portion of the snap-frozen liver was homogenized in Pierce IP buffer (Thermo Scientific, 87787) including 1×HALT™ Protease and Phosphatase Inhibitor Cocktail (Thermo Scientific, 78441) to make a 50 mg tissue/ml lysis buffer solution. Protein concentrations were determined using a DIRECT DETECT™ infrared spectrometer (Millipore). GFP protein analysis was performed on a PEGGY SUE™ Simple Western System (Protein Simple, 004-800) using a 12-230 kDa PEGGY SUE or SALLY SUE Separation Module (Protein Simple, SM-S001), or a WES™ Simple Western System (Protein Simple, 004-600) using a 12-230 kDa Jess or Wes Separation Module, 8×25 capillary cartridges (Protein Simple, SM-W004). All R-actin protein detection was performed using the WES™ Simple Western System and components. The results of these experiments are shown FIG. 3 (AAV-CDS) and FIG. 4 (AAV-A, AAV-B, and AAV-C).

To evaluate relative percent knockdown of endogenous levels of the human PNPLA3 transcript by PNPLA3 siRNA molecules, a homozygous hPNPLA3^{I148M} knock-in (hPNPLA3^{I148M} KI) mouse model was used (developed by Amgen; colony maintained at Charles River Laboratories, Inc.). In this model, the wild type mouse Pnpla3 gene was replaced by the full-length coding region of the human PNPLA3^{I148M} gene while utilizing the mouse transcriptional regulatory promoter region to drive expression.

Single housed, male hPNPLA3^{I148M} KI mice, typically 8-10 weeks of age, were maintained on a standard chow diet ad libitum until the start of the study, at which point they were converted to a modified rodent obesity diet supplemented with hydrogenated vegetable oil, added fructose, and enriched with 0.2% added cholesterol (Envigo, TD.190883 or TD.200212; the latter is an irradiated version of the former). After two weeks on the diet, body weights were recorded, and the mice were randomized into groups of 5-6 mice based on even weight distribution. The mice were subcutaneously injected with a single dose of PNPLA3 siRNA molecules at 1 or 3 mg/kg of animal diluted in phosphate buffered saline (Thermo Fisher Scientific, 14190-136) or saline only. After 28 days post-treatment, mice were euthanized, and the livers collected from the animals and snap-frozen in liquid nitrogen. A portion of the liver was processed for purified RNA and for some groups a portion was processed for protein analysis.

Due to the relatively low endogenous expression of PNPLA3 transcript, mRNA analysis from KI mouse livers was prepared as described but analyzed by treating with a High-Capacity cDNA Reverse Transcription kit (Applied Biosystems, 4368813) and run on a QIAcuity Eight Platform Digital PCR System (Qiagen, 911056) which is more sensitive than traditional qPCR.

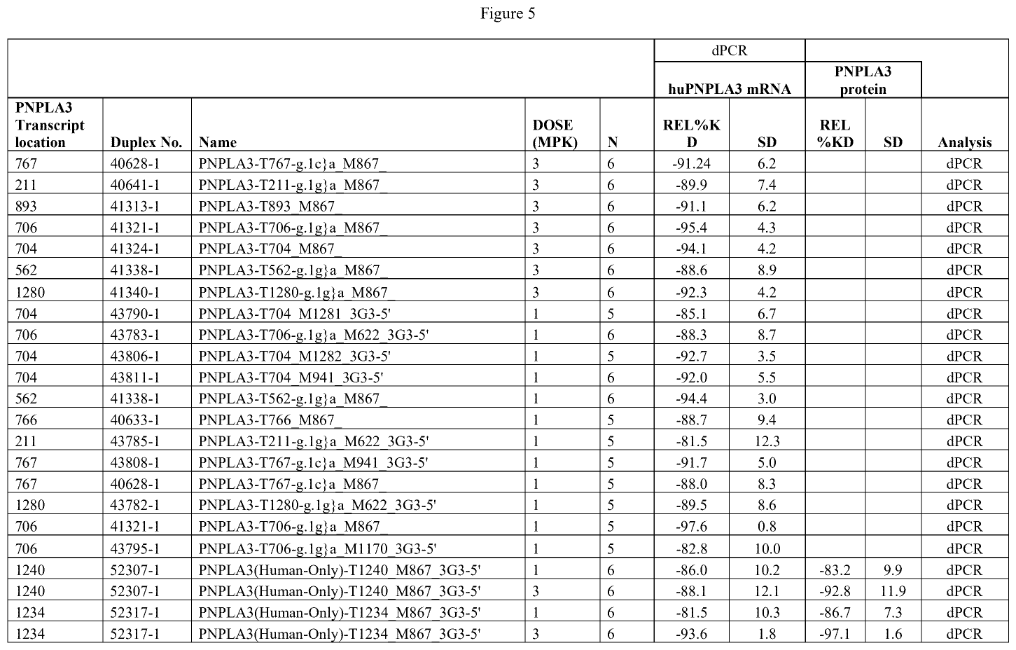

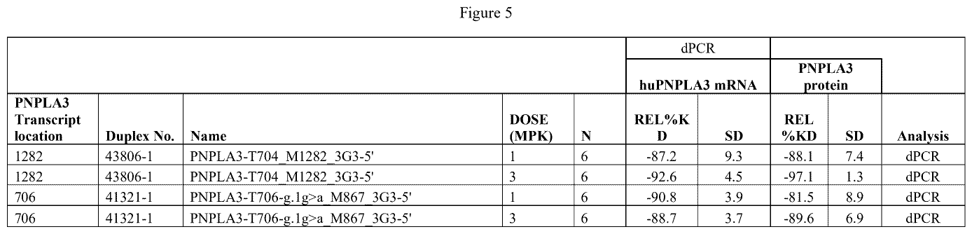

For targeted measurement of hPNPLA3 protein, approximately 100 to 200 mg of frozen liver tissue from each mouse was homogenized independently in cold PIERCE™ IP Lysis Buffer (Thermo Fisher, 87787). Samples were treated with 1×PIERCE™ Protease Inhibitor Tablets (Thermo Fisher, A32963) and BENZONASE® Nuclease (Millipore-Sigma, E1014). Human PNPLA3 protein (peptide: VSDGENVLVSDFR (SEQ ID NO: 3598); New England Peptide) was captured using immunoaffinity (IA) techniques and an anti-human PNPLA3 mAb (developed by Amgen Inc.), trypsin-digested on beads, and analyzed by liquid chromatography—with tandem mass spectrometry (LC-MS/MS; Thermo Fisher, Orbitrap FUSION™ LUMOS™ TRIBRID™ Mass Spectometer). Reagent-free LC MS/MS was used to measure mouse glyceraldehyde 3-phosphate dehydrogenase (mGAPDH; peptide: LISWYDNEYGYSNR (SEQ ID NO: 3599); New England Peptide), a housekeeping protein, in liver tissue recovered from each animal. The peptide signals across all samples were determined using the LC-MS data analysis software, ANALYST® (SCIEX, v1.6.2). The results of the knock-in experiments are shown in FIG. 5.

siRNA triggers that cluster within certain regions of the PNPLA3 coding sequence and demonstrated efficacy in vivo were selected for further investigation. Two selected siRNA molecules (“PNPLA3-T704_M1282_3G3-5′” (Duplex No. 43806-1) and “PNPLA3-T706-g.1g>a_M867_3G3-5′” (Duplex No. 41321-1) in FIG. 5) selectively target both human and cynomolgus PNPLA3 mRNA, while the other two selected siRNA molecules (“PNPLA3(Human-Only)-T1234_M867_3G3-5′” (Duplex No. 52317-1) and “PNPLA3 (Human-Only)-T1240_M867_3G3-5′” (Duplex No. 52307-1 in FIG. 5) target only human PNPLA3 mRNA. The sequences of the sense and antisense strands of each of the selected siRNA molecules is set forth in Table 6.

TABLE 6

| | | | Sense | Antisense | Sense | Antisense |
|---|---|---|---|---|---|---|
| | Duplex | PNPLA3 | SEQ ID NO: | SEQ ID NO: | SEQ ID NO: | SEQ ID NO: |
| siRNA | No. | Location | (Unmodified) | (Unmodified) | (Modified) | (Modified) |
| PNPLA3- | 43806-1 | 704 | 151 | 655 | 403 | 907 |
| T704_M1282_3G3-5′ | | | | | | |
| PNPLA3-T706-g.1g > | 41321-1 | 706 | 90 | 594 | 342 | 846 |
| a_M867_3G3-5′ | | | | | | |
| PNPLA3(Human-Only)- | 52317-1 | 1234 | 263 | 767 | 515 | 1019 |
| T1234_M867_3G3-5′ | | | | | | |
| PNPLA3(Human-Only)- | 52307-1 | 1240 | 253 | 757 | 505 | 1009 |
| T1240_M867_3G3-5 | | | | | | |

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

1. An RNAi construct comprising a sense strand and an antisense strand, wherein the antisense strand comprises a region that is complementary to a Patatin-Like Phospholipase Domain Containing 3 (PNPLA3) mRNA sequence comprising any one of SEQ ID NOs: 1-60, and wherein the RNAi construct inhibits the expression of PNPLA3.

2. The RNAi construct of claim 1, which comprises a region having at least 15 contiguous nucleotides differing by no more than 3 nucleotides from an antisense sequence selected from SEQ ID NOs: 565-1068 and SEQ ID NOs: 2329-3588.

3. The RNAi construct of claim 1, wherein the antisense strand hybridizes to a PNPLA3 mRNA sequence comprising any one of SEQ ID NOs: 1-60.

4. The RNAi construct of claim 1, wherein the sense strand comprises a sequence that is sufficiently complementary to the sequence of the antisense strand to form a duplex region of about 15 to about 30 base pairs in length.

5. The RNAi construct of claim 4, wherein the duplex region is about 17 to about 24 base pairs in length.

6. The RNAi construct of claim 4, wherein the duplex region is about 19 to about 21 base pairs in length.

7.-9. (canceled)

10. The RNAi construct of claim 4, wherein the sense strand and the antisense strand are each about 15 to about 30 nucleotides in length.

11. (canceled)

12. The RNAi construct of claim 10, wherein the sense strand and the antisense strand are each about 20 to about 25 nucleotides in length.

13. (canceled)

14. The RNAi construct of claim 1, which comprises at least one blunt end.

15. The RNAi construct of claim 1, which comprises at least one nucleotide overhang of 1 to 4 unpaired nucleotides.

16. The RNAi construct of claim 15, wherein the nucleotide overhang has two unpaired nucleotides.

17. The RNAi construct of claim 15, wherein the RNAi construct comprises a nucleotide overhang at the 3′ end of the sense strand, the 3′ end of the antisense strand, or the 3′ end of both the sense strand and the antisense strand.

18. The RNAi construct of claim 15, wherein the nucleotide overhang comprises a 5′-UU-3′ dinucleotide or a 5′-dTdT-3′ dinucleotide.

19. The RNAi construct of claim 1, wherein the RNAi construct comprises at least one modified nucleotide.

20. The RNAi construct of claim 19, wherein the modified nucleotide is a 2′-modified nucleotide.

21. The RNAi construct of claim 19, wherein the modified nucleotide is a 2′-fluoro modified nucleotide, a 2′-O-methyl modified nucleotide, a 2′-O-methoxyethyl modified nucleotide, a 2′-O-allyl modified nucleotide, a bicyclic nucleic acid (BNA), a glycol nucleic acid, an inverted base, or combinations thereof.

22. The RNAi construct of claim 21, wherein the modified nucleotide is a 2′-O-methyl modified nucleotide, a 2′-O-methoxyethyl modified nucleotide, a 2′-fluoro modified nucleotide, or combinations thereof.

23. The RNAi construct of claim 19, wherein all of the nucleotides in the sense and antisense strands are modified nucleotides.

24. The RNAi construct of claim 23, wherein the modified nucleotides are 2′-O-methyl modified nucleotides, 2′-fluoro modified nucleotides, or combinations thereof.

25. The RNAi construct of claim 1, which comprises at least one phosphorothioate internucleotide linkage.

26. The RNAi construct of claim 25, wherein the RNAi construct comprises two consecutive phosphorothioate internucleotide linkages at the 3′ end of the antisense strand.

27. The RNAi construct of claim 25, wherein the RNAi construct comprises two consecutive phosphorothioate internucleotide linkages at both the 3′ and 5′ ends of the antisense strand and two consecutive phosphorothioate internucleotide linkages at the 5′ end of the sense strand.

28. The RNAi construct of claim 1, wherein the antisense strand comprises a sequence selected from SEQ ID NOs: 565-1068 and SEQ ID NOs: 2329-3588.

29. The RNAi construct of claim 28, wherein the sense strand comprises a sequence selected from SEQ ID NOs: 61-564 and SEQ ID NOs: 1069-2328.

30. The RNAi construct of claim 1, wherein the RNAi construct is a duplex compound comprising an antisense strand and a sense strand, wherein the antisense strand comprises a sequence selected from SEQ ID NOs: 565-1068 and SEQ ID NOs: 2329-3588 and the sense strand comprises a sequence selected from SEQ ID NOs: 61-564 and SEQ ID NOs: 1069-2328.

31. The RNAi construct of claim 30, which comprises:
a. an antisense strand comprising SEQ ID NO: 594 and a sense strand comprising SEQ ID NO: 90;
b. an antisense strand comprising SEQ ID NO: 846 and a sense strand comprising SEQ ID NO: 342;
c. an antisense strand comprising SEQ ID NO: 655 and a sense strand comprising SEQ ID NO: 151;
d. an antisense strand comprising SEQ ID NO: 907 and a sense strand comprising SEQ ID NO: 403;
e. an antisense strand comprising SEQ ID NO: 757 and a sense strand comprising SEQ ID NO: 253;
f. an antisense strand comprising SEQ ID NO: 1009 and a sense strand comprising SEQ ID NO: 505;
g. an antisense strand comprising SEQ ID NO: 767 and a sense strand comprising SEQ ID NO: 263; or
h. an antisense strand comprising SEQ ID NO: 1019 and a sense strand comprising SEQ ID NO: 515.

32. The RNAi construct of claim 1, wherein the RNAi construct reduces the expression level of PNPLA3 in liver cells following incubation with the RNAi construct as compared to the PNPLA3 expression level in liver cells that have been incubated with a control RNAi construct.

33. The RNAi construct of claim 32, wherein the liver cells are Hep3B or HepG2 cells.

34.-37. (canceled)

38. The RNAi construct of claim 1, further comprising a ligand that binds to one or more proteins expressed on the surface of liver cells.

39. A composition comprising the RNAi construct of claim 1 and a pharmaceutically acceptable carrier, excipient, or diluent.

40. A method for reducing the expression of PNPLA3 in a patient in need thereof comprising administering to the patient the RNAi construct of claim 1.

41. A method for reducing the expression of PNPLA3 in a patient in need thereof comprising administering to the patient the composition of claim 39.

42. The method of claim 40, wherein the expression level of PNPLA3 in hepatocytes is reduced in the patient following administration of the RNAi construct as compared to the PNPLA3 expression level in a patient not receiving the RNAi construct.

43. The method of claim 40, wherein the patient suffers from nonalcoholic fatty liver disease (NAFLD).

44. The method of claim 43, wherein the patient suffers from non-alcoholic steatohepatitis (NASH).

45. (canceled)
